(19) United States
(12) Patent Application Publication
(10) Pub. No.: US 2022/0257655 A1
(43) Pub. Date: Aug. 18, 2022

(54) ENGINEERED OFF-THE-SHELF IMMUNE CELLS AND METHODS OF USE THEREOF

(71) Applicants: The Regents of the University of California, Oakland, CA (US); University of Southern California, Los Angeles, CA (US)

(72) Inventors: Yu Jeong Kim, Los Angeles, CA (US); Yan-Ruide Li, Los Angeles, CA (US); Pin Wang, Los Angeles, CA (US); Lili Yang, Los Angeles, CA (US); Jiaji Yu, Los Angeles, CA (US)

(73) Assignees: The Regents of the University of California, Oakland, CA (US); University of Southern California, Los Angeles, CA (US)

(21) Appl. No.: 17/618,240

(22) PCT Filed: Jun. 12, 2020
(86) PCT No.: PCT/US2020/037486

§ 371 Date: Dec. 10, 2021

Related U.S. Application Data
(60) Provisional application No. 62/946,788, filed on Dec. 11, 2019.
(60) Provisional application No. 62/946,747, filed on Dec. 11, 2019.
(60) Provisional application No. 62/860,613, filed on Jun. 12, 2019.
(60) Provisional application No. 62/860,644, filed on Jun. 12, 2019.
(60) Provisional application No. 62/860,667, filed on Jun. 12, 2019.

(51) Int. Cl. A61K 35/17; C12N 5/0783; C12N 15/62; C12N 15/11; C12N 9/22; A61P 37/06; A61P 35/00

(52) U.S. Cl. CPC A61K 35/17; C12N 2501/58; C12N 15/625; C12N 15/11; C12N 9/22; A61P 37/06; A61P 35/00; C12N 2506/03; C12N 2310/20; C12N 2800/80; C12N 2501/2303; C12N 2501/2306; C12N 2501/2307; C12N 2501/125; C12N 2501/21; C12N 2501/14; C12N 2501/145; C12N 2501/26; C12N 5/0638

(57) ABSTRACT

Aspects of the present disclosure relate to methods and compositions related to the preparation of immune cells, including engineered immune cells. Certain embodiments of the disclosure include compositions, cells, and methods related to engineered invariant natural killer T (iNKT) cells for off-the-shelf use for clinical therapy. The iNKT cells may be produced from hematopoietic stem progenitor cells and may be suitable for allogeneic cellular therapy because they are HLA negative. In some aspects, the cells have imaging and suicide targeting capabilities.

This application claims the benefit of U.S. Provisional Patent Application No. 62/860,613, filed Jun. 12, 2019; U.S. Provisional Patent Application No. 62/860,644, filed Jun. 12, 2019; U.S. Provisional Patent Application No. 62/860,667, filed Jun. 12, 2019; U.S. Provisional Patent Application No. 62/946,747, filed Dec. 11, 2019; and U.S. Provisional Patent Application No. 62/946,788, filed Dec. 11, 2019; which are expressly incorporated by reference herein in their entirety.

### BACKGROUND

### 1. Field of the Invention

Embodiments of the disclosure concern at least the fields of immunology, cell biology, molecular biology, and medicine, including at least cancer medicine.

### 2. Description of Related Art

Cancer affects tens of millions of people worldwide and is a leading threat to public health in the United States. Despite the existing therapies, cancer patients still suffer from the ineffectiveness of these treatments, their toxicities, and the risk of relapse. Novel therapies for cancer are therefore in desperately needed. Over the past decade, immunotherapy has become the new-generation cancer medicine. In particular, cell-based cellular therapies have shown great promise. An outstanding example is the chimeric antigen receptor (CAR)-engineered adoptive T cells therapy, which targets certain blood cancers at impressive efficacy.

However, most of the current protocols for treatment consist of autologous adoptive cell transfer, wherein immune cells collected from a patient are manufactured and used to treat this single patient. Such an approach is costly, manufacture labor intensive, and difficult to broadly deliver to all patients in need. Allogenic immune cellular products that can be manufactured at a large-scale and can be readily distributed to treat a higher number of patients therefore are in great demand.

Despite existing therapies, cancer patients still suffer from the ineffectiveness of these treatments, their toxicities, and the risk of relapse. Novel therapies for diseases, such as cancer and autoimmune diseases, are therefore in desperate demand. The present disclosure provides solutions to a long-felt need for therapies, but also therapies that can be delivered or distributed more widely.

### SUMMARY OF THE DISCLOSURE

Embodiments are provided to address the need for new therapies, more particularly, the need for cellular therapies that are not hampered by the challenges posed for individualizing therapy using autologous cells. The ability to manufacture a therapeutic cell population or a cell population that can be used to create a therapeutic cell population “off-the-shelf” increases the availability and usefulness of new cellular therapies.

Embodiments of the disclosure are directed to methods for generating or preparing a population of immune cells. The immune cells may be, for example, NK cells, T cells, iNKT cells, or other immune cells. In some embodiments, the immune cells are iNKT cells. In some embodiments, the immune cells are CD4+ helper T cells, regulatory T (Treg) cells, CD8+ cytotoxic T cells, gamma-delta T cells, mucosal associated invariant T (MAIT) cells, and other innate and adaptive T cells. Accordingly, aspects of the disclosure relate to a method of preparing a population of T cells comprising: a) selecting stem or progenitor cells; b) introducing one or more nucleic acids encoding at least one T-cell receptor (TCR); and c) culturing the cells to induce the differentiation of the cells into T cells; wherein a), b), and/or c) exclude contacting the cells with a feeder cell or a population of feeder cells. In some embodiments, in c), the cells are cultured in a culture that is feeder-free. In some embodiments, the stem or progenitor cells comprise CD34+ cells. In some embodiments, the stem or progenitor cells have been cultured in a medium comprising one or more of IL-3, IL-7, IL-6, SCF, MCP-4, EPO, TPO, FLT3L, and/or retronectin. In some embodiments, the stem or progenitor cells have been cultured on a surface that has been coated with retronectin, DLL4, DLL1, and/or VCAM1. In some embodiments, the cells have been cultured in medium comprising one or more of 5-50 ng/ml hIL-3, 5-50 ng/ml IL-7, 0.5-5 ng/ml MCP-4, IL-6, 5-50 ng/ml hSCF, EPO, 5-50 ng/ml hTPO, and/or 10-100 ng/ml hFLT3L. In some embodiments, the cells have been cultured in medium comprising one or more of 10 ng/ml hIL-3, 20-25 ng/ml IL-7, 1 ng/ml MCP-4, IL-6, 15-50 ng/ml hSCF, EPO, 5-50 ng/ml hTPO, and/or 50 ng/ml hFLT3L. In some embodiments, the cells have been cultured with one or more of IL-3, IL-7, IL-6, SCF, EPO, TPO, FLT3L, and/or retronectin for 12-72 hours. In some embodiments, the TCR comprises an iNKT TCR. In some embodiments, the TCR comprises an antigen-specific (e.g., cancer-antigen specific) TCR. In some embodiments, the TCR comprises a TCR that specifically recognizes the NY-ESO-1 antigen. In some embodiments, the NY-ESO-1 antigen comprises NY-ESO-1₁₅₇₋₁₆₅. In some embodiments, c) comprises culturing the cells in a differentiation and/or expansion medium. In some embodiments, c) comprises contacting the cells with one or more of DLL1, DLL4, VCAM1, VCAM5, and/or retronectin. In some embodiments, the one or more of DLL1, DLL4, VCAM1, VCAM5, and/or retronectin is coated on a tissue culture plate or microbead surface. In some embodiments, the one or more of DLL1, DLL4, VCAM1, VCAM5, and/or retronectin are coated on the tissue culture plate using a coating composition comprising 0.1-10 μg/ml DLL4 and 0.01-1 μg/ml VCAM1. In some embodiments, the one or more of DLL1, DLL4, VCAM1, VCAM5, and/or retronectin are coated using a coating composition comprising 0.5 μg/ml DLL4 and 0.1 μg/ml VCAM1. In some embodiments, the expansion or differentiation medium comprises one or more of Iscove's MDM, serum albumin, insulin, transferrin, and/or 2-mercaptoethanol. In some embodiments, the expansion or differentiation medium comprises one or more of ascorbic acid, human serum, B27 supplement, glutamax, Flt3L, IL-7, MCP-4, IL-6, TPO, and SCF. In some embodiments, the expansion or differentiation medium comprises one or more of 50-500 μM ascorbic acid, human serum, 1-10% B27 supplement, 0.1-10% glutamax, 2-50 ng/ml Flt3L, 2-50 ng/ml IL-7, 0.1-1 ng/ml MCP-4, 0-10 ng/ml IL-6, 0.5-50 ng/ml TPO, and 1.5-50 ng/ml SCF. In some embodiments, the expansion or differentiation medium comprises one or more of 100 μM ascorbic acid, human serum, 4% B27 supplement, 1% glutamax, 2-50 ng/ml Flt3L, 2-50 ng/ml IL-7, 0.1-1 ng/ml MCP-4, 0-10 ng/ml IL-6, 0.5-50 ng/ml TPO, and 1.5-50 ng/ml SCF. In some embodiments, the method further comprises stimulation and/or expansion of the cells. In some embodiments, stimulation or expansion of the cells comprises contacting the cells with an antigen that specifically binds to the TCR. In some embodiments, stimulation or expansion of the cells comprises contacting the cells with an anti-CD3, anti-CD2, and/or anti-CD28 antibody or antigen binding fragment thereof. In some embodiments, wherein stimulation or expansion of the cells comprises culturing the cells in an expansion medium. In some embodiments, the method comprises stimulation and/or expansion of the cells by contacting the cells with α-GC. In some embodiments, the method further comprises contacting the cells with one or both of IL-15 and IL-7 and/or wherein the expansion medium comprises one or both of IL-15 or IL-7. In some embodiments, the expansion medium comprises 5-100 ng/ml IL-7 and/or 5-100 ng/ml IL-15. In some embodiments, the expansion medium comprises 10 ng/ml IL-7 and/or 50 ng/ml IL-15. In some embodiments, the method further comprises contacting the cells with one or more of human serum antibody, Glutamax, a buffer, an antimicrobial agent, and N-acetyl-L-cysteine; and/or wherein the expansion medium comprises one or more of human serum antibody, Glutamax, a buffer, an antimicrobial agent, and N-acetyl-L-cysteine. In some embodiments, the method further comprises activation of the cells by contacting the cells with anti-CD3 and/or anti-CD28-coated beads. In some embodiments, the method further comprises transferring a nucleic acid comprising a CAR molecule and/or HLA-E gene into the cells. In some embodiments, the nucleic acid comprising the CAR molecule and/or HLA-E gene is transferred into the cell by retroviral infection. In some embodiments, the nucleic acid molecule comprises a CAR molecule. In some embodiments, the CAR is specific for BCMA, CD19, CD20, or NY-ESO. In some embodiments, the method further comprises contacting the cells with retronectin. In some embodiments, a, b, c, or the entire method excludes contacting the cells with a population of feeder cells. In some embodiments, a, b, c, or the entire method excludes contacting the cells with a population of stromal cells. In some embodiments, a, b, c, or the entire method excludes contacting the cells with a notch ligand or fragment thereof.

Further embodiments concern an engineered invariant natural killer T (iNKT) cell or a population of engineered iNKT cells. Accordingly, aspects of the disclosure relate to an engineered invariant natural killer T (iNKT) cell that expresses at least one invariant natural killer (iNKT) T-cell receptor (TCR) and wherein the cell comprises one or more of: high levels of NKG2D; low or undetectable expression of KIR; and high levels of Granzyme B. Further aspects relate to a population of engineered iNKT cells that express at least one iNKT TCR and wherein the population of cells comprise one or more of: at least 50% of cells with high levels of NKG2D; less than 2% of cells with high levels if KIR; at least 67% of cells with high levels of Granzyme B. Further aspects relate to a method of preparing the iNKT cells of the disclosure, wherein the method comprises a) selecting CD34+ cells from a plurality of hematopoietic stem or progenitor cells; b) introducing one or more nucleic acids encoding at least one human invariant natural killer (iNKT) T-cell receptor (TCR); and c) culturing the cells to induce the differentiation of the cells into iNKT cells.

Yet further aspects relate to a cell or population of cells produced by a method of the disclosure. Also provided is a method of treating a patient with engineered cells (e.g., engineered T cells, iNKT cells, etc.) comprising administering to the patient cells or a population of cells of the disclosure. Further aspects relate to a method for treating cancer in a patient comprising administering the cell(s) of the disclosure. Additional aspects relate to a method for treating graft versus host disease (GVHD) comprising administering the cell(s) of the disclosure.

In some embodiments, the population of cells comprise at least, at most, or about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) of cells with high levels of NKG2D. In some embodiments, the population of cells comprise less than, at most, at least, or about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50% of cells (or any derivable range therein) with high levels if KIR. In some embodiments, the population of cells comprise less than, at most, at least, or about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% (or any derivable range therein) of cells with high levels of Ganzyme B. The terms “high” or “low” levels or expression with respect to the cellular markers described herein may be in comparison to a T cell that is not an iNKT cell, a naturally occurring T cell, a naturally occurring iNKT cell, or a cell type described herein.

In some embodiments, the cells further comprise a chimeric antigen receptor (CAR). In some embodiments, the CAR specifically binds to BCMA. In some embodiments, the CAR specifically binds to CD19. In some embodiments, the cells further comprise exogenous expression of HLA-E. In some embodiments, the cells further comprise an exogenous nucleic acid encoding a polypeptide comprising all or a fragment of a suicide gene, HLA-E, a CAR, and/or an iNKT TCR. In some embodiments, the genome of the cell has been altered to eliminate surface expression of at least one HLA-I or HLA-II molecule. In some embodiments, the invariant TCR gene product is an alpha TCR gene product. In some embodiments, the invariant TCR gene product is a beta TCR gene product. In some embodiments, both an alpha TCR gene product and a beta TCR gene product are expressed. In some embodiments, the exogenous suicide gene product or HLA-E gene product and/or the exogenous nucleic acid(s) has one or more codons optimized for expression in the cell. In some embodiments, the suicide gene product is herpes simplex virus thymidine kinase (HSV-TK), purine nucleoside phosphorylase (PNP), cytosine deaminase (CD), carboxypetidase G2, cytochrome P450, linamarase, beta-lactamase, nitroreductase (NTR), carboxypeptidase A, or inducible caspase 9. In some embodiments, the suicide gene is enzyme-based. In some embodiments, the suicide gene encodes thymidine kinase (TK) or inducible caspase 9. In some embodiments, the TK gene is a viral TK gene. In some embodiments, the TK gene is a herpes simplex virus TK gene. In some embodiments, the suicide gene product is activated by a substrate. In some embodiments, the substrate is ganciclovir, penciclovir, or a derivative thereof.

In some embodiments, culturing the cells to induce the differentiation of the cells into iNKT cells comprises a culture that is feeder-free. In some embodiments, the iNKT TCR specifically binds to α-GC. In some embodiments, the method further comprises stimulation and/or expansion of the cells by contacting the cells with an antigen that specifically binds to the iNKT TCR. In some embodiments, the method comprises stimulation and/or expansion of the cells by contacting the cells with α-GC. In some embodiments, the method further comprises contacting the cells with IL-15. In some embodiments, the method further comprises contacting the cells with one or more of human serum antibody, Glutamax, a buffer, an antimicrobial agent, and N-acetyl-L-cysteine. In some embodiments, the method further comprises activation of the cells by contacting the cells with anti-CD3 and/or anti-CD28-coated beads. In some embodiments, the method further comprises transferring a nucleic acid comprising a CAR molecule and/or HLA-E gene into the cells. In some embodiments, the nucleic acid comprising the CAR molecule and/or HLA-E gene is transferred into the cell by retroviral infection. In some embodiments, the method further comprises contacting the cells with retronectin. In some embodiments, the CD34+ cells are isolated from a healthy subject and/or a subject not having cancer. In some embodiments, a, b, c, or the entire method excludes contacting the cells with a population of feeder cells. In some embodiments, a, b, c, or the entire method excludes contacting the cells with a population of stromal cells. In some embodiments, a, b, c, or the entire method excludes contacting the cells with a notch ligand or fragment thereof.

Further aspects of the disclosure relate to an engineered invariant natural killer T (iNKT) cell that expresses at least one invariant natural killer (iNKT) T-cell receptor (TCR) and a chimeric antigen receptor (CAR) comprising: a) an extracellular binding domain; b) a single transmembrane domain; and c) a single cytoplasmic region comprising a primary intracellular signaling domain, wherein the at least one iNKT TCR is expressed from an exogenous nucleic acid and/or from an endogenous invariant TCR gene that is under the transcriptional control of a recombinantly modified promoter region. In some embodiments, the extracellular binding domain comprises a BCMA-binding domain. In some embodiments, the extracellular binding domain comprises a CD19-binding domain.

Further aspects relate to a method of preparing a population of engineered chimeric antigen receptor (CAR) invariant natural killer T (iNKT) cells comprising: a) selecting CD34+ cells from a plurality of hematopoietic stem or progenitor cells; b) introducing one or more nucleic acids encoding at least one human invariant natural killer (iNKT) T-cell receptor (TCR); c) eliminating surface expression of one or more HLA-I and/or HLA-II molecules in the isolated human CD34+ cells; d) culturing isolated CD34+ cells expressing iNKT TCR to produce iNKT cells; and e) introducing a nucleic acid encoding a CAR into the iNKT cells. In some embodiments, the CAR is a BCMA-CAR. In some embodiments, the CAR is a CD19-CAR.

Further aspects relate to a method for treating cancer in a patient having cancer, the method comprising administering to the patient the engineered iNKT cells or populations of cells of the disclosure. In some embodiments, the cancer is a lymphoma. In some embodiments, the cancer is a B-cell lymphoma. In other embodiments, the cancer is a cancer described herein.

In some embodiments, the CAR further comprises a spacer between the extracellular domain and the transmembrane domain. In some embodiments, the spacer comprises a CD8 hinge. In some embodiments, the transmembrane domain comprises a transmembrane domain from CD8. In some embodiments, the cytoplasmic region further comprises a costimulatory domain. In some embodiments, the costimulatory domain comprises a 4-1BB polypeptide. In some embodiments, the intracellular signaling domain comprises a CD3-zeta polypeptide. In some embodiments, the CAR molecule comprises SEQ ID NO:72. In some embodiments, the spacer comprises SEQ ID NO:83. In some embodiments, the CAR comprises an scFv. In some embodiments, the scFv comprises SEQ ID NO:82. In some embodiments, the transmembrane domain comprises SEQ ID NO:84. In some embodiments, the costimulatory domain comprises SEQ ID NO:85. In some embodiments, the intracellular signaling domain comprises SEQ ID NO:86. In some embodiments, the CAR molecule further comprises a self-cleaving peptide. In some embodiments, the self-cleaving peptide comprises SEQ ID NO:87. In some embodiments, the CAR molecule further comprises a therapeutic control. In some embodiments, the therapeutic control comprises EGFR. In some embodiments, the therapeutic control comprises truncated EGFR. In some embodiments, the therapeutic control is cleaved from the CAR molecule.

In some embodiments, the nucleic acid encoding the CAR molecule is introduced into the cell using a recombinant vector. In some embodiments, the recombinant vector is a viral vector. In some embodiments, the viral vector is a lentivirus, a retrovirus, an adeno-associated virus (AAV), a herpesvirus, or adenovirus. In some embodiments, the viral vector comprises a retroviral vector.

Any embodiment discussed in the context of a cell can be applied to a population of such cells. In particular embodiments, an engineered iNKT cell comprises a nucleic acid comprising 1, 2, and/or 3 of the following: i) all or part of an invariant alpha T-cell receptor coding sequence; ii) all or part of an invariant beta T-cell receptor coding sequence, or iii) a suicide gene. In further embodiments, there is an engineered iNKT cell comprising a nucleic acid having a sequence encoding: i) all or part of an invariant alpha T-cell receptor; ii) all or part of an invariant beta T-cell receptor, and/or iii) a suicide gene product. In some embodiments, the engineered iNKT cell comprises a nucleic acid under the control of a heterologous promoter, which means the promoter is not the same genomic promoter that controls the transcription of the nucleic acid. It is contemplated that the engineered iNKT cell comprises an exogenous nucleic acid comprising one or more coding sequences, some or all of which are under the control of a heterologous promoter in many embodiments described herein.

It is specifically noted that any embodiment discussed in the context of a CAR embodiment, a particular cell embodiment, or a cell population embodiment may be employed with respect to any other CAR, cell, or cell population embodiment. Moreover, any embodiment employed in the context of a specific method may be implemented in the context of any other methods described herein. Furthermore, aspects of different methods described herein may be combined so as to achieve other methods, as well as to create or describe the use of any cells or cell populations. It is specifically contemplated that aspects of one or more embodiments may be combined with aspects of one or more other embodiments described herein. Furthermore, any method described herein may be phrased to set forth one or more uses of cells or cell populations described herein. For instance, use of engineered iNKT cells or an iNKT cell population can be set forth from any method described herein.

In a particular embodiment, there is an engineered invariant natural killer T (iNKT) cell that expresses at least one invariant natural killer T-cell receptor (iNKT TCR) wherein the at least one iNKT TCR is expressed from an exogenous nucleic acid and/or from an endogenous invariant TCR gene that is under the transcriptional control of a recombinantly modified promoter region. In some embodiments, the cell or population of cells further comprise an exogenous suicide gene product or a nucleic acid encoding for a suicide gene. An iNKT TCR refers to a “TCR that recognizes lipid antigen presented by a CD1d molecule.” In some embodiments, the iNKT TCR specifically binds to alpha-galactosylceramide (α-GC). It may include an alpha-TCR, a beta-TCR, or both. In some cases, the TCR utilized can belong to a broader group of “invariant TCR”, such as a MAIT cell TCR, GEM cell TCR, or gamma/delta TCR, resulting in HSC-engineered MAIT cells, GEM cells, or gamma/delta T cells, respectively.

In certain embodiments, there are engineered iNKT cell and T cell populations. In a particular embodiment, there is an engineered T cell, such as an engineered iNKT or other T cell population comprising: engineered clonal cells comprising either an altered genomic T-cell receptor sequence or an exogenous nucleic acid encoding an invariant T-cell receptor (TCR) and lacking expression of one or more HLA-I or HLA-II genes. An “altered genomic T-cell receptor sequence” means a sequence that has been altered by recombinant DNA technology. The term “clonal” cells refers to cells engineered to express a clonal transgenic TCR. In some embodiments, the clonal cells are from the same progenitor cell. It is contemplated that in some embodiments, there is a population of mixed clonal cells meaning the population comprises clonal cells that are from a set of progenitor cells; the set may be, be at least or be at most 10, 20, 30, 40, 50, 60 70, 80, 90, 100, 200, 300, 400, 500, 600, 700, 800, 900, 1000 or more progenitor cells (or any range derivable therein) meaning the cells in the population are progeny of the set of progenitor cells initially transfected/infected. In cases of cells comprising an exogenous nucleic acid or an altered genomic DNA sequence clonal cells may arise from an ancestor cell in which the exogenous nucleic acid was introduced. Some embodiments concern a population of clonal cells, meaning the population comprises progeny cells that arose from the same ancestor cell. It is contemplated that some populations of cells may contain a mix of different clonal cells, meaning the population arose from different ancestor cells that contain an exogenous nucleic acid but that may differ in a discernable way, such as the integration site for the exogenous nucleic acid. A nucleic acid sequence that has been introduced into a cell (alone or as part of a longer nucleic acid sequence) and becomes integrated such that progeny cells contain the integrated nucleic acid sequence is considered an exogenous nucleic acid. An introduced nucleic acid sequence that is maintained extrachromosomally is also considered an exogenous nucleic acid.

In embodiments where part of an alpha T-cell receptor or part of an beta T-cell receptor are utilized, it is contemplated that embodiments involve a functional part of an alpha T-cell receptor or a functional part of an beta T-cell receptor such that the cell expressing both of them is a functional T cell at least based on an assay that evaluates the ability to recognize lipid antigen presented by a CD1d molecule.

In some embodiments, a nucleic acid comprises a sequence that is, is at least, or is at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% identical (or any range derivable therein) to a sequence encoding 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 amino acids or contiguous amino acid residues of an iNKT TCR-alpha or iNKT TCR-beta polypeptide (or any range derivable therein).

In certain embodiments, a suicide gene is enzyme-based, meaning the gene product of the suicide gene is an enzyme and the suicide function depends on enzymatic activity. One or more suicide genes may be utilized in a single cell or clonal population. In some embodiments, the suicide gene encodes herpes simplex virus thymidine kinase (HSV-TK), purine nucleoside phosphorylase (PNP), cytosine deaminase (CD), carboxypetidase G2, cytochrome P450, linamarase, beta-lactamase, nitroreductase (NTR), carboxypeptidase A, or inducible caspase 9. Methods in the art for suicide gene usage may be employed, such as in U.S. Pat. No. 8,628,767, U.S. Patent Application Publication 20140369979, U.S. 20140242033, and U.S. 20040014191, all of which are incorporated by reference in their entirety. In further embodiments, a TK gene is a viral TK gene, .i.e., a TK gene from a virus. In particular embodiments, the TK gene is a herpes simplex virus TK gene. In some embodiments, the suicide gene product is activated by a substrate. Thymidine kinase is a suicide gene product that is activated by ganciclovir, penciclovir, or a derivative thereof. In certain embodiments, the substrate activating the suicide gene product is labeled in order to be detected. In some instances, the substrate that may be labeled for imaging. In some embodiments, the suicide gene product may be encoded by the same or a different nucleic acid molecule encoding one or both of TCR-alpha or TCR-beta. In certain embodiments, the suicide gene is sr39TK or inducible caspase 9. In alternative embodiments, the cell does not express an exogenous suicide gene.

In additional embodiments, a cell is lacking or has reduced surface expression of at least one HLA-I or HLA-II molecule. In some embodiments, the lack of surface expression of HLA-I and/or HLA-II molecules is achieved by disrupting the genes encoding individual HLA-I/II molecules, or by disrupting the gene encoding B2M (beta 2 microglobulin) that is a common component of all HLA-I complex molecules, or by discrupting the genes encoding CIITA (the class II major histocompatibility complex transactivator) that is a critical transcription factor controlling the expression of all HLA-II genes. In specific embodiments, the cell lacks the surface expression of one or more HLA-I and/or HLA-II molecules, or expresses reduced levels of such molecules by (or by at least) 50, 60, 70, 80, 90, 100% (or any range derivable therein). In some embodiments, the HLA-I or HLA-II are not expressed in the iNKT cell because the cell was manipulated by gene editing. In some embodiments, the gene editing involved is CRISPR-Cas9. Instead of Cas9, CasX or CasY may be involved. Zinc finger nuclease (ZFN) and TALEN are other gene editing technologies, as well as Cpfl, all of which may be employed. In other embodiments, the iNKT cell comprises one or more different siRNA or miRNA molecules targeted to reduce expression of HLA-I/II molecules, B2M, and/or CIITA.

In some embodiments, a T cell comprises a recombinant vector or a nucleic acid sequence from a recombinant vector that was introduced into the cells. In certain embodiments the recombinant vector is or was a viral vector. In further embodiments, the viral vector is or was a lentivirus, a retrovirus, an adeno-associated virus (AAV), a herpesvirus, or adenovirus. It is understood that the nucleic acid of certain viral vectors integrate into the host genome sequence.

In some embodiments, a cell was not exposed to media comprising animal serum. In further embodiments, a cell is or was frozen. In some embodiments, the cell has previously been frozen and wherein the cell is stable at room temperature for at least one hour. In some embodiments, the cell has previously been frozen and wherein the cell is stable at room temperature for at least 1, 2, 3, 4, 5, 6, 7, 8, 9, 10, 15, or 20 hours (or any derivable range therein.

In certain embodiments, a cell or a population of cells in a solution comprises dextrose, one or more electrolytes, albumin, dextran, and/or DMSO. In a further embodiments, the cell is in a solution that is sterile, nonpyogenic, and isotonic.

In certain embodiments, a T cell has been or is activated. In specific embodiments, the T cells is an iNKT cells and wherein the iNKT cells have been activated with alpha-galactosylceramide (α-GC).

In embodiments involving multiple cells, a cell population may comprise, comprise at least, or comprise at most about 10², 10³, 10⁴′, 10⁵, 10⁶, 10⁷′, 10⁸, 10⁹, 10¹⁰, 10¹¹, 10¹², 10¹³, 10¹⁴, 10¹⁵ cells or more (or any range derivable therein), which are engineered iNKT cells in some embodiments. In some cases, a cell population comprises at least about 10⁶-10¹² engineered iNKT cells. It is contemplated that in some embodiments, that a population of cells with these numbers is produced from a single batch of cells and are not the result of pooling batches of cells separately produced.

In specific embodiments, there is a T cell population, such as iNKT cells, comprising: clonal cells comprising one or more exogenous nucleic acids encoding a T-cell receptor (TCR) and a thymidine kinase suicide gene product, wherein the clonal cells have been engineered not to express functional beta-2-microglobulin (B2M), and/or class II, major histocompatibility complex, or transactivator (CIITA) and wherein the cell population is at least about 10⁶-10¹² total cells and comprises at least about 10²-10⁶ engineered cells. In certain instances, the cells are frozen in a solution.

A number of embodiments concern methods of preparing a T cell or a population of cells, particularly a population in which some are all the cells are clonal. In certain embodiments, a cell population comprises cells in which at least or at most 50%, 55%, 60%, 65%, 70%, 75%, 80%, 85%, 90%, 95%, 96%, 97%, 98%, 99%, 100% (or any range derivable therein) of the cells are clonal, i.e., the percentage of cells that have been derived from the same ancestor cell as another cell in the population. In other embodiments, a cell population comprises a cell population that is comprised of cells arising from, from at least, or from at most 1, 2, 3, 4, 5, 6, 7, 8, 9, 10, 11, 12, 13, 14, 15, 16, 17, 18, 19, 20, 21, 22, 23, 24, 25, 26, 7, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 (or any range derivable therein) different parental cells.

Methods for preparing, making, manufacturing, and/or using engineered T cells and cell populations are provided. Methods include 1, 2, 3, 4, 5, 6, 7, 8, 9, 10, 11, 12, 13, 14, 15 or more of the following steps in embodiments: obtaining hematopoietic cells; obtaining hematopoietic progenitor cells; obtaining progenitor cells capable of becoming one or more hematopoietic cells; obtaining progenitor cells capable of becoming T cells, such as iNKT cells; selecting cells from a population of mixed cells using one or more cell surface markers; selecting CD34+ cells from a population of cells; isolating CD34+ cells from a population of cells; separating CD34+ and CD34− cells from each other; selecting cells based on a cell surface marker other than or in addition to CD34; introducing into cells one or more nucleic acids encoding a T-cell receptor (TCR); infecting cells with a viral vector encoding a T-cell receptor (TCR); transfecting cells with one or more nucleic acids encoding a T-cell receptor (TCR); transfecting cells with an expression construct encoding a T-cell receptor (TCR); integrating an exogenous nucleic acid encoding a T-cell receptor (TCR) into the genome of a cell; introducing into cells one or more nucleic acids encoding a suicide gene product; infecting cells with a viral vector encoding a suicide gene product; transfecting cells with one or more nucleic acids encoding a suicide gene product; transfecting cells with an expression construct encoding a suicide gene product; integrating an exogenous nucleic acid encoding a suicide gene product into the genome of a cell; introducing into cells one or more nucleic acids encoding one or more polypeptides and/or nucleic acid molecules for gene editing; infecting cells with a viral vector encoding one or more polypeptides and/or nucleic acid molecules for gene editing; transfecting cells with one or more nucleic acids encoding one or more polypeptides and/or nucleic acid molecules for gene editing; transfecting cells with an expression construct encoding one or more polypeptides and/or nucleic acid molecules for gene editing; integrating an exogenous nucleic acid encoding one or more polypeptides and/or nucleic acid molecules for gene editing; editing the genome of a cell; editing the promoter region of a cell; editing the promoter and/or enhancer region for a TCR gene; eliminating the expression one or more genes; eliminating expression of one or more HLA-I/II genes in the isolated human CD34+ cells; transfecting into a cell one or more nucleic acids for gene editing; culturing isolated or selected cells; expanding isolated or selected cells; culturing cells selected for one or more cell surface markers; culturing isolated CD34+ cells expressing a TCR; expanding isolated CD34+ cells; culturing cells under conditions to produce or expand iNKT cells; culturing cells in a feeder-free system; culturing cells in an artificial thymic organoid (ATO) system to produce T cells; culturing cells in serum-free medium; culturing cells in an ATO system, wherein the ATO system comprises a 3D cell aggregate comprising a selected population of stromal cells that express a Notch ligand and a serum-free medium. It is specifically contemplated that one or more steps may be excluded in an embodiment.

In some embodiments, there are methods of preparing a population of clonal or engineered BCMA-CAR iNKT cells comprising: a) selecting CD34+ cells from human peripheral blood cells (PBMCs); b) introducing one or more nucleic acids encoding a human T-cell receptor (TCR); c) eliminating surface expression of one or more HLA-I/II genes in the isolated human CD34+ cells; and, d) culturing isolated CD34+ cells expressing iNKT TCR in an artificial thymic organoid (ATO) system to produce iNKT cells; and e) introducing a nucleic acid encoding a BCMA-CAR into the iNKT cells, wherein the ATO system comprises a 3D cell aggregate comprising a selected population of stromal cells that express a Notch ligand and a serum-free medium.

In some embodiments, the method further comprises contacting the cells with IL-15 in an amount sufficient for the expansion of the cell population. In some embodiments, the stem or progenitor cells or the CD34+ cells that are used to make the iNKT cells comprise less than 5×10⁸ cells. In some embodiments, the stem or progenitor cells or the CD34+ cells that are used to make the iNKT cells comprise less than 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹²¹², 8×10¹², 9×10¹², 1×10³, 2×10³, 3×10³, 4×10³, 5×10³, 6×10³, 7×10³, 8×10¹, 9×10¹, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, 1×10¹⁶, 2×10¹⁶, 3×10¹⁶, 4×10¹⁶, 5×10¹⁶, 6×10¹⁶, 7×10¹⁶, 8×10¹⁶, or 9×10¹⁶ cells, or any derivable range therein.

In some embodiments of the disclosure, at least 10, 20, 30, 40, 50, 60, 70, 80, 90, 100, 125, 150, 175, 200, 225, 250, 275, 300, 325, 350, 375, 400, 425, 450, 475, 500, 550, or 600 (or any derivable range therein) doses are produced by the methods of the disclosure. In some embodiments, each dose comprises 1×10⁷ to 1×10⁹ engineered iNKT cells. In some embodiments, each dose comprises at least, at most, or exactly 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹²¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, 1×10¹⁶, 2×10¹⁶, 3×10¹⁶, 4×10¹⁶, 5×10¹⁶, 6×10¹⁶, 7×10¹⁶, 8×10¹⁶, or 9×10¹⁶ cells (or any derivable range therein). In some embodiments, cells that may be used to create engineered iNKT cells are hematopoietic progenitor stem cells. Cells may be from peripheral blood mononuclear cells (PBMCs), bone marrow cells, fetal liver cells, embryonic stem cells, cord blood cells, induced pluripotent stem cells (iPS cells), or a combination thereof. In some embodiments, the iNKT cell is derived from a hematopoietic stem cell. In some embodiments, the cell is derived from a G-CSF mobilized CD34+ cells. In some embodiments, the cell is derived from a cell from a human patient that does not have cancer. In some embodiments, the cell doesn't express an endogenous TCR.

In some embodiments, methods comprise isolating CD34− cells or separating CD34− and CD34+ cells. While embodiments involve manipulating the CD34+ cells further, CD34− cells may be used in the creation of iNKT cells. Therefore, in some embodiments, the CD34− cells are subsequently used, and may be saved for this purpose.

Certain methods involve culturing selected CD34+ cells in media prior to introducing one or more nucleic acids into the cells. Culturing the cells can include incubating the selected CD34+ cells with media comprising one or more growth factors. In some embodiments, one or more growth factors comprise c-kit ligand, flt-3 ligand, and/or human thrombopoietin (TPO). In further embodiments, the media includes c-kit ligand, flt-3 ligand, and TPO. In some embodiments, the concentration of the one or more growth factors is between about 5 ng/ml to about 500 ng/ml with respect to either each growth factor or the total of any and all of these particular growth factors. The concentration of a component or the combination of multiple components in media can be about, at least about, or at most about 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500 (or any range derivable) ng/ml or g/ml or more.

In some embodiments, a nucleic acid may comprise a nucleic acid sequence encoding an α-TCR and/or a β-TCR, as discussed herein. In certain embodiments, one nucleic acid encodes both the α-TCR and the β-TCR. In additional embodiments, a nucleic acid further comprises a nucleic acid sequence encoding a suicide gene product. In some embodiments, a nucleic acid molecule that is introduced into a selected CD34+ cell encodes the α-TCR, the β-TCR, and the suicide gene product. In other embodiments, a method also involves introducing into the selected CD34+ cells a nucleic acid encoding a suicide gene product, in which case a different nucleic acid molecule encodes the suicide gene product than a nucleic acid encoding at least one of the TCR genes.

As discussed, in some embodiments the iNKT cells do not express the HLA-I and/or HLA-II molecules on the cell surface, which may be achieved by discrupting the expression of genes encoding beta-2-microglobulin (B2M), transactivator (CIITA), or HLA-I and HLA-II molecules. In certain embodiments, methods involve eliminating surface expression of one or more HLA-I/II molecules in the isolated human CD34+ cells. In particular embodiments, eliminating expression may be accomplished through gene editing of the cell's genomic DNA. Some methods include introducing CRISPR and one or more guide RNAs (gRNAs) corresponding to B2M or CIITA into the cells. In particular embodiments, CRISPR or the one or more gRNAs are transfected into the cell by electroporation or lipid-mediated transfection. Consequently, methods may involve introducing CRISPR and one or more gRNAs into a cell by transfecting the cell with nucleic acid(s) encoding CRISPR and the one or more gRNAs. A different gene editing technology may be employed in some embodiments.

Similarly, in some embodiments, one or more nucleic acids encoding the TCR receptor are introduced into the cell. This can be done by transfecting or infecting the cell with a recombinant vector, which may or may not be a viral vector as discussed herein. The exogenous nucleic acid may incorporate into the cell's genome in some embodiments.

In some embodiments, cells are cultured in serum-free medium. In certain embodiments, the serum-free medium further comprises externally added ascorbic acid. In particular embodiments, methods involve adding ascorbic acid medium. In further embodiments, the serum-free medium further comprises 1, 2, 3, 4, 5, 6, 7, 8, 9, 10, 11, 12, 13, 14, 15, or all 16 (or a range derivable therein) of the following externally added components: FLT3 ligand (FLT3L), interleukin 7 (IL-7), stem cell factor (SCF), thrombopoietin (TPO), stem cell factor (SCF), IL-2, IL-4, IL-6, IL-15, IL-21, TNF-alpha, TGF-beta, interferon-gamma, interferon-lambda, TSLP, thymopentin, pleotrophin, or midkine. In additional embodiments, the serum-free medium comprises one or more vitamins. In some cases, the serum-free medium includes 1, 2, 3, 4, 5, 6, 7, 8, 9, 10, 11, or 12 of the following vitamins (or any range derivable therein): comprise biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B12, or a salt thereof. In certain embodiments, medium comprises or comprise at least biotin, DL alpha tocopherol acetate, DL alpha-tocopherol, vitamin A, or combinations or salts thereof. In additional embodiments, serum-free medium comprises one or more proteins. In some embodiments, serum-free medium comprises 1, 2, 3, 4, 5, 6 or more (or any range derivable therein) of the following proteins: albumin or bovine serum albumin (BSA), a fraction of BSA, catalase, insulin, transferrin, superoxide dismutase, or combinations thereof. In other embodiments, serum-free medium comprises 1, 2, 3, 4, 5, 7, 8, 9, 10, or 11 of the following compounds: corticosterone, D-Galactose, ethanolamine, glutathione, L-carnitine, linoleic acid, linolenic acid, progesterone, putrescine, sodium selenite, or triodo-I-thyronine, or combinations thereof. In further embodiments, serum-free medium comprises a B-27® supplement, xeno-free B-27® supplement, GS21™ supplement, or combinations thereof. In additional embodiments, serum-free medium comprises or further comprises amino acids, monosaccharides, and/or inorganic ions. In some aspects, serum-free medium comprises 1, 2, 3, 4, 5, 6, 7, 8, 9, 10, 11, 12, or 13 of the following amino acids: arginine, cysteine, isoleucine, leucine, lysine, methionine, glutamine, phenylalanine, threonine, tryptophan, histidine, tyrosine, or valine, or combinations thereof. In other aspects, serum-free medium comprises 1, 2, 3, 4, 5, or 6 of the following inorganic ions: sodium, potassium, calcium, magnesium, nitrogen, or phosphorus, or combinations or salts thereof. In additional aspects, serum-free medium comprises 1, 2, 3, 4, 5, 6 or 7 of the following elements: molybdenum, vanadium, iron, zinc, selenium, copper, or manganese, or combinations thereof.

In some methods, cells are cultured in an artificial thymic organoid (ATO) system. The ATO system involves a three-dimensional (3D) cell aggregate, which is an aggregate of cells. In certain embodiments, the 3D cell aggregate comprises a selected population of stromal cells that express a Notch ligand. In some embodiments, a 3D cell aggregate is created by mixing CD34+ transduced cells with the selected population of stromal cells on a physical matrix or scaffold. In further embodiments, methods comprise centrifuging the CD34+ transduced cells and stromal cells to form a cell pellet that is placed on the physical matrix or scaffold. In certain embodiments, stromal cells express a Notch ligand that is an intact, partial, or modified DLL1, DLL4, JAG1, JAG2, or a combination thereof. In further embodiments, the Notch ligand is a human Notch ligand. In other embodiments, the Notch ligand is human DLL1. In some methods, cells are not cultured in an ATO system. In some embodiments, cells are cultured in a feeder-free system.

In further aspects, the ratio between stromal cells and CD34+ cells is about, at least about, or at most about 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:25, 1:30, 1:35, 1:40, 1:45, 1:50 (or any range derivable therein). In specific embodiments, the ratio between stromal cells and CD34+ cells is about 1:5 to 1:20. In particular embodiments, the stromal cells are a murine stromal cell line, a human stromal cell line, a selected population of primary stromal cells, a selected population of stromal cells differentiated from pluripotent stem cells in vitro, or a combination thereof. In certain embodiments, stroma cells are a selected population of stromal cells differentiated from hematopoietic stem or progenitor cells in vitro. Co-culturing of CD34+ cells and stromal cells may occur for about, at least about, or at most about 1, 2, 3, 4, 5, 6, 7 days and/or 1, 2, 3, 4, 5, 6, 7, 8, 9, 10, 11, 12, 13, 14, 15, 16, 17, 18, 19, 20, 21, 22, 23, 24, or more weeks (or any range derivable therein). The stromal cells are irradiated prior to co-culturing in some embodiments.

In some embodiments, feeder cells used in methods comprise CD34− cells. These CD34-cells may be from the same population of cells selected for CD34+ cells. In additional embodiments, cells may be activated. In certain embodiments, methods comprise activating iNKT cells. In specific embodiments, iNKT cells have been activated and expanded with alpha-galactosylceramide (α-GC). Cells may be incubated or cultured with α-GC so as to activate and expand them. In some embodiments, feeder cells have been pulsed with α-GC.

In some methods, iNKT cells lacking surface expression of one or more HLA-I or -II molecules are selected. In some aspects, selecting iNKT cells lacking surface expression of HLA-I and/or HLA-II molecules protects these cells from depletion by recipient immune cells.

Cells may be used immediately or they may be stored for future use. In certain embodiments, cells that are used to create iNKT cells are frozen, while produced iNKT cells may be frozen in some embodiments. In some aspects, cells are in a solution comprising dextrose, one or more electrolytes, albumin, dextran, and DMSO. In other embodiments, cells are in a solution that is sterile, nonpyrogenic, and isotonic.

The number of cells produced by a production cycle may be about, at least about, or at most about 10², 10³, 10⁴′, 10⁵, 10⁶, 10⁷′, 10⁸, 10⁹, 10¹⁰, 10¹¹, 10¹², 10¹³, 10¹⁴, 10¹⁵ cells or more (or any range derivable therein), which are engineered iNKT cells in some embodiments. In some cases, a cell population comprises at least about 10⁶-10¹² engineered iNKT cells. It is contemplated that in some embodiments, that a population of cells with these numbers is produced from a single batch of cells and are not the result of pooling batches of cells separately produced—i.e., from a single production cycle.

In some embodiments, a cell population is frozen and then thawed. The cell population may be used to create engineered iNKT cells or they may comprise engineered iNKT cells.

Engineered iNKT cells may be used to treat a patient. In some embodiments, methods include introducing one or more additional nucleic acids into the cell population, which may or may not have been previously frozen and thawed. This use provides one of the advantages of creating an off-the-shelf iNKT cell. In particular embodiments, the one or more additional nucleic acids encode one or more therapeutic gene products. Examples of therapeutic gene products include at least the following: 1. Antigen recognition molecules, e.g. CAR (chimeric antigen receptor) and/or TCR (T cell receptor); 2. Co-stimulatory molecules, e.g. CD28, 4-1BB, 4-1BBL, CD40, CD40L, ICOS; and/or 3. Cytokines, e.g. IL-1α, IL-1β, IL-2, IL-4, IL-6, IL-7, IL-9, IL-15, IL-12, IL-17, IL-21, IL-23, IFN-7, TNF-α, TGF-β, G-CSF, GM-CSF; 4. Transcription factors, e.g. T-bet, GATA-3, RORyt, FOXP3, and Bcl-6. Therapeutic antibodies are included, as are chimeric antigen receptors, single chain antibodies, monobodies, humanized, antibodies, bi-specific antibodies, single chain FV antibodies or combinations thereof.

In some embodiments, there are methods of preparing a cell population comprising engineered invariant natural killer (iNKT) T cells comprising: a) selecting CD34+ cells from human peripheral blood cells (PBMCs); b) culturing the CD34+ cells with medium comprising growth factors that include c-kit ligand, flt-3 ligand, and human thrombopoietin (TPO); c) transducing the selected CD34+ cells with a lentiviral vector comprising a nucleic acid sequence encoding α-TCR, β-TCR, thymidine kinase, and a suicide gene such as sr39TK; d) introducing into the selected CD34+ cells Cas9 and gRNA for beta 2 microglobulin (B2M) and/or CTIIA to disrupt expression of B2M and/or CTIIA; e) culturing the transduced cells for 2-12 (such as 2-10 or 6-12) weeks with an irradiated stromal cell line expressing an exogenous Notch ligand to expand iNKT cells in a 3D aggregate cell culture; f) selecting iNKT cells lacking surface expression of HLA-I and/or HLA-II molecules; and, g) culturing the selected iNKT cells with irradiated feeder cells loaded with (α-GC.

In some embodiments, there are engineered iNKT cells produced by a method comprising: a) selecting CD34+ cells from human peripheral blood cells (PBMCs); b) culturing the CD34+ cells with medium comprising growth factors that include c-kit ligand, flt-3 ligand, and human thrombopoietin (TPO); c) transducing the selected CD34+ cells with a lentiviral vector comprising a nucleic acid sequence encoding α-TCR, β-TCR, thymidine kinase, and a reporter gene product; d) introducing into the selected CD34+ cells Cas9 and gRNA for beta 2 microglobulin (B2M) and/or CTIIA to eliminate expression of B2M or CTIIA; e) culturing the transduced cells for 2-10 weeks with an irradiated stromal cell line expressing an exogenous Notch ligand to expand iNKT cells in a 3D aggregate cell culture; f) selecting iNKT cells lacking expression of B2M and/or CTIIA; and, g) culturing the selected iNKT cells with irradiated feeder cells.

The methods of the disclosure may produce a population of cells comprising at least 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, 1×10²⁰, or 1×10²¹ (or any derivable range therein) cells that may express a marker or have a high or low level of a certain marker as described herein. The cell population number may be one that is achieved without cell sorting based on marker expression or without cell sorting based on NK marker expression or without cell sorting based on T-cell marker expression. Furthermore, the population of cells achieved may be one that comprises at least 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, 1×10²⁰, or 1×10²¹ (or any derivable range therein) cells that is made within a certain time period such as a time period that is at least, at most, or exactly 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41 days or 7, 8, 9, 10, 11, 12, 13, 14, 15, 16, 17, 18, 19, 20, 21, 22, 23, 24, 25, 26, 27, 28, 29 or 30 weeks (or any derivable range therein). The high or low levels of marker expression, such as NK activators, inhibitors, or cytotoxic molecules may relate to high expression as determined by FACS analysis. In some embodiments, the high levels are relative to a non-NK cell or a non-iNKT cell, or a cell that is not a T cell. In some embodiments, high levels or low levels are determined from FACS analysis.

Methods of treating patients with an iNKT cell or cell population are also provided. In certain embodiments, the patient has cancer. In some embodiments, the patient has a disease or condition involving inflammation or autoimmunity that is associated with cancer or a cancer treatment. In some embodiments, the patient has a disease or condition involving inflammation or autoimmunity that is not associated with cancer or a cancer treatment. In particular aspects, the cells or cell population are allogeneic with respect to the patient. In additional embodiments, the patient does not exhibit signs of rejection or depletion of the cells or cell population. Some therapeutic methods further include administering to the patient a stimulatory molecule (e.g. α-GC, alone or loaded onto APCs) that activates iNKT cells, or a compound that initiates the suicide gene product.

In some embodiments, the cancer being treated comprises multiple myeloma. In some embodiments, the cancer being treated is leukemia. In some embodiments, the cells are derived from a patient without cancer. In some embodiments, the method further comprises administration of an additional agent. In some embodiments, the additional agent comprises an IL-6R antibody or an IL-1R antagonist. In some embodiments, the IL-6R antibody comprises Tocilizumab or the IL-1R antagonist comprises anakinra. In some embodiments, the additional agent comprises a cytokine antagonist for the treatment of cytokine release syndrome. In some embodiments, the additional agent comprises corticosteroids or an inhibitor of one or more of IL-2R, IL-1R, MCP-1, MIP1B, and TNF-alpha. In some embodiments, the additional agent comprises infliximab, adalimumab, golimumab, certolizumab, or emapalumab.

In some embodiments, the additional agent comprises an antigen that is specifically bound by the iNKT TCR, such as the exogenous iNKT TCR.

In some embodiments, the antigen comprises α-GC. In some embodiments, the patient has received a prior cancer therapy. In some embodiments, the prior therapy was toxic and/or was not effective. In some embodiments, the patient experimentce at least 1, 2, 3, 4, or 5 adverse events of immune related adverse events in response to the prior cancer therapy. In some embodiments, the prior therapy comprises one or more of a proteasome inhibitor, an immunomodulatory agent, an anti-CD38 antibody, or CAR-T cell therapy.

In some embodiments, the cancer comprises BCMA+ malignant cells. In some embodiments, the cancer comprises BCMA+ malignant B cells. In some embodiments, the cancer comprises CD19+ malignant cells.

Treatment of a cancer patient with the iNKT cells may result in tumor cells of the cancer patient being killed after administering the cells or cell population to the patient. Combination treatments with iNKT cells and standard therapeutic regimens or other immunotherapy regimen(s) may be employed. It is contemplated that the methods and compositions include exclusion of any of the embodiments described herein.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.”

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

The following drawings form part of the present specification and are included to further demonstrate certain aspects of the present disclosure. The disclosure may be better understood by reference to one or more of these drawings in combination with the detailed description of specific embodiments presented herein.

FIG. 1 illustrates a schematic of an example of production and use of an off-the-shelf universal hematopoietic stem cell (HSC)-engineered iNKT (^{U}HSC-iNKT) cell adoptive therapy.

FIGS. 2A-2D concern generation of human HSC-engineered iNKT cells in a BLT (human bone marrow-liver-thymus engrafted NOD/SCID/γc^{-/-} mice) humanized mouse model. (A) Example of an experimental design. (B) FACS plots of spleen cells. HSC-iNKT^{BLT}: human HSC-engineered iNKT cells generated in BLT mice. hTc: human conventional T cells. FIGS. 2C-2D show generation of human HSC-engineered NY-ESO-1 specific conventional T cells in an Artificial Thymic Organoid (ATO) in vitro culture system. (C) Example of an experimental design. (D) Cell yield (n=3-6). **P<0.01, by Student's t test.

FIGS. 3A-3D demonstrate an initial CMC study in which there is generation of human HSC-engineered iNKT cells in a robust and high-yield two-stage ATO-αGC in vitro culture system. (HSC-iNKT^{ATO} cells were studied as a therapeutic surrogate.) HSC-iNKT^{ATO}: human HSC-engineered iNKT cells generated in ATO culture.) (A) A 2-stage ATO-αGC in vitro culture system. ATO: Artificial Thymic Organoid; αGC: alpha-Galactosylceramide, a potent agonist ligand that specifically stimulates iNKT cells. (B) Generation of HSC-iNKT^{ATO} cells at the ATO culture stage. 6B11 is a monoclonal antibody that specifically binds to iNKT TCR. (C) Expansion of HSC-iNKT^{ATO} cells at the PBMC/αGC culture stage. (D) HSC-iNKT^{ATO} cell outputs.

FIGS. 4A-4B provide an initial pharmacology study of the phenotype and functionality of human HSC-engineered iNKT cells. (HSC-iNKT^{ATO} and HSC-iNKT^{BLT} cells were studied as therapeutic surrogates.) (A) Surface FACS staining. (B) Intracellular FACS staining. PBMC-iNKT: endogenous iNKT cells expanded in vitro from healthy donor PBMCs; PBMC-Tc: endogenous conventional T cells from healthy donor PBMCs.

FIGS. 5A-5K provide an initial efficacy study of Tumor Killing Efficacy of Human HSC-Engineered iNKT cells. (HSC-iNKT^{ATO} and HSC-iNKT^{BLT} cells were studied as therapeutic surrogates.) (A-F) Blood cancer model. (A) MM.1S-hCD1d-FG human multiple myeloma (MM) cell line. (B) In vitro tumor killing assay. (C) Luciferase activity analysis of the in vitro tumor killing (n=3). (D) In vivo tumor killing assay using an NSG mouse human MM metastasis model. (E-F) Live animal bioluminescence imaging (BLI) analysis of the in vivo tumor killing. Representative BLI images of day 14 (E) and the time course measurement of total body luminescence (TBL; F) are shown (n=3-4). (5G-5K) Solid tumor model. (G) A375-hCD1d-FG human melanoma cell line. (H) In vivo tumor killing assay using an NSG mouse human melamona solid tumor model. (I) Tumor weight (day 25). (J) FACS plots showing the HSC-iNKT^{BLT} cell infiltration into the tumor site (day 25). (K) Quantification of J (n=4). **P<0.01, ***P<0.001, by Student's t test.

FIGS. 6A-6C show an initial safety study of Toxicology/Tumorigenicity. (HSC-iNKT^{BLT} cells were studied as a therapeutic surrogate.) (A) Mouse body weight (n=9-10). ns, not significant, by Student's t test. (B) Mouse survival rate (n=9-10). (C) Mouse pathology. Various tissues were collected and analyzed by the UCLA Pathology Core (n=9-10).

FIGS. 7A-7D provide an initial safety study of sr39TK gene for PET imaging and safety control. (HSC-iNKT^{BLT} cells were studied as a therapeutic surrogate.) (A) Experimental design. (B) PET/CT images of the BLT-iNKT^{TK} mice prior to and post GCV treatment (n=4-5). (C) FACS plots showing the effective and specific depletion of HSC-iNKT^{BLT} cells post GCV treatment (n=4-5). (D) Quantification of the FACS plots in C (n=4-5). ns, not significant; **P<0.01; by Student's t test.

FIGS. 8A-8E illustrate an example of a manufacturing process to produce the ^{U}HSC-iNKT cells. (A) Experimental design. (B) Lenti/iNKT-sr39TK vector-mediated iNKT TCR expression in HSCs. (C) CRISPR-Cas9/B2M-CIITA-gRNAs complex-mediated knockout of the HLA-I/II expression in HSCs. (D) 2M2/Tü39 mAb-mediated MACS negative-selection of HLA-I/II^{neg} cells. (E) 6B11 mAb-mediated MACS positive-selection of HSC-iNKT^{ATO} cells;

FIGS. 9A-9E provide an example of a mechanism of action (MOA) Study. (A) Possible mechanisms used by iNKT cells to target tumor. (B-C) Study of CD1d/TCR-mediated direct killing of tumor cells. (B) Experimental design; (C) Killing of MM.1S-hCD1d-FG human multiple myeloma cells (n=3). (D-E) Study of CD1d-independent targeting of tumor cells through activating NK cells. (D) Experimental design; (E) Killing of K562 tumor cells (n=2). Irradiated PBMCs loaded with αGC were used as antigen-presenting cells (APCs) ns, not significant, *P<0.05, **P<0.01, ****P<0.0001, by one-way ANOVA.

FIGS. 10A-10G demonstrate safety considerations. (A) Possible GvHD and HvG responses and the engineered safety control strategies. (B) An in vitro mixed lymphocyte culture (MLC) assay for the study of GvHD responses. (C) IFN-γ production in MLC assay showing no GvHD response induced by HSC-iNKT^{ATO} cells (n=3). PBMCs from 3 different healthy donors were included as responders. (D) An in vitro mixed lymphocyte culture (MLC) assay for the study of HvG response. (E) IFN-γ production in MLC assay showing minor HvG responses against HSC-iNKT^{ATO} cells (n=3). PBMCs from 2 different healthy donors were used in the experiment. (F) HSC-iNKT^{BLT} cells were resistant to killing by mismatched-donor NK cells in an in vitro mixed NK/iNKT culture. (G) An in vivo mixed lymphocyte adoptive transfer (MLT) assay to study the GvHD and HvD responses. ns, not significant, **P<0.01, ***P<0.001, ****P<0.0001, by one-way ANOVA.

FIGS. 11A-11G demonstrate examples of Combination therapy. (A) Experimental design to study the ^{U}HSC-iNKT cell therapy in combination with the checkpoint blockade therapy. (B) ^{UHSC}CAR-iNKT cell. (C) A375-hCD1d-hCD19-FG human melanoma cell line. (D) Experimental design to study the anti-tumor efficacy of the ^{UHSC}CAR-iNKT cells. (E) ^{UHSC}TCR-iNKT cells. (F) A375-hCD1d-A2/ESO-FG human melanoma cell line. (G) Experimental design to study the anti-tumor efficacy of the ^{UHSC}TCR-iNKT cells.

FIG. 12 illustrates an example of a Pharmacokinetics/Pharmacodynamics (PK/PD) study.

FIG. 13 shows one example of an iNKT-sr39TK Lentiviral vector.

FIG. 14 illustrates one example of a cell manufacturing process for production of ^{U}HSC-iNKT cells.

FIG. 15 shows HSC-Engineered Off-The-Shelf Universal BCMA CAR-iNKT (^{U}BCAR-iNKT) cell therapy for MM.

FIGS. 16A-16G. Pilot CMC Study. ^{U}BCAR-iNKT cells were studied as the therapeutic candidate. (A) A 2-stage in vitro culture system. ATO: Artificial Thymic Organoid; αGC: alpha-galactosylceramide, a potent agonist lipid antigen that specifically stimulates iNKT cells; BCMA-CAR: B-cell maturation antigen-targeting chimeric antigen receptor. (B) Gene modification rates of HSCs. (C) Generation of HSC-iNKT cells in ATO culture. 6B11 is a monoclonal antibody that specifically binds to human iNKT TCR. (D) Expansion of HSC-iNKT cells with αGC. 2M2 is a monoclonal antibody recognizing B2M; Tü39 is a monoclonal antibody recognizing HLA-DR, DP, DQ. (E) MACS purification of HLA-I/II-negative universal HSC-iNKT (^{U}HSC-iNKT) cells. (F) Generation of ^{U}BCAR-iNKT cells through BCMA-CAR engineering and IL-15 expansion. BCMA-CAR-engineered peripheral blood conventional T (BCAR-T) cells were generated in parallel as a control. AY13 is a monoclonal antibody recognizing the tEGFR marker co-expressed with BCMA-CAR. (G) ^{U}BCAR-iNKT cell outputs. Note ^{U}BCAR-iNKT production was confirmed using G-CSF-mobilized CD34⁺ HSCs of two different donors.

FIG. 17. Pilot Pharmacology Study. ^{U}BCAR-iNKT cells were studied as the therapeutic candidate. FACS plots were presented, showing the phenotype and functionality of ^{U}BCAR-iNKT cells, in comparison with that of BCAR-iNKT (HLA-I/II-positive BCMA-CAR engineered HSC-iNKT) cells and BCAR-T (BCMA-CAR engineered peripheral blood T) cells.

FIGS. 18A-18E. Pilot In Vitro Efficacy and MOA Study. ^{U}BCAR-iNKT cells were studied as the therapeutic candidate. (A) In vitro direct tumor cell killing assay. (B) MM.1S-hCD1d-FG human multiple myeloma cell line and tumor cell killing mechanisms. (C) Co-expression of BCMA and CD1d on MM.1S-hCD1d-FG cell line, mimicking that on primary MM tumor cells. BM: bone marrow. (D) Tumor killing efficacy of ^{U}BCAR-iNKT cells (n=4). (E) CAR/TCR dual tumor killing mechanism of ^{U}BCAR-iNKT cells (n=4). PBMC-T: peripheral blood T cells (no CAR); ^{U}HSC-iNKT: HLA-I/II-negative universal HSC-engineered iNKT cells (no CAR).

FIGS. 19A-19E. Pilot In Vivo Efficacy and Safety Study. BCAR-iNKT cells were studied as a therapeutic surrogate. (A) Experimental design. (B) Representative BLI images collected on day 40 (n=4). (C) Quantification of BLI images over time (n=4). TBL, total body luminescence. (D) Survival curve (n=4). (E) Representative immunohistology images showing anti-human CD3-stained tissue sections from day 60 experimental mice (n=4). Arrows indicate tissue-infiltrating CD3⁺ human T cells.

FIGS. 20A-20E. Pilot Immunogenicity Study. ^{U}BCAR-iNKT cells were studied as the therapeutic candidate. (A) Possible GvHD and HvG responses and the engineered safety control strategies. (B) An in vitro mixed lymphocyte culture (MLC) assay for the study of GvHD responses. (C) IFN-γ production in MLC assay showing no GvHD response induced by ^{U}BCAR-iNKT cells (n=4). PBMCs from 3 different healthy donors were used as stimulators. N, no PBMC stimulator. (D) An in vitro MLC assay for the study of HvG responses. (E) IFN-γ production in MLC assay showing no HvG responses against ^{U}BCAR-iNKT cells. PBMCs from 3 different healthy donors were tested as responders. Data from one representative donor were shown (n=3).

FIGS. 21A-21D. Pilot Safety Study—sr39TK gene for PET imaging and safety control. ^{U}BCAR-iNKT cells were studied as the therapeutic candidate. (A) In vitro GCV killing assay using ^{U}BCAR-iNKT cells. Cell counts at day 4 post-GCV treatment were shown (n=5). GCV: ganciclovir, a drug selectively kills cells expressing the sr39TK suicide gene. (B-D) In vivo PET imaging and GCV killing assay using BLT-iNKT^{TK} mice (described in FIG. 2A). (B) Experimental design. (C) Representative PET/CT images of the BLT-iNKT^{TK} mice pre- and post-GCV treatment (n=4-5). (D) Quantification of FACS data showing the effective and specific depletion of HSC-iNKT cells in BLT-iNKT^{TK} mice post-GCV treatment (n=4-5).

FIGS. 22A-22C. Proposed CMC Study. (A) Overview of the CMC design. (B) Projection of the three developmental stages to translate the ^{U}BCAR-iNKT cellular product into clinics. The proposed TRAN1-11597 project is at the pre-IND stage that is circled. (C) Flow diagram showing the proposed pre-IND manufacturing process and In Process Control (IPC) and Product Releasing Assays.

FIGS. 23A-23G. In Vitro Generation of Allogenic HSC-Engineered iNKT (AlloHSC-iNKT) Cells. (A) Experimental design to generate AlloHSC-iNKT cells in vitro. HSC, hematopoietic stem cell; CB, cord blood; PBSC, periphery blood stem cell; αGC, α-galactosylceramide; Lenti/iNKT-sr39TK, lentiviral vector encoding iNKT TCR gene and sr39TK suicide/PET imaging gene. (B-E) FACS monitoring of AlloHSC-iNKT cell generation. (B) Intracellular expression of Inkt TCR (identified as Vβ11+) in CD34+ HSC cells at 72 hours post lentivector transduction. (C) Generation of iNKT cells (identified as iNKT TCR+TCRαβ+ cells) during Stage 1 ATO differentiation culture. A 6B11 monoclonal antibody was used to stain iNKT TCR. (D) Expansion of iNKT cells during Stage 2 αGC expansion culture. (E) Expression of CD4/CD8 co-receptors on AlloHSC-iNKT cells during Stage 1 and Stage 2 cultures. DN, CD4/CD8 double negative; CD4 SP, CD4 single positive; DP, CD4/CD8 double positive; CD8 SP, CD8 single positive. (F) Single cell TCR sequencing analysis of ^{Allo}HSC-iNKT cells. Healthy donor periphery blood mononuclear cell (PBMC)-derived conventional αβ T (PBMC-Tc) and iNKT (PBMC-iNKT) cells were included as controls. The relative abundance of each unique T cell receptor sequence among the total unique sequences identified for individual cells was represented by a pie slice. (G) Table summarizing experiments that have successfully generated ^{Allo}HSC-iNKT cells. Representative of 1 (F) and over 10 experiments (A-E).

FIGS. 24A-24I. Characterization and Gene profiling of ^{Allo}HSC-iNKT Cells. (A-B) FACS characterization of ^{Allo}HSC-iNKT cells. (A) Surface marker expression. (B) Intracellular cytokine and cytotoxic molecule production. PBMC-iNKT and PBMC-Tc cells were included as controls. (C-D) Antigen responses of ^{Allo}HSC-iNKT cells. ^{Allo}HSC-iNKT cells were cultured for 7 days, in the presence or absence of αGC (denoted as αGC or Vehicle, respectively). (C) Cell growth curve (n=3). (D) ELISA analysis of cytokine production (IFN-γ, TNF-α, IL-2, IL-4 and IL-17) at day 3 post αGC stimulation (n=3). (E-I) Deep RNAseq analysis of ^{Allo}HSC-iNKT cells generated from CB or PBSC-derived CD34⁺ HSCs (n=3 for each). Healthy donor PBMC-derived conventional CD8⁺ αβ T (PBMC-αβTc; n=8), CD8⁺ iNKT (PBMC-iNKT; n=3), γδ T (PBMC-γδT; n=6), and NK (PBMC-NK; n=2) cells were included as controls. (E) Principal component analysis (PCA) plot showing the ordination of all six cell types. (F-I) Heatmaps showing the expression of selected genes related to transcription factors (F), HLA molecules (G), immune checkpoint molecules (H), and NK activating receptors and NK inhibitory receptors (I), and for all six cell types. Representative of 1 (E-I) and 3 (A-D) experiments. Data are presented as the mean±SEM. ns, not significant, *P<0.05, **P<0.01, **P<0.001, ****P<0.0001, by Student's t test.

FIGS. 25A-25K. Tumor Targeting of ^{Allo}HSC-iNKT Cells Through NK Pathway. (A-B) FACS analysis of surface NK marker expression and intracellular cytotoxic molecule production by ^{Allo}HSC-iNKT cells. PBMC-NK cells were included as a control. (B) Quantification of killer cell immunoglobulin-like receptors (KIR) expression on ^{Allo}HSC-iNKT cells, in comparison with PBMC-NK and PBMC-iNKT cells (n=7-9). (C-E) In vitro direct killing of human tumor cells by ^{Allo}HSC-iNKT cells. PBMC-NK cells were included as a control. Both fresh and frozen-thawed cells were studied. Five human tumor cell lines were studied: A375 (melanoma), K562 (myelogenous leukemia), H292 (lung cancer), PC3 (prostate cancer), and MM.1S (multiple myeloma). All tumor cell lines were engineered to express firefly luciferase and green fluorescence protein dual reporters (FG). (C) Experimental design. (D) Tumor killing data of A375-FG human melanoma cells at 24-hours (n=4). (E) Tumor killing data of K562-FG human myelogenous leukemia cells at 24-hours (n=4). (F-H) Tumor killing mechanisms of ^{Allo}HSC-iNKT cells. NKG2D and DNAM-1 mediated pathways were studied. (F) Experimental design. (G) Tumor killing data of A375-FG human melanoma cells at 24-hours (tumor:iNKT ratio 1:2) (n=4). (H) Tumor killing data of K562-FG human myelogenous leukemia cells at 24-hours (tumor:iNKT ratio 1:1) (n=4). (I-K) In vivo anti-tumor efficacy of ^{Allo}HSC-iNKT cells in an A375-FG human melanoma xenograft NSG mouse model. (I) Experimental design. BLI, live animal bioluminescence imaging. (J) BLI images showing tumor loads in experimental mice over time. (K) Tumor size measurements over time (n=4-5). Representative of 3 experiments. Data are presented as the mean±SEM. ns, not significant, *P<0.05, **P<0.01, **P<0.001, ****P<0.0001, by 1-way ANOVA. See also FIG. 30.

FIGS. 26A-26L. Tumor Targeting of ^{Allo}HSC-iNKT Cells Engineered with CAR. (A) Experimental design to generate BCMA CAR-engineered ^{Allo}HSC-iNKT (^{Allo}BCAR-iNKT) cells in vitro. BCMA, B-cell maturation antigen; CAR, chimeric antigen receptor; BCAR, BCMA CAR; Retro/BCAR-EGFR, retroviral vector encoding a BCMA CAR gene as well as an epidermal growth factor receptor (EGFR) gene. (B) FACS detection of BCAR expression (identified as EGFR⁺) on ^{Allo}BCAR-iNKT at 72-hours post retrovector transduction. Healthy donor PBMC T cells transduced with the same Retro/BCAR-EGFR vector were included as a staining control (denoted as BCAR-T cells). (C-H) In vitro killing of human multiple myeloma cells by ^{Allo}BCAR-iNKT cells. MM.1S-CD1d-FG, human MM.1S cell line engineered to overexpress human CD1d as well as firefly luciferase and green florescence dual reporters. PBMC-T, BCAR-T, and ^{Allo}HSC-iNKT cells were included as effector cell controls. (C) Experimental design. (D) FACS analysis of BCMA and CD1d expression on MM.1S-CD1d-FG cells. Primary bone marrow (BM) sample from MM patient was included as a control. (E) Diagram showing the triple tumor-killing mechanisms of ^{Allo}BCAR-iNKT cells, mediated by NK activating receptors, iNKT TCR, and BCAR. (F) Tumor killing at 8-hours (Effector:tumor ratio 5:1) (n=4). (G) ELISA analysis of IFN-γ production at 24-hours (n=3). (H) Tumor killing with titrated effector:tumor (E:T) ratios at 24-hours (n=4). (I-L) In vivo antitumor efficacy of ^{Allo}BCAR-iNKT cells in a MM.1S-CD1d-FG human multiple myeloma xenograft NSG mouse model. Tumor-bearing mice injected with BCAR-T cells or no cells (Vehicle) were included as controls. (I) Experimental design. (J) BLI images showing tumor loads in experimental mice over time. (K) Quantification of (J) (n=4). (L) Kaplan-Meier survival curves of experimental mice over a period of 4 months post tumor challenge (n=4). Representative of 2 (I-L) and 3 (A-H) experiments. Data are presented as the mean±SEM. ns, not significant, *P<0.05, **P<0.01, **P<0.001, ****P<0.0001, by Student's t test (H), or by one-way ANOVA (F, G, K), or by log rank (Mantel-Cox) test adjusted for multiple comparisons (J). See also FIG. 31.

FIGS. 27A-27H. Safety Study of ^{Allo}HSC-iNKT Cells. (A-B) Studying the graft-versus-host (GvH) response of ^{Allo}BCAR-iNKT cells using an in vitro mixed lymphocyte culture (MLC) assay. BCAR-T cells were included as a responder cell control. (A) Experimental design. PBMCs from 4 different healthy donors were used as stimulator cells. (B) ELISA analysis of IFN-γ production at day 4 (n=4). N, no stimulator cells. (C-E) Immunohistology analysis of tissue sections from experimental mice described in FIG. 26I-26L. (C) Hematoxylin and eosin staining. Blank indicates tissue sections collected from tumor-free NSG mice. Arrows point to mononuclear cell infiltrates. Bars: 200 μm. (D) Anti-human CD3 staining. CD3 staining is shown in brown. Bars: 100 μm. (E) Quantification of (D) (n=4). (F-H) In vivo controlled depletion of ^{Allo}HSC-iNKT cells via GCV treatment. GCV, ganciclovir. (F) Experimental design. (G) FACS detection of ^{Allo}HSC-iNKT cells in the liver, spleen, and lung of NSG mice at day 5. (H) Quantification of (G) (n=4). Representative of 2 experiments. Data are presented as the mean±SEM. ns, not significant, *P<0.05, **P<0.01, **P<0.001, ****P<0.0001, by one-way ANOVA (B) or by Student's t test (E, H). See also FIG. 32.

FIGS. 28A-28I. Immunogenicity of ^{Allo}HSC-iNKT Cells. (A-E) Studying allogenic NK cell response against ^{Allo}HSC-iNKT cells using an in vitro MLC assay. ^{Allo}HSC-iNKT cells were co-cultured with donor-mismatched PBMC-NK cells. PBMC-iNKT and PBMC-Tc cells were included as controls. (A) Experimental design. (B) FACS monitoring of live cell compositions over time. (C) Quantification of (B) (n=3). (D) FACS detection of ULBP expression. (E) Quantification of (D) (n=5-6). (F-I) Studying allogenic T cell response against ^{Allo}HSC-iNKT cells using an in vitro MLC assay. Irradiated ^{Allo}HSC-iNKT cells (as stimulators) were co-cultured with donor-mismatched PBMC cells (as responders). Irradiated PBMC-iNKT and PBMC-Tc cells were included as stimulator cell controls. (F) Experimental design. PBMCs from 3 different healthy donors were used as responders. (G) ELISA analysis of IFN-γ production at day 4 (n=3). (H) FACS detection of HLA-I and II expression. (I) Quantification of HLA-II⁺ cells from (H) (n=5-6). Representative of 3 experiments. Data are presented as the mean±SEM. ns, not significant, *P<0.05, **P<0.01, **P<0.001, ****P<0.0001, by one-way ANOVA.

FIGS. 29A-29M. Generation and Characterization of HLA-I/II-Negative Universal iNKT (^{U}HSC-iNKT) Cells. (A) Experimental design to generate ^{U}HSC-iNKT and BCMA CAR-engineered ^{U}HSC-iNKT (^{U}BCAR-iNKT) cells. gRNA, guide RNA. CRISPR, clusters of regularly interspaced short palindromic repeats; Cas 9, CRISPR associated protein 9; B2M, beta-2-microglobulin; CIITA, class II major histocompatibility complex transactivator. (B-E) FACS monitoring of ^{U}HSC-iNKT and ^{U}BCAR-iNKT cell generation. (B) Intracellular expression of iNKT TCR (identified as Vβ11⁺) and surface ablation of HLA-I/II (identified as B2M⁻HLA-DR⁻) in CD34⁺ HSCs cells at day 5 (72 hours post lentivector transduction and 48 hours post CRISPR/Cas9 gene editing). (C) Generation of iNKT cells (identified as iNKT TCR⁺TCRαβ⁺ cells) during Stage 1 ATO differentiation culture. (D) Purification of HLA-I/II-negative ^{U}HSC-iNKT cells using a 2-step MACS sorting strategy. (E) BCAR expression (identified as EGFR⁺) on ^{U}BCAR-iNKT cells. Healthy donor PBMC T cells transduced with the same Retro/BCAR-EGFR vector were included as a staining control (denoted as BCAR-T cells). (F-G) Studying allogenic T cell response against ^{U}BCAR-iNKT cells using an in vitro MLC assay. Irradiated ^{U}BCAR-iNKT cells (as stimulators) were co-cultured with donor-mismatched PBMC cells (as responders). Irradiated ^{Allo}BCAR-iNKT and conventional BCAR-T cells were included as stimulator cell controls. (F) Experimental design. PBMCs from 3 different healthy donors were used as responders. (G) ELISA analysis of IFN-γ production at day 4 (n=3). (H-I) Studying allogenic NK cell response against ^{U}HSC-iNKT cells using an in vitro MLC assay. ^{U}HSC-iNKT cells were co-cultured with donor-mismatched PBMC-NK cells. PBMC-Tc cells were included as a control. (H) Experimental design. (I) FACS quantification of live ^{U}HSC-iNKT and PBMC-Tc cells (n=3). (J-M) In vivo anti-tumor efficacy of ^{U}BCAR-iNKT cells in an MM.1S-CD1d-FG human multiple myeloma xenograft NSG mouse model. (J) Experimental design. (K) BLI images showing tumor loads in experimental mice over time. (L) Quantification of (K) (n=5). (M) Kaplan-Meier survival curves of experimental mice over a period of 4 months post tumor challenge (n=5). Representative of 1 (J-M) and 3 (B-I) experiments. Data are presented as the mean±SEM. ns, not significant, ****P<0.0001, by one-way ANOVA (G, I, L), or by log rank (Mantel-Cox) test adjusted for multiple comparisons (M). See also FIG. 28 and FIG. 33.

FIGS. 30A-30I. Tumor Targeting of ^{Allo}HSC-iNKT Cells Through NK Pathway; Related to FIG. 25. A) Schematics showing the engineered A375-FG, K562-FG, H292-FG, PC3-FG and MM.1S-FG cell lines. Fluc, firefly luciferase; EGFP, enhanced green fluorescent protein. (B-D) In vitro direct killing of human tumor cells by ^{Allo}HSC-iNKT cells (related to FIG. 25C-25E). PBMC-NK cells were included as a control. Both fresh and frozen-thawed cells were studied. Tumor killing data of H292-FG human lung cancer cells (B), PC3-FG human prostate cancer cells (C), and MM.1S-FG human multiple myeloma cells (D) were shown at 24-hours (n=4 for each). (E-G) Tumor killing mechanisms of ^{Allo}HSC-iNKT cells (related to main FIG. 25F-25H). NKG2D and DNAM-1 mediated pathways were studied. Tumor killing data of H292-FG (tumor:iNKT ratio 1:2), PC3-FG (tumor:iNKT ratio 1:10), and MM.1S-FG (tumor:iNKT ratio 1:15) were shown at 24-hours (n=4 for each). (H-I) In vivo anti-tumor efficacy of ^{Allo}HSC-iNKT cells in an A375-FG human melanoma xenograft NSG mouse model (related to main FIG. 25I-25K). (H) BLI measurements of tumor loads over time (n=4 or 5). (I) Measurements of tumor weight at the terminal harvest on day 18 (n=4 or 5). Representative of 3 experiments. Data are presented as the mean±SEM. ns, not significant, *P<0.05, **P<0.01, ***P<0.001, ****P<0.0001, by 1-way ANOVA (B-G, I) or by Student's t test (H).

FIGS. 31A-31E. Tumor Targeting of ^{Allo}HSC-iNKT Cells Engineered with CAR; Related to FIG. 26. (A) Schematics showing BCMA-CAR design. SP, spacer; TM, transmembrane. (B-C) FACS characterization of ^{Allo}BCAR-iNKT cells. (B) Surface marker expression. (C) Intracellular cytokine and cytotoxic molecule production. BCAR-T cells were included as a control. (D-E) Anti-tumor effector function of ^{Allo}HSC-iNKT cells. (D) FACS detection of CD69, perforin and granzyme B of iNKT cells at 24-hours post co-culturing with MM.1S-CD1d-FG tumor cells. (E) Quantification of (E) (n=3). Representative of 3 experiments. Data are presented as the mean±SEM. ns, not significant, *P<0.05, **P<0.01, ***P<0.001, ****P<0.0001, by 1-way ANOVA.

FIGS. 32A-32J. Safety study of ^{Allo}HSC-iNKT cells; Related to FIG. 27. (A) Quantification of infiltrating area in tissue sections (related to FIG. 27C) (n=4). (B) In vitro GCV killing assay using ^{Allo}HSC-iNKT cells. Cell counts at day 4 post GCV treatment (n=6). (C-D) Studying the graft-versus-host (GvH) response of ^{Allo}HSC-iNKT cells using an in vitro mixed lymphocyte culture (MLC) assay. PBMC-Tc cells were included as a responder cell control. (C) Experimental design. PBMCs from 4 different healthy donors were used as stimulator cells. (D) ELISA analysis of IFN-γ production at day 4 (n=4). (E-J) Studying the GvH response of ^{Allo}HSC-iNKT cells using NSG mouse model. Donor-matched PBMCs were included as a control. (E) Experimental design. ^{Allo}HSC-iNKT cells were tested. (F) Kaplan-Meier survival curves of experimental mice over time (n=5). (G) Anti-human CD3 staining of tissue sections from experimental mice. CD3 is shown in brown. Bars: 100 km. (H) Quantification of (G) (n=4). (I) Experimental design. ^{Allo}HSC-iNKT cells mixed with donor-matched T cell-depleted PBMC were tested. (J) Kaplan-Meier survival curves of experimental mice over time (n=5). Representative of 2 experiments. Data are presented as the mean±SEM. ns, not significant, *P<0.05, **P<0.01, ***P<0.001, ****P<0.0001, by Student's t test (A, H), or by 1-way ANOVA (B, D), or by log rank (Mantel-Cox) test adjusted for multiple comparisons (F, J).

FIGS. 33A-33I. Characterization of ^{U}HSC-iNKT Cells; Related to FIG. 29. (A) FACS detection of surface marker expression, and Intracellular cytokine and cytotoxic molecule production by ^{U}BCAR-iNKT cells. ^{Allo}BCAR-iNKT and BCAR-T cells were included as controls. (B-C) Studying the GvH response of ^{u}BCAR-iNKT cells using an in vitro mixed lymphocyte culture (MLC) assay. BCAR-T cells were included as a responder cell control. (B) Experimental design. PBMCs from 3 different healthy donors were used as stimulator cells. (C) ELISA analysis of IFN-γ production at day 4 (n=4). (D) In vitro GCV killing assay using ^{U}BCAR-iNKT cells. Cell counts at day 4 post GCV treatment (n=6). (E) Studying allogenic T cell response against ^{U}BCAR-iNKT cells using an in vitro MLC assay. ELISA analysis of IFN-γ production at day 4 (related to main FIGS. 29F and 29G) (n=3). (F) Studying allogenic NK cell response against ^{U}HSC-iNKT cells using an in vitro MLC assay. FACS monitoring of live cell compositions over time (related to main FIGS. 29H and 29I). (G-I) In vitro killing of human multiple myeloma MM.1S-CD1d-FG cells by ^{U}BCAR-iNKT cells. PBMC-T, BCAR-T, and ^{U}HSC-iNKT cells were included as effector cell controls. (G) Experimental design. (H) Tumor killing at 16-hours (E:T ratio 2:1) (n=4). (I) Tumor killing with titrated E:T ratios at 24-hours (n=4). Representative of 3 experiments. Data are presented as the mean±SEM. ns, not significant, *P<0.05, **P<0.01, ***P<0.001, ****P<0.0001, by 1-way ANOVA (C-E, H), or by Student's t test (I).

FIG. 34. MM Relapse in BCAR-T Cell-Treated Tumor-Bearing Mice; Related to FIG. 29. BLI images showing MM tumor relapse at multiple organs, including spine, skull, femur, spleen, liver, and gut at 70 days post BCAR-T cells infusion. Representative of 2 experiments.

FIGS. 35A-35F. CMC Study—iTARGET, ^{U}iTARGET, and CAR-iTARGET Cells. (A-B) A feeder-free ex vivo differentiation culture method to generate monoclonal iTARGET cells from PBSCs (A) or cord blood (CB) HSCs (B). By combining with HLA-I/II gene editing, iTARGET cells can be engineered to be HLA-I/II-negative, resulting in Universal iTARGET (^{U}iTARGET) cells. ^{U}iTARGET cells can be further engineered with CAR to become ^{U}CAR-iTARGET cells. An HLA-E gene can be included in the CAR gene-delivery vector to achieve HLA-E expression on ^{U}CAR-iTARGET cells. The end cellular product, ^{U}CAR-iTARGET cells, are HLA-I/II-negative HLA-E-positive and therefore are suitable for allogeneic adoptive transfer. Note the high numbers of iTARGET cells and their derivatives that can be generated from PBSCs or CB HSCs of a single random healthy donor. (C-D) Development of iTARGET cells at Stage 1 and expansion of differentiated iTARGET cells at Stage 2, from PBSCs (C) or CB HSCs (D). (E) Generation of ^{U}iTARGET cells through combining iTARGET cell culture with CRISPR B2M/CIITA gene-editing. (F) Generation of CAR-iTARGET cells through combining iTARGET cell culture with CAR-engineering. Generation of conventional CAR-T cells from healthy donor peripheral blood T (PBMC-T) cells were included as a control. Note the similar CAR-engineering rate for generating CAR-iTARGET cells and CAR-T cells.

FIG. 36. Pharmacology study of iTARGET and ^{U}iTARGET cells. Representative FACS plots are presented, showing the analysis of phenotype (surface markers) and functionality (intracellular production of effector molecules) of iTARGET and ^{U}iTARGET cells. Native human iNKT (PBMC-iNKT) cells, conventional αβ T (PBMC-T) cells, and NK (PBMC-NK) cells isolated and expanded from healthy donor peripheral blood were included as controls.

FIG. 37. Pharmacology study of BCMA CAR-engineered iTARGET (BCAR-iTARGET) cells. Representative FACS plots are presented, showing the analysis of phenotype (surface markers) and functionality (intracellular production of effector molecules) of BCAR-iTARGET cells. BCMA CAR-engineered conventional αβ T (BCAR-T) cells generated through BCMA CAR-engineering of healthy donor peripheral blood T cells were included as a control.

FIGS. 38A-38C. In Vitro Efficacy and MOA Study of iTARGET Cells. (A) Experimental design of the in vitro tumor cell killing assay. Three engineered human tumor cell lines were used in this study, including a human multiple myeloma cell line MM.1S-hCD1d-FG, a human melanoma cell line A375-hCD1d-FG, and a human chronic myelogenous leukemia cancer cell line K562-hCD1d-FG. (B) Tumor killing efficacy of iTARGET cells against MM.1S-hCD1d-FG tumor cells (n=4), (C) Tumor killing efficacy of iTARGET cells against A375-hCD1d-FG and K562-hCD1d-FG tumor cells (n=3). Data are presented as the mean±SEM. ns, not significant, *P<0.05, **P<0.01, ***P<0.001, ****P<0.0001, by 1-way ANOVA.

FIGS. 39A-39F. In Vitro Efficacy and MOA Study of BCMA CAR-Engineered iTARGET (BCAR-iTARGET) Cells. (A) Experimental design of the in vitro tumor cell killing assay. (B) Schematics showing the engineered MM.1S-hCD1d-FG human multiple myeloma cell line and the A375-hCD1d-FG human melanoma cell line. (C) Tumor killing efficacy of BCAR-iTARGET cells against A375-hCD1d-FG melanoma cells (n=3). (D) Tumor killing efficacy of BCAR-iTARGET cells against MM.1S-hCD1d-FG melanoma cells. BCAR-T cells were included as a control. N=4. (E) Tumor killing efficacy of BCAR-iTARGET cells against MM.1S-hCD1d-FG melanoma cells in the absence or presence of a cognate glycolipid antigen αGC. BCAR-T cells and non-CAR-engineered PBMC-T cells and iTARGET cells were included as controls. N=4. (F) Diagram showing the triple-mechanisms that can be deployed by CAR-iTARGET cells targeting tumor cells, including CAR-mediated, iNKT TCR-mediated, and NK receptor-mediated paths. Data are presented as the mean±SEM. ns, not significant, *P<0.05, **P<0.01, ****P<0.0001, by 1-way ANOVA.

FIGS. 40A-40E. Immunogenicity Study. (A) Possible GvHD and HvG responses and the engineered safety control strategies. (B) An in vitro mixed lymphocyte culture (MLC) assay for the study of GvHD responses. (C) IFN-γ production in MLC assay showing no GvHD response induced by both iTARGET and ^{U}iTARGET cells (n=4). PBMCs from 2 mismatched healthy donors were used as stimulators. N, no PBMC stimulator. (D) An in vitro MLC assay for the study of HvG responses. (E) IFN-γ production in MLC assay showing significantly reduced HvG responses against ^{U}iTARGET cells. PBMCs from 2 mismatched healthy donors were tested as responders. Data from one representative donor were shown (n=4). Data are presented as the mean±SEM. ns, not significant, *P<0.05, **P<0.01, ****P<0.0001, by 1-way ANOVA.

FIGS. 41A-41D. Safety Study—sr39TK Gene for PET Imaging and Safety Control. (A) In vitro GCV killing assay using iTARGET cells. Cell counts at day 4 post-GCV treatment were shown (n=5). GCV: ganciclovir, a drug selectively kills cells expressing the sr39TK suicide gene. (B-D) In vivo PET imaging and GCV killing assay using BLT-iNKT^{TK} mice. (B) Experimental design. (C) Representative PET/CT images of the BLT-iNKT^{TK} mice pre- and post-GCV treatment (n=4-5). (D) Quantification of FACS data showing the effective and specific depletion of HSC-iNKT cells in BLT-iNKT^{TK} mice post-GCV treatment (n=4-5). Data are presented as the mean±SEM. ns, not significant, **P<0.01, ****P<0.0001, by 1-way ANOVA (A) or by Student's t test (D).

FIG. 42. Property of human iNKT cell products generated using various methods. Representative FACS plots are presented, showing the property of human iNKT cell products generated from human PBMC culture, from ATO-iNKT cell culture, and from iTARGET cell culture.

FIGS. 43A-43C. CMC Study—esoTARGET and ^{U}esoTARGET Cells. (A) A feeder-free ex vivo differentiation culture method to generate monoclonal esoTARGET cells from cord blood (CB) HSCs. By combining with HLA-I/II gene editing, esoTARGET cells can be engineered to be HLA-I/II-negative, resulting in Universal esoTARGET (^{U}esoTARGET) cells that are suitable for allogeneic adoptive transfer. Note the high numbers of ^{U}esoTARGET cells that can be generated from CB HSCs of a single random healthy donor. (B) Development of esoTARGET cells at Stage 1 and expansion of differentiated esoTARGET cells at Stage 2. Note the highly pure and homogenous esoTARGET cell product. (C) Generation of ^{U}esoTARGET cells through combining esoTARGET cell culture with CRISPR B2M/CIITA gene-editing.

FIGS. 44A-44C. Pharmacology study of esoTARGET cells. Representative FACS plots are presented, showing the analysis of phenotype (surface markers; A and B) and functionality (intracellular production of effector molecules; C) of esoTARGET cells. Native conventional αβ T (PBMC-T) cells expanded from healthy donor peripheral blood were included as controls. (A) FACS plots showing the surface expression of effector T cell markers on esoTARGET cells. Note that compared to the native PBMC-Tc cells, esoTARGET cells were homogenous and mono-specific (hTCRαβ⁺HLA-A2 ESO Dextramer⁺), more active (CD69^{hi}CD62L^{lo}), and interestingly, also less “exhausted” (CTLA-4^{lo}PD-1^{lo}). (B) FACS plots showing the expression of NK markers on esoTARGET cells. Note that compared to the native PBMC-Tc cells, esoTARGET cells expressed higher levels of NK markers (CD56⁺), NK functional receptors (CD16+/−), and NK activation receptors (NKG2D^{hi}DNAM-1^{hi}). (C) FACS plots showing the intracellular production of cytokines in esoTARGET cells. Note that compared to the native PBMC-Tc cells, esoTARGET cells produced significantly higher levels of effector cytokines (IL-2, IFN-γ, TNF-α) and cytotoxic molecules (Granzyme B and Perforin).

FIGS. 45A-45F. In Vitro Efficacy and MOA Study of esoTARGET Cells. (A) Experimental design of an in vitro tumor cell killing assay. (B) Schematic showing the engineered A375-A2-ESO-FG cell line. A375 is a human melanoma cell line. A375-A2-ESO-FG was generated by engineering the parental A375 cell line to stably overexpress HLA-A2, NY-ESO-1, and firefly luciferase and enhanced green fluorescence protein dual reporters. (C) Tumor killing efficacy of esoTARGET cells against NY-ESO-1⁺ A375-A2-ESO-FG tumor cells (n=4). esoT, human peripheral blood conventional αβ T cells engineered to express the same transgenic esoTCR as that expressed by the esoTARGET cells. Note that esoTARGET cells effectively killed NY-ESO-1⁺ tumor cells, at an efficacy comparable to or better than that of native conventional T (esoT) cells. (D-F) Tumor killing efficacy of esoTARGET cells against NY-ESO-1⁻ tumor cells (n=4). Three tumor cell lines were studied, an A375 human melanoma cell line, an MM.1S human multiple myeloma cell line, and a K562 human chronic myelogenous leukemia cancer cell line. All three tumor cell lines were engineered to express firefly luciferase and enhanced green fluorescence protein dual reporters, denoted as A375-FG, MM.1S-FG, and K562-FG. Note that esoTARGET cells killed all three NY-ESO-1⁻ tumor cell lines at certainly efficacy. Taken together, these results indicate that esoTARGET cells are equipped with dual tumor-killing functions, through an esoTCR/antigen-induced path, and through an esoTCR/antigen-independent path (likely NK path). Data are presented as the mean±SEM. ns, not significant, ****P<0.0001, by 1-way ANOVA (C) or by Student's t test (D, E, F).

FIGS. 46A-46B. Safety Study of esoTARGET cells. The GvHD responses of esoTARGET cells were evaluated using an In Vitro Mixed Lymphocytes Culture (MLC) assay. (A) Experimental design. (B) IFN-γ production in MLC assay, showing minimal alloreactivity of esoTARGET cells in contrast to that of the esoT cells (n=3). esoT, allogeneic peripheral blood conventional αβ T cells engineered to express esoTCR. These results indicate that esoTARGET cells exhibit low alloreactivity and are suitable for developing off-the-shelf cellular products. Data are presented as the mean±SEM. ns, not significant, ****P<0.0001, by 1-way ANOVA.

FIGS. 47A-47C. In Vivo Efficacy Study of BCAR-iTARGET Cells. (A) Experimental design to study the in vivo antitumor efficacy of BCAR-iTARGET cells in a human multiple myeloma (MM) xenograft NSG mouse model. (B-C) Live animal bioluminescence imaging (BLI) analysis of tumor growth. (B) Tumor growth. TBL, total body luminescence. (C) Representative BLI images. N=2. Data are presented as the mean±SEM.

FIGS. 48A-48C. In Vivo Efficacy Study of esoTARGET Cells. (A) Experimental design to study the in vivo antitumor efficacy of esoTARGET cells in a human melanoma xenograft NSG mouse model. (B) Control A375-FG tumor growth (n=5-6). (C) Target A375-A2-ESO-FG tumor growth (n=5-6). Data are presented as the mean±SEM. ns, not significant, *P<0.05, ***P<0.001, ****P<0.0001, by Student's t test.

FIGS. 49A-49D. CMC Study-iTANK and CAR-iTANK Cells. (A-B) A feeder-free ex vivo differentiation culture method to generate monoclonal iNKT TCR-Armed NK (iTANK) cells from PBSCs (A) or cord blood (CB) HSCs (B). By combining with HLA-I/II gene editing, iTANK cells can be engineered to be HLA-I/II-negative, resulting in Universal iTANK (^{U}iTANK) cells. ^{U}iTANK cells can be further engineered with CAR to become ^{U}CAR-iTANK cells. An HLA-E gene can be included in the CAR gene-delivery vector to achieve HLA-E expression on ^{U}CAR-iTANK cells. The end cellular product, ^{U}CAR-iTANK cells, are HLA-I/II-negative HLA-E-positive and therefore are suitable for allogeneic adoptive transfer. Note the high numbers of iTANK cells and their derivatives that can be generated from PBSCs or CB HSCs of a single random healthy donor. (C) Development of iTANK cells at Stage 1 and expansion of differentiated iTANK cells at Stage 2. Data from PBSCs were shown. (D) Generation of CAR-iTANK cells through combining iTANK cell culture with CAR-engineering. A BCMA CAR was used.

FIG. 50. Property of human NK cell products generated using various methods. Representative FACS plots are presented, showing the property of iTANK cell product in comparison with that of native human NK cell products generated from human PBMC culture.

FIGS. 51A-51C. Pharmacology study of CAR-iTANK cells. Representative FACS plots are presented, showing the analysis of phenotype (surface markers; A and B) and functionality (intracellular production of effector molecules; C) of CAR-iTANK cells. CAR-engineered peripheral blood conventional αβ T cells (CAR-T) were included as a control. CAR referred to BCMA CAR. (A) FACS plots showing the surface expression of effector T cell markers on CAR-iTANK cells. Note that compared to conventional CAR-T cells, CAR-iTANK cells expressed minimal levels of HLA-II. CAR-iTANK cells were also more active (CD69^{hi}CD62L^{lo}), and interestingly, also less “exhausted” (PD-1^{lo}). (B) FACS plots showing the expression of NK markers on iTANK cells. Note that compared to the conventional CAR-T, CAR-iTANK cells expressed higher levels of NK markers (CD56^{hi}) and NK activation receptors (NKG2D^{hi}). (C) FACS plots showing the intracellular production of cytokines in CAR-iTANK cells. Note that compared to the conventional CAR-T cells, CAR-iTANK cells produced significantly higher levels of effector cytokines (IL-2, IFN-γ, TNF-α) and cytotoxic molecules (Granzyme B and Perforin).

FIGS. 52A-52F. In Vitro Efficacy and MOA Study—CAR-iTANK Cells. (A) Experimental design of an in vitro tumor cell killing assay. CAR referred to BCMA CAR. (B) Schematic showing the engineered MM.1S-hCD1d-FG cell line. MM.1S is a human multiple myeloma cell line (BCMA+). MM.1S-hCD1d-FG was generated by engineering the parental MM.1S cell line to stably overexpress human CD1d, as well as the firefly luciferase and enhanced green fluorescence protein dual reporters. (C) Schematic showing the engineered A375-hCD1d-FG cell line. A375 is a human melanoma cell line (BCMA⁻). A375-hCD1d-FG was generated by engineering the parental A375 cell line to stably overexpress human CD1d, as well as the firefly luciferase and enhanced green fluorescence protein dual reporters. (D) Tumor killing efficacy of iTANK cells against MM.1S-hCD1d-FG tumor cells (n=3). Note the lack of tumor cell killing by iTANK cells (not engineered with CAR). (E) Tumor killing efficacy of CAR-iTANK cells against MM.1S-hCD1d-FG tumor cells (n=4). CAR-engineered peripheral blood conventional αβ T (CAR-T) cells were included as a control. Note that CAR-iTANK cells killed tumor cells more efficiently than CAR-T cells. (F) Tumor killing efficacy of CAR-iTANK cells against A375-hCD1d-FG tumor cells (n=4). CAR-T cells were included as a control. Note that unlike CAR-T cells, CAR-iTANK cells effectively killed BCMA⁻ tumor cells. Taken together, these results showed that CAR-iTANK cells can effectively kill tumors, through both CAR-induced and CAR-independent (likely through NK path) mechanisms. And that for CAR-induced killing, CAR-iTANK cells are of higher efficacy than conventional CAR-T cells. Data are presented as the mean±SEM. ns, not significant, ***P<0.001, ****P<0.0001, by Student's t test (D) or by 1-way ANOVA.

FIGS. 53A-53B. CMC Study-esoTANK Cells. (A) A feeder-free ex vivo differentiation culture method to generate monoclonal esoTANK cells from cord blood (CB) HSCs. By combining with HLA-I/II gene editing, esoTANK cells can be engineered to be HLA-I/II-negative, resulting in Universal esoTANK (^{U}esoTANK) cells that are suitable for allogeneic adoptive transfer. Note the high numbers of ^{U}esoTANK cells that can be generated from CB HSCs of a single random healthy donor. (B) Development of esoTANK cells at Stage 1 and expansion of differentiated esoTANK cells at Stage 2. Note the highly pure and homogenous esoTANK cell product.

FIGS. 54A-54C. Pharmacology study of esoTANK cells. Representative FACS plots are presented, showing the analysis of phenotype (surface markers; A and B) and functionality (intracellular production of effector molecules; C) of esoTANK cells. Native conventional αβ T (PBMC-T) cells expanded from healthy donor peripheral blood were included as controls. (A) FACS plots showing the surface expression of effector T cell markers on esoTANK cells. Note that compared to the native PBMC-Tc cells, esoTANK cells were homogenous and mono-specific (hTCRαβ⁺HLA-A2 ESO Dextramer⁺), more active (CD69^{hi}CD62L^{lo}), and interestingly, also less “exhausted” (CTLA-4^{lo}PD-1^{lo}). (B) FACS plots showing the expression of NK markers on esoTANK cells. Note that compared to the native PBMC-Tc cells, esoTANK cells expressed higher levels of NK markers (CD56⁺), NK functional receptors (CD16^{+/-}), and NK activation receptors (NKG2D^{hi}DNAM-1^{hi}). (C) FACS plots showing the intracellular production of cytokines in esoTANK cells. Note that compared to the native PBMC-Tc cells, esoTANK cells produced significantly higher levels of effector cytokines (IL-2, IFN-γ, TNF-α) and cytotoxic molecules (Granzyme B and Perforin).

FIGS. 55A-55F. In Vitro Efficacy and MOA Study of esoTANK Cells. (A) Experimental design of an in vitro tumor cell killing assay. (B) Schematic showing the engineered A375-A2-ESO-FG cell line. A375 is a human melanoma cell line. A375-A2-ESO-FG was generated by engineering the parental A375 cell line to stably overexpress HLA-A2, NY-ESO-1, and firefly luciferase and enhanced green fluorescence protein dual reporters. (C) Tumor killing efficacy of esoTANK cells against NY-ESO-1⁺ A375-A2-ESO-FG tumor cells (n=4). Note that esoTANK cells effectively killed NY-ESO-1⁺ tumor cells. (D-F) Tumor killing efficacy of esoTARGET cells against NY-ESO-1⁻ tumor cells (n=4). Three tumor cell lines were studied, an A375 human melanoma cell line, an MM.1S human multiple myeloma cell line, and a K562 human chronic myelogenous leukemia cancer cell line. All three tumor cell lines were engineered to express firefly luciferase and enhanced green fluorescence protein dual reporters, denoted as A375-FG, MM.1S-FG, and K562-FG. Note that esoTANK cells killed all three NY-ESO-1⁻ tumor cell lines at certainly efficacy. Taken together, these results indicate that esoTANK cells are equipped with dual tumor-killing functions, through an esoTCR/antigen-induced path, and through an esoTCR/antigen-independent path (likely NK path). Data are presented as the mean±SEM. ***P<0.001, ****P<0.0001, by Student's t test.

FIGS. 56A-56B. Safety Study of esoTANK cells. The GvHD responses of esoTARGET cells were evaluated using an In Vitro Mixed Lymphocytes Culture (MLC) assay. (A) Experimental design. (B) IFN-γ production in MLC assay, showing minimal alloreactivity of esoTANK cells in contrast to that of the esoT cells (n=3). esoT, allogeneic peripheral blood conventional αβ T cells engineered to express esoTCR. These results indicate that esoTANK cells exhibit low alloreactivity and are suitable for developing off-the-shelf cellular products. Data are presented as the mean±SEM. ns, not significant, ****P<0.0001, by 1-way ANOVA.

FIGS. 57A-57C. Generation of IL-15-enhanced BCAR-iTARGET (^{IL-15}BCAR-iTARGET) cells. (A) Experimental design to generate the IL15BCAR-iTARGET cell product. (B) Schematics of Lenti/BCAR-iNKT-IL15 and Lenti/BCAR-iNKT lentivectors. (C) FACS plots showing the detection of IL15BCAR-iTARGET (hTCRβ+6B11+) cells in cell culture over time. 6B11 is a monoclonal antibody that specifically stains human iNKT TCR. BCAR-iTARGET cells were included as a control.

FIGS. 58A-58E. In vitro antitumor efficacy of ^{IL15}BCAR-iTARGET cells. (A) Experimental design to study the killing of MM.1S-hCD1d-FG human multiple myeloma cells by ^{IL15}BCAR-iTARGET cells. (B) Schematic of a engineered human multiple myeloma cell line (MM.1S-hCD1d-FG). (C) Diagram showing the NK/TCR/CAR-mediated triple tumor killing mechanisms performed by ^{IL15}BCAR-iTARGET cells. (D) Tumor killing efficacy of ^{IL15}BCAR-iTARGET and BCAR-iTARGET cells against MM.1S-hCD1d-FG tumor cells (n=5). (E) FACS detection of activation markers and cytotoxic molecules expression in ^{IL15}BCAR-iTARGET cells and BCAR-iTARGET cells co-cultured with MM.1S-hCD1d-FG tumor cells. Data are presented as the mean±SEM. ns, not significant, *P<0.05, **P<0.01, ***P<0.001, ****P<0.0001, by 1-way ANOVA.

FIGS. 59A-59F. In vivo antitumor efficacy of IL15BCAR-iTARGET cells. (A) Experimental design. (B) Tumor loads measured by BLI in experimental mice over time. (C) Quantification of B (n=3-4). (D) Quantification of tumor load at day 34. (E) FACS plots showing iTARGET cell persistency at day 34 in peripheral blood. (F) Quantification of (E). Data are presented as the mean±SEM. ns, not significant, ****P<0.0001, by 1-way ANOVA.

FIGS. 60A-60D. Construction of gene-delivery lentivectors. (A) Schematic of the Lenti/iNKT-sr39TK lentivector. (B) Schematic of the Lenti/iNKT-CAR19 and Lenti/iNKT-BCAR lentivectors. (C) Titers of the indicated lentivectors, measured by transducing an HEK-293T-CD3 cell line. Note the comparable titers. (D) FACS analyses of CD34+ HSCs transduced with the indicated lentivectors. Note the Lenti/iNKT-CAR19 and Lenti/iNKT-BCAR vectors mediated efficient co-expression of the iNKT TCR and CAR genes. Vβ11 stained iNKT TCRs, while Fab stained CARs.

FIGS. 61A-61G. Generation of HSC-engineered allogeneic iNKT (^{Allo}iNKT), CAR-iNKT (^{Allo}CAR-iNKT), and ^{Allo}BCAR-iNKT cells. (A) Schematic of the experimental design to generate ^{Allo}iNKT cell product. (B) FACS plots showing the detection of ^{Allo}iNKT cells (gated as CD3+6B11+ cells) in cell culture over time. (C) Schematic of the experimental design to generate ^{Allo}CAR19-iNKT cell product. (D) FACS plots showing the detection of ^{Allo}CAR19-iNKT cells (gated as CD3⁺6B11⁺ cells) in cell culture over time. (E) Schematic of the experimental design to generate ^{Allo}BCAR-iNKT cell product. (F) FACS plots showing the detection of ^{Allo}BCAR-iNKT cells (gated as CD3⁺6B11⁺ cells) in cell culture over time. (G) Table showing the cell yields.

FIGS. 62A-62E. Phenotype and functionality of ^{Allo}CAR-iNKT cells. (A) FACS plots showing the co-expression of iNKT TCRs (6B11⁺) and CARs (Fab⁺) on ^{Allo}CAR-iNKT cells. (B) Analysis of TCR Vα and Vβ CDR3 VDJ sequences of ^{Allo}iNKT, ^{Allo}CAR-iNKT, PBMC-iNKT and PBMC-T cells. The relative abundance of each unique TCR sequence among the total unique sequences identified for the sample is represented by a pie slice. Note the lack of randomly recombined endogenous TCRs in ^{Allo}iNKT and ^{Allo}CAR-iNKT cells. (C) FACS plots showing the expression of surface markers and intracellular effector molecules in ^{Allo}CAR-iNKT cells. (D) Expansion of ^{Allo}BCAR-iNKT cells in response to antigen (αGC) stimulation (n=3). (E) Expansion of ^{Allo}CAR19-iNKT cells in response to antigen (αGC) stimulation (n=3). Data are presented as the mean±SEM. ***P<0.001, ****P<0.0001, by Student's t test.

FIGS. 63A-63C. In vitro efficacy and MOA study—^{Allo}iNKT cells. (A) In vitro killing of MM.1S-CD1d-FG human multiple myeloma cells by ^{Allo}iNKT cells (n=4). (B) In vitro killing of A375-CD1d-FG human melanoma cells by ^{Allo}iNKT cells (n=3). (C) In vitro killing of K562-CD1d-FG human leukemia cells by ^{Allo}iNKT cells (n=3). Data are presented as the mean±SEM. ns, not significant, *P<0.05, **P<0.01, ***P<0.001, ****P<0.0001, by 1-way ANOVA.

FIGS. 64A-64D. In vitro efficacy and MOA study—^{Allo}BCAR-iNKT cells. (A) Diagram showing the NK/TCR/CAR-mediated triple tumor killing mechanisms utilized by ^{Allo}BCAR-iNKT cells. (B) In vitro killing of MM.1S-CD1d-FG human multiple myeloma cells by ^{Allo}BCAR-iNKT cells (n=3). (C) IFN-production from (B) (n=3). (D) In vitro killing of MM.1S-CD1d-FG human multiple myeloma cells by ^{Allo}BCAR-iNKT cells compared to that of conventional BCAR-T cells (n=4). Data are presented as the mean±SEM. ns, not significant, *P<0.05, **P<0.01, ***P<0.001, ****P<0.0001, by 1-way ANOVA (B, C) or by Student's t test (D).

FIGS. 65A-65B. In vitro antitumor efficacy and MOA study—AlloCAR19-iNKT cells. (A) In vitro killing of CD19⁺ Raji-CD1d-FG human B-cell lymphoma cells by ^{Allo}CAR19-iNKT cells (n=3). (B) In vitro killing of CD19⁺ Raji-CD1d-FG human B-cell lymphoma cells by ^{Allo}CAR19-iNKT cells compared to that of conventional CAR19-T cells (n=3). Data are presented as the mean±SEM. ns, not significant, **P<0.01, ***P<0.001, ****P<0.0001, by 1-way ANOVA (A) or by Student's t test (B).

FIGS. 66A-66G. In vivo antitumor efficacy and safety study—^{Allo}BCAR-iNKT cells. (A) Experimental design. (B) Tumor loads measured by BLI in experimental mice over time. (C) Quantification of (B) (n=5). (D) Kaplan-Meier analysis of mouse survival rate (n=5). (E) FACS analyses of the surface expression of PD-1 and intracellular production of Granzyme-B and IFN-γ in ^{Allo}BCAR-iNKT and control BCAR-T cells isolated from the liver of the experimental mice (n=4). (F-G) FACS analyses of the biodistribution of ^{Allo}BCAR-iNKT cells (F) versus conventional BCAR-T cells (G) in experimental mice (n=4). Data are presented as the mean±SEM. ns, not significant, *P<0.05, ***P<0.001, ****P<0.0001, by Student's t test (C, E) or by log rank (Mantel-Cox) test adjusted for multiple comparisons (D).

FIGS. 67A-67D. Immunogenicity study—^{Allo}BCAR-iNKT cells. (A-B) Graft-versus-host (GvH) response. (A) Experimental design. (B) IFN-γ production (n=3). PBMCs from 4 random healthy donors were included as stimulators. (C-D) Host-versus-graft (HvG) response. (C) Experimental design. (D) IFN-γ production (n=3). PBMCs from 4 random healthy donors were included as responders. Data are presented as the mean±SEM. ns, not significant, ****P<0.0001, by 1-way ANOVA.

FIGS. 68A-68D. Technological innovations that enable the development of a ^{U}BCAR-iNKT cell product.

FIGS. 69A-69G. Generation and characterization of allogeneic HLA-I/II-negative “universal” BCAR-iNKT (^{U}BCAR-iNKT) cells. (A) Experimental design to generate ^{U}BCAR-iNKT cells. (B) FACS plots showing the detection of ^{U}BCAR-iNKT cells (gated as CD3⁺6B11⁺ cells) in cell culture over time. (C) FACS plots showing the co-expression of iNKT TCR, CAR, and HLA-E on the ^{U}BCAR-iNKT cell product. (D) FACS plots showing the lack of HLA-I/II expression on a large portion of ^{U}BCAR-iNKT cells (unsorted). Conventional PBMC-derived BCAR-T cells and non-HLA gene-edited ^{Allo}BCAR-iNKT cells were included as controls. (E) Quantification of (D). N=4. (F-G) Immunogenicity of ^{U}BCAR-iNKT cells. (F) Experimental design to study the host-versus-graft (HvG) response of ^{U}BCAR-iNKT cells using a Mixed-Lymphocyte Culture (MLC) assay. (G) IFN-γ production (n=3). PBMCs from 4 random healthy donors were included as responders. Data are presented as the mean±SEM. ns, not significant, *P<0.05, **P<0.01, ***P<0.001, ****P<0.0001, by 1-way ANOVA.

FIGS. 70A-70E. In vitro generation and gene profiling of off-the-shelf allogenic HSC-engineered NY-ESO-1-specific T (^{Allo}esoT) cells. (A) Schematic design to generate ^{Allo}esoT cells in in vitro off-the-shelf HSC-based TCR-engineered T cell generation system. (B) FACS detection of intracellular expression of HLA-A*02:01-NY-ESO-1₁₅₇₋₁₆₅-specific TCR (identified as Vβ13.1⁺) in CD34⁺ HSC cells 72 h post lentivector transduction. (C) Representative kinetics of ^{Allo}esoT cell development and differentiation from CD34⁺ HSCs at the indicated weeks. ^{Allo}esoT cells were gated as Vβ13.1⁺CD3⁺. (D) Yield of ^{Allo}esoT cells from 8 different CB donors. (E) Analysis of TCR Vα and Vβ CDR3 VDJ sequences of ^{Allo}esoT, and conventional αβ T (PBMC-T) cells. The relative abundance of each unique T cell receptor sequence among the total unique sequences identified for the sample is represented by a pie slice. Representative of over 10 experiments. See also FIG. 73.

FIGS. 71A-710. Characterization and anti-tumor capacity of ^{Allo}esoT. (A) Characterization of ^{Allo}esoT. FACS plots showing the expression of surface markers, intracellular cytokines, and cytotoxic molecules from ^{Allo}esoT cells (identified as Vβ13.1⁺CD3⁺) compared to PBMC-esoT cells (identified as Vβ13.1⁺CD3⁺). (B) Antigen responses of ^{Allo}esoT cells. ^{Allo}esoT cells were expanded in the presence or absence of NY-ESO-1₁₅₇₋₁₆₅ peptide (ESOp) for 7 days. Growth curve of ^{Allo}esoT expansion over time (n=3). (C-G) Studying the NY-ESO-1-specific killing of multiple tumor cell lines by ^{Allo}esoT cells compared to PBMC-esoT cells. (C) Experimental design. (D-E) Luciferase activity analysis of in vitro tumor killing of A375-Fluc and A375-A2-ESO-Fluc (n=4). E:T, effector/target ratio. (F-G) PC3-A2-ESO-Fluc tumor killing data (n=4). E:T, effector/target ratio. (H-O) Studying in vivo anti-tumor efficacy of ^{Allo}esoT cells against solid tumor in a human melanoma (A375-A2-ESO-Fluc) xenograft mouse model. (H) Experimental design. (I) Measurement of tumor size over time (n=4). (J) Kaplan-Meier analysis of mouse survival rate (n=7 or 8). (K) Biodistribution of PBMC-esoT quantified by terminal FACS analysis. (L) Biodistribution of ^{Allo}esoT quantified by terminal FACS analysis. (M) PD-1 expression quantification of tumor infiltrating lymphocytes (n=4). (N) Intracellular cytotoxic molecule expression of in vivo persistent T cells in liver (n=4). (O) Intracellular cytokines expression of in vivo persistent T cells in liver (n=4). Representative of 3 experiments. See also FIGS. 74-76. Data are presented as the mean±SEM. ns, not significant, *P<0.05, **P<0.01, **P<0.001, ****P<0.0001, by One-way ANOVA (D, E, F, G, I, K, L, M, N and O), or by log rank (Mantel-Cox) test adjusted for multiple comparisons (J).

FIGS. 72A-72Q. Safety study of ^{Allo}esoT and reducing immunogenicity through gene editing. (A-B) An in vitro mixed lymphocyte reaction (MLR) assay for the study of GvH responses of ^{Allo}esoT cells in comparison of conventional PBMC-esoT cells. (A) Experimental design. (B) ELISA analysis of IFN-γ in the supernatants of MLR assay (n=3), showing no GvH response induced by ^{Allo}esoT cells. PBMCs from 3 different healthy donors were included as stimulators. (C-D) An in vitro mixed lymphocyte reaction (MLR) assay for host-versus-graft (HvG) responses of ^{Allo}esoT cells compared to PBMC-esoT cells. (C) Experimental design. (D) ELISA analysis of IFN-γ in the supernatants of MLR assay (n=3), showing less HvG response induced by ^{Allo}esoT cells. PBMCs from 3 different healthy donors were included as responders. (E-G) Immunohistology analysis of tissue sections from experimental mice. (E) Hematoxylin and eosin staining. White dashed lines highlight area with mononuclear cell infiltration. (F) Anti-human CD3 staining. CD3 is shown in red. (E) Quantification of (F) (n=5). (H) Schematic design to generate HLA-I/II-reduced universal HSC-engineered NY-ESO-1-specific T (^{U}esoT) cells in off-the-shelf HSC-based TCR-engineered T cell generation system. (I) Kinetics of ^{U}esoT cells development and differentiation from CD34⁺ HSCs at the indicated week. ^{U}esoT cells were gated as Vβ13.1⁺CD3⁺. (J) FACS plots showing the HLA-I&II expression of ^{U}esoT in comparison with ^{Allo}esoT. (K) Characterization of ^{U}esoT. FACS plots showing the expression of surface markers, intracellular cytokines, and cytotoxic molecules from ^{U}esoT cells (identified as Vβ13.1⁺ CD3⁺) compared to PBMC-esoT cells (identified as Vβ13.1⁺ CD3⁺). (L) Studying the NY-ESO-1-specific killing of PC3-A2-ESO-Fluc by ^{U}esoT cells compared to ^{Allo}esoT cells and PBMC-esoT cells (n=4). (M-N) Quantification of reduced HLA-I (M) and HLA-II (N) expression on ^{U}esoT cells compared to ^{Allo}esoT and PBMC-esoT (n=5). (O-P) ELISA analysis of IFN-γ in the supernatants of MLR assay (n=3), showing reduced HvG response induced by ^{U}esoT cells. PBMCs from 2 different healthy donors were included as stimulators. (Q) ^{U}esoT (HLA-E expressing) resist NK killing compared to ^{Allo}esoT with HLA-I&II gene editing in coculture with NK cells (n=3). Representative of 2 experiments. See also FIGS. 77 and 78. Data are presented as the mean±SEM. ns, not significant, *P<0.05, **P<0.01, **P<0.001, ****P<0.0001, by 1-way ANOVA (B) or by Student's t test (E, H).

FIGS. 73A-73E. The generation of off-the-shelf allogenic HSC-engineered NY-ESO-1-specific T (^{Allo}esoT) cells; related to FIG. 70. (A) Design of the Lentiviral vector carrying two version of NY-ESO-1-specific TCR. HLA-A2*01-NY-ESO-1157-165-specific clone is denoted as 1G4, HLA-B7*02-NY-ESO-160-72-specific clone is denoted as 1E4. (B) Representative titer of lentivirus packaged with indicated vectors. (C) Representative kinetics of ^{Allo}esoT(B7) cell development and differentiation from CD34⁺ HSCs at the indicated weeks. ^{Allo}esoT(B7) cells were gated as ESO60-72HLA-B7 Dextramer CD3⁺. (D-E) TCR-engineered T cell generation in the off-the-shelf HSC-based system is independent of matching MHC expression. (D) Generation of ^{Allo}esoT cells with HLA-A2- and HLA-A2⁺ CB HSC donors. (E) Generation of ^{Allo}esoT(B7) cells with HLA-B7− CB HSC donor. Representative of 3 experiments (C and E) and 8 experiments (D).

FIGS. 74A-74B. Characterization of ^{Allo}esoT; related to FIG. 71. (A-B) Characterization of ^{Allo}esoT. FACS plots showing the expression of surface markers (A), intracellular cytokines, and cytotoxic molecules (B) from ^{Allo}esoT cells (identified as Vβ13.1⁺ CD3⁺) compared to PBMC-esoT cells (identified as Vβ13.1⁺ CD3⁺). Representative of 8 experiments.

FIGS. 75A-75G. In vitro antigen response and tumor killing capacity of ^{Allo}esoT; related to FIG. 71. (A-C) Antigen responses of ^{Allo}esoT cells. ^{Allo}esoT cells were expanded in the presence or absence of NY-ESO-1157-165 peptide (ESOp) for 7 days. ELISA analysis of cytokines: (A) IFN-γ, (B) TNF-α, and (C) IL-2 production at day 3 (n=3). (D-E) Studying the HLA-B7 restricted NY-ESO-1-specific killing of multiple tumor cell lines by ^{Allo}esoT(B7) cells compared to PBMC-esoT cells. (D) Luciferase activity analysis of in vitro tumor killing of A375-Fluc and A375-A2-ESO-Fluc (n=4). (E) In vitro tumor killing of PC3-Fluc and PC3-A2-ESO-Fluc (n=4). (F) In vitro tumor killing of K562-Fluc. (G) In vitro tumor killing of MM.1S-Fluc. Representative of 6 experiments.

FIGS. 76A-76F. In vivo anti-tumor capacity of ^{Allo}esoT, related to FIG. 71. (A-D) Studying in vivo anti-tumor efficacy of ^{Allo}esoT cells against solid tumor in a human melanoma (A375-A2-ESO-Fluc) xenograft mouse model. (A) Quantification of tumor weight at the terminal analysis (n=4). (B) Intracellular cytotoxic molecule expression of in vivo persistent T cells in liver (n=4). (C-D) Intracellular cytokines expression of in vivo persistent T cells in liver (n=4). (E-F) Studying in vivo anti-tumor efficacy of ^{Allo}esoT cells against solid tumor in a human melanoma (PC3-A2-ESO-Fluc) xenograft mouse model. (E) Experimental design. (F) Measurement of tumor size over time (n=4). Representative of 4 experiments.

FIGS. 77A-77E. Safety characterization of ^{Allo}esoT; related to FIG. 72. (A) HLA-I expression of ^{Allo}esoT compared to PBMC-esoT. (B) HLA-II expression of ^{Allo}esoT compared to PBMC-esoT. (C-E) Immunohistology analysis of tissue sections from experimental mice. Quantification of mononuclear cell infiltration in H&E staining pictures (n=5).

FIGS. 78A-78D. The generation and characterization of ^{U}esoT; related to FIG. 72. (A) Design of the Lentiviral vector carrying esoTCR (clone 1G4), HLA-E and sr39TK. (B) Representative titer of virus packaged with indicated lentivectors. (C) FACS detection of intracellular expression of esoTCR (identified as Vβ13.1⁺) and HLA-E in CD34⁺ HSC cells 72 h post lentivector transduction. (D) Characterization of ^{U}esoT. FACS plots showing the expression of surface markers and intracellular cytokines from ^{U}esoT cells (identified as Vβ13.1⁺ CD3⁺) compared to PBMC-esoT cells (identified as Vβ13.1⁺ CD3⁺). Representative of 3 experiments.

FIGS. 79A-79B. Generation of HSC-iNKT in BLT mice. (A) Experimental design to generate HSC-iNKT cells in a BLT humanized mouse model. (B) Time-course FACS monitoring of human immune cells (gated as hCD45+ cells), human ab T cells (gated as hCD45+hTCRab+ cells), and human iNKT cells (gated as hCD45+hTCRab+6B11+ cells) in the peripheral blood of BLT-iNKT mice and control BLT mice post-HSC transfer (n=9-10).

FIGS. 80A-C. Generation of off-the-shelf ^{Allo}HSC-iNKT cells in an ATO culture system. (A) Experimental design to generate AlloHSC-iNKT cells in vitro. (B) Generation of iNKT cells (identified as iNKT TCR⁺TCRαβ⁺ cells) during Stage 1 ATO differentiation culture. A 6B11 monoclonal antibody was used to stain iNKT TCR. (C) Expansion of iNKT cells during Stage 2 αGC expansion culture.

FIGS. 81A-81B. ^{Allo}HSC-iNKT cells reduce T cell alloreaction in the Mixed Lymphocyte Reaction (MLR). (A) Studying the function of iNKT cells in the in vitro MLR assay (iNKT:R:S ration 1:1:25). (B) IFN-γ secretion was significantly decreased on the addition of CD4-iNKT cells to the baseline MLR. (n=3) Data are presented as the mean±SEM. ns, not significant, **P<0.01, ***P<0.001, by 1-way ANOVA test.

FIGS. 82A-82C. ^{Allo}HSC-iNKT cells target allogenic myeloid APCs. (A) Experimental design. (B) FACS detection of human dendritic cells (DCs) (gated as CD11c⁺CD14⁺) in MLR assays. (C) Quantification of A (n=3). Data are presented as the mean±SEM. ns, not significant, *P<0.05, **P<0.01, **P<0.001, ****P<0.0001.

FIGS. 83A-83D. The effect of HSC-iNKT cells on reduction of GvHD in NSG mice. (A) Experimental design to study the effect of HSC-iNKT cells on reduction of GvHD. 1×10⁷ PBMCs or 1×10⁷ PBMCs mixed with 1×10⁷ HSC-iNKT cells were i.v. injected into NSG mice at day 0. (B) Weekly R.O. bleeding. (C) Survival curve. (D) Repeated survival curve. Data were presented as the mean±SEM. ns, not significant, *P<0.05, **P<0.01, by Student's t test

FIGS. 84A-84C. The effect of HSC-iNKT cells on reduction of immune cell-infiltration in major organs. (A) Experimental design to study the effect of HSC-iNKT cells on reduction of immune cell-infiltration in major organs including lung, liver, heart, kidney and spleen. 1×10⁷ PBMCs or 1×10⁷ PBMCs mixed with 1×10⁷ HSC-iNKT cells were i.v. injected into NSG mice at day 0. (B) Immunohistology analysis of tissue sections from experimental mice. CD3 is shown in brown. Arrows point to CD3⁺ cell infiltrates. (C) Quantification of (B) (n=5). Data were presented as the mean±SEM. ns, not significant, *P<0.05, **P<0.01, by Student's t test

FIGS. 85A-85B. The effect of HSC-iNKT cells on reduction of GvHD in NSG mice. (A) Experimental design to study the effect of HSC-iNKT cells on reduction of GvHD. 1×10⁷ PBMCs or 1×10⁷ DCs mixed with 1×10⁷ HSC-iNKT cells were i.v. injected into NSG mice at day 0. (B) Experimental design to study the effect of HSC-iNKT cells on reduction of GvHD. 1×107 PBMCs or 1×10⁷ DC-depleted PBMCs mixed with 1×10⁷ HSC-iNKT cells were i.v. injected into NSG mice at day 0.

FIGS. 86A-86D. AML tumor cell killing capacity by HSC-iNKT cells. (A) Experimental design to study U937 human AML killing of ^{Allo}HSC-iNKT cells. (B) Tumor killing data from (A) at 24 hours (n=4). (C) Experimental design to study HL60 human AML killing of ^{Allo}HSC-iNKT cells. (D) Tumor killing data from (A) at 24 hours (n=4). Data were presented as the mean±SEM. ns, not significant, ****P<0.0001, by 1-way ANOVA.

FIGS. 87A-87B. AML tumor cell killing capacity by HSC-iNKT cells. (A) Experimental design to study U937 human AML CD1d dependent killing of ^{Allo}HSC-iNKT cells. (B) Tumor killing data from (A) at 24 hours (n=4) (E:T=1:5). Data were presented as the mean±SEM. ns, not significant, ****P<0.0001, by 1-way ANOVA.

FIGS. 88A-88F. AML tumor cell killing capacity by HSC-iNKT cells. (A) Experimental design to study U937 human AML killing of ^{Allo}HSC-iNKT cells. (B) Tumor killing data from (A) at 24 hours (n=4). (C) Experimental design to study U937 human AML killing of PBMCs. (D) Tumor killing data from (C) at 12 hours (n=4). (E) Experimental design to study U937 human AML killing of PBMC and ^{Allo}HSC-iNKT cells. (F) Tumor killing data from (E) at 24 hours (n=4). Data were presented as the mean±SEM. ns, not significant, ****P<0.0001, by 1-way ANOVA.

FIGS. 89A-89F. AML tumor cell killing capacity by HSC-iNKT cells. (A) Experimental design to study HL60 human AML killing of ^{Allo}HSC-iNKT cells. (B) Tumor killing data from (A) at 24 hours (n=4). (C) Experimental design to study HL60 human AML killing of PBMCs. (D) Tumor killing data from (C) at 12 hours (n=4). (E) Experimental design to study HL60 human AML killing of PBMC and ^{Allo}HSC-iNKT cells. (F) Tumor killing data from (E) at 24 hours (n=4). Data were presented as the mean±SEM. ns, not significant, ****P<0.0001, by 1-way ANOVA.

FIGS. 90A-90D. In vivo antitumor efficacy of HSC-iNKT cells against AML in human xenograft mouse model. (A) Experimental design to study in vivo antitumor efficacy of HSC-iNKT cells using an U937-FG human AML xenograft NSG mouse model. 1×10⁶ U937-FG cells were i.v. injected into the NSG mice at day 0, and 1×10⁷ PBMCs or 1×10⁷ PBMCs mixed with 2×10⁷ HSC-iNKT cells were i.v. injected into NSG mice at day 3. (B) BLI images showing tumor loads in experimental mice over time. (C) Quantification of (B) (n=5-8). (D) Kaplan-Meier analysis of mouse survival rate (n=5-8). Data were presented as the mean±SEM. ns, not significant, **P<0.01, ****P<0.0001, by 1-way ANOVA (C) or by log rank (Mantel-Cox) test adjusted for multiple comparisons (D).

### DETAILED DESCRIPTION

T cells, such as conventional and non-conventional (i.e. iNKT or NK T cells) play a central role in mediating and orchestrating immune responses against cancer; therefore they are attractive therapeutic targets for treating cancer and other diseases. Natural killer (NK) cells are part of the innate immune system which mediates short-lived rapid immune responses against malignant cells without prior sensitization and more importantly they play a critical role in tumor immunosurveillance. Recently, NK-based immunotherapy has shown promising promises, offering an alternative to conventional T cell based therapies. NK cells have the great potential to be an allogenic off-the-shelf cellular therapeutic candidate, as they display several unique therapeutic features: 1) They do not require strict HLA matching, thus reducing the risk of graft-versus-host disease (GVHD); (2) they have ability to detect malignant cells independent of antibodies and MHC, resulting in first-line immune response; 3) they have underlying mechanisms for inducing target cell death such as it releases cytotoxic molecules such as perforin and granzymes, activate apoptotic receptors on cancer cells leading to cell death and interact with cytotoxic T cells to release cytotoxic cytokines. Despite their therapeutic potentials, current approaches to NK cell therapy have been limited in part by challenges with large scale production of highly purified NK cells.

Currently, human NK cells are freshly isolated from human peripheral blood. Additionally, NK cell enrichment can be achieved by the negative selection of NK cells from peripheral blood mononuclear cells (PBMC) using the magnetic bead-based method, followed by the positive selection of these cells using flow-cytometric cell sorting. Then, NK cells are can be further expanded by supplementing proper cytokines. Although expansion can be achieved by this method, the expansion fold is limited due to the low numbers of NK cells in peripheral blood mononuculear cells (PBMC). Another method includes the generation of NK cells from HSC derived either from bone marrow (BM) or UCB. The culture requires the use of stromal cells of mouse origin as ‘feeder layer’ in order to generate NK cells from HSCs. However, the use of mouse feeder cells can risk of xenogeneic contamination and is challenging to comply with GMP regulations.

A novel method that can reliably generate large quantities of a homogenous population of NK cells with a feeder-free differentiation system is thus pivotal to developing an off-the-shelf NK cell therapy.

T cells recognize antigens through their surface T cell receptor (TCR) molecules. All TCR molecules displayed by a T cell are encoded by a single TCR gene (comprising two genes encoding two subunits of a TCR molecules; referred to as a TCR gene in this material). The TCR gene of a T cell is generated through a random genomic V/D/J recombination process during T cell development, and therefore is unique for each T cell. Based on the genomic components of their TCR genes, T cells can be divided into two large categories, alpha-beta T (αβ T) cells and gamma-delta T (γδ T) cells. Alpha-beta T cells can be further divided into subtypes: 1) conventional αβ T cells that include CD4⁺ helper T cells (CD4 T cells; or T_{H} cells) and CD8⁺ cytotoxic T cells (CD8 T cells; or CTL) cells; and 2) unconventional αβ T cells that include Type 1 invariant natural killer T (iNKT) cells, Type 2 natural killer T (Type 2 NKT) cells, and mucosal associated invariant T (MAIT) cells, and others.

Conventional αβ CD8 T (CD8 T) cells: CD8 T cells recognize protein peptide antigens presented by polymorphic major histocompatibility complex (MHC) Class I molecules. CD8 T cells are potent cytotoxic cells for killing target pathogenic cells. CD8 T cells are also named cytotoxic T lymphocytes (CTLs).

Conventional αβ CD4 T (CD4 T) cells: CD4 T cells recognize protein peptide antigens presented by polymorphic MHC Class II molecules. CD4 T cells are helper T (T_{H}) cells orchestrating the immune responses. Based on their specialized functions, CD4 T cells can be classified into further subtypes: T_{H}1, T_{H}2, T_{H}17, T_{FH}, T_{H}9, T_{REG}, and more.

Type 1 invariant natural killer T (iNKT) cells: iNKT cells recognize glycolipid antigens presented by a non-polymorphic non-classical MHC Class I-like molecule CD1d. Consequently, iNKT cells do not cause graft-versus-host disease (GvHD) when adoptively transferred into allogeneic recipients. iNKT TCR comprises an invariant alpha chain (Vα14-Jα18 in mouse; Vα24-Jα18 in human), and a limited selection of beta chains (predominantly Vβ8/Vβ7/Vβ2 in mouse; predominantly Vβ 11 in human). Both mouse and human iNKT cells respond to a synthetic agonist glycolipid ligand, alpha-Galactosylceramide (αGC, or (α-GC, or α-GalCer).

Type 2 natural killer T (NKT) cells: Type 2 NKT cells are also restricted to CD1d. Type 2 NKT cells have a more diverse TCR repertoire and their antigens are less well defined.

A feeder-free ex vivo differentiation culture method is uncovered to generate off-the-shelf monoclonal TCR-armed Gene-Engineered T (TARGET) and natural killer (TANK) cells with high purity and yield.

The production procedure includes 1) genetic modification of HSCs to express a selected monoclonal TCR gene; 2) ex vivo differentiation of genetically modified HSCs into monoclonal TCR-armed T or NK cells without feeder cells; and 3) In vitro/ex vivo expansion of cells. Expansion methods also include TCR stimulation (e.g. with TCR-cognate antigens or anti-CD3/CD28 antibodies). The cell culture methods and compositions described herein can be combined with HLA-I/II gene-editing and HLA-E gene-engineering to product HLA-I/II-negative HLA-E-positive Universal cells, that are suitable for allogeneic adoptive transfer and therefore can be utilized as off-the-shelf cellular product.

In addition to the antigen-specificity endowed by the monoclonal TCR, the cells can be further engineered to express additional targeting molecules to enhance their disease-targeting capacity. Such targeting molecules can be Chimeric Antigen Receptors (CARs), other T cell receptors (TCRs), natural or synthetic receptors/ligands, or others. The resulting ^{U}CAR-cells, ^{U}TCR-cells, or ^{U}X-cells can then be utilized for off-the-shelf disease-targeting cellular therapy.

The cells and their derivatives can also be further engineered to overexpress genes encoding T cell stimulatory factors, or to disrupt genes encoding T cell inhibitory factors, resulting in functionally enhanced cells and derivatives.

HSCs refer to human CD34⁺ hematopoietic progenitor and stem cells, that can be isolated from cord blood or G-CSF-mobilized peripheral blood (CB HSCs or PBSCs), or derived from embryonic or induced pluripotent stem cells (ES-HSCs or iPS-HSCs). The selected monoclonal TCR gene can encode a conventional αβ TCR (a CD4 TCR or a CD8 TCR), an invariant NKT (iNKT) TCR, a non-invariant NKT TCR, a MAIT TCR, a γδ TCR, or other TCRs.

### I. Definitions

The present disclosure encompasses, in some embodiments, “HSC-iNKT cells”, invariant natural killer T (iNKT) cells engineered from hematopoietic stem cells (HSCs) and/or hematopoietic progenitor cells (HPCs), and methods of making and using thereof. As used herein, “HSCs” is used to refer to HSCs, HPCs, or both HSCs and HPCs.

The term “therapeutically effective amount” as used herein refers to an amount that is effective to alleviate, ameliorate, or prevent at least one symptom or sign of a disease or condition to be treated.

The term “exogenous TCR” refers to a TCR gene or TCR gene derivative that is transferred (i.e. by way of gene transfer/transduction/transfection techniques) into the cell or is the progeny of a cell that has received a transfer of a TCR gene or gene derivative. The exogenous TCR genes are inserted into the genome of the recipient cell. In some embodiments, the insertion is random insertion. Random insertion of the TCR gene is readily achieved by methods known in the art. In some embodiments, the TCR genes are inserted into an endogenous loci (such as an endogenous TCR gene loci). In some embodiments, the cells comprise one or more TCR genes that are inserted at a loci that is not the endogenous loci. In some embodiments, the cells further comprise heterologous sequences such as a marker or resistance gene.

The term “chimeric antigen receptor” or “CAR” refers to engineered receptors, which graft an arbitrary specificity onto an immune effector cell. These receptors are used to graft the specificity of a monoclonal antibody onto a T cell; with transfer of their coding sequence facilitated by retroviral or lentiviral vectors. The receptors are called chimeric because they are composed of parts from different sources. The most common form of these molecules are fusions of single-chain variable fragments (scFv) derived from monoclonal antibodies, fused to CD3-zeta transmembrane and endodomain; CD28 or 41BB intracellular domains, or combinations thereof. Such molecules result in the transmission of a signal in response to recognition by the scFv of its target. An example of such a construct is 14g2a-Zeta, which is a fusion of a scFv derived from hybridoma 14g2a (which recognizes disialoganglioside GD2). When T cells express this molecule (as an example achieved by oncoretroviral vector transduction), they recognize and kill target cells that express GD2 (e.g. neuroblastoma cells). To target malignant B cells, investigators have redirected the specificity of T cells using a chimeric immunoreceptor specific for the B-lineage molecule, CD19. The variable portions of an immunoglobulin heavy and light chain are fused by a flexible linker to form a scFv. This scFv is preceded by a signal peptide to direct the nascent protein to the endoplasmic reticulum and subsequent surface expression (this is cleaved). A flexible spacer allows the scFv to orient in different directions to enable antigen binding. The transmembrane domain is a typical hydrophobic alpha helix usually derived from the original molecule of the signalling endodomain which protrudes into the cell and transmits the desired signal.

The term “antigen” refers to any substance that causes an immune system to produce antibodies against it, or to which a T cell responds. In some embodiments, an antigen is a peptide that is 5-50 amino acids in length or is at least, at most, or exactly 5, 10, 15, 20, 25, 30, 35, 40, 45, 50, 55, 60, 65, 70, 75, 80, 85, 90, 95, 100, 125, 150, 175, 200, 250, or 300 amino acids, or any derivable range therein.

The term “allogeneic to the recipient” is intended to refer to cells that are not isolated from the recipient. In some embodiments, the cells are not isolated from the patient. In some embodiments, the cells are not isolated from a genetically matched individual (such as a relative with compatible genotypes).

The term “inert” refers to one that does not result in unwanted clinical toxicity. This could be either on-target or off-target toxicity. “Inertness” can be based on known or predicted clinical safety data.

The term “xeno-free (XF)” or “animal component-free (ACF)” or “animal free,” when used in relation to a medium, an extracellular matrix, or a culture condition, refers to a medium, an extracellular matrix, or a culture condition which is essentially free from heterogeneous animal-derived components. For culturing human cells, any proteins of a non-human animal, such as mouse, would be xeno components. In certain aspects, the xeno-free matrix may be essentially free of any non-human animal-derived components, therefore excluding mouse feeder cells or Matrigel™. Matrigel™ is a solubilized basement membrane preparation extracted from the Engelbreth-Holm-Swarm (EHS) mouse sarcoma, a tumor rich in extracellular matrix proteins to include laminin (a major component), collagen IV, heparin sulfate proteoglycans, and entactin/nidogen.

The term “defined,” when used in relation to a medium, an extracellular matrix, or a culture condition, refers to a medium, an extracellular matrix, or a culture condition in which the nature and amounts of approximately all the components are known.

A “chemically defined medium” refers to a medium in which the chemical nature of approximately all the ingredients and their amounts are known. These media are also called synthetic media. Examples of chemically defined media include TeSR™.

Cells are “substantially free” of certain reagents or elements, such as serum, signaling inhibitors, animal components or feeder cells, exogenous genetic elements or vector elements, as used herein, when they have less than 10% of the element(s), and are “essentially free” of certain reagents or elements when they have less than 1% of the element(s). However, even more desirable are cell populations wherein less than 0.5% or less than 0.1% of the total cell population comprise exogenous genetic elements or vector elements.

A culture, matrix or medium are “essentially free” of certain reagents or elements, such as serum, signaling inhibitors, animal components or feeder cells, when the culture, matrix or medium respectively have a level of these reagents lower than a detectable level using conventional detection methods known to a person of ordinary skill in the art or these agents have not been extrinsically added to the culture, matrix or medium. The serum-free medium may be essentially free of serum.

“Peripheral blood cells” refer to the cellular components of blood, including red blood cells, white blood cells, and platelets, which are found within the circulating pool of blood.

“Hematopoietic stem and progenitor cells” or “hematopoietic precursor cells” refers to cells that are committed to a hematopoietic lineage but are capable of further hematopoietic differentiation and include hematopoietic stem cells, multipotential hematopoietic stem cells (hematoblasts), myeloid progenitors, megakaryocyte progenitors, erythrocyte progenitors, and lymphoid progenitors. “Hematopoietic stem cells (HSCs)” are multipotent stem cells that give rise to all the blood cell types including myeloid (monocytes and macrophages, neutrophils, basophils, eosinophils, erythrocytes, megakaryocytes/platelets, dendritic cells), and lymphoid lineages (T-cells, B-cells, NK-cells). In this disclosure, HSCs refer to both “hematopoietic stem and progenitor cells” and “hematopoietic precursor cells”.

The hematopoietic stem and progenitor cells may or may not express CD34. The hematopoietic stem cells may co-express CD133 and be negative for CD38 expression, positive for CD90, negative for CD45RA, negative for lineage markers, or combinations thereof. Hematopoietic progenitor/precursor cells include CD34(+)/CD38(+) cells and CD34(+)/CD45RA(+)/lin(−)CD10+(common lymphoid progenitor cells), CD34(+)CD45RA(+)lin(−) CD10(−)CD62L(hi) (lymphoid primed multipotent progenitor cells), CD34(+)CD45RA(+)lin(−) CD10(−)CD123+(granulocyte-monocyte progenitor cells), CD34(+)CD45RA(−)lin(−)CD10(−) CD123+(common myeloid progenitor cells), or CD34(+)CD45RA(−)lin(−)CD10(−)CD123-(megakaryocyte-erythrocyte progenitor cells).

A “vector” or “construct” (sometimes referred to as gene delivery or gene transfer “vehicle”) refers to a macromolecule, complex of molecules, or viral particle, comprising a polynucleotide to be delivered to a host cell, either in vitro or in vivo. The polynucleotide can be a linear or a circular molecule.

A “plasmid”, a common type of a vector, is an extra-chromosomal DNA molecule separate from the chromosomal DNA which is capable of replicating independently of the chromosomal DNA. In certain cases, it is circular and double-stranded.

By “expression construct” or “expression cassette” is meant a nucleic acid molecule that is capable of directing transcription. An expression construct includes, at the least, a promoter or a structure functionally equivalent to a promoter. Additional elements, such as an enhancer, and/or a transcription termination signal, may also be included.

The term “exogenous,” when used in relation to a protein, gene, nucleic acid, or polynucleotide in a cell or organism refers to a protein, gene, nucleic acid, or polynucleotide which has been introduced into the cell or organism by artificial means, or in relation a cell refers to a cell which was isolated and subsequently introduced to other cells or to an organism by artificial means. An exogenous nucleic acid may be from a different organism or cell, or it may be one or more additional copies of a nucleic acid which occurs naturally within the organism or cell. An exogenous cell may be from a different organism, or it may be from the same organism. By way of a non-limiting example, an exogenous nucleic acid is in a chromosomal location different from that of natural cells, or is otherwise flanked by a different nucleic acid sequence than that found in nature.

The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

A “gene,” “polynucleotide,” “coding region,” “sequence,” “segment,” “fragment,” or “transgene” which “encodes” a particular protein, is a nucleic acid molecule which is transcribed and optionally also translated into a gene product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the gene sequence.

The term “cell” is herein used in its broadest sense in the art and refers to a living body which is a structural unit of tissue of a multicellular organism, is surrounded by a membrane structure which isolates it from the outside, has the capability of self-replicating, and has genetic information and a mechanism for expressing it. Cells used herein may be naturally-occurring cells or artificially modified cells (e.g., fusion cells, genetically modified cells, etc.).

As used herein, the term “stem cell” refers to a cell capable of self-replication and pluripotency or multipotency. Typically, stem cells can regenerate an injured tissue. Stem cells herein may be, but are not limited to, embryonic stem (ES) cells, induced pluripotent stem cells or tissue stem cells (also called tissue-specific stem cell, or somatic stem cell).

“Embryonic stem (ES) cells” are pluripotent stem cells derived from early embryos. An ES cell was first established in 1981, which has also been applied to production of knockout mice since 1989. In 1998, a human ES cell was established, which is currently becoming available for regenerative medicine.

Unlike ES cells, tissue stem cells have a limited differentiation potential. Tissue stem cells are present at particular locations in tissues and have an undifferentiated intracellular structure. Therefore, the pluripotency of tissue stem cells is typically low. Tissue stem cells have a higher nucleus/cytoplasm ratio and have few intracellular organelles. Most tissue stem cells have low pluripotency, a long cell cycle, and proliferative ability beyond the life of the individual. Tissue stem cells are separated into categories, based on the sites from which the cells are derived, such as the dermal system, the digestive system, the bone marrow system, the nervous system, and the like. Tissue stem cells in the dermal system include epidermal stem cells, hair follicle stem cells, and the like. Tissue stem cells in the digestive system include pancreatic (common) stem cells, liver stem cells, and the like. Tissue stem cells in the bone marrow system include hematopoietic stem cells, mesenchymal stem cells, and the like. Tissue stem cells in the nervous system include neural stem cells, retinal stem cells, and the like.

“Induced pluripotent stem cells,” commonly abbreviated as iPS cells or iPSCs, refer to a type of pluripotent stem cell artificially prepared from a non-pluripotent cell, typically an adult somatic cell, or terminally differentiated cell, such as fibroblast, a hematopoietic cell, a myocyte, a neuron, an epidermal cell, or the like, by introducing certain factors, referred to as reprogramming factors.

As used herein, “isolated” for example, with respect to cells and/or nucleic acids means altered or removed from the natural state through human intervention.

“Pluripotency” refers to a stem cell that has the potential to differentiate into all cells constituting one or more tissues or organs, or particularly, any of the three germ layers: endoderm (interior stomach lining, gastrointestinal tract, the lungs), mesoderm (muscle, bone, blood, urogenital), or ectoderm (epidermal tissues and nervous system). “Pluripotent stem cells” used herein refer to cells that can differentiate into cells derived from any of the three germ layers, for example, direct descendants of totipotent cells or induced pluripotent cells.

By “operably linked” with reference to nucleic acid molecules is meant that two or more nucleic acid molecules (e.g., a nucleic acid molecule to be transcribed, a promoter, and an enhancer element) are connected in such a way as to permit transcription of the nucleic acid molecule. “Operably linked” with reference to peptide and/or polypeptide molecules is meant that two or more peptide and/or polypeptide molecules are connected in such a way as to yield a single polypeptide chain, i.e., a fusion polypeptide, having at least one property of each peptide and/or polypeptide component of the fusion. The fusion polypeptide is particularly chimeric, i.e., composed of heterologous molecules.

Embodiments of the disclosure concern HSC cells engineered to function as iNKT cells with an NKT cell T cell receptor (TCR) and that also have imaging and suicide targeting capabilities and are resistant to host immune cell-targeted depletion. In some embodiments, such cells are generated in an Artificial Thymic Organoid (ATO) in vitro culture system that supports the differentiation of the TCR-engineered HSCs into clonal T cells at high-efficiency and high yield. In some embodiments, such cells are not generated in an ATO culture system. In some embodiments, such cells are generated using a culture system that does not comprise feeder cells (i.e. is “feeder free”).

### II. Universal Hematopoietic Stem Cell (HSC) Engineered Invariant NKT Cells (^{U}HSC-iNKT Cells)

Embodiments of the disclosure utilize cells (such as HSCs) that are modified to function as invariant NKT cells and that are engineered to have one or more characteristics that render the cells suitable for universal use (use for individuals other than the individual from which the original cells were obtained) without deleterious immune reaction in a recipient of the cells. The present disclosure encompasses engineered invariant natural killer T (iNKT) cells comprising a nucleic acid comprising i) all or part of an iNKT alpha T-cell receptor gene; ii) all or part of an iNKT beta T-cell receptor gene, and iii) a suicide gene, wherein the genome of the cell has been altered to eliminate surface expression of at least one HLA-I or HLA-II molecule.

### III. Detailed Description of the Cell Culture Method

A. TARGET Cell Culture Method Embodiments

1. Stage 1: TARGET Cell Differentiation

In some embodiments, fresh or frozen/thawed CD34+ HSCs are cultured in stem cell culture media (base medium supplemented with cytokine cocktails including IL-3, IL-7, IL-6, SCF, EPO, TPO, FLT3L, and others) for 12-72 hours in flasks coated with retronectin, followed by addition of the TCR gene-delivery vector, and culturing for an additional 12-48 hours.

In some embodiments, TCR gene-modified HSCs are then differentiated into TARGET cells in a differentiation medium over a period of 4-10 weeks without feeders. Non-tissue culture-treated plates are coated with a TARGET Culture Coating (TARGETc) Material (DLL-1/4, VCAM-1/5, retronectin, and others). CD34+ HSCs are suspended in a TARGET Expansion (TARGETe) Medium (base medium containing serum albumin, recombinant human insulin, human transferrin, 2-mercaptoethanol, SCF, TPO, IL-3, IL-6, Flt3 ligand, human LDL, UM171, and additives), seeded into the coated wells of a plate, and cultured for 3-7 days. TARGETe Medium is refreshed every 3-4 days. Cells are then collected and suspended in a TARGET Maturation (TARGETm) Medium (base medium containing serum albumin, recombinant human insulin, human transferrin, 2-mercaptoethanol, SCF, TPO, IL-3, IL-6, IL-7, IL-15, Flt3 ligand, ascorbic acid, and additives). TMM is refreshed 1-2 times per week.

2. Stage 2: TARGET Cell Expansion

In some embodiments, differentiated TARGET cells are stimulated with TCR cognate antigens (proteins, peptides, lipids, phosphor-antigens, small molecules, and others) or non-specific TCR stimulatory reagents (anti-CD3/anti-CD28 antibodies or antibody-coated beads, Concanavalin A, PMA/Ionomycin, and others), and expanded for up to 1 month in T cell culture media. The culture can be supplemented with T cell supporting cytokines (IL-2, IL-7, IL-15, and others).

3. TARGET Cell Derivatives

In some embodiments, TARGET cells can be further engineered to express additional transgenes. In one embodiment, such transgenes encode disease targeting molecules such as chimeric antigen receptors (CARs), T-cell receptors (TCRs), and other native or synthetic receptor/ligands. In another embodiment, such transgenes can encode T cell regulatory proteins such as IL-2, IL-7, IL-15, IFN-γ, TNF-α, CD28, 4-1BB, OX40, ICOS, FOXP3, and others. Transgenes can be introduced into post-expansion TARGET cells or their progenitor cells (HSCs, newly differentiated TARGET cells, in-expansion TARGET cells) at various culture stages.

In some embodiments, TARGET cells can be further engineered to disrupt selected genes using gene editing tools (CRISPR, TALEN, Zinc-Finger, and others). In one embodiment, disrupted genes encode T cell immune checkpoint inhibitors (PD-1, CTLA-4, TIM-3, LAG-3, and others). Deficiency of these negative regulatory genes may enhance the disease fighting capacity of TARGET cells, making them resistance to disease-induced anergy and tolerance.

In some embodiments, TARGET cells or enhanced TARGET cells can be further engineered to make them suitable for allogeneic adoptive transfer, thereby suitable for serving as off-the-shelf cellular products. In one embodiment, genes encoding MHC molecules or MHC expression/display regulatory molecules [MHC molecules, B2M, CIITA (Class II transcription activator control induction of MHC class II mRNA expression), and others]. Lack of MHC molecule expression on TARGET cells makes them resistant to allogeneic host T cell-mediated depletion. In another embodiment, MHC class-I deficient TARGET cells will be further engineered to overexpress an HLA-E gene that will endow them resistant to host NK cell-mediated depletion.

TARGET cells and derivatives can be used freshly or cryopreserved for further usage. Moreover, various intermediate cellular products generated during TARGET cell culture can be paused for cryopreservation, stored and recovered for continued production.

4. Novel Features and Advantages

Aspects of the present disclosure provide an in vitro differentiation method that does not require xenogeneic feeder cells. This new method greatly improves the process for the scale-up production and GMP-compatible manufacturing of therapeutic cells for human applications.

The cell products, TARGET cells, display phenotypes/functionalities distinct from that of their native counterpart T cells as well as their counterpart T cells generated using other ex vivo culture methods (e.g. ATO culture method), making TARGET cells unique cellular products.

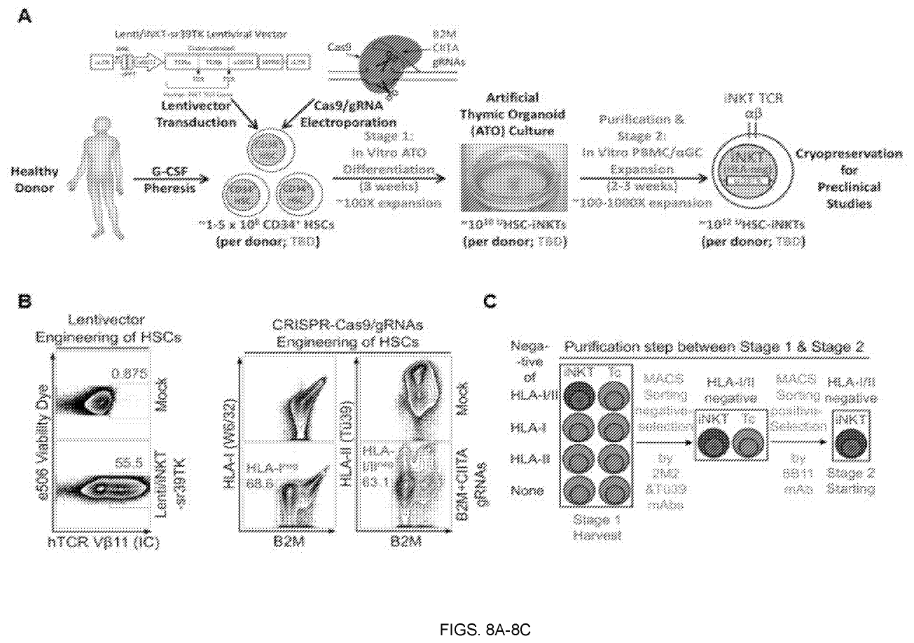

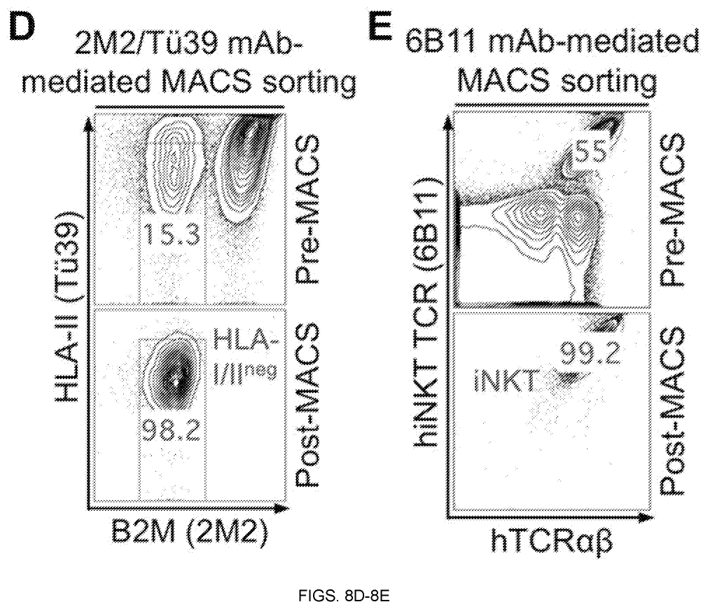

Unique features of the TARGET cell differentiation culture include: 1) It is Ex Vivo and Feeder-Free. 2) It does not support TCR V/D/J recombination, so no randomly rearranged endogenous TCRs, thereby no GvHD risk. 3) It supports the synchronized differentiation of transgenic TARGET cells, thereby eliminating the presence of un-differentiated progenitor cells and other lineages of bystander immune cells. 4) As a result, the TARGET cell product comprises a homogenous and pure population of monoclonal TCR-armed T cells. No escaped random T cells, no other lineages of immune cells, and no un-differentiated progenitor cells. Therefore, no need for a purification step. 5) High yield. About 10¹² TARGET cells (1,000-10,000 doses) can be generated from PBSCs of a healthy donor, and about 10¹¹ TARGET cells (100-1,000 doses) can be generated from CB HSCs of a healthy donor. 6) Unique phenotype of TARGET cells-transgenic TCR+endogenousTCR-CD3+. (Note: These unique features of the TARGET cell differentiation culture distinct it from other methods to generate off-the-shelf T cell products, including the healthy donor PBMC-based T cell culture, the ATO culture, and the others. See FIG. 8.)

5. Example Cell Culture Medium

Provided is an example of cell culture media which may be used to generate engineered immune cells of the present disclosure.

a. Stem Cell Culture Stage (D0-D2)

Base media: X-VIVO15™ (Lonza)

Supplements: hFlt3-L 50 ng/ml, hSCF 50 ng/ml, hTPO 50 ng/ml, hIL-3 10 ng/ml

b. Lymphoid Progenitor Expansion Stage (W1-W2)

Base media: StemSpan™ SFEM II (Stem Cell Technologies). Contains: Iscove's MDM, Bovine serum albumin, Recombinant human insulin, Human transferrin (iron-saturated), 2-Mercaptoethanol, Supplements

Coating material: StemSpan™ Lymphoid Differentiation Coating Material (100×) (Stemcell Technologies). Contains: hDLL4 (50 ug/ml), hVCAM1 (10 ug/ml), Other supplements

Supplements: StemSpan™ Lymphoid Progenitor Expansion Supplement (10×) (Stemcell Technologies). Contains: hFlt3L (20 ng/ml), hIL-7 (25 ng/ml), hMCP-4 (1 ng/ml), hTPO (5 ng/ml), hSCF (15 ng/ml), Other supplements

c. T Cell Progenitor Maturation Stage (W3-W4)

Base media: StemSpan™ SFEM II (Stem Cell Technologies). Contains: Iscove's MDM, Bovine serum albumin, Recombinant human insulin, Human transferrin (iron-saturated), 2-Mercaptoethanol, Supplements

Coating material: StemSpan™ Lymphoid Differentiation Coating Material (100×) (Stemcell Technologies). Contains: hDLL4 (50 ug/ml), hVCAM1 (10 ug/ml), Other supplements

Supplements: StemSpan™ Lymphoid Progenitor Expansion Supplement (10×) (Stemcell Technologies). Contains: hFlt3L (20 ng/ml), hIL-7 (25 ng/ml), Other supplements

d. T Cell Activation Stage (W5)

Base media: StemSpan™ SFEM II (Stem Cell Technologies). Contains: Iscove's MDM, Bovine serum albumin, Recombinant human insulin, Human transferrin (iron-saturated), 2-Mercaptoethanol, Supplements

Coating material: StemSpan™ Lymphoid Differentiation Coating Material (100×) (Stemcell Technologies). Contains: hDLL4 (50 ug/ml), hVCAM1 (10 ug/ml), Other supplements

Supplements:

1) StemSpan™ Lymphoid Progenitor Expansion Supplement (10×) (Stemcell Technologies). Contains: hFlt3L (20 ng/ml), hIL-7 (20 ng/ml), hIL-15 (10 ng/ml), Other supplements

2) ImmunoCult™ Human CD3/CD28/CD2 T Cell Activator (Stemcell Technologies). Contains: ahCD3 Ab clone:OKT3 (1 ug/ml), ahCD28 Ab clone:CD28.2 (1 ug/ml), ahCD2 Ab clone: RPA-2.10 (1 ug/ml)

e. T Cell Expansion Stage (W6)

Base media: T Cell Medium. Contains: X-vivo15 serum-free medium (Lonza, Allendale N.J.), 5% (vol/vol) GemCell human serum antibody AB, (Gemini Bio Products, West Sacramento Calif.), 1% (vol/vol) Glutamax-100X (Gibco Life Technologies), 10 mM HEPES buffer (Corning), 1% (vol/vol) penicillin/streptomycin (Corning), 12.25 mM N-Acetyl-L-cysteine (Sigma)

Supplements: hIL7 (10 ng/ml), hIL15 (50 ng/ml)

Other key materials: 100 ng/ml α-Galactosylceramide (KRN7000) (Avanti Polar Lipids, SKU #867000P-1 mg), ahCD3 Ab clone:OKT3 (5 ug/ml), ahCD28 Ab clone:CD28.2 (5 ug/ml)

B. TANK Cell Culture Method Embodiments

1. Stage 1: TANK Cell Differentiation

In some embodiments, fresh or frozen/thawed CD34⁺ HSCs are cultured in stem cell culture media (base medium supplemented with cytokine cocktails including IL-3, IL-7, IL-6, SCF, EPO, TPO, FLT3L, and others) for 12-72 hours in flasks coated with retronectin, followed by addition of the TCR gene-delivery vector, and culturing for an additional 12-48 hours.

In some embodiments, TCR gene-modified HSCs are then differentiated into TANK cells in a differentiation medium over a period of 2-4 weeks without feeders. Non-tissue culture-treated plates are coated with a TANK Culture Coating (TANKc) Material (DLL-1/4, VCAM-1/5, retronectin, and others). CD34⁺ HSCs are suspended in a TANK Expansion (TANKe) Medium (base medium containing B27 supplement, ascorbic acid, Glutamax, human serum AB/albumin, Flt3 ligand, IL-6, IL-7, SCF, TPO, EPO, leukemia inhibitory factor, GM-CSF, and others), seeded into the coated wells of a plate, and cultured for 7-10 days. TANKe medium is refreshed every 3-5 days. Cells are then collected and suspended in a TANK Maturation (TANKm) Medium (base medium containing B27 supplement, ascorbic acid, Glutamax, human serum AB/albumin, Flt3 ligand, IL-6, IL-7, IL-15, SCF, TPO, leukemia inhibitory factor, and others) and cultured for another 7-10 days. TANKm medium is refreshed every 3-5 days.

2. Stage 2: TANK Cell Expansion

In some embodiments, differentiated TANK cells are stimulated with TCR cognate antigens (proteins, peptides, lipids, phosphor-antigens, small molecules, and others) or non-specific TCR stimulatory reagents (anti-CD3/anti-CD28 antibodies or antibody-coated beads, Concanavalin A, PMA/Ionomycin, and others), and expanded for up to 1 month in T cell culture media. The culture can be supplemented with T cell supporting cytokines (IL-2, IL-7, IL-15, and others).

3. TANK Cell Derivatives

In some embodiments, TANK cells can be further engineered to express additional transgenes. In one embodiment, such transgenes encode disease targeting molecules such as chimeric antigen receptors (CARs), T-cell receptors (TCRs), and other native or synthetic receptor/ligands. In another embodiment, such transgenes can encode T cell regulatory proteins such as IL-2, IL-7, IL-15, IFN-γ, TNF-α, CD28, 4-1BB, OX40, ICOS, FOXP3, and others. Transgenes can be introduced into post-expansion TANK cells or their progenitor cells (HSCs, newly differentiated TANK cells, in-expansion TANK cells) at various culture stages.

In some embodiments, TANK cells can be further engineered to disrupt selected genes using gene editing tools (CRISPR, TALEN, Zinc-Finger, and others). In one embodiment, disrupted genes encode T cell immune checkpoint inhibitors (PD-1, CTLA-4, TIM-3, LAG-3, and others). Deficiency of these negative regulatory genes may enhance the disease fighting capacity of TANK cells, making them resistance to disease-induced anergy and tolerance.

In some embodiments, TANK cells or enhanced TANK cells can be further engineered to make them suitable for allogeneic adoptive transfer, thereby suitable for serving as off-the-shelf cellular products. In one embodiment, genes encoding MHC molecules or MHC expression/display regulatory molecules [MHC molecules, B2M, CIITA (Class II transcription activator control induction of MHC class II mRNA expression), and others]. Lack of MHC molecule expression on TANK cells makes them resistant to allogeneic host T cell-mediated depletion. In another embodiment, MHC class-I deficient TANK cells will be further engineered to overexpress an HLA-E gene that will endow them resistant to host NK cell-mediated depletion.

TANK cells and derivatives can be used freshly or cryopreserved for further usage. Moreover, various intermediate cellular products generated during TANK cell culture can be paused for cryopreservation, stored and recovered for continued production.

4. Novel Features and Advantages

This new method fits for the scale-up production and GMP-compatible manufacturing of therapeutic natural killer cells for human applications.

The cell products, TANK cells, represent a novel type of NK cells that follow a distinct development path and display distinct phenotypes/functionalities differed from native human NK cells expanded from peripheral blood or NK cells generated using other ex vivo culture methods (e.g. iPS cell-derived NK cells or CB-derived NK cells).

Unique features of the TANK cell culture method and include: 1) Designer TANK cell differentiation culture medium that supports the differentiation of TANK cells in 2-3 weeks (much faster than TARGET cell differentiation culture and ATO T cell differentiation culture). 2) It does not support TCR V/D/J recombination, so no randomly rearranged endogenous TCRs, thereby no GvHD risk. 3) It supports the synchronized differentiation of transgenic TANK cells, thereby eliminating the presence of un-differentiated progenitor cells and other lineages of immune cells. 4) As a result, the TANK cell product comprises a homogenous and pure population of monoclonal TCR-armed T cells. No escaped random T cells, no other lineages of immune cells, and no un-differentiated progenitor cells. Therefore, no need for a purification step. 5) High yield. About 10¹² TANK cells (1,000-10,000 doses) can be generated from PBSCs of a healthy donor, and about 10¹¹ TANK cells (100-1,000 doses) can be generated from CB HSCs of a healthy donor. (Note: These unique features of the TANK cell differentiation culture distinct it from other methods to generate NK cell products, including the healthy donor PBMC-based NK cell culture, CB-derived NK cell culture, iPS-derived NK cell culture, and the others.)

5. Example Cell Culture Medium

Provided is an example of cell culture media which may be used to generate engineered immune cells of the present disclosure.

a. Stem Cell Culture Stage (D0-D2)

Base media: X-VIVO15™ (Lonza)

Supplements: hFlt3-L 50 ng/ml, hSCF 50 ng/ml, hTPO 50 ng/ml, hIL-3 10 ng/ml

b. Expansion Stage (W1)

Base media: StemSpan™ SFEM II (Stem Cell Technologies). Contains: Iscove's MDM, Bovine serum albumin, Recombinant human insulin, Human transferrin (iron-saturated), 2-Mercaptoethanol, Supplements

Coating material: hDLL4 (50 ug/ml), hVCAM1 (10 ug/ml)

Supplements: 100 uM Ascorbic Acids. 5% human serum AB (Gemini CAT #800-120). 4% XenoFree B27 (ThermoFisher Scientific, #17504044), 1% Glutamax (ThermoFisher Scientific, #35050-061), hFlt3L (50 ng/ml), hIL-7 (50 ng/ml), hMCP-4 (ing/ml), hIL-6 (10 ng/ml), hTPO (50 ng/ml), hSCF (50 ng/ml), Other supplements

c. Maturation Stage (W2)

Base media: StemSpan™ SFEM II (Stem Cell Technologies). Contains: Iscove's MDM, Bovine serum albumin, Recombinant human insulin, Human transferrin (iron-saturated), 2-Mercaptoethanol, Supplements

Coating material: hDLL4 (50 ug/ml), hVCAM1 (10 ug/ml)

Supplements: 100 uM Ascorbic Acids. 5% human serum AB (Gemini CAT #800-120). 4% XenoFree B27 (ThermoFisher Scientific, #17504044), 1% Glutamax (ThermoFisher Scientific, #35050-061), hFlt3L (50 ng/ml), hIL-7 (50 ng/ml), hIL-15 (50 ng/ml), Other Supplements

d. Activation Stage (W3)

Base media: StemSpan™ SFEM II (Stem Cell Technologies). Contains: Iscove's MDM, Bovine serum albumin, Recombinant human insulin, Human transferrin (iron-saturated), 2-Mercaptoethanol, Supplements

Coating material: hDLL4 (50 ug/ml), hVCAM1 (10 ug/ml)

Supplements: 100 uM Ascorbic Acids. 5% human serum AB (Gemini CAT #800-120). 4% XenoFree B27 (ThermoFisher Scientific, #17504044), 1% Glutamax (ThermoFisher Scientific, #35050-061), hFlt3L (50 ng/ml), hIL-7 (50 ng/ml), hIL-15 (50 ng/ml), Other Supplements

Antibody activators: ahCD3 Ab clone:OKT3 (1 ug/ml), ahCD28 Ab clone:CD28.2 (1 ug/ml), ahCD2 Ab clone: RPA-2.10 (1 ug/ml)

e. Expansion Stage (W4)

Base media: T Cell Medium. Contains: X-vivo15 serum-free medium (Lonza, Allendale N.J.), 5% (vol/vol) GemCell human serum antibody AB, (Gemini Bio Products, West Sacramento Calif.), 1% (vol/vol) Glutamax-100X (Gibco Life Technologies), 10 mM HEPES buffer (Corning), 1% (vol/vol) penicillin/streptomycin (Corning), 12.25 mM N-Acetyl-L-cysteine (Sigma)

Supplements: hIL7 (10 ng/ml), hIL15 (50 ng/ml)

Other key materials: 100 ng/ml α-Galactosylceramide (KRN7000) (Avanti Polar Lipids, SKU #867000P-1 mg), ahCD3 Ab clone:OKT3 (5 ug/ml), ahCD28 Ab clone:CD28.2 (5 ug/ml)

### IV. iNKT Cells

In particular embodiments, engineered iNKT cells of the disclosure are produced from other types of cells to facilitate their activity as iNKT cells. iNKT cells are a small subpopulation of αβ T lymphocytes that have several unique features that make them useful for off-the-shelf cellular therapy, including at least for cancer therapy. Non-iNKT cells are engineered to function as iNKT cells because of the following advantages of iNKT cells:

1) iNKT cells have the remarkable capacity to target multiple types of cancer independent of tumor antigen- and MHC-restrictions (Fujii et al., 2013). iNKT cells recognize glycolipid antigens presented by non-polymorphic CD1d, which frees them from MHC-restriction. Although the natural ligands of iNKT cells remain to be identified, it is suggested that iNKT cells can recognize certain conserved glycolipid antigens derived from many tumor tissues. iNKT cells can be stimulated through recognizing these glycolipid antigens that are either directly presented by CD1d⁺ tumor cells, or indirectly cross-presented by tumor infiltrating antigen-presenting cells (APCs) like macrophages or dendritic cells (DCs) in case of CD1d⁻ tumors. Thus, iNKT cells can respond to both CD1d⁺ and CD1d⁻ tumors.

2) iNKT cells can employ multiple mechanisms to attack tumor cells (Vivier et al., 2012; Fujii et al., 2013). iNKT cells can directly kill CD1d⁺ tumor cells through cytotoxicity, but their most potent anti-tumor activities come from their immune adjuvant effects. iNKT cells remain quiescent prior to stimulation, but after stimulation, they immediately produce large amounts of cytokines, mainly IFN-γ. IFN-γ activates NK cells to kill MHC-negative tumor target cells. Meanwhile, iNKT cells also activate DCs that then stimulate CTLs to kill MHC-positive tumor target cells. Therefore, iNKT cell-induced anti-tumor immunity can effectively target multiple types of cancer independent of tumor antigen- and MHC-restrictions, thereby effectively blocking tumor immune escape and minimizing the chance of tumor recurrence.

3) iNKT cells do not cause graft-versus-host disease (GvHD). Because iNKT cells do not recognize mismatched MHC molecules and protein autoantigens, these cells are not expected to cause GvHD. This notion is strongly supported by clinical data analyzing donor-derived iNKT cells in blood cancer patients receiving allogeneic bone marrow or peripheral blood stem cell transplantation. These clinical data showed that the levels of engrafted allogenic iNKT cells in patients correlated positively with graft-versus-leukemia effects and negatively with GvHD (Haraguchi et al., 2004; de Lalla et al., 2011).

4) iNKT cells can be engineered to avoid host-versus-graft (HvG) depletion. The availability of powerful gene-editing tools like the CRISPR-Cas9 system make it possible to genetically modify iNKT cells to make them resistant to host immune cell-targeted depletion: knockout of beta 2-microglobulin (B2M) gene will ablate HLA-I molecule expression on iNKT cells to avoid host CD8⁺ T cell-mediated killing; knockout of CIITA gene will ablate HLA-II molecule expression on iNKT cells to avoid CD4⁺ T cell-mediated killing. Both B2M and CIITA genes are approved good targets for the CRISPR-Cas9 system in human primary cells (Ren et al., 2017; Abrahimi et al., 2015). Ablation of HLA-I expression on iNKT cells may make them targets of host NK cells. However, iNKT cells seem to naturally resist allogenic NK cell killing. Nonetheless, if necessary, the concern can be addressed by delivering into iNKT cells an NK-inhibitory gene like HLA-E. Accordingly, embodiments of the disclosure relate to cells that lack B2M and/or CIITA genes.

5) iNKT cells have strong relevance to cancer. There is compelling evidence to suggest a significant role of iNKT cells in tumor surveillance in mice, in which iNKT cell defects predispose them to cancer and the adoptive transfer or stimulation of iNKT cells can provide protection against cancer (Vivier et al., 2012; Berzins et al., 2011). In humans, iNKT cell frequency is decreased in patients with solid tumors (including melanoma, colon, lung, breast, and head and neck cancers) and blood cancers (including leukemia, multiple myeloma, and myelodysplastic syndromes), while increased iNKT cell numbers are associated with a better prognosis (Berzins et al., 2011). There are also instances wherein the administration of α-GalCer-loaded DCs and ex vivo expanded autologous iNKT cells has led to promising clinical benefits in patients with lung cancer and head and neck cancer, although the increases of iNKT cells have been transient and the clinical benefits have been short-term, likely due to the limited number of iNKT cells used for transfer and the depletion of these cells thereafter (Fujii et al., 2012; Yamasaki et al., 2011). Therefore, it is plausible to propose that an “off-the-shelf” iNKT cellular product enabling the transfer into patients sufficient iNKT cells at multiple doses may provide patients with the best chance to exploit the full potential of iNKT cells to battle their diseases.

However, the development of an allogenic off-the-shelf iNKT cellular product is greatly hindered by their availability—these cells are of extremely low number and high variability in humans (˜0.001-1% in human blood), making it very difficult to grow therapeutic numbers of iNKT cells from blood cells of allogenic human donors. A novel method that can reliably generate homogenous population of iNKT cells at large quantity is thus key to developing an off-the-shelf iNKT cell therapy.

Given this lack of sufficient amounts of iNKT cells for clinical applications, embodiments of the disclosure encompass the engineering of non-iNKT cells such that the resultant engineered cell functions as an iNKT cell. In specific embodiments, the cells that function as iNKT cells are further modified to have one or more desired characteristics. In specific embodiments, non-iNKT cells are modified genetically through transduction of the non-iNKT cell to express an iNKT T cell receptor (TCR).

In embodiments of the disclosure, iNKT cells produced from other types of cells are engineered to have one or more characteristics to render them suitable for universal use. In specific embodiments, a cell is genetically modified to contain at least one exogenous invariant natural killer T cell receptor (iNKT TCR) nucleic acid molecule. In some embodiments, the cell is a hematopoietic stem cell. In some embodiments, the cell is a hematopoietic progenitor cell. In some embodiments, the cell is a human cell. In some embodiments, the cell is a CD34⁺ cell. In some embodiments, the cell is a human CD34+ cell. In some embodiments, the cell is a recombinant cell. In some embodiments, the cell is of a cultured strain.

In some embodiments, the iNKT TCR nucleic acid molecule is from a human invariant natural killer T cell. In some embodiments, the iNKT TCR nucleic acid molecule comprises one or more nucleic acid sequences obtained from a human iNKT TCR. In some embodiments, the iNKT TCR nucleic acid sequence can be obtained from any subset of iNKT cells, such as the CD4/DN/CD8 subsets or the subsets that produce Th1, Th2, or Th17 cytokines, and includes double negative iNKT cells. In some embodiments, the iNKT TCR nucleic acid sequence is obtained from an iNKT cell from a donor who had or has a cancer such as melanoma, kidney cancer, lung cancer, prostate cancer, breast cancer, lymphoma, leukemia, a hematological malignancy, and the like. In some embodiments, the iNKT TCR nucleic acid molecule has a TCR-alpha sequence from one iNKT cell and a TCR-beta sequence from a different iNKT cell. In some embodiments, the iNKT cell from which the TCR-alpha sequence is obtained and the iNKT cell from which the TCR-beta sequence is obtained are from the same donor. In some embodiments, the donor of the iNKT cell from which the TCR-alpha sequence is obtained is different from the donor of the iNKT cell from which the TCR-beta sequence is obtained. In some embodiments, the TCRalpha sequence and/or the TCR-beta sequence are codon optimized for expression. In some embodiments, the TCR-alpha sequence and/or the TCR-beta sequence are modified to encode a polypeptide having one or more amino acid substitutions, deletions, and/or truncations compared to the polypeptide encoded by the unmodified sequence. In some embodiments, the iNKT TCR nucleic acid molecule encodes a T cell receptor that recognizes alpha-galactosylceramide (alpha-GalCer) presented on CD1d. In some embodiments, the iNKT TCR nucleic acid molecule comprises one or more sequences selected from the group consisting of

(SEQ ID NO: 1)
gtgggcgatagaggttcagccttagggaggctgcattttggagctgggactcagctgattgtcatacctgacatc;
(SEQ ID NO: 2)
gccagcggtgatgctcggggggggggaaataccctctattttggaaaaggaagccggctcattgttgtagaggat;
(SEQ ID NO: 3)
gccagcggggggacagtccattctggaaatacgctctattttggagaaggaagccggctcattgttgtagaggat;
(SEQ ID NO: 4)
gccagcggtgatacgggacaaacaaacacagaagtcttctttggtaaaggaaccagactcacagttgtagaggat;
(SEQ ID NO: 5)
gccagcggtgaggggacagcaaacacagaagtcttctttggtaaaggaaccagactcacagttgtagaggat;
(SEQ ID NO: 6)
gccagcggtgaggcagggaacacagaagtcttctttggtaaaggaaccagactcacagttgtagaggat;
(SEQ ID NO: 7)
gtgagcgacagaggctcaaccctggggaggctatactttggaagaggaactcagttgactgtctggcctgatatccag;
(SEQ ID NO: 8)
agcagtgacctccgaggacagaacacagatacgcagtattttggcccaggcacccggctgacagtgctcgaggac;
(SEQ ID NO: 9)
agcagtgaattaaaggaaacaggggttcaagagacccagtacttcgggccaggcacgcggctcctggtgctcgaggac;
(SEQ ID NO: 10)
agcagtgtatctcagggcggcactgaagctttctttggacaaggcaccagactcacagttgtagaggac;
(SEQ ID NO: 11)
agcagtgtatctcagggcggcactgaagctttctttggacaaggcaccagactcacagttgtagaggac;
(SEQ ID NO: 12)
agcagtgaccggacaggcgtgaacactgaagctttctttggacaaggcaccagactcacagttgtagaggac;
(SEQ ID NO: 13)
agcagtgaaccggacagggggggggctgaagctttctttggacaaggcaccagactcacagttgtagaggac;
(SEQ ID NO: 14)
atgaaaaagcatctgacgaccttcttggtgattttgtggctttatttttatagggggaatggcaaaaaccaagtggagcagagtcctcagtccct
gatcatcctggagggaaagaactgcactcttcaatgcaattatacagtgagccccttcagcaacttaaggtggtataagcaagatactggga
gaggtcctgtttccctgacaatcatgactttcagtgagaacacaaagtcgaacggaagatatacagcaactctggatgcagacacaaagcaa
agctctctgcacatcacagcctcccagctcagcgattcagcctcctacatctgtgtggtgagcgacagaggctcaaccctggggaggctata
ctttggaagaggaactcagttgactgtctggcctgatatccagaaccctgaccctgccgtgtaccagctgagagactctaaatccagtgaca
agtctgtctgcctattcaccgattttgattctcaaacaaatgtgtcacaaagtaaggattctgatgtgtatatcacagacaaaactgtgctagaca
tgaggtctatggacttcaagagcaacagtgctgtggcctggagcaacaaatctgactttgcatgtgcaaacgccttcaacaacagcattattc
cagaagacaccttcttccccagcccagaaagttcctgtgatgtcaagctggtcgagaaaagctttgaaacagatacgaacctaaactttcaaa
acctgtcagtgattgggttccgaatcctcctcctgaaagtggccgggtttaatctgctcatgacgctgcggctgtggtccagctga;
(SEQ ID NO: 15)
atgaaaaagcatctgacaacattcctggtcattctgtggctgtacttctaccgaggcaacggcaaaaatcaggtggagcagtccccacagtc
cctgatcattctggaggggaagaactgcactctgcagtgtaattacaccgtgtctccctttagtaacctgcgctggtataaacaggacaccgg
acgaggacccgtgagcctgacaatcatgactttctcagagaacacaaagagcaatggacggtacaccgctacactggacgcagataccaa
acagagctccctgcacatcacagcatctcagctgtcagatagcgcctcctacatttgcgtggtctctgaccgagggagtaccctgggccgac
tgtattttggaagggggacccagctgacagtgtggcccgacatccagaacccagatcccgccgtctaccagctgcgcgacagcaagtcta
gtgataaaagcgtgtgcctgttcacagactttgattctcagactaatgtctctcagagtaaggacagtgacgtgtacattactgacaaaaccgt
cctggatatgaggagcatggacttcaagtcaaacagcgccgtggcttggtcaaacaagagcgacttcgcatgcgccaatgcttttaacaatt
caatcattccagaggataccttctttcctagcccagaatcaagctgtgacgtgaagctggtcgagaaaagtttcgaaactgataccaacctga
attttcagaacctgtctgtgatcggcttcagaatcctgctgctgaaggtcgccggctttaatctgctgatgacactgagactgtggtcctcttga;
(SEQ ID NO: 16)
atgactatcaggctcctctgctacatgggcttttattttctgggggcaggcctcatggaagctgacatctaccagaccccaagataccttgttat
agggacaggaaagaagatcactctggaatgttctcaaaccatgggccatgacaaaatgtactggtatcaacaagatccaggaatggaacta
cacctcatccactattcctatggagttaattccacagagaagggagatctttcctctgagtcaacagtctccagaataaggacggagcattttc
ccctgaccctggagtctgccaggccctcacatacctctcagtacctctgtgccagc,
(SEQ ID NO: 17)
atgaccatccggctgctgtgctacatgggcttctattttctgggggcaggcctgatggaagccgacatctaccagactcccagatacctggtc
atcggaaccgggaagaaaattacactggagtgttcccagacaatgggccacgataagatgtactggtatcagcaggaccctgggatggaa
ctgcacctgatccattactcctatggcgtgaactctaccgagaagggcgacctgagcagcgaatccaccgtctctcgaattaggacagagc
actttcctctgactctggaaagcgcccgaccaagtcatacatcacagtacctgtgcgctagc;
(SEQ ID NO: 18)
gtagcggttgggccccaagagacccagtacttcgggccaggcacgcggctcctggtgctc;
(SEQ ID NO: 19)
gtggcagtcggacctcaggagacccagtacttcggacccggcacccgcctgctggtgctg;
(SEQ ID NO: 20)
agtgggccagggtacgagcagtacttcgggccgggcaccaggctcacggtcaca;
(SEQ ID NO: 21)
tcaggacccggctacgagcagtatttcggccccggaactcggctgaccgtgacc;
(SEQ ID NO: 22)
agtccccaattaaacactgaagctttctttggacaaggcaccagactcacagttgta;
(SEQ ID NO: 23)
tctccacagctgaacaccgaggccttcttcgggcagggcacaaggcttaccgtggtg;
(SEQ ID NO: 24)
agtgaattgcgggcgctcgggcccagctcctataattcacccctccactttgggaacgggaccaggctcactgtgaca;
(SEQ ID NO: 25)
tccgaactccgagccctggggcctagctcctacaatagccccctgcactttggcaacggaaccaggctgacggtcacc;
(SEQ ID NO: 26)
agtgaacagg ggactactgcgggagctttctttggacaaggcaccagactcacagttgta;
(SEQ ID NO: 27)
tccgaacagggaaccacagcaggagccttcttcggtcagggaacaagactgacagtcgtg;
(SEQ ID NO: 28)
agtgagtcacgacatgcgacaggaaacaccatatattttggagagggaagttggctcactgttgta;
(SEQ ID NO: 29)
agcgagagcaggcacgcaaccgggaacaccatatactttggcgagggctcctggctgactgtggtg;
(SEQ ID NO: 30)
agtgtacccgggaacgacaggggcaatgaaaaactgattttggcagtggaacccagctctctgtcttg,
(SEQ ID NO: 31)
tccgtgcctggcaacgatagaggtaacgagaagctgtttttcggatccggcacacagctgtctgtcctg;
(SEQ ID NO: 32)
agtgaaggggggggccttaagctagccaaaaacattcagtacttcggcgccgggacccggctctcagtgctg;
(SEQ ID NO: 33)
agtgagggagggggactgaagctggctaagaatattcagtacttcggcgccggcactagactgtctgtgctg;
(SEQ ID NO: 34)
agtgaattcgcctcttcggtacgtggaaacaccatatattttggagagggaagttggctcactgttgta;
(SEQ ID NO: 35)
tctgagttcgcgagcagcgtccggggtaataccatttacttcggggaaggcagctggctgaccgtggtg;
(SEQ ID NO: 36)
agtgcggcattaggccgggagacccagtacttcgggccaggcacgcggctcctggtgctc;
(SEQ ID NO: 37)
tctgcagcccttggccgagagactcagtacttcggccctggcacaagactgctcgtgctc;
(SEQ ID NO: 38)
agtgcctccgggggtgaatcctacgagcagtacttcgggccgggcaccaggctcacggtcaca;
(SEQ ID NO: 39)
agcgcctccggaggagagtcatacgaacagtatttcggccctggcacacgcctcactgtgacc;
(SEQ ID NO: 40)
agcggtcgggtctcggggggcgattccctcatagcgtttctaggccaagagacccagtacttcgggccaggcacgcggctcctggtgctc;
(SEQ ID NO: 41)
tcaggacgagtgtccggaggggatagcctcatcgcatttctggggcaggaaactcagtacttcggacccggaacacgcctcctggtgctg;
(SEQ ID NO: 42)
agtgtacccgggaacgacaggggcaatgaaaaactgttttttggcagtggaacccagctctctgtcttg;
SEQ ID NO: 43)
tccgtgcctggcaacgatagaggtaacgagaagctgtttttcggatccggcacacagctgtctgtcctg;
(SEQ ID NO: 44)
gaggacctgaacaaggtgttcccacccgaggtcgctgtgtttgagccatcagaagcagagatctcccacacccaaaaggccacactggtg
tgcctggccacaggcttcttccctgaccacgtggagctgagctggtgggtgaatgggaaggaggtgcacagtggggtcagcacggaccc
gcagcccctcaaggagcagcccgccctcaatgactccagatactgcctgagcagccgcctgagggtctcggccaccttctggcagaacc
cccgcaaccacttccgctgccaagtccagttctacgggctctcggagaatgacgagtggacccaggatagggccaaacccgtcacccag
atcgtcagcgccgaggcctggggtagagcagactgtggctttacctcggtgtcctaccagcaaggggtcctgtctgccaccatcctctatga
gatcctgctagggaaggccaccctgtatgctgtgctggtcagcgcccttgtgttgatggccatggtcaagagaaaggatttctga;
AND
(SEQ ID NO: 45)
gaggacctgaataaggtgttcccccctgaggtggctgtctttgaaccaagtgaggcagaaatttcacatacacagaaagccaccctggtgtg
cctggctaccggcttctttcccgatcacgtggagctgagctggtgggtcaacggcaaggaagtgcatagcggagtctccacagacccaca
gcccctgaaagagcagcctgctctgaatgattccagatactgcctgtctagtagactgcgggtgtctgccaccttctggcagaacccaagga
atcatttcagatgtcaggtgcagttttatggcctgagcgagaacgatgaatggactcaggacagggctaagccagtgacccagatcgtcag
cgcagaggcctggggaagagcagactgcgggtttacaagcgtgagctatcagcagggcgtcctgagcgccacaatcctgtacgaaattct
gctgggaaaggccactctgtatgctgtgctggtctccgctctggtgctgatggcaatggtcaagcggaaagatttctga.

In some embodiments, the iNKT TCR nucleic acid molecule encodes a polypeptide comprising an amino acid sequence selected from the group consisting of: MKKHLTTFLVILWLYFYRGNGKNQVEQSPQSLIILEGKNCTLQCNYTVSPFSNLRWYKQ DTGRGPVSLTIMTFSENTKSNGRYTATLDADTKQSSLHITASQLSDSASYICVVSDRGST LGRLYFGRGTQLTVWPDIQNPDPAVYQLRDSKSSDKSVCLFTDFDSQTNVSQSKDSDVY ITDKTVLDMRSMDFKSNSAVAWSNKSDFACANAFNNSIIPEDTFFPSPESSCDVKLVEKS FETDTNLNFQNLSVIGFRILLLKVAGFNLLMTLRLWSS (SEQ ID NO:46); MTIRLLCYMGFYFLGAGLMEADIYQTPRYLVIGTGKKITLECSQTMGHDKMYWYQQDP GMELHLIHYSYGVNSTEKGDLSSESTVSRIRTEHFPLTLESARPSHTSQYLCAS (SEQ ID NO:47); VAVGPQETQYFGPGTRLLVL (SEQ ID NO:48); SGPGYEQYFGPGTRLTVT (SEQ ID NO:49); SPQLNTEAFFGQGTRLTVV (SEQ ID NO:50); SELRALGPSSYNSPLHFGNGTRLTVT (SEQ ID NO:51); SEQGTTAGAFFGQGTRLTVV (SEQ ID NO:52); SESRHATGNTIYFGEGSWLTVV (SEQ ID NO:53); SVPGNDRGNEKLFFGSGTQLSVL (SEQ ID NO:54); SEGGGLKLAKNIQYFGAGTRLSVL (SEQ ID NO:55); SEFASSVRGNTIYFGEGSWLTVV (SEQ ID NO:56); SAALGRETQYFGPGTRLLVL (SEQ ID NO:57); SASGGESYEQYFGPGTRLTVT (SEQ ID NO:58); SGRVSGGDSLIAFLGQETQYFGPGTRLLVL (SEQ ID NO:59); SVPGNDRGNEKLFFGSGTQLSVL (SEQ ID NO:60); and EDLNKVFPPEVAVFEPSEAEISHTQKATLVCLATGFFPDHVELSWWVNGKEVHSGVSTD PQPLKEQPALNDSRYCLSSRLRVSATFWQNPRNHFRCQVQFYGLSENDEWTQDRAKPV TQIVSAEAWGRADCGFTSVSYQQGVLSATILYEILLGKATLYAVLVSALVLMAMVKRK DF (SEQ ID NO:61). In some embodiments, the engineered cell lacks exogenous oncogenes, such as Oct4, Sox2, Klf, c-Myc, and the like.

In some embodiments, the engineered cell is a functional iNKT cell. In some embodiments, the engineered cell is capable of producing one or more cytokines and/or chemokines such as IFN-gamma, TNF-alpha, TGF-beta, GM-CSF, IL-2, IL-4, IL-5, IL-6, IL-10, IL-13, IL-15, IL-17, IL-21, RANTES, Eotaxin, MIP-1-alpha, MIP-1-beta, and the like. In some embodiments, the engineered cell is capable of producing IL-15.

Donor HSPCs can be obtained from the bone marrow, peripheral blood, amniotic fluid, or umbilical cord blood of a donor. The donor can be an autologous donor, i.e., the subject to be treated with the HSPC-iNKT cells, or an allogenic donor, i.e., a donor who is different from the subject to be treated with the HSPC-iNKT cells. In embodiments where the donor is an allogenic donor, the tissue (HLA) type of the allogenic donor preferably matches that of the subject being treated with the HSPC-iNKT cells derived from the donor HSPCs.

According to the present disclosure, an HSPC is transduced with one or more exogenous iNKT TCR nucleic acid molecules. As used herein, an “iNKT TCR nucleic acid molecule” includes a nucleic acid molecule that encodes an alpha chain of an iNKT T cell receptor (TCR-alpha-), a beta chain of an iNKT T cell receptor (TCR-beta), or both. As used herein, an “iNKT T cell receptor” is one that is expressed in an iNKT cell and recognizes alpha-GalCer presented on CD1d. TCR-alpha and TCR-beta sequences of iNKT TCRs can be cloned and/or recombinantly engineered using methods in the art. For example, an iNKT cell can be obtained from a donor and the TCR-alpha and -beta genes of the iNKT cell can be cloned as described herein. The iNKT TCR to be cloned can be obtained from any mammalian including humans, non-human primates such monkeys, mice, rats, hamsters, guinea pigs, and other rodents, rabbits, cats, dogs, horses, bovines, sheep, goat, pigs, and the like. In some embodiments, the iNKT TCR to be cloned is a human iNKT TCR. In some embodiments, the iNKT TCR clone comprises human iNKT TCR sequences and non-human iNKT TCR sequences.

In some embodiments, the cloned TCR can have a TCR-alpha chain from one iNKT cell and a TCR-beta chain from a different iNKT cell. In some embodiments, the iNKT cell from which the TCR-alpha chain is obtained and the iNKT cell from which the TCR-beta chain is obtained are from the same donor. In some embodiments, the donor of the iNKT cell from which the TCR-alpha chain is obtained is different from the donor of the iNKT cell from which the TCR-beta chain is obtained. In some embodiments, the sequence encoding the TCR-alpha chain and/or the sequence encoding the TCR-beta chain of a TCR clone is modified. In some embodiments, the modified sequence may encode the same polypeptide sequence as the unmodified TCR clone, e.g., the sequence is codon optimized for expression. In some embodiments, the modified sequence may encode a polypeptide that has a sequence that is different from the unmodified TCR clone, e.g., the modified sequence encodes a polypeptide sequence having one or more amino acid substitutions, deletions, and/or truncations.

In particular embodiments, iNKT cells produced from HSPCs cells are further modified to have one or more characteristics, including to render the cells suitable for allogeneic use or more suitable for allogeneic use than if the cells were not further modified to have one or more characteristics. The present disclosure encompasses iNKT cells that are suitable for allogeneic use, if desired. In some embodiments, the iNKT cells are non-alloreactive and express an exogenous iNTK TCR. These cells are useful for “off the shelf” cell therapies and do not require the use of the patient's own iNKT or other cells. Therefore, the current methods provide for a more cost-effective, less labor-intensive cell immunotherapy.

In some embodiments, iNKT cells are engineered to be HLA-negative to achieve safe and successful allogeneic engraftment without causing graft-versus-host disease (GvHD) and being rejected by host immune cells (HvG rejection). In specific embodiments, allogeneic HSC-iNKT cells do not express endogenous TCRs and do not cause GvHD, because the expression of the transgenic iNKT TCR gene blocks the recombination of endogenous TCRs through allelic exclusion. In particular embodiments, allogeneic iNKT cells do not express HLA-I and/or HLA-II molecules on cell surface and resist host CD8⁺ and CD4⁺ T cell-mediated allograft depletion and sr39TK immunogen-targeting depletion.

Thus, in certain embodiments the engineered iNKT cells do not express surface HLA-I or -II molecules, achieved through disruption of genes encoding proteins relevant to HLA-I/II expression, including but not limited to beta-2-microglobulin (B2M), major histocompatibility complex II transactivator (CIITA), or HLA-I/II molecules. In some cases, the HLA-I or HLA-II are not expressed on the surface of iNKT cells because the cells were manipulated by gene editing, which may or may not involve CRISPR-Cas9.

In cases wherein the iNKT cells have been modified to exhibit one or more characteristics of any kind, the iNKT cells may comprise nucleic acid sequences from a recombinant vector that was introduced into the cells. The vector may be a non-viral vector, such as a plasmid, or a viral vector, such as a lentivirus, a retrovirus, an adeno-associated virus (AAV), a herpesvirus, or adenovirus.

The iNKT cells of the disclosure may or may not have been exposed to one or more certain conditions before, during, or after their production. In specific cases, the cells are not or were not exposed to media that comprises animal serum. The cells may be frozen. The cells may be present in a solution comprising dextrose, one or more electrolytes, albumin, dextran, and/or DMSO. Any solution in which the cells are present may be a solution that is sterile, nonpyogenic, and isotonic. The cells may have been activated and expanded by any suitable manner, such as activated with alpha-galactosylceramide (α-GC), for example.

Aspects of the disclosure relate to engineered iNKT cells. In some embodiments, the cell comprises a genomic mutation. In some embodiments, the genomic mutation comprises a mutation of one or more endogenous genes in the cell's genome, wherein the one or more endogenous genes comprise the B2M, CIITA, TRAC, TRBC1, or TRBC2 gene. In some embodiments, the mutation comprises a loss of function mutation. In some embodiments, the inhibitor is an expression inhibitor. In some embodiments, the inhibitor comprises an inhibitory nucleic acid. In some embodiments, the inhibitory nucleic acid comprises one or more of a siRNA, shRNA, miRNA, or an antisense molecule. In some embodiments, the cells comprise an activity inhibitor. In some embodiments, following modification the cell is deficient in any detectable expression of one or more of B2M, CIITA, TRAC, TRBC1, or TRBC2 proteins. In some embodiments, the cell comprises an inhibitor or genomic mutation of B2M. In some embodiments, the cell comprises an inhibitor or genomic mutation of CIITA. In some embodiments, the cell comprises an inhibitor or genomic mutation of TRAC. In some embodiments, the cell comprises an inhibitor or genomic mutation of TRBC1. In some embodiments, the cell comprises an inhibitor or genomic mutation of TRBC2. In some embodiments, at least 90% of the genomic DNA encoding B2M, CIITA, TRAC, TRBC1, and/or TRBC2 is deleted. In some embodiments, at least or at most 5, 10, 20, 30, 40, 50, 60, 70, 80, 90, 95, 99, or 100% (or any range derivable therein) of the genomic DNA encoding B2M, CIITA, TRAC, TRBC1, and/or TRBC2 is deleted. In other embodiments, a deletion, insertion, and/or substitution is made in the genomic DNA. In some embodiments, the cell is a progeny of the human stem or progenitor cell.

The iNKT cells that are modified to be HLA-negative may be genetically modified by any suitable manner. The genetic mutations of the disclosure, such as those in the CIITA and/or B2M genes can be introduced by methods known in the art. In certain embodiments, engineered nucleases may be used to introduce exogenous nucleic acid sequences for genetic modification of any cells referred to herein. Genome editing, or genome editing with engineered nucleases (GEEN) is a type of genetic engineering in which DNA is inserted, replaced, or removed from a genome using artificially engineered nucleases, or “molecular scissors.” The nucleases create specific double-stranded break (DSBs) at desired locations in the genome, and harness the cell's endogenous mechanisms to repair the induced break by natural processes of homologous recombination (HR) and nonhomologous end-joining (NHEJ). Non-limiting engineered nucleases include: Zinc finger nucleases (ZFNs), Transcription Activator-Like Effector Nucleases (TALENs), the CRISPR/Cas9 system, and engineered meganuclease re-engineered homing endonucleases. Any of the engineered nucleases known in the art can be used in certain aspects of the methods and compositions.

The engineered iNKT cells may be modified using methods that employ RNA interference. It is commonly practiced in genetic analysis that in order to understand the function of a gene or a protein function one interferes with it in a sequence-specific way and monitors its effects on the organism. However, in some organisms it is difficult or impossible to perform site-specific mutagenesis, and therefore more indirect methods have to be used, such as silencing the gene of interest by short RNA interference (siRNA). However, gene disruption by siRNA can be variable and incomplete. Genome editing with nucleases such as ZFN is different from siRNA in that the engineered nuclease is able to modify DNA-binding specificity and therefore can in principle cut any targeted position in the genome, and introduce modification of the endogenous sequences for genes that are impossible to specifically target by conventional RNAi. Furthermore, the specificity of ZFNs and TALENs are enhanced as two ZFNs are required in the recognition of their portion of the target and subsequently direct to the neighboring sequences.

Meganucleases may be employed to modify engineered iNKT cells. Meganucleases, found commonly in microbial species, have the unique property of having very long recognition sequences (>14 bp) thus making them naturally very specific. This can be exploited to make site-specific DSB in genome editing; however, the challenge is that not enough meganucleases are known, or may ever be known, to cover all possible target sequences. To overcome this challenge, mutagenesis and high throughput screening methods have been used to create meganuclease variants that recognize unique sequences. Others have been able to fuse various meganucleases and create hybrid enzymes that recognize a new sequence. Yet others have attempted to alter the DNA interacting aminoacids of the meganuclease to design sequence specific meganucelases in a method named rationally designed meganuclease (U.S. Pat. No. 8,021,867, incorporated herein by reference). Meganuclease have the benefit of causing less toxicity in cells compared to methods such as ZFNs likely because of more stringent DNA sequence recognition; however, the construction of sequence specific enzymes for all possible sequences is costly and time consuming as one is not benefiting from combinatorial possibilities that methods such as ZFNs and TALENs utilize. So there are both advantages and disadvantages.

As opposed to meganucleases, the concept behind ZFNs and TALENs is more based on a non-specific DNA cutting enzyme which would then be linked to specific DNA sequence recognizing peptides such as zinc fingers and transcription activator-like effectors (TALEs). One way was to find an endonuclease whose DNA recognition site and cleaving site were separate from each other, a situation that is not common among restriction enzymes. Once this enzyme was found, its cleaving portion could be separated which would be very non-specific as it would have no recognition ability. This portion could then be linked to sequence recognizing peptides that could lead to very high specificity. An example of a restriction enzyme with such properties is FokI. Additionally FokI has the advantage of requiring dimerization to have nuclease activity and this means the specificity increases dramatically as each nuclease partner would recognize a unique DNA sequence. To enhance this effect, FokI nucleases have been engineered that can only function as heterodimers and have increased catalytic activity. The heterodimer functioning nucleases would avoid the possibility of unwanted homodimer activity and thus increase specificity of the DSB.

Although the nuclease portion of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers typically happen in repeats that are 3 bp apart and are found in diverse combinations in a variety of nucleic acid interacting proteins such as transcription factors. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Zinc fingers have been more established in these terms and approaches such as modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries among other methods have been used to make site specific nucleases.

Thus, embodiments of the disclosure may or may not include the targeting of endogenous sequences to reduce or knock out expression of one or more certain endogenous sequences. In specific embodiments, disruption of one or more of the following genes may block the rearrangement of endogenous TCRs. To produce guide RNAs or siRNAs, for example, to target the noted genes below, their sequences are provided below as examples:

B-2 microglobin (B2M) (also known as IMD43) is located at 15q21.1 and has the following mRNA sequence:

(SEQ ID NO: 62)
agtggaggcgtcgcgctggcgggcattcctgaagctgacagcattcgggccgagatgtctcgctccgtggccttagctgtgctcgcgctac
tctctctttctggcctggaggctatccagcgtactccaaagattcaggtttactcacgtcatccagcagagaatggaaagtcaaatttcctgaat
tgctatgtgtctgggtttcatccatccgacattgaagttgacttactgaagaatggagagagaattgaaaaagtggagcattcagacttgtctttc
agcaaggactggtctttctatctcttgtactacactgaattcacccccactgaaaaagatgagtatgcctgccgtgtgaaccatgtgactttgtc
acagcccaagatagttaagtggggtaagtcttacattcttttgtaagctgctgaaagttgtgtatgagtagtcatatcataaagctgctttgatata
aaaaaggtctatggccatactaccctgaatgagtcccatcccatctgatataaacaatctgcatattgggattgtcagggaatgttcttaaagat
cagattagtggcacctgctgagatactgatgcacagcatggtttctgaaccagtagtttccctgcagttgagcagggagcagcagcagcact
tgcacaaatacatatacactcttaacacttcttacctactggcttcctctagcttttgtggcagcttcaggtatatttagcactgaacgaacatctca
agaaggtataggcctttgtttgtaagtcctgctgtcctagcatcctataatcctggacttctccagtactttctggctggattggtatctgaggcta
gtaggaagggcttgttcctgctgggtagctctaaacaatgtattcatgggtaggaacagcagcctattctgccagccttatttctaaccattttag
acatttgttagtacatggtattttaaaagtaaaacttaatgtcttccttattttctccactgtctttttcatagatcgagacatgtaagcagcatcatgg
aggtaagtattgaccttgagaaaatgatttgtttcactgtcctgaggactatttatagacagctctaacatgataaccctcactatgtggagaac
attgacagagtaacattttagcagggaaagaagaatcctacagggtcatgttcccttctcctgtggagtggcatgaagaaggtgtatggcccc
aggtatggccatattactgaccctctacagagagggcaaaggaactgccagtatggtattgcaggataaaggcaggtggttacccacattac
ctgcaaggctttgatctttcttctgccatttccacattggacatctctgctgaggagagaaaatgaaccactcttttcctttgtataatgttgttttatt
cttcagacagaagagaggagttatacagctctgcagacatcccattcctgtatggggactgtgtttgcctcttagaggttcccaggccactag
aggagataaagggaaacagattgttataacttgatataatgatactataatagatgtaactacaaggagctccagaagcaagagagaggga
ggaacttggacttctctgcatctttagttggagtccaaaggcttttcaatgaaattctactgcccagggtacattgatgctgaaaccccattcaaa
tctcctgttatattctagaacagggaattgatttgggagagcatcaggaaggtggatgatctgcccagtcacactgttagtaaattgtagagcc
aggacctgaactctaatatagtcatgtgttacttaatgacggggacatgttctgagaaatgcttacacaaacctaggtgttgtagcctactacac
gcataggctacatggtatagcctattgctcctagactacaaacctgtacagcctgttactgtactgaatactgtgggcagttgtaacacaatggt
aagtatttgtgtatctaaacatagaagttgcagtaaaaatatgctattttaatcttatgagaccactgtcatatatacagtccatcattgaccaaaac
atcatatcagcattttttcttctaagattttgggagcaccaaagggatacactaacaggatatactctttataatgggtttggagaactgtctgcag
ctacttcttttaaaaaggtgatctacacagtagaaattagacaagtttggtaatgagatctgcaatccaaataaaataaattcattgctaaccttttt
cttttcttttcaggtttgaagatgccgcatttggattggatgaattccaaattctgcttgcttgctttttaatattgatatgcttatacacttacactttat
gcacaaaatgtagggttataataatgttaacatggacatgatcttctttataattctactttgagtgctgtctccatgtttgatgtatctgagcaggtt
gctccacaggtagctctaggagggctggcaacttagaggtggggagcagagaattctcttatccaacatcaacatcttggtcagatttgaact
cttcaatctcttgcactcaaagcttgttaagatagttaagcgtgcataagttaacttccaatttacatactctgcttagaatttgggggaaaatttag
aaatataattgacaggattattggaaatttgttataatgaatgaaacattttgtcatataagattcatatttacttcttatacatttgataaagtaaggc
atggttgtggttaatctggtttatttttgttccacaagttaaataaatcataaaacttga.

Human class II major histocompatibility complex transactivator (CIITA) gene is located at 16p13.13 with an mRNA sequence:

(SEQ ID NO: 63)
ggttagtgatgaggctagtgatgaggctgtgtgcttctgagctgggca
tccgaaggcatccttggggaagctgagggcacgaggaggggctgccag
actccgggagctgctgcctggctgggattcctacacaatgcgttgcct
ggctccacgccctgctgggtcctacctgtcagagccccaaggcagctc
acagtgtgccaccatggagttggggcccctagaaggtggctacctgga
gcttcttaacagcgatgctgaccccctgtgcctctaccacttctatga
ccagatggacctggctggagaagaagagattgagctctactcagaacc
cgacacagacaccatcaactgcgaccagttcagcaggctgttgtgtga
catggaaggtgatgaagagaccagggaggcttatgccaatatcgcgga
actggaccagtatgtcttccaggactcccagctggagggcctgagcaa
ggacattttcaagcacataggaccagatgaagtgatcggtgagagtat
ggagatgccagcagaagttgggcagaaaagtcagaaaagacccttccc
agaggagcttccggcagacctgaagcactggaagccagctgagccccc
cactgtggtgactggcagtctcctagtgggaccagtgagcgactgctc
caccctgccctgcctgccactgcctgcgctgttcaaccaggagccagc
ctccggccagatgcgcctggagaaaaccgaccagattcccatgccttt
ctccagttcctcgttgagctgcctgaatctccctgagggacccatcca
gtttgtccccaccatctccactctgccccatgggctctggcaaatctc
tgaggctggaacaggggtctccagtatattcatctaccatggtgaggt
gccccaggccagccaagtaccccctcccagtggattcactgtccacgg
cctcccaacatctccagaccggccaggctccaccagccccttcgctcc
atcagccactgacctgcccagcatgcctgaacctgccctgacctcccg
agcaaacatgacagagcacaagacgtcccccacccaatgcccggcagc
tggagaggtctccaacaagcttccaaaatggcctgagccggtggagca
gttctaccgctcactgcaggacacgtatggtgccgagcccgcaggccc
ggatggcatcctagtggaggtggatctggtgcaggccaggctggagag
gagcagcagcaagagcctggagcgggaactggccaccccggactgggc
agaacggcagctggcccaaggaggcctggctgaggtgctgttggctgc
caaggagcaccggcggccgcgtgagacacgagtgattgctgtgctggg
caaagctggtcagggcaagagctattgggctggggcagtgagccgggc
ctgggcttgtggccggcttccccagtacgactttgtcttctctgtccc
ctgccattgcttgaaccgtccgggggatgcctatggcctgcaggatct
gctcttctccctgggcccacagccactcgtggcggccgatgaggtttt
cagccacatcttgaagagacctgaccgcgttctgctcatcctagacgg
cttcgaggagctggaagcgcaagatggcttcctgcacagcacgtgcgg
accggcaccggcggagccctgctccctccgggggctgctggccggcct
tttccagaagaagctgctccgaggttgcaccctcctcctcacagcccg
gccccggggccgcctggtccagagcctgagcaaggccgacgccctatt
tgagctgtccggcttctccatggagcaggcccaggcatacgtgatgcg
ctactttgagagctcagggatgacagagcaccaagacagagccctgac
gctcctccgggaccggccacttcttctcagtcacagccacagccctac
tttgtgccgggcagtgtgccagctctcagaggccctgctggagcttgg
ggaggacgccaagctgccctccacgctcacgggactctatgtcggcct
gctgggccgtgcagccctcgacagcccccccggggccctggcagagct
ggccaagctggcctgggagctgggccgcagacatcaaagtaccctaca
ggaggaccagttcccatccgcagacgtgaggacctgggcgatggccaa
aggcttagtccaacacccaccgcgggccgcagagtccgagctggcctt
ccccagcttcctcctgcaatgcttcctgggggccctgtggctggctct
gagtggcgaaatcaaggacaaggagctcccgcagtacctagcattgac
cccaaggaagaagaggccctatgacaactggctggagggcgtgccacg
ctttctggctgggctgatcttccagcctcccgcccgctgcctgggagc
cctactcgggccatcggcggctgcctcggtggacaggaagcagaaggt
gcttgcgaggtacctgaagcggctgcagccggggacactgcgggcgcg
gcagctgctggagctgctgcactgcgcccacgaggccgaggaggctgg
aatttggcagcacgtggtacaggagctccccggccgcctctcttttct
gggcacccgcctcacgcctcctgatgcacatgtactgggcaaggcctt
ggaggcggcgggccaagacttctccctggacctccgcagcactggcat
ttgcccctctggattggggagcctcgtgggactcagctgtgtcacccg
tttcagggctgccttgagcgacacggtggcgctgtgggagtccctgca
gcagcatggggagaccaagctacttcaggcagcagaggagaagttcac
catcgagcctttcaaagccaagtccctgaaggatgtggaagacctggg
aaagcttgtgcagactcagaggacgagaagttcctcggaagacacagc
tggggagctccctgctgttcgggacctaaagaaactggagtttgcgct
gggccctgtctcaggcccccaggctttccccaaactggtgcggatcct
cacggccttttcctccctgcagcatctggacctggatgcgctgagtga
gaacaagatcggggacgagggtgtctcgcagctctcagccaccttccc
ccagctgaagtccttggaaaccctcaatctgtcccagaacaacatcac
tgacctgggtgcctacaaactcgccgaggccctgccttcgctcgctgc
atccctgctcaggctaagcttgtacaataactgcatctgcgacgtggg
agccgagagcttggctcgtgtgcttccggacatggtgtccctccgggt
gatggacgtccagtacaacaagttcacggctgccggggcccagcagct
cgctgccagccttcggaggtgtcctcatgtggagacgctggcgatgtg
gacgcccaccatcccattcagtgtccaggaacacctgcaacaacagga
ttcacggatcagcctgagatgatcccagctgtgctctggacaggcatg
ttctctgaggacactaaccacgctggaccttgaactgggtacttgtgg
acacagctcttctccaggctgtatcccatgagcctcagcatcctggca
cccggcccctgctggttcagggttggcccctgcccggctgcggaatga
accacatcttgctctgctgacagacacaggcccggctccaggctcctt
tagcgcccagttgggtggatgcctggtggcagctgcggtccacccagg
agccccgaggccttctctgaaggacattgcggacagccacggccaggc
cagagggagtgacagaggcagccccattctgcctgcccaggcccctgc
caccctggggagaaagtacttctttttttttatttttagacagagtct
cactgttgcccaggctggcgtgcagtggtgcgatctgggttcactgca
acctccgcctcttgggttcaagcgattcttctgcttcagcctcccgag
tagctgggactacaggcacccaccatcatgtctggctaatttttcatt
tttagtagagacagggttttgccatgttggccaggctggtctcaaact
cttgacctcaggtgatccacccacctcagcctcccaaagtgctgggat
tacaagcgtgagccactgcaccgggccacagagaaagtacttctccac
cctgctctccgaccagacaccttgacagggcacaccgggcactcagaa
gacactgatgggcaacccccagcctgctaattccccagattgcaacag
gctgggcttcagtggcagctgcttttgtctatgggactcaatgcactg
acattgttggccaaagccaaagctaggcctggccagatgcaccagccc
ttagcagggaaacagctaatgggacactaatggggcggtgagagggga
acagactggaagcacagcttcatttcctgtgtcttttttcactacatt
ataaatgtctctttaatgtcacaggcaggtccagggtttgagttcata
ccctgttaccattttggggtacccactgctctggttatctaatatgta
acaagccaccccaaatcatagtggcttaaaacaacactcacattta.

Human T cell receptor alpha chain (TRAC) mRNA sequence is as follows:

(SEQ ID NO: 64)
ttttgaaacccttcaaaggcagagacttgtccagcctaacctgcctgctgctcctagctcctgaggctcagggcccttggcttctgtccgctct
gctcagggccctccagcgtggccactgctcagccatgctcctgctgctcgtcccagtgctcgaggtgatttttaccctgggaggaaccaga
gcccagtcggtgacccagcttggcagccacgtctctgtctctgaaggagccctggttctgctgaggtgcaactactcatcgtctgttccacca
tatctcttctggtatgtgcaataccccaaccaaggactccagcttctcctgaagtacacatcagcggccaccctggttaaaggcatcaacggtt
ttgaggctgaatttaagaagagtgaaacctccttccacctgacgaaaccctcagcccatatgagcgacgcggctgagtacttctgtgctgtga
gtgatctcgaaccgaacagcagtgcttccaagataatctttggatcagggaccagactcagcatccggccaaatatccagaaccctgaccct
gccgtgtaccagctgagagactctaaatccagtgacaagtctgtctgcctattcaccgattttgattctcaaacaaatgtgtcacaaagtaagg
attctgatgtgtatatcacagacaaaactgtgctagacatgaggtctatggacttcaagagcaacagtgctgtggcctggagcaacaaatctg
actttgcatgtgcaaacgccttcaacaacagcattattccagaagacaccttcttccccagcccagaaagttcctgtgatgtcaagctggtcga
gaaaagctttgaaacagatacgaacctaaactttcaaaacctgtcagtgattgggttccgaatcctcctcctgaaagtggccgggtttaatctg
ctcatgacgctgcggctgtggtccagctgagatctgcaagattgtaagacagcctgtgctccctcgctccttcctctgcattgcccctcttctcc
ctctccaaacagagggaactctcctacccccaaggaggtgaaagctgctaccacctctgtgcccccccggtaatgccaccaactggatcct
acccgaatttatgattaagattgctgaagagctgccaaacactgctgccaccccctctgttcccttattgctgcttgtcactgcctgacattcacg
gcagaggcaaggctgctgcagcctcccctggctgtgcacattccctcctgctccccagagactgcctccgccatcccacagatgatggatc
ttcagtgggttctcttgggctctaggtcctggagaatgttgtgaggggtttattatttttaatagtgttcataaagaaatacatagtattcttcttctca
agacgtggggggaaattatctcattatcgaggccctgctatgctgtgtgtctgggcgtgttgtatgtcctgctgccgatgccttcattaaaatga
tttggaa.

Human T cell receptor beta chain (TRBC1) mRNA sequence is as follows:

(SEQ ID NO: 65)
tgcatcctagggacagcatagaaaggaggggcaaagtggagagagagcaacagacactgggatggtgaccccaaaacaatgagggcc
tagaatgacatagttgtgcttcattacggcccattcccagggctctctctcacacacacagagcccctaccagaaccagacagctctcagag
caaccctggctccaacccctcttccctttccagaggacctgaacaaggtgttcccacccgaggtcgctgtgtttgagccatcagaagcagag
atctcccacacccaaaaggccacactggtgtgcctggccacaggcttcttccccgaccacgtggagctgagctggtgggtgaatgggaag
gaggtgcacagtggggtcagcacggacccgcagcccctcaaggagcagcccgccctcaatgactccagatactgcctgagcagccgcc
tgagggtctcggccaccttctggcagaacccccgcaaccacttccgctgtcaagtccagttctacgggctctcggagaatgacgagtggac
ccaggatagggccaaacccgtcacccagatcgtcagcgccgaggcctggggtagagcaggtgagtggggcctggggagatgcctgga
ggagattaggtgagaccagctaccagggaaaatggaaagatccaggtagcagacaagactagatccaaaaagaaaggaaccagcgcac
accatgaaggagaattgggcacctgtggttcattcttctcccagattctcagcccaacagagccaagcagctgggtcccctttctatgtggcct
gtgtaactctcatctgggtggtgccccccatccccctcagtgctgccacatgccatggattgcaaggacaatgtggctgacatctgcatggca
gaagaaaggaggtgctgggctgtcagaggaagctggtctgggcctgggagtctgtgccaactgcaaatctgactttacttttaattgcctatg
aaaataaggtctctcatttattttcctctccctgctttctttcagactgtggctttacctcgggtaagtaagcccttccttttcctctccctctctcatgg
ttcttgacctagaaccaaggcatgaagaactcacagacactggagggtggagggtgggagagaccagagctacctgtgcacaggtaccc
acctgtccttcctccgtgccaacagtgtcctaccagcaaggggtcctgtctgccaccatcctctatgagatcctgctagggaaggccaccctg
tatgctgtgctggtcagcgcccttgtgttgatggccatggtaagcaggagggcaggatggggccagcaggctggaggtgacacactgaca
ccaagcacccagaagtatagagtccctgccaggattggagctgggcagtagggagggaagagatttcattcaggtgcctcagaagataac
ttgcacctctgtaggatcacagtggaagggtcatgctgggaaggagaagctggagtcaccagaaaacccaatggatgttgtgatgagcctt
actatttgtgtggtcaatgggccctactactttctctcaatcctcacaactcctggctcttaataacccccaaaactttctcttctgcaggtcaaga
gaaaggatttctgaaggcagccctggaagtggagttaggagcttctaacccgtcatggtttcaatacacattcttcttttgccagcgcttctgaa
gagctgctctcacctctctgcatcccaatagatatccccctatgtgcatgcacacctgcacactcacggctgaaatctccctaacccaggggg
accttagcatgcctaagtgactaaaccaataaaaatgttctggtctggcctgactctgacttgtgaatgtctggatagctccttggctgtctctga
actccctgtgactctccccattcagtcaggatagaaacaagaggtattcaaggaaaatgcagactcttcacgtaagagggatgaggggccc
accttgagatcaatagcag.

Human TRBC2 T cell receptor beta constant 2 (TCRB2) sequence is as follows:

(SEQ ID NO: 66)
atggcgtagtccccaaagaacgaggacctagtaacataattgtgcttcattatggtcctttcccggccttctctctcacacatacacagagccc
ctaccaggaccagacagctctcagagcaaccctagccccattacctcttccctttccagaggacctgaaaaacgtgttcccacccgaggtcg
ctgtgtttgagccatcagaagcagagatctcccacacccaaaaggccacactggtgtgcctggccacaggcttctaccccgaccacgtgga
gctgagctggtgggtgaatgggaaggaggtgcacagtggggtcagcacagacccgcagcccctcaaggagcagcccgccctcaatga
ctccagatactgcctgagcagccgcctgagggtctcggccaccttctggcagaacccccgcaaccacttccgctgtcaagtccagttctac
gggctctcggagaatgacgagtggacccaggatagggccaaacctgtcacccagatcgtcagcgccgaggcctggggtagagcaggtg
agtggggcctggggagatgcctggaggagattaggtgagaccagctaccagggaaaatggaaagatccaggtagcggacaagactaga
tccagaagaaagccagagtggacaaggtgggatgatcaaggttcacagggtcagcaaagcacggtgtgcacttcccccaccaagaagca
tagaggctgaatggagcacctcaagctcattcttccttcagatcctgacaccttagagctaagctttcaagtctccctgaggaccagccataca
gctcagcatctgagtggtgtgcatcccattctcttctggggtcctggtttcctaagatcatagtgaccacttcgctggcactggagcagcatga
gggagacagaaccagggctatcaaaggaggctgactttgtactatctgatatgcatgtgtttgtggcctgtgagtctgtgatgtaaggctcaat
gtccttacaaagcagcattctctcatccatttttcttcccctgttttctttcagactgtggcttcacctccggtaagtgagtctctcctttttctctctat
ctttcgccgtctctgctctcgaaccagggcatggagaatccacggacacaggggcgtgagggaggccagagccacctgtgcacaggtac
ctacatgctctgttcttgtcaacagagtcttaccagcaaggggtcctgtctgccaccatcctctatgagatcttgctagggaaggccaccttgta
tgccgtgctggtcagtgccctcgtgctgatggccatggtaaggaggagggtgggatagggcagatgatgggggcaggggatggaacatc
acacatgggcataaaggaatctcagagccagagcacagcctaatatatcctatcacctcaatgaaaccataatgaagccagactggggaga
aaatgcagggaatatcacagaatgcatcatgggaggatggagacaaccagcgagccctactcaaattaggcctcagagcccgcctcccct
gccctactcctgctgtgccatagcccctgaaaccctgaaaatgttctctcttccacaggtcaagagaaaggattccagaggctagctccaaaa
ccatcccaggtcattcttcatcctcacccaggattctcctgtacctgctcccaatctgtgttcctaaaagtgattctcactctgcttctcatctccta
cttacatgaatacttctctcttattctgtttccctgaagattgagctcccaacccccaagtacgaaataggctaaaccaataaaaaattgtgtgttg
ggcctggttgcatttcaggagtgtctgtggagttctgctcatcactgacctatcttctgatttagggaaagcagcattcgcttggacatctgaagt
gacagccctctttctctccacccaatgctgctttctcctgttcatcctgatggaagtctcaacaca.

Inhibitory nucleic acids or any ways of inhibiting gene expression of CIITA and/or B2M known in the art are contemplated in certain embodiments. Examples of an inhibitory nucleic acid include but are not limited to siRNA (small interfering RNA), short hairpin RNA (shRNA), double-stranded RNA, an antisense oligonucleotide, a ribozyme and a nucleic acid encoding thereof. An inhibitory nucleic acid may inhibit the transcription of a gene or prevent the translation of a gene transcript in a cell. An inhibitory nucleic acid may be from 16 to 1000 nucleotides long, and in certain embodiments from 18 to 100 nucleotides long. The nucleic acid may have nucleotides of at least or at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 50, 60, 70, 80, 90 or any range derivable therefrom. An siRNA naturally present in a living animal is not “isolated,” but a synthetic siRNA, or an siRNA partially or completely separated from the coexisting materials of its natural state is “isolated.” An isolated siRNA can exist in substantially purified form, or can exist in a non-native environment such as, for example, a cell into which the siRNA has been delivered.

Inhibitory nucleic acids are well known in the art. For example, siRNA and double-stranded RNA have been described in U.S. Pat. Nos. 6,506,559 and 6,573,099, as well as in U.S. Patent Publications 2003/0051263, 2003/0055020, 2004/0265839, 2002/0168707, 2003/0159161, and 2004/0064842, all of which are herein incorporated by reference in their entirety.

Particularly, an inhibitory nucleic acid may be capable of decreasing the expression of the protein or mRNA by at least 10%, 20%, 30%, or 40%, more particularly by at least 50%, 60%, or 70%, and most particularly by at least 75%, 80%, 90%, 95% or more or any range or value in between the foregoing.

In further embodiments, there are synthetic nucleic acids that are protein inhibitors. An inhibitor may be between 17 to 25 nucleotides in length and comprises a 5′ to 3′ sequence that is at least 90% complementary to the 5′ to 3′ sequence of a mature mRNA. In certain embodiments, an inhibitor molecule is 17, 18, 19, 20, 21, 22, 23, 24, or 25 nucleotides in length, or any range derivable therein. Moreover, an inhibitor molecule has a sequence (from 5′ to 3′) that is or is at least 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the 5′ to 3′ sequence of a mature mRNA, particularly a mature, naturally occurring mRNA, such as a mRNA to B2M, CIITA, TRAC, TRBC1, or TRBC2. One of skill in the art could use a portion of the probe sequence that is complementary to the sequence of a mature mRNA as the sequence for an mRNA inhibitor. Moreover, that portion of the probe sequence can be altered so that it is still 90% complementary to the sequence of a mature mRNA.

In some embodiments, the iNKT cells or progenitor or stem cells may comprise one or more suicide genes. In cases wherein the engineered iNKT cells comprise one or more suicide genes for subsequent depletion upon need, the suicide gene may be of any suitable kind. The iNKT cells of the disclosure may express a suicide gene product that may be enzyme-based, for example. Examples of suicide gene products include herpes simplex virus thymidine kinase (HSV-TK), purine nucleoside phosphorylase (PNP), cytosine deaminase (CD), carboxypetidase G2, cytochrome P450, linamarase, beta-lactamase, nitroreductase (NTR), carboxypeptidase A, or inducible caspase 9. Thus, in specific cases, the suicide gene may encode thymidine kinase (TK). In specific cases, the TK gene is a viral TK gene, such as a herpes simplex virus TK gene. In particular embodiments, the suicide gene product is activated by a substrate, such as ganciclovir penciclovir, or a derivative thereof.

In specific embodiments, the suicide gene is sr39TK, and examples of corresponding sequences are as follows:

sr39TK cDNA sequence (codon-optimized):

(SEQ ID NO: 67)
atgcctacactgctgcgggtgtacatcgatggccctcacggcatgggcaagaccacaaccacacagctgctggtggccctgggcagcag
ggacgatatcgtgtacgtgccagagcccatgacatattggcgcgtgctgggagcatccgagacaatcgccaacatctacaccacacagca
cagactggatcagggagagatctccgccggcgacgcagcagtggtcatgaccagcgcccagatcacaatgggcatgccatatgcagtga
ccgacgccgtgctggcacctcacatcggaggagaggcaggctctagccacgcaccaccccctgccctgacaatctttctggatcggcacc
ctatcgccttcatgctgtgctacccagccgccagatatctgatgggcagcatgaccccacaggccgtgctggccttcgtggccctgatccca
cccaccctgccaggaacaaatatcgtgctgggcgccctgccagaggacaggcacatcgatagactggccaagaggcagcgccccgga
gagcggctggacctggcaatgctggcagcaatcaggagagtgtacggcctgctggccaacaccgtgcggtatctgcagtgtggaggctc
ctggagagaggactggggacagctgtctggaacagcagtgcctccacagggagcagagccacagtccaatgcaggacctaggccaca
catcggcgataccctgttcacactgtttcgcgcaccagagctgctggcacctaacggcgatctgtacaacgtgttcgcatgggcactggacg
tgctggcaaagcggctgagatctatgcacgtgttcatcctggactacgaccagagcccagccggctgtagagatgccctgctgcagctgac
aagcggcatggtgcagacccacgtgaccacacccggctctattccaacaatctgcgacctggctaggacctttgcaagagaaatgggcga
agctaactga

sr39TK amino acid sequence:

(SEQ ID NO: 68)
MPTLLRVYIDGPHGMGKTTTTQLLVALGSRDDIVYVPEPMTYWRVLGASE
TIANIYTTQHRLDQGEISAGDAAVVMTSAQITMGMPYAVTDAVLAPHIGG
EAGSSHAPPPALTIFLDRHPIAFMLCYPAARYLMGSMTPQAVLAFVALIP
PTLPGTNIVLGALPEDRHIDRLAKRQRPGERLDLAMLAAIRRVYGLLANT
VRYLQCGGSWREDWGQLSGTAVPPQGAEPQSNAGPRPHIGDTLFTLFRAP
ELLAPNGDLYNVFAWALDVLAKRLRSMHVFILDYDQSPAGCRDALLQLTS
GMVQTHVTTPGSIPTICDLARTFAREMGEAN.

In some embodiments, the engineered iNKT cells are able to be imaged or otherwise detected. In particular cases, the cells comprise an exogenous nucleic acid encoding a polypeptide that has a substrate that may be labeled for imaging, and the imaging may be fluorescent, radioactive, colorimetric, and so forth. In specific cases, the cells are detected by positron emission tomography. The cells in at least some cases express sr39TK gene that is a positron emission tomography (PET) reporter/thymidine kinase gene that allows for tracking of these genetically modified cells with PET imaging and elimination of these cells through the sr39TK suicide gene function.

Encompassed by the disclosure are populations of engineered iNKT cells. In particular aspects, iNKT clonal cells comprise an exogenous nucleic acid encoding an iNKT T-cell receptor (T-cell receptor) and lack surface expression of one or more HLA-I or HLA-II molecules. The iNKT cells may comprise an exogenous nucleic acid encoding a suicide gene, including an enzyme-based suicide gene such as thymidine kinase (TK). The TK gene may be a viral TK gene, such as a herpes simplex virus TK gene. In the cells of the population the suicide gene may be activated by a substrate, such as ganciclovir, penciclovir, or a derivative thereof, for example. The cells may comprise an exogenous nucleic acid encoding a polypeptide that has a substrate that may be labeled for imaging, and in some cases a suicide gene product is the polypeptide that has a substrate that may be labeled for imaging. In specific aspects, the suicide gene is sr39TK.

In certain embodiments of the iNKT cell population, the iNKT cells do not express surface HLA-I or -II molecules because of disrupted expression of genes encoding beta-2-microglobulin (B2M), major histocompatibility complex class II transactivator (CIITA), and/or HLA-I or HLA-II molecules, for example. The HLA-I or HLA-II molecules are not expressed on the cell surface of iNKT cells because the cells were manipulated by gene editing, in specific cases. The gene editing may or may not involve CRISPR-Cas9.

In particular cases for the iNKT cell population, the iNKT cells comprise nucleic acid sequences from a recombinant vector that was introduced into the cells, such as a viral vector (including at least a lentivirus, a retrovirus, an adeno-associated virus (AAV), a herpesvirus, or adenovirus).

In certain embodiments, the cells of the iNKT cell population may or may not have been exposed to, or are exposed to, one or more certain conditions. In certain cases, for example, the cells of the population not exposed or were not exposed to media that comprises animal serum. The cells of the population may or may not be frozen. In some cases the cells of the population are in a solution comprising dextrose, one or more electrolytes, albumin, dextran, and/or DMSO. The solution may comprise dextrose, one or more electrolytes, albumin, dextran, and DMSO. The cells may be in a solution that is sterile, nonpyogenic, and isotonic. In specific cases the iNKT cells have been activated, such as activated with alpha-galactosylceramide (α-GC). In specific aspects, the cell population comprises at least about 10²-10⁶ clonal cells. The cell population may comprise at least about 10⁶-10¹² total cells, in some cases.

In particular embodiments there is an invariant natural killer T (iNKT) cell population comprising: clonal iNKT cells comprising one or more exogenous nucleic acids encoding an iNKT T-cell receptor (T-cell receptor) and a thymidine kinase suicide, wherein the clonal iNKT cells have been engineered not to express functional beta-2-microglobulin (B2M), major histocompatibility complex class II transactivator (CIITA), and/or HLA-I and HLA-II molecules and wherein the cell population is at least about 10⁶-10¹² total cells and comprises at least about 10²-10⁶ clonal cells. In some cases the cells are frozen in a solution.

### V. CAR Embodiments

A. Antigen Binding Regions

The antigen-binding region may be a single-chain variable fragment (scFv) derived from an antigen-specific antibody. In some embodiments, the antigen-binding region is a BCMA-binding region. In some embodiments, the antigen-binding region is a CD19-binding region. In some embodiments, the antigen-binding region is a NY-ESO-1-binding region. “Single-chain Fv” or “scFv” antibody fragments comprise the V_{H} and V_{L} domains of an antibody, wherein these domains are present in a single polypeptide chain. In some embodiments, the antigen-binding domain further comprises a peptide linker between the VH and VL domains, which may facilitate the scFv forming the desired structure for antigen binding.

The variable regions of the antigen-binding domains of the polypeptides of the disclosure can be modified by mutating amino acid residues within the VH and/or VL CDR 1, CDR 2 and/or CDR 3 regions to improve one or more binding properties (e.g., affinity) of the antibody. The term “CDR” refers to a complementarity-determining region that is based on a part of the variable chains in immunoglobulins (antibodies) and T cell receptors, generated by B cells and T cells respectively, where these molecules bind to their specific antigen. Since most sequence variation associated with immunoglobulins and T cell receptors is found in the CDRs, these regions are sometimes referred to as hypervariable regions. Mutations may be introduced by site-directed mutagenesis or PCR-mediated mutagenesis and the effect on antibody binding, or other functional property of interest, can be evaluated in appropriate in vitro or in vivo assays. Preferably conservative modifications are introduced and typically no more than one, two, three, four or five residues within a CDR region are altered. The mutations may be amino acid substitutions, additions or deletions.

Framework modifications can be made to the antibodies to decrease immunogenicity, for example, by “backmutating” one or more framework residues to the corresponding germline sequence.

It is also contemplated that the antigen binding domain may be multi-specific or multivalent by multimerizing the antigen binding domain with VH and VL region pairs that bind either the same antigen (multi-valent) or a different antigen (multi-specific).

The binding affinity of the antigen binding region, such as the variable regions (heavy chain and/or light chain variable region), or of the CDRs may be at least 10⁻⁵M, 10⁻⁶M, 10⁻⁷M, 10⁻⁸M, 10⁻⁹M, 10⁻¹⁰M, 10⁻¹¹M, 10⁻¹²M, or 10⁻¹³M. In some embodiments, the K_{D} of the antigen binding region, such as the variable regions (heavy chain and/or light chain variable region), or of the CDRs may be at least 10⁻⁵M, 10⁻⁶M, 10⁻⁷M, 10⁻⁸M, 10⁻⁹M, 10⁻¹⁰M, 10⁻¹¹M, 10⁻¹²M, or 10⁻¹³M (or any derivable range therein).

Binding affinity, K_{A}, or K_{D} can be determined by methods known in the art such as by surface plasmon resonance (SRP)-based biosensors, by kinetic exclusion assay (KinExA), by optical scanner for microarray detection based on polarization-modulated oblique-incidence reflectivity difference (OI-RD), or by ELISA.

In some embodiments, the antigen-binding region is humanized. In some embodiments, the polypeptide comprising the humanized binding region has equal, better, or at least 80, 81, 82, 83, 84, 85, 86, 87, 88, 89, 90, 91, 92, 93, 94, 95, 96, 97, 98, 99, 100, 101, 102, 103, 104, 104, 106, 106, 108, 109, 110, 115, or 120% binding affinity or expression level in host cells, compared to a polypeptide comprising a non-humanized binding region, such as a binding region from a mouse.

### VI. Formulations and Culture of the Cells

In particular embodiments, the iNKT cells and/or precursors thereto may be specifically formulated and/or they may be cultured in a particular medium (whether or not they are present in an in vitro culture system) at any stage of a process of generating the iNKT cells. The cells may be formulated in such a manner as to be suitable for delivery to a recipient without deleterious effects.

The medium in certain aspects can be prepared using a medium used for culturing animal cells as their basal medium, such as any of AIM V, X-VIVO-15, NeuroBasal, EGM2, TeSR, BME, BGJb, CMRL 1066, Glasgow MEM, Improved MEM Zinc Option, IMDM, Medium 199, Eagle MEM, aMEM, DMEM, Ham, RPMI-1640, and Fischer's media, as well as any combinations thereof, but the medium may not be particularly limited thereto as far as it can be used for culturing animal cells. Particularly, the medium may be xeno-free or chemically defined.

The medium can be a serum-containing or serum-free medium, or xeno-free medium. From the aspect of preventing contamination with heterogeneous animal-derived components, serum can be derived from the same animal as that of the stem cell(s). The serum-free medium refers to medium with no unprocessed or unpurified serum and accordingly, can include medium with purified blood-derived components or animal tissue-derived components (such as growth factors).

The medium may contain or may not contain any alternatives to serum. The alternatives to serum can include materials which appropriately contain albumin (such as lipid-rich albumin, bovine albumin, albumin substitutes such as recombinant albumin or a humanized albumin, plant starch, dextrans and protein hydrolysates), transferrin (or other iron transporters), fatty acids, insulin, collagen precursors, trace elements, 2-mercaptoethanol, 3′-thiolgiycerol, or equivalents thereto. The alternatives to serum can be prepared by the method disclosed in International Publication No. 98/30679, for example (incorporated herein in its entirety). Alternatively, any commercially available materials can be used for more convenience. The commercially available materials include knockout Serum Replacement (KSR), Chemically-defined Lipid concentrated (Gibco), and Glutamax (Gibco).

In further embodiments, the medium may be a serum-free medium that is suitable for cell development. For example, the medium may comprise B-27® supplement, xeno-free B-27® supplement (available at world wide web at thermofisher.com/us/en/home/technical-resources/media-formulation.250.html), NS21 supplement (Chen et al., J Neurosci Methods, 2008 Jun. 30; 171(2): 239-247, incorporated herein in its entirety), GS21™ supplement (available at world wide web at amsbio.com/B-27.aspx), or a combination thereof at a concentration effective for producing T cells from the 3D cell aggregate.

In certain embodiments, the medium may comprise one, two, three, four, five, six, seven, eight, nine, ten, 11, 12, 13, 14, 15, 16, 17, 18, 19, 20 or more of the following: Vitamins such as biotin; DL Alpha Tocopherol Acetate; DL Alpha-Tocopherol; Vitamin A (acetate); proteins such as BSA (bovine serum albumin) or human albumin, fatty acid free Fraction V; Catalase; Human Recombinant Insulin; Human Transferrin; Superoxide Dismutase; Other Components such as Corticosterone; D-Galactose; Ethanolamine HCl; Glutathione (reduced); L-Carnitine HCl; Linoleic Acid; Linolenic Acid; Progesterone; Putrescine 2HCl; Sodium Selenite; and/or T3 (triodo-I-thyronine).

In some embodiments, the medium further comprises vitamins. In some embodiments, the medium comprises 1, 2, 3, 4, 5, 6, 7, 8, 9, 10, 11, 12, or 13 of the following (and any range derivable therein): biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B12, or the medium includes combinations thereof or salts thereof. In some embodiments, the medium comprises or consists essentially of biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, and vitamin B12. In some embodiments, the vitamins include or consist essentially of biotin, DL alpha tocopherol acetate, DL alpha-tocopherol, vitamin A, or combinations or salts thereof. In some embodiments, the medium further comprises proteins. In some embodiments, the proteins comprise albumin or bovine serum albumin, a fraction of BSA, catalase, insulin, transferrin, superoxide dismutase, or combinations thereof. In some embodiments, the medium further comprises one or more of the following: corticosterone, D-Galactose, ethanolamine, glutathione, L-carnitine, linoleic acid, linolenic acid, progesterone, putrescine, sodium selenite, or triodo-I-thyronine, or combinations thereof. In some embodiments, the medium comprises one or more of the following: a B-27® supplement, xeno-free B-27® supplement, GS21™ supplement, or combinations thereof. In some embodiments, the medium comprises or further comprises amino acids, monosaccharides, inorganic ions. In some embodiments, the amino acids comprise arginine, cystine, isoleucine, leucine, lysine, methionine, glutamine, phenylalanine, threonine, tryptophan, histidine, tyrosine, or valine, or combinations thereof. In some embodiments, the inorganic ions comprise sodium, potassium, calcium, magnesium, nitrogen, or phosphorus, or combinations or salts thereof. In some embodiments, the medium further comprises one or more of the following: molybdenum, vanadium, iron, zinc, selenium, copper, or manganese, or combinations thereof. In certain embodiments, the medium comprises or consists essentially of one or more vitamins discussed herein and/or one or more proteins discussed herein, and/or one or more of the following: corticosterone, D-Galactose, ethanolamine, glutathione, L-carnitine, linoleic acid, linolenic acid, progesterone, putrescine, sodium selenite, or triodo-I-thyronine, a B-27® supplement, xeno-free B-27® supplement, GS21™ supplement, an amino acid (such as arginine, cystine, isoleucine, leucine, lysine, methionine, glutamine, phenylalanine, threonine, tryptophan, histidine, tyrosine, or valine), monosaccharide, inorganic ion (such as sodium, potassium, calcium, magnesium, nitrogen, and/or phosphorus) or salts thereof, and/or molybdenum, vanadium, iron, zinc, selenium, copper, or manganese.

In further embodiments, the medium may comprise externally added ascorbic acid. The medium can also contain one or more externally added fatty acids or lipids, amino acids (such as non-essential amino acids), vitamin(s), growth factors, cytokines, antioxidant substances, 2-mercaptoethanol, pyruvic acid, buffering agents, and/or inorganic salts.

One or more of the medium components may be added at a concentration of at least, at most, or about 0.1, 0.5, 1, 2, 3, 4, 5, 10, 15, 20, 25, 30, 35, 40, 45, 50, 55, 60, 65, 70, 75, 80, 85, 90, 95, 100, 150, 180, 200, 250 ng/L, ng/ml, μg/ml, mg/ml, or any range derivable therein.

The medium used may be supplemented with at least one externally added cytokine at a concentration from about 0.1 ng/mL to about 500 ng/mL, more particularly 1 ng/mL to 100 ng/mL, or at least, at most, or about 0.1, 0.5, 1, 2, 3, 4, 5, 10, 15, 20, 25, 30, 35, 40, 45, 50, 55, 60, 65, 70, 75, 80, 85, 90, 95, 100, 150, 180, 200, 250 ng/L, ng/ml, μg/ml, mg/ml, or any range derivable therein. Suitable cytokines, include but are not limited to, FLT3 ligand (FLT3L), interleukin 7 (IL-7), stem cell factor (SCF), thrombopoietin (TPO), IL-2, IL-4, IL-6, IL-15, IL-21, TNF-alpha, TGF-beta, interferon-gamma, interferon-lambda, TSLP, thymopentin, pleotrophin, and/or midkine. Particularly, the culture medium may include at least one of FLT3L and IL-7. More particularly, the culture may include both FLT3L and IL-7.

Other culturing conditions can be appropriately defined. For example, the culturing temperature can be about 20 to 40° C., such as at least, at most, or about 20, 21, 22, 23, 24, 25, 26, 27, 28, 29, 30, 31, 32, 33, 34, 35, 36, 37, 38, 39, 40° C. (or any range derivable therein), though the temperature may be above or below these values. The CO₂ concentration can be about 1, 2, 3, 4, 5, 6, 7, 8, 9, or 10% (or any range derivable therein), such as about 2% to 10%, for example, about 2 to 5%, or any range derivable therein. The oxygen tension can be at least or about 1, 5, 8, 10, 20%, or any range derivable therein.

In specific embodiments, the allogeneic HSC-engineered HLA-negative iNKT cells are specifically formulated. They may or may not be formulated as a cell suspension. In specific cases they are formulated in a single dose form. They may be formulated for systemic or local administration. In some cases the cells are formulated for storage prior to use, and the cell formulation may comprise one or more cryopreservation agents, such as DMSO (for example, in 5% DMSO). The cell formulation may comprise albumin, including human albumin, with a specific formulation comprising 2.5% human albumin. The cells may be formulated specifically for intravenous administration; for example, they are formulated for intravenous administration over less than one hour. In particular embodiments the cells are in a formulated cell suspension that is stable at room temperature for 1, 2, 3, or 4 hours or more from time of thawing.

In some embodiments, the method further comprises priming the T cells. In some embodiments, the T cells are primed with antigen presenting cells. In some embodiments, the antigen presenting cells present tumor antigens.

In particular embodiments, the exogenous TCR of the iNKT cells may be of any defined antigen specificity. In some embodiments, it can be selected based on absent or reduced alloreactivity to the intended recipient (examples include certain virus-specific TCRs, xeno-specific TCRs, or cancer-testis antigen-specific TCRs). In the example where the exogenous TCR is non-alloreactive, during T cell differentiation the exogenous TCR suppresses rearrangement and/or expression of endogenous TCR loci through a developmental process called allelic exclusion, resulting in T cells that express only the non-alloreactive exogenous TCR and are thus non-alloreactive. In some embodiments, the choice of exogenous TCR may not necessarily be defined based on lack of alloreactivity. In some embodiments, the endogenous TCR genes have been modified by genome editing so that they do not express a protein. Methods of gene editing such as methods using the CRISPR/Cas9 system are known in the art and described herein.

In some embodiments, the isolated iNKT cell or population thereof comprise a one or more chimeric antigen receptors (CARs). Examples of tumor cell antigens to which a CAR may be directed include at least 5T4, 8H9, αᵥβ₆ integrin, BCMA, B7-H3, B7-H6, CAIX, CA9, CD19, CD20, CD22, CD30, CD33, CD38, CD44, CD44v6, CD44v7/8, CD70, CD123, CD138, CD171, CEA, CSPG4, EGFR, EGFR family including ErbB2 (HER2), EGFRvIII, EGP2, EGP40, ERBB3, ERBB4, ErbB3/4, EPCAM, EphA2, EpCAM, folate receptor-a, FAP, FBP, fetal AchR, FRc, GD2, G250/CAIX, GD3, Glypican-3 (GPC3), Her2, IL-13Rα2, Lambda, Lewis-Y, Kappa, KDR, MAGE, MCSP, Mesothelin, Mucd, Mucl6, NCAM, NKG2D Ligands, NY-ESO-1, PRAME, PSC1, PSCA, PSMA, ROR1, SP17, Survivin, TAG72, TEMs, carcinoembryonic antigen, HMW-MAA, AFP, CA-125, ETA, Tyrosinase, MAGE, laminin receptor, HPV E6, E7, BING-4, Calcium-activated chloride channel 2, Cyclin-B1, 9D7, EphA3, Telomerase, SAP-1, BAGE family, CAGE family, GAGE family, MAGE family, SAGE family, XAGE family, NY-ESO-1/LAGE-1, PAME, SSX-2, Melan-A/MART-1, GP100/pmel17, TRP-1/-2, P. polypeptide, MC1R, Prostate-specific antigen, β-catenin, BRCA1/2, CML66, Fibronectin, MART-2, TGF-βRII, or VEGF receptors (e.g., VEGFR2), for example. The CAR may be a first, second, third, or more generation CAR. The CAR may be bispecific for any two nonidentical antigens, or it may be specific for more than two nonidentical antigens.

### VII. Additional Modifications and Polypeptide Embodiments

Additionally, the polypeptides of the disclosure may be chemically modified. Glycosylation of the polypeptides can be altered, for example, by modifying one or more sites of glycosylation within the polypeptide sequence to increase the affinity of the polypeptide for antigen (U.S. Pat. Nos. 5,714,350 and 6,350,861).

It is contemplated that a region or fragment of a polypeptide of the disclosure or a nucleic acid of the disclosure encoding for a polypeptide that may have an amino acid sequence that has, has at least or has at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 or more amino acid substitutions, contiguous amino acid additions, or contiguous amino acid deletions with respect to any of SEQ ID NOS:46-61 or 81-88 or with respect to the polypeptide encoded by any of SEQ ID NOS:1-45 or 62-66.

Alternatively, a region or fragment of a polypeptide of the disclosure may have an amino acid sequence that comprises or consists of an amino acid sequence that is, is at least, or is at most 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% (or any range derivable therein) identical to any of SEQ ID NOS:46-61 or 81-88 or with respect to the polypeptide encoded by any of SEQ ID NOS:1-45 or 62-66. Moreover, in some embodiments, a region or fragment comprises an amino acid region of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 or more contiguous amino acids starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 in any of SEQ ID NOS:46-61 or 81-88 or with respect to the polypeptide encoded by any of SEQ ID NOS:1-45 or 62-66 (where position 1 is at the N-terminus of the SEQ ID NO or the N terminus of the polypeptide encoded by the SEQ ID NO). The polypeptides of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000, 1500, or 2000 or more contiguous amino acids or nucleic acids, or any range derivable therein, of any of SEQ ID NOS:46-61 or 81-88 or with respect to the polypeptide encoded by any of SEQ ID NOS:1-45 or 62-66.

The polypeptides of the disclosure may include at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 substitutions (or any range derivable therein).

The substitution may be at amino acid position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 650, 700, 750, 800, 850, 900, 1000, 1500, or 2000 (or any derivable range therein) of any of SEQ ID NOS:46-61 or 81-88 or with respect to the polypeptide encoded by any of SEQ ID NOS:1-45 or 62-66.

The polypeptides described herein may be of a fixed length of at least, at most, or exactly 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more amino acids (or any derivable range therein) of SEQ ID NOS:46-61 or 81-88 or with respect to the polypeptide encoded by any of SEQ ID NOS:1-45 or 62-66.

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

Proteins may be recombinant, or synthesized in vitro. Alternatively, a non-recombinant or recombinant protein may be isolated from bacteria. It is also contemplated that bacteria containing such a variant may be implemented in compositions and methods. Consequently, a protein need not be isolated.

The term “functionally equivalent codon” is used herein to refer to codons that encode the same amino acid, such as the six codons for arginine or serine, and also refers to codons that encode biologically equivalent amino acids.

It also will be understood that amino acid and nucleic acid sequences may include additional residues, such as additional N- or C-terminal amino acids, or 5′ or 3′ sequences, respectively, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

The following is a discussion based upon changing of the amino acids of a protein to create an equivalent, or even an improved, second-generation molecule. For example, certain amino acids may be substituted for other amino acids in a protein structure without appreciable loss of interactive binding capacity. Structures such as, for example, an enzymatic catalytic domain or interaction components may have amino acid substituted to maintain such function. Since it is the interactive capacity and nature of a protein that defines that protein's biological functional activity, certain amino acid substitutions can be made in a protein sequence, and in its underlying DNA coding sequence, and nevertheless produce a protein with like properties. It is thus contemplated by the inventors that various changes may be made in the DNA sequences of genes without appreciable loss of their biological utility or activity.

In other embodiments, alteration of the function of a polypeptide is intended by introducing one or more substitutions. For example, certain amino acids may be substituted for other amino acids in a protein structure with the intent to modify the interactive binding capacity of interaction components. Structures such as, for example, protein interaction domains, nucleic acid interaction domains, and catalytic sites may have amino acids substituted to alter such function. Since it is the interactive capacity and nature of a protein that defines that protein's biological functional activity, certain amino acid substitutions can be made in a protein sequence, and in its underlying DNA coding sequence, and nevertheless produce a protein with different properties. It is thus contemplated by the inventors that various changes may be made in the DNA sequences of genes with appreciable alteration of their biological utility or activity.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. It is understood that an amino acid can be substituted for another having a similar hydrophilicity value and still produce a biologically equivalent and immunologically equivalent protein.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known and include: arginine and lysine; glutamate and aspartate; serine and threonine; glutamine and asparagine; and valine, leucine and isoleucine.

In specific embodiments, all or part of proteins described herein can also be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Alternatively, recombinant DNA technology may be employed wherein a nucleotide sequence that encodes a peptide or polypeptide is inserted into an expression vector, transformed or transfected into an appropriate host cell and cultivated under conditions suitable for expression.

One embodiment includes the use of gene transfer to cells, including microorganisms, for the production and/or presentation of proteins. The gene for the protein of interest may be transferred into appropriate host cells followed by culture of cells under the appropriate conditions. A nucleic acid encoding virtually any polypeptide may be employed. The generation of recombinant expression vectors, and the elements included therein, are discussed herein. Alternatively, the protein to be produced may be an endogenous protein normally synthesized by the cell used for protein production.

### VIII. Methods of Producing the iNKT Cells

iNKT cells may be produced by any suitable method(s). The method(s) may utilize one or more successive steps for one or more modifications to cells and/or utilize one or more simultaneous steps for one or more modifications to cells. In specific embodiments, a starting source of cells are modified to become functional as iNKT cells followed by one or more steps to add one or more additional characteristics to the cells, such as the ability to be imaged, and/or the ability to be selectively killed, and/or the ability to be able to be used allogeneically. In specific embodiments, at least part of the process for generating iNKT cells occurs in a specific in vitro culture system. An example of a specific in vitro culture system is one that allows differentiation of certain cells at high efficiency and high yield. In specific embodiments the in vitro culture system is an artificial thymic organoid (ATO) system. In further specific embodiments, the in vitro culture system excludes one or more of an ATO system, a 3-dimensional culture system, a stromal cell feeder layer, and a notch ligand or fragment thereof.

In specific cases, iNKT cells may be generated by the following: 1) genetic modification of donor HSCs to express iNKT TCRs (for example, via lentiviral vectors) and to eliminate expression of HLA-I/II molecules (for example, via CRISPR/Cas9-based gene editing); 2) in vitro differentiation into iNKT cells via an ATO culture, 3) in vitro iNKT cell purification and expansion, and 4) formulation and cryopreservation and/or use. In some embodiments, iNKT cells are generated without the use of an ATO culture (e.g., via a “feeder-free” culture system disclosed herein).

Some embodiments of the disclosure provide methods of preparing a population of clonal invariant natural killer T (iNKT) cells comprising: a) selecting CD34+ cells from human peripheral blood cells (PBMCs); b) introducing one or more nucleic acids encoding a human iNKT T-cell receptor (TCR); c) eliminating expression of one or more HLA-I/II genes in the isolated human CD34+ cells; and, d) culturing isolated CD34+ cells expressing iNKT TCR in an artificial thymic organoid (ATO) system to produce iNKT cells, wherein the ATO system comprises a 3D cell aggregate comprising a selected population of stromal cells that express a Notch ligand and a serum-free medium. The method may further comprise isolating CD34− cells. In alternative embodiments, other culture systems than the ATO system is employed, such as. The method may further comprise isolating CD34− cells. In some embodiments, a 2-D culture system or other forms of 3-D culture systems (e.g., FTOC-like culture, metrigel-aided culture) are applied.

Specific aspects of the disclosure relate to a cell culture system that may be 2 or 3 dimensional to produce iNKT cells from less differentiated cells such as embryonic stem cells, pluripotent stem cells, hematopoietic stem or progenitor cells, induced pluripotent stem (iPS) cells, or stem or progenitor cells. Stem cells of any type may be utilized from various resources, including at least fetal liver, cord blood, and peripheral blood CD34+ cells (either G-CSF-mobilized or non-G-CSF-mobilized), for example.

In some embodiments, the system involves using serum-free medium. In certain aspects, the system uses a serum-free medium that is suitable for cell development for culturing of a three-dimensional cell aggregate. Such a system produces sufficient amounts of iNKT cells. In embodiments of the disclosure, the cells or cell aggregate is cultured in a serum-free medium comprising insulin for a time period sufficient for the in vitro differentiation of stem or progenitor cells to iNKT cells or precursors to iNKT cells.

Embodiments of a cell culture composition may comprise a culture that uses highly-standardized, serum-free components and a stromal cell line to facilitate robust and highly reproducible T cell differentiation from human HSCs. In certain embodiments, cell differentiation in the culture closely mimicked endogenous thymopoiesis and, in contrast to monolayer co-cultures, supported efficient positive selection of functional iNKT. Certain aspects of the culture compositions use serum-free conditions, avoid the use of human thymic tissue or proprietary scaffold materials, and facilitate positive selection and robust generation of fully functional, mature human iNKT cells from source cells.

In some embodiments, the culture system may comprise the co-culture of human HSC with stromal cells expressing a Notch ligand, in the presence of an optimized medium containing FLT3 ligand (FLT3L), interleukin 7 (IL-7), B27, and ascorbic acid. Conditions that permit culture at the air-fluid interface may also be present. It has been determined that combinatorial signaling from soluble factors (cytokines, ascorbic acid, B27 components, and stromal cell-derived factors) together with 3D cell-cell interactions between hematopoietic and stromal cells, facilitates human T lineage commitment, positive selection, and efficient differentiation into functional, mature T cells.

In some embodiments, the cell culture is created by mixing CD34+ transduced cells with the selected population of stromal cells on a physical matrix or scaffold. The method may further comprise centrifuging the CD34+ transduced cells and stromal cells to form a cell pellet that is placed on the physical matrix or scaffold. The Notch ligand expressed by the stromal cells may be intact, partial, or modified DLL1, DLL4, JAG1, JAG2, or a combination thereof. In specific cases, the Notch ligand is a human Notch ligand, such as human DLL1, for example.

The culture system utilized to produce the iNKT cells may have a certain ratio of stromal cells to CD34+ cells. In specific cases, the ratio between stromal cells and CD34+ cells is about 1:5 to 1:20. The stromal cells may be a murine stromal cell line, a human stromal cell line, a selected population of primary stromal cells, a selected population of stromal cells differentiated from pluripotent stem cells in vitro, or a combination thereof. The stroma cells may be a selected population of stromal cells differentiated from hematopoietic stem or progenitor cells in vitro.

In methods of preparing a population of clonal iNKT cells, selecting iNKT cells lacking surface expression of HLA-I and HLA-II molecules may comprise contacting the iNKT cells with magnetic beads that bind to and positively select for iNKT cells and negatively select for HLA-I/II-negative cells. In specific embodiments, the magnetic beads are coated with monoclonal antibodies recognizing human iNKT TCRs, HLA-I molecules, or HLA-II molecules. In particular embodiments, the monoclonal antibodies are Clone 6B11 (recognizing human TCR Vα24-Jα18 thus recognizing human iNKT invariant TCR alpha chain), Clone 2M2 (recognizing human B2M thus recognizing cell surface-displayed human HLA-I molecules), Clone W6/32 (recognizing HLA-A,B,C thus recognizing human HLA-I molecules), and Clone Tü39 (recognizing human HLA-DR, DP, DQ thus recognizing human HLA-II molecules).

Cells produced by the preparation methods may be frozen. The produced cells may be in a solution comprising dextrose, one or more electrolytes, albumin, dextran, and DMSO. The solution may be sterile, nonpyogenic, and isotonic.

In particular embodiments, the culture system utilizes feeder cells that may comprise CD34⁻ cells. In some embodiments, the culture system does not use feeder cells.

Preparation methods may further comprise activating and expanding the selected iNKT cells; for example, the selected iNKT cells have been activated with alpha-galactosylceramide (α-GC). The feeder cells may have been pulsed with α-GC.

Preparation methods of the disclosure may produce a population of clonal iNKT cells comprising at least about 10²-10⁶ clonal iNKT cells. The method may produce a cell population comprising at least about 10⁶-10¹²total cells. The produced cell population may be frozen and then thawed. In some cases of the preparation method, the method further comprises introducing one or more additional nucleic acids into the frozen and thawed cell population, such as the one or more additional nucleic acids encoding one or more therapeutic gene products, for example.

In specific embodiments, there may be provided a method of a 3D or 2D culture composition, as developed, involves aggregation of the MS-5 murine stromal cell line transduced with human DLL1 (MS5-hDLL1, hereafter) with CD34⁺ HSPCs isolated from human cord blood, bone marrow, or G-CSF mobilized peripheral blood. Up to 1×10⁶ HSPCs are mixed with MS5-hDLL1 cells at an optimized ratio (typically 1:10 HSPCs to stromal cells).

For example, aggregation can be achieved by centrifugation of the mixed cell suspension (“compaction aggregation”) followed by aspiration of the cell-free supernatant. In particular embodiments, the cell pellet may then be aspirated as a slurry in 5-10 ul of a differentiation medium and transferred as a droplet onto 0.4 um nylon transwell culture inserts, which are floated in a well of differentiation medium, allowing the bottom of the insert to be in contact with medium and the top with air.

For example, the differentiation medium may comprise RPMI-1640, 5 ng/ml human FLT3L, 5 ng/ml human IL-7, 4% Serum-Free B27 Supplement, and 30 uM L-ascorbic acid. Medium may be completely replaced every 3-4 days from around the culture inserts. During the first 2 weeks of culture, cell aggregates may self-organize as ATOs, and early T cell lineage commitment and differentiation occurs. In certain aspects, cells are cultured for at least 6 weeks to allow for optimal T cell differentiation. Retrieval of hematopoietic cells from cell culture can be achieved by disaggregating cells by pipetting.

Variations in the protocol permit the use of alternative components with varying impact on efficacy, specifically:

Base medium RPMI may be substituted for several commercially available alternatives (e.g. IMDM)

The stromal cell line used is MS-5, a previously described murine bone marrow cell line (Itoh et al, 1989), however MS-5 may be substituted for similar murine stromal cell lines (e.g. OP9, S17), human stromal cell lines (e.g. HS-5, HS-27a), primary human stromal cells, or human pluripotent stem cell-derived stromal cells.

The stromal cell line is transduced with a lentivirus encoding human DLL1 cDNA; however the method of gene delivery, as well as the Notch ligand gene, may be varied. Alternative Notch ligand genes include DLL4, JAG1, JAG2, and others. Notch ligands also include those described in U.S. Pat. Nos. 7,795,404 and 8,377,886, which are herein incorporated by reference. Notch ligands further include Delta 1, 3, and 4 and Jagged 1, 2.

The type and source of HSCs may include bone marrow, cord blood, peripheral blood, thymus, or other primary sources; or HSCs derived from human embryonic stem cells (ESC) or induced pluripotent stem cells (iPSC).

Cytokine conditions can be varied: e.g. levels of FLT3L and IL-7 may be changed to alter T cell differentiation kinetics; other hematopoietic cytokines such as Stem Cell Factor (SCF/KIT ligand), thrombopoietin (TPO), IL-2, IL-15 may be added.

Genetic modification may also be introduced to certain components to generate antigen-specific T cells, and to model positive and negative selection. Examples of these modifications include: transduction of HSCs with a lentiviral vector encoding an antigen-specific T cell receptor (TCR) or chimeric antigen receptor (CAR) for the generation of antigen-specific, allelically excluded naïve T cells; transduction of HSCs with gene/s to direct lineage commitment to specialized lymphoid cells. For example, transduction of HSCs with an invariant natural killer T cell (iNKT) associated TCR to generate functional iNKT cells in cell culture or ATO; transduction of the stromal cell line (e.g., MS5-hDLL1) with human MHC genes (e.g. human CD1d gene) to enhance positive selection and maturation of both TCR engineered or non-engineered T cells in cell culture; and/or transduction of the stromal cell line with an antigen plus costimulatory molecules or cytokines to enhance the positive selection of CAR T cells in culture.

In producing the engineered iNKT cells, CD34+ cells from human peripheral blood cells (PBMCs) may be modified by introducing certain exogenous gene(s) and by knocking out certain endogenous gene(s). The methods may further comprise culturing selected CD34+ cells in media prior to introducing one or more nucleic acids into the cells. The culturing may comprise incubating the selected CD34+ cells with medium comprising one or more growth factors, in some cases, and the one or more growth factors may comprise c-kit ligand, flt-3 ligand, and/or human thrombopoietin (TPO), for example. The growth factors may or may not be at a certain concentration, such as between about 5 ng/ml to about 500 ng/ml/.

In particular methods the nucleic acid(s) to be introduced into the cells are one or more nucleic acids that comprise a nucleic acid sequence encoding an α-TCR and a β-TCR. The methods may further comprise introducing into the selected CD34+ cells a nucleic acid encoding a suicide gene. In specific aspects, one nucleic acid encodes both the α-TCR and the β-TCR, or one nucleic acid encodes the α-TCR, the β-TCR, and the suicide gene. The suicide gene may be enzyme-based, such as thymidine kinase (TK) including a viral TK gene such as one from herpes simplex virus TK gene. The suicide gene may be activated by a substrate, such as ganciclovir, penciclovir, or a derivative thereof. The cells may be engineered to comprise an exogenous nucleic acid encoding a polypeptide that has a substrate that may be labeled for imaging. In some cases, a suicide gene product is a polypeptide that has a substrate that may be labeled for imaging, such as sr39TK.

The cells may be engineered to lack surface expression of HLA-I and/or HLA-II molecules, for example by discrupting the functional expression of genes encoding beta-2-microglobulin (B2M), major histocompatibility complex class II transactivator (CIITA), and/or HLA-I and HLA-II molecules. In the production methods, eliminating surface expression of one or more HLA-I/II molecules in the isolated human CD34+ cells may comprise introducing CRISPR and one or more guide RNAs (gRNAs) corresponding to B2M, CIITA, or individual HLA-I or HLA-II molecules into the cells. CRISPR or the one or more gRNAs are transfected into the cell by electroporation or lipid-mediated transfection in some cases. In specific embodiments, the nucleic acid encoding the TCR receptor is introduced into the cell using a recombinant vector such as a viral vector including at least a lentivirus, a retrovirus, an adeno-associated virus (AAV), a herpesvirus, or adenovirus, for example.

In manufacturing the engineered iNKT cells, the cells may be present in a particular serum-free medium, including one that comprises externally added ascorbic acid. In specific aspects, the serum-free medium further comprises externally added FLT3 ligand (FLT3L), interleukin 7 (IL-7), stem cell factor (SCF), thrombopoietin (TPO), stem cell factor (SCF), thrombopoietin (TPO), IL-2, IL-4, IL-6, IL-15, IL-21, TNF-alpha, TGF-beta, interferon-gamma, interferon-lambda, TSLP, thymopentin, pleotrophin, midkine, or combinations thereof. The serum-free medium may further comprise vitamins, including biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B12, or combinations thereof or salts thereof. The serum-free medium may further comprise one or more externally added (or not) proteins, such as albumin or bovine serum albumin, a fraction of BSA, catalase, insulin, transferrin, superoxide dismutase, or combinations thereof. The serum-free medium may further comprise corticosterone, D-Galactose, ethanolamine, glutathione, L-carnitine, linoleic acid, linolenic acid, progesterone, putrescine, sodium selenite, or triodo-I-thyronine, or combinations thereof. The serum-free medium may comprise a B-27® supplement, xeno-free B-27® supplement, GS21™ supplement, or combinations thereof. Amino acids (including arginine, cysteine, isoleucine, leucine, lysine, methionine, glutamine, phenylalanine, threonine, tryptophan, histidine, tyrosine, or valine, or combinations thereof), monosaccharides, and/or inorganic ions (including sodium, potassium, calcium, magnesium, nitrogen, or phosphorus, or combinations or salts thereof, for example) may be present in the serum-free medium. The serum-free medium may further comprise molybdenum, vanadium, iron, zinc, selenium, copper, or manganese, or combinations thereof.

Cell culture conditions may be provided for the culture of 3D cell aggregates described herein and for the production of T cells and/or positive/negative selection thereof. In certain aspects, starting cells of a selected population may comprise at least or about 10⁴, 10⁵, 10⁶, 10⁷, 10⁸, 10⁹, 10¹⁰, 10¹¹, 10¹², 10¹³ cells or any range derivable therein. The starting cell population may have a seeding density of at least or about 10, 10¹, 10², 10³, 10⁴, 10⁵, 10⁶, 10⁷, 10⁸ cells/ml, or any range derivable therein.

A culture vessel used for culturing the 3D cell aggregates or progeny cells thereof can include, but is particularly not limited to: flask, flask for tissue culture, dish, petri dish, dish for tissue culture, multi dish, micro plate, micro-well plate, multi plate, multi-well plate, micro slide, chamber slide, tube, tray, CellSTACK® Chambers, culture bag, and roller bottle, as long as it is capable of culturing the stem cells therein. The stem cells may be cultured in a volume of at least or about 0.2, 0.5, 1, 2, 5, 10, 20, 30, 40, 50 ml, 100 ml, 150 ml, 200 ml, 250 ml, 300 ml, 350 ml, 400 ml, 450 ml, 500 ml, 550 ml, 600 ml, 800 ml, 1000 ml, 1500 ml, or any range derivable therein, depending on the needs of the culture. In a certain embodiment, the culture vessel may be a bioreactor, which may refer to any device or system that supports a biologically active environment. The bioreactor may have a volume of at least or about 2, 4, 5, 6, 8, 10, 15, 20, 25, 50, 75, 100, 150, 200, 500 liters, 1, 2, 4, 6, 8, 10, 15 cubic meters, or any range derivable therein.

The culture vessel can be cellular adhesive or non-adhesive and selected depending on the purpose. The cellular adhesive culture vessel can be coated with any of substrates for cell adhesion such as extracellular matrix (ECM) to improve the adhesiveness of the vessel surface to the cells. The substrate for cell adhesion can be any material intended to attach stem cells or feeder cells (if used). The substrate for cell adhesion includes collagen, gelatin, poly-L-lysine, poly-D-lysine, laminin, and fibronectin and mixtures thereof for example Matrigel™, and lysed cell membrane preparations.

Various defined matrix components may be used in the culturing methods or compositions. For example, recombinant collagen IV, fibronectin, laminin, and vitronectin in combination may be used to coat a culturing surface as a means of providing a solid support for pluripotent cell growth, as described in Ludwig et al. (2006a; 2006b), which are incorporated by reference in its entirety.

A matrix composition may be immobilized on a surface to provide support for cells. The matrix composition may include one or more extracellular matrix (ECM) proteins and an aqueous solvent. The term “extracellular matrix” is recognized in the art. Its components include one or more of the following proteins: fibronectin, laminin, vitronectin, tenascin, entactin, thrombospondin, elastin, gelatin, collagen, fibrillin, merosin, anchorin, chondronectin, link protein, bone sialoprotein, osteocalcin, osteopontin, epinectin, hyaluronectin, undulin, epiligrin, and kalinin. Other extracellular matrix proteins are described in Kleinman et al., (1993), herein incorporated by reference. It is intended that the term “extracellular matrix” encompass a presently unknown extracellular matrix that may be discovered in the future, since its characterization as an extracellular matrix will be readily determinable by persons skilled in the art.

In some aspects, the total protein concentration in the matrix composition may be about 1 ng/mL to about 1 mg/mL. In some embodiments, the total protein concentration in the matrix composition is about 1 μg/mL to about 300 μg/mL. In more preferred embodiments, the total protein concentration in the matrix composition is about 5 μg/mL to about 200 μg/mL.

The extracellular matrix (ECM) proteins may be of natural origin and purified from human or animal tissues. Alternatively, the ECM proteins may be genetically engineered recombinant proteins or synthetic in nature. The ECM proteins may be a whole protein or in the form of peptide fragments, native or engineered. Examples of ECM protein that may be useful in the matrix for cell culture include laminin, collagen I, collagen IV, fibronectin and vitronectin. In some embodiments, the matrix composition includes synthetically generated peptide fragments of fibronectin or recombinant fibronectin.

In still further embodiments, the matrix composition includes a mixture of at least fibronectin and vitronectin. In some other embodiments, the matrix composition preferably includes laminin.

The matrix composition preferably includes a single type of extracellular matrix protein. In some embodiments, the matrix composition includes fibronectin, particularly for use with culturing progenitor cells. For example, a suitable matrix composition may be prepared by diluting human fibronectin, such as human fibronectin sold by Becton, Dickinson & Co. of Franklin Lakes, N.J. (BD) (Cat #354008), in Dulbecco's phosphate buffered saline (DPBS) to a protein concentration of 5 μg/mL to about 200 μg/mL. In a particular example, the matrix composition includes a fibronectin fragment, such as RetroNectin®. RetroNectin® is a ˜63 kDa protein of (574 amino acids) that contains a central cell-binding domain (type III repeat, 8,9,10), a high affinity heparin-binding domain II (type III repeat, 12,13,14), and CS1 site within the alternatively spliced IIICS region of human fibronectin.

In some other embodiments, the matrix composition may include laminin. For example, a suitable matrix composition may be prepared by diluting laminin (Sigma-Aldrich (St. Louis, Mo.); Cat #L6274 and L2020) in Dulbecco's phosphate buffered saline (DPBS) to a protein concentration of 5 μg/ml to about 200 μg/ml.

In some embodiments, the matrix composition is xeno-free, in that the matrix is or its component proteins are only of human origin. This may be desired for certain research applications. For example in the xeno-free matrix to culture human cells, matrix components of human origin may be used, wherein any non-human animal components may be excluded. In certain aspects, Matrigel™ may be excluded as a substrate from the culturing composition. Matrigel™ is a gelatinous protein mixture secreted by mouse tumor cells and is commercially available from BD Biosciences (New Jersey, USA). This mixture resembles the complex extracellular environment found in many tissues and is used frequently by cell biologists as a substrate for cell culture, but it may introduce undesired xeno antigens or contaminants.

In certain embodiments, cells containing an exogenous nucleic acid may be identified in vitro or in vivo by including a marker in the expression vector or the exogenous nucleic acid. Such markers would confer an identifiable change to the cell permitting easy identification of cells containing the expression vector. Generally, a selection marker may be one that confers a property that allows for selection. A positive selection marker may be one in which the presence of the marker allows for its selection, while a negative selection marker is one in which its presence prevents its selection. An example of a positive selection marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selection markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes as negative selection markers such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art.

Selectable markers may include a type of reporter gene used in laboratory microbiology, molecular biology, and genetic engineering to indicate the success of a transfection or other procedure meant to introduce foreign DNA into a cell. Selectable markers are often antibiotic resistance genes; cells that have been subjected to a procedure to introduce foreign DNA are grown on a medium containing an antibiotic, and those cells that can grow have successfully taken up and expressed the introduced genetic material. Examples of selectable markers include: the Abicr gene or Neo gene from Tn5, which confers antibiotic resistance to geneticin.

A screenable marker may comprise a reporter gene, which allows the researcher to distinguish between wanted and unwanted cells. Certain embodiments of the present invention utilize reporter genes to indicate specific cell lineages. For example, the reporter gene can be located within expression elements and under the control of the ventricular- or atrial-selective regulatory elements normally associated with the coding region of a ventricular- or atrial-selective gene for simultaneous expression. A reporter allows the cells of a specific lineage to be isolated without placing them under drug or other selective pressures or otherwise risking cell viability.

Examples of such reporters include genes encoding cell surface proteins (e.g., CD4, HA epitope), fluorescent proteins, antigenic determinants and enzymes (e.g., β-galactosidase). The vector containing cells may be isolated, e.g., by FACS using fluorescently-tagged antibodies to the cell surface protein or substrates that can be converted to fluorescent products by a vector encoded enzyme.

In specific embodiments, the reporter gene is a fluorescent protein. A broad range of fluorescent protein genetic variants have been developed that feature fluorescence emission spectral profiles spanning almost the entire visible light spectrum. Mutagenesis efforts in the original Aequorea victoria jellyfish green fluorescent protein have resulted in new fluorescent probes that range in color from blue to yellow, and are some of the most widely used in vivo reporter molecules in biological research. Longer wavelength fluorescent proteins, emitting in the orange and red spectral regions, have been developed from the marine anemone, Discosoma striata, and reef corals belonging to the class Anthozoa. Still other species have been mined to produce similar proteins having cyan, green, yellow, orange, and deep red fluorescence emission. Developmental research efforts are ongoing to improve the brightness and stability of fluorescent proteins, thus improving their overall usefulness.

The cells in certain embodiments can be made to contain one or more genetic alterations by genetic engineering of the cells either before or after differentiation (US 2002/0168766). A cell is said to be “genetically altered”, “genetically modified” or “transgenic” when an exogenous nucleic acid or polynucleotide has been transferred into the cell by any suitable means of artificial manipulation, or where the cell is a progeny of the originally altered cell that has inherited the polynucleotide. For example, the cells can be processed to increase their replication potential by genetically altering the cells to express telomerase reverse transcriptase, either before or after they progress to restricted developmental lineage cells or terminally differentiated cells (U.S. Patent Application Publication 2003/0022367).

In certain embodiments, cells containing an exogenous nucleic acid construct may be identified in vitro or in vivo by including a marker in the expression vector, such as a selectable or screenable marker. Such markers would confer an identifiable change to the cell permitting easy identification of cells containing the expression vector, or help enrich or identify differentiated cardiac cells by using a tissue-specific promoter. For example, in the aspects of cardiomyocyte differentiation, cardiac-specific promoters may be used, such as promoters of cardiac troponin I (cTnI), cardiac troponin T (cTnT), sarcomeric myosin heavy chain (MHC), GATA-4, Nkx2.5, N-cadherin, 01-adrenoceptor, ANF, the MEF-2 family of transcription factors, creatine kinase MB (CK-MB), myoglobin, or atrial natriuretic factor (ANF). In aspects of neuron differentiation, neuron-specific promoters may be used, including but not limited to, TuJ-1, Map-2, Dcx or Synapsin. In aspects of hepatocyte differentiation, definitive endoderm- and/or hepatocyte-specific promoters may be used, including but not limited to, ATT, Cyp3a4, ASGPR, FoxA2, HNF4a or AFP.

Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to blasticidin, neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

In embodiments wherein cells are genetically modified, such as to add or reduce one or more features, the genetic modification may occur by any suitable method. For example, any genetic modification compositions or methods may be used to introduce exogenous nucleic acids into cells or to edit the genomic DNA, such as gene editing, homologous recombination or nonhomologous recombination, RNA-mediated genetic delivery or any conventional nucleic acid delivery methods. Non-limiting examples of the genetic modification methods may include gene editing methods such as by CRISPR/CAS9, zinc finger nuclease, or TALEN technology.

Genetic modification may also include the introduction of a selectable or screenable marker that aid selection or screen or imaging in vitro or in vivo. Particularly, in vivo imaging agents or suicide genes may be expressed exogenously or added to starting cells or progeny cells. In further aspects, the methods may involve image-guided adoptive cell therapy.

### SPECIFIC EMBODIMENTS

In a specific embodiments of the disclosure there is provided a method of preparing a cell population comprising clonal invariant natural killer (iNKT) T cells comprising: a) selecting CD34+ cells from human peripheral blood cells (PBMCs); b) culturing the CD34+ cells with medium comprising growth factors that include c-kit ligand, flt-3 ligand, and human thrombopoietin (TPO) c) transducing the selected CD34+ cells with a lentiviral vector comprising a nucleic acid sequence encoding α-TCR, β-TCR, and thymidine kinase; d) introducing into the selected CD34+ cells Cas9 and gRNA for beta 2 microglobulin (B2M) and/or CTIIA to disrupt expression of B2M or CTIIA genes thus eliminating the surface expression of HLA-I and/or HLA-II molecules; e) culturing the transduced cells for 2-12 (or 2-10 or 6-12) weeks with an irradiated stromal cell line expressing an exogenous Notch ligand to expand iNKT cells in a 3D aggregate cell culture; f) selecting iNKT cells lacking surface expression of HLA-I/II molecules; and, g) culturing the selected iNKT cells with irradiated feeder cells. In particular embodiments, 10⁸-10¹³ iNKT cells are prepared from the selected CD34+ cells.

Thus, the disclosure encompasses an advanced HSC-based iNKT cell therapy that is universal and off-the-shelf. Specifically, one can harvest G-CSF-mobilized CD34⁺ HSCs from healthy donors or from a cell repository. From a single donor, about 1-5×10⁸ HSCs can be collected. In specific cases, these HSCs are engineered in vitro with a Lenti/iNKT-sr39TK lentiviral vector and a CRISPR-Cas9/B2M-CIITA-gRNAs complex, then are differentiated into iNKT cells in an artificial thymic organoid (ATO) culture in 8 weeks. The iNKT cells may then be purified and further expanded in vitro for another 2-4 weeks, followed by cryopreservation and lot release. In specific aspects, about 10¹² iNKT cells are generated from HSCs of a single donor, which can be formulated into 1,000 to 10,000 doses (at ˜10⁸-10⁹ cells per dose, for example). The resulting cryopreserved cellular product, engineered iNKT cells, can then be readily stored and distributed to treat cancer patients off-the-shelf through allogenic adoptive cell transfer. Because iNKT cells can target multiple types of cancer without tumor antigen- and major histocompatibility complex (MHC)-restrictions, the iNKT therapy is useful as a universal cancer therapy for treating multiple cancers and a large population of cancer patients, thus addressing the unmet medical need (Vivier et al., 2012; Berzins et al., 2011). Particularly, the disclosed iNKT therapy is useful to treat the many types of cancer that have been clinically implicated to be subject to iNKT cell regulation, including blood cancers (leukemia, multiple myeloma, and myelodysplastic syndromes), and solid tumors (melanoma, colon, lung, breast, and head and neck cancers) (Berzins et al., 2011).

The scientific embodiments underlying the iNKT therapy are: 1) the lentiviral vector-mediated expression of a human iNKT T cell receptor (TCR) gene programs HSCs to differentiate into iNKT cells; 2) the inclusion of an sr39TK PET imaging/suicide gene allows for the monitoring of iNKT cells in patients using PET imaging, as well as the depletion of these cells through ganciclovir (GCV) administration in case of a safety need; 3) the CRISPR-Cas9/B2M-CIITA-gRNAs-based gene editing of HSCs knocks out the B2M and CIITA genes, resulting in an HLA-I/II-negative cellular product suitable for allogenic infusion; 4) the ATO culture system supports the efficient development of human iNKT cells in vitro; 5) the manufacturing process is of high yield and high purity. The Examples section herein provides data supporting these scientific embodiments.

In specific cases, the manufacturing of iNKT involves: 1) collection of G-CSF-mobilized leukopak; 2) purification of G-CSF-leukopak into CD34⁺ HSCs; 3) transduction of HSCs with lentiviral vector Lenti/iNKT-sr39TK; 4) gene editing of B2M and CIITA via CRISPR/Cas9; 5) in vitro differentiation into iNKT cells via ATO; 6) purification of iNKT cells; 7) in vitro cell expansion; 8) cell collection, formulation and cryopreservation. In a certain embodiment, there are two drug substances (Lenti/iNKT-sr39TK vector and iNKT cells), and the final drug product may be the formulated and cryopreserved iNKT in infusion bags, in specific cases.

Provided herein are examples of efficient protocols to generate iNKT cells. Demonstrated herein is an efficient gene editing of HSCs to ablate the cell surface expression of class I HLA via knockout of B2M. Taking advantage of the multiplex editing CRISPR/Cas9, one can also simultaneously disrupt cell surface class II HLA expression via knockout of the gene for the class II transactivator (CIITA), a key regulator of HLA-II expression (Steimle et al., 1994), for example using a validated gRNA sequence (Abrahimi et al., 2015). Thus, incorporating this gene editing step to disrupt cell surface HLA-I and HLA-II expression and the microbeads purification step, the inventors will generate iNKT cells. Flow cytometric analysis may be used to measure the purity and the surface phenotypes of these engineered iNKT cells. The cell purity may be characterized by TCR Vα24⁺Jα18⁺HLA-I⁻HLA-II⁻. In specific embodiments, this iNKT cell population is CD45RO⁺CD161⁺, indicative of memory and NK phenotypes, and contains both CD4⁺CD8⁻(CD4 single-positive), CD4⁻CD8⁺(CD8 single-positive), and CD4⁻CD8⁻ (double-negative, DN) (Kronenberg and Gapin, 2002). CD62L expression may be analyzed, as a recent study indicated that its expression is associated with in vivo persistence of iNKT cells and their antitumor activity (Tian et al., 2016). One can compare these phenotypes of iNKT with that iNKT from PBMCs. RNAseq may be employed to perform comparative gene expression analysis on iNKT and PBMC iNKT cells.

IFN-γ production and cytotoxicity assays may be used to assess the functional properties of iNKT, using PBMC iNKT as the benchmark control. iNKT cells may be simulated with irradiated PBMCs that have been pulsed with αGC and supernatants harvested from one day stimulation may be subjected to IFN-γ ELISA (Smith et al., 2015). Intracellular cytokine staining (ICCS) of IFN-γ may be performed as well on iNKT cells after 6-hour stimulation. The cytotoxicity assay may be conducted by incubating effector iNKT cells with αGC-loaded A375.CD1d target cells engineered to expression luciferase and GFP for 4 hours and cytotoxicity may be measured by a plate reader for its luminescence intensity. Because sr39TK is introduced as a PET/suicide gene, one can verify its function by incubating iNKT with ganciclovir (GCV) and cell survival rate may be measured by a MTT assay and an Annexin V-based flow cytometric assay, for example.

One can perform pharmacokinetics/Pharmacodynamics (PK/PD) studies. The PK/PD studies can determine in vivo in animal models the following: 1) expansion kinetics and persistence of infused iNKT; 2) biodistribution of iNKT in various tissues/organs; 3) ability of iNKT to traffic to tumors and how this filtration relates to tumor growth. One can utilize immunodeficient NSG mice bearing A375.CD1d (A375.CD1d) tumors as the solid tumor animal model. Two cell dose groups (1×10⁶ and 10×10⁶; n=8) may be investigated. The tumors may be inoculated (s.c.) on day −4 and the baseline PET imaging and bleeding may be conducted on day 0. Subsequently, iNKT cells may be infused intravenously (i.v.) and monitored by 1) PET imaging in live animals on days 7 and 21; 2) periodic bleeding on days 7, 14 and 21; 3) end-point tissue collection after animal termination on day 21. Cell collected from various bleedings may be analyzed by flow cytometry; iNKT cells should be CD161⁺6B11⁺. One can examine the expression of other markers such as CD45RO, CD62L, and CD4 to see how iNKT subsets vary over the time. PET imaging via sr39TK will allow one to track the presence of iNKT cells in tumors and other tissues/organs such as bone, liver, spleen, thymus, etc. At the end of the study, tumors and mouse tissues including spleen, liver, brain, heart, kidney, lung, stomach, bone marrow, ovary, intestine, etc., may be harvested for qPCR analysis to examine the distribution of iNKT cells.

One can characterize a mechanism of action (MOA) for the cells. iNKT cells are known to target tumor cells through either direct killing, or through the massive release of IFN-γ to direct NK and CD8 T cells to eradicate tumors (Fujii et al., 2013). An in vitro pharmacological study provides evidence of direct cytotoxicity. Here one can investigate the roles of NK and CD8 T cells in assisting antitumor reactivity in vivo. Tumor-bearing NSG mice (A375.CD1d or MM.1S.Luc) may be infused with either iNKT alone (a dose chosen based on above in vivo study) or in combination with PBMCs (mismatched donor, 5×10⁶); owing to the MHC negativity of iNKT, no allogenic immune response may occur between iNKT and unrelated PBMCs. Tumor growth may be monitored and compared between with and without PBMC groups (n=8 per group). If a greater antitumor response is observed from the combination group, it may indicate that components in PBMCs, for example NK and/or CD8 T cells, play a role to boost therapeutic efficacy, in specific embodiments. To further determine their individual roles, PBMCs with depletion of NK (via CD56 beads), CD8 T cells (via CD8 beads), or myeloid (via CD14 beads) cells, may be co-infused along with iNKT cells into tumor-bearing mice. Immune checkpoint inhibitors such as PD-1 and CTLA-4 have been suggested to regulate iNKT cell function (Pilones et al., 2012; Durgan et al., 2011). Through adding anti-PD-1 or anti-CTLA-4 treatment to the iNKT therapy, one can determine how these molecules modulate iNKT therapy and provide information on the design of combination cancer therapy.

Particular vectors may be utilized for the production of iNKT cells and/or their use. One can utilize a vector for genetic engineering of HSCs into iNKT cells such as an HIV-1 derived lentiviral vector Lenti/iNKT-sr39TK encoding a human iNKT TCR gene along with an sr39TK PET imaging/suicide gene. Components of this third generation self-inactivating (SIN) vector are: 1) 3′ self-inactivating long-term repeats (ALTR); 2) Ψ region vector genome packaging signal; 3) Rev Responsive Element (RRE) to enhance nuclear export of unspliced vector RNA; 4) central PolyPurine Tract (cPPT) to facilitate unclear import of vector genomes; 5) expression cassette of the α chain gene (TCRα) and β chain gene (TCRβ) of a human iNKT TCR, as well as the PET/suicide gene sr39TK (Gscheng et al., 2014) driven by internal promoter from the murine stem cell virus (MSCV). The iNKT TCRα and TCRβ and sr39TK genes are all codon-optimized and linked by 2A self-cleaving sequences (T2A and P2A) to achieve their optimal co-expression (Gscheng et al., 2014).

Regarding quality control of the vector, a series of QC assays may be performed to ensure that the vector product is of high quality. Those standard assays such as vector identity, vector physical titer, and vector purity (sterility, mycoplasma, viral contaminants, replication-competent lentivirus (RCL) testing, endotoxin, residual DNA and benzonase) may be conducted at IU VPF and provided in the Certificate of Analysis (COA). Additional QC assays that may be performed include 1) the transduction/biological titer (by transducing HT29 cells with serial dilutions and performing ddPCR, ≥1×10⁶ TU/ml); 2) the vector provirus integrity (by sequencing the vector-integrated portion of genomic DNA of transduced HT29 cells, same to original vector plasmid sequence); 3) the vector function. The vector function may be measured by transducing human PBMC T cells (Chodon et al., 2014). The expression of iNKT TCR gene may be detected by staining with the 6B11 specific for iNKT TCR (Montoya et al., 2007). The functionality of expressed iNKT TCRs will be analyzed by IFN-γ production in response to aGalCer stimulation (Watarai et al., 2008). The expression and functionality of sr39TK gene may be analyzed by penciclovir update assay and GCV killing assay (Gschweng et al., 2014. The stability of the vector stock (stored in −80 freezer) may be tested every 3 months by measuring its transduction titer.

### IX. Cell Manufacturing and Product Formulation

Provided herein are example processes that may be used in combination with embodiments of the disclosure for manufacturing iNKT cells. In specific embodiments, iNKT cells are the key drug substance that functions as “living drug” to target and fight disease in a mammal, including fight tumor cells, for example. In particular embodiments, they are generated by in vitro differentiation and expansion of genetically modified donor HSCs. Data demonstrates a novel and efficient protocol to produce the cells in a laboratory scale, and in specific embodiments the cells are made as an “off-the-shelf” cell product in a GMP-comparable manufacturing process. In specific cases, production scale is 10¹² cells per batch, which is estimated to treat 1000-10,000 patients.

An example of a cell manufacturing process that may be used in conjunction with embodiments of the disclosure or as alternatives is provided. Step 1 is to harvest donor G-CSF-mobilized PBSCs in blood collection facilities, which has become a routine procedure in many hospitals (Deotare et al., 2015). One can obtain fresh PBSCs in Leukopaks from the HemaCare for this project; HemaCare has IRB-approved collection protocols and donor consents and can support clinical trials and commercial product manufacturing. Step 2 is to enrich CD34⁺ HSCs from PBSCs using a CliniMACS system; one can use such a system located at the UCLA GMP facility to complete this step and one can yield at least 10⁸ CD34⁺ cells, in specific aspects. CD34-cells may be collected and stored as well (they may be used as PBMC feeder in Step 7).

Step 3 involves the HSC culture and vector transduction. CD34⁺ cells may be cultured in X-VIVO15 medium supplemented with 1% HAS (USP) and growth factor cocktails (c-kit ligand, flt-3 ligand and tpo; 50 ng/ml each) for 12 hrs in flasks coated with retronectin, followed by addition of the Lenti/iNKT-sr39TK vector for additional 8 hrs (Gschweng et al., 2014). Vector integration copies (VCN) may be measured by sampling ˜50 colonies formed in the methylcellulose assay for transduced cells and the average vector copy number per cell may be determined using ddPCR (Nolta et al., 1994). In specific cases the procedure is optimized and >50% transduction is routinely achieved with VCN=1-3 per cell.

Step 4 is to utilize the powerful CRISPR/Cas9 multiplex gene editing method to target the genomic loci of both B2M and CIITA in HSCs and disrupt their gene expression (Ren et al., 2017; Liu et al., 2017), and iNKT cells derived from edited HSCs will lack the MHC/HLA expression, thereby avoiding the rejection by the host immune system. Initial data has demonstrated the success of the B2M disruption for CD34⁺ HSCs with high efficiency (˜75% by flow analysis) via electroporation of Cas9/B2M-gRNA. B2M/CIITA double knockout may be achieved by electroporation of a mixture of RNPs (Cas9/B2M-gRNA and Cas9/CIITA-gRNA (Abrahimi et al., 2015)). One can optimize and validate this process (Gundry et al., 2016) by varying electroporation parameters, ratios of two RNPs, stem cell culture time (24, 48, or 72 hrs post-transduction) prior to electroporation, etc; one can use the high fidelity Cas9 protein (Slaymaker et al., 2016; Tsai and Joung, 2016) from IDT to minimize the “off-target” effect. Exemplary evaluation parameters may be viability, deletion (indel) frequency (on-target efficiency) measured by a T7E1 assay and next-generation sequencing (NGS) targeting the B2M and CIITA sites, MHC expression by flow cytometry, and hematopoietic function of edited HSCs measured by the colony formation unit (CFU) assay.

Step 5 is to in vitro differentiate modified CD34⁺ HSCs into iNKT cells (for example via the artificial thymic organoid (ATO) culture). Initial studies have shown that functional iNKT cells can be efficiently generated from HSCs engineered to express iNKT TCRs. Building upon this data, one can test and validate an 8-week, GMP-compatible ATO culture process to produce 10¹⁰ iNKT cells from 10⁸ modified CD34⁺ HSCs. ATO involves pipetting a cell slurry (5 μl) containing mixture of HSCs (5×10⁴) and irradiated (80 Gy) MS5-hDLL1 stromal cells (10⁶) as a drop format onto a 0.4-μm Millicell transwell insert, followed by placing the insert into a 6-well plate containing 1 ml RB27 medium; medium may be changed every 4 days for 8 weeks. Considering 3 ATOs per insert, approximately 170 six-well plates for each batch production may be utilized. One can use an automated programmable pipetting/dispensing system (epMontion 5070f from Eppendorf) placed in biosafety cabinet for plating ATO droplets and medium exchange; a 2-hr operation may be needed for completing 170 plates each round. At the end of ATO culture, iNKT cells may be harvested and characterized. In specific embodiments a component of ATO is the MS5-hDLL1 stromal cell line that is constructed by lentiviral transduction to express human DLL1 followed by cell sorting. In preparation for certain GMP processes, one can perform a single cell clonal selection process on this polyclonal cell population to establish several clonal MS5-hDLL1 cell lines, from which one can choose an efficient one (evaluated by ATO culture) and use it to generate a master cell bank. Such a bank may be used to supply irradiated stromal cells for future clinical grade ATO culture.

Step 6 is to purify iNKT cells using the CliniMACS system. This step purification is to deplete MHCI⁺ and MHCII⁺ cells and enrich iNKT⁺ cells. Anti-MHCI and anti-MHCII beads may be prepared by incubating Miltenyi anti-Biotin beads with commercially available biotinylated anti-MHCI (clone W6/32, HLA-A, B, C), anti-B2M (clone 2M2), and anti-MHCII (clone Tu39, HLA-DR, DP, DQ), and anti-TCR Vα24-Jα18 (clone 6B11). 6B11 directly-coated microbeads are also available from Miltenyi; anti-iNKT beads are available from Miltenyi Biotec. Harvested iNKT cells may be labeled by anti-MHC bead mixtures and washed twice and MHCI⁺ and/or MHCII⁺ cells may be depleted using the CliniMACS depletion program; if necessary, this depletion step can be repeated to further remove residual MHC⁺ cells. Subsequently, iNKT cells may be further purified using the standard anti-iNKT beads and the CliniMACS enrichment program. The cell purity may be measured by flow cytometry, for example.

Step 7 is to expand purified iNKT cells in vitro. Starting from 10¹⁰ cells, one can expand into 10¹² iNKT cells using an already validated PBMC feeder-based in vitro expansion protocol (Yamasaki et al., 2011; Heczey et al., 2014). One can evaluate a G-Rex-based bioprocess for this cell expansion. G-Rex is a cell growth flask with a gas-permeable membrane at the bottom allowing more efficient gas exchange; A G-Rex500M flask has the capacity to support a 100-fold cell expansion in 10 days (Vera et al., 2010; Bajgain et al., 2014; Jin et al., 2012). The stored CD34⁻ cells (used as feeder cells) from the Step 1 may be thawed, pulsed with αGalCer (100 ng/ml), and irradiated (40 Gy). iNKT cells may be mixed with irradiated feeder cells (1:4 ratio), seeded into G-Rex flasks (1.25×10⁸ iNKT each, 80 flasks), and allowed to expand for 2 weeks. IL-2 (200 U/ml) will be added every 2-3 days and one medium exchange will occur at day 7; all medium manipulation may be achieved by peristaltic pumps. This expansion process is GMP-compatible because a similar PBMC feeder-based expansion procedure (termed rapid expansion protocol) has been already utilized to produce therapeutic T cells for many clinical trials (Dudley et al., 2008; Rosenberg et al., 2008).

Step 8 is to formulate the harvested iNKT cells from Step 7 (the active drug component) into cell suspension for direct infusion. After at least 3 rounds of extensive washing, cells from Step 7 may be counted and suspended into an infusion/cold storage-compatible solution (10⁷-10⁸ cells/ml), which is composed of Plasma-Lyte A Injection (31.25% v/v), Dextrose and Sodium Chloride Injection (31.25% v/v), Human Albumin (20% v/v), Dextran 40 in Dextrose Inject (10%, v/v) and Cryoserv DMSO (7.5%, v/v); this solution has been used to formulate tisagenlecleucel, an approved T cell product from Novartis (Grupp et al., 2013). Once filled into FDA-approved freezing bags (such as CryoMACS freezing bags from Miltenyi Biotec), the product may be frozen in a controlled rate freezer and stored in a liquid nitrogen freezer. One can perform validation and/or optimization studies by measuring viability and recovery to ensure that this formulation is appropriate for an iNKT cell product.

Various IPC assays such as cell counting, viability, sterility, mycoplasma, identity, purity, VCN, etc.) may be incorporated into the proposed bioprocess to ensure a high-quality production. Testing may include the following: 1) appearance (color, opacity); 2) cell viability and count; 3) identity and VCN by qPCR for iNKT TCR; 4) purity by iNKT positivity and B2M negativity; 5) endotoxins; 6) sterility; 7) mycoplasma; 8) potency measured by IFN-γ release in response to αGalCer stimulation; 9) RCL (replication-competent lentivirus) (Cornetta et al, 2011). Most of these assays are either standard biological assays or specific assays unique to this product. Product stability testing may be performed by periodically thawing LN-stored bags and measuring their cell viability, purity, recovery, potency (IFN-γ release) and sterility. In particular embodiments, the product is stable for at least one year.

### X. Source of Starting Cells

Starting cells such as pluripotent stem cells or hematopoietic stem or progenitor cells may be used in certain compositions or methods for differentiation along a selected T cell lineage. Stromal cells may be used to co-culture with the stem or progenitor cells. In some embodiments, stromal cells are not used to co-culture with the stem or progenitor cells.

B. Stromal Cells

Stromal cells are connective tissue cells of any organ, for example in the bone marrow, thymus, uterine mucosa (endometrium), prostate, and the ovary. They are cells that support the function of the parenchymal cells of that organ. Fibroblasts (also known as mesenchymal stromal cells/MSC) and pericytes are among the most common types of stromal cells.

The interaction between stromal cells and tumor cells is known to play a major role in cancer growth and progression. In addition, by regulating locally cytokine networks (e.g. M-CSF, LIF), bone marrow stromal cells have been described to be involved in human haematopoiesis and inflammatory processes.

Stromal cells in the bone marrow, thymus, and other hematopoietic organs regulate hematopoietic and immune cell development though cell-cell ligand-receptor interactions and through the release of soluble factors including cytokines and chemokines. Stromal cells in these tissues form niches that regulate stem cell maintenance, lineage specification and commitment, and differentiation to effector cell types.

Stroma is made up of the non-malignant host cells. Stromal cells also provides an extracellular matrix on which tissue-specific cell types, and in some cases tumors, can grow.

C. Hematopoietic Stem and Progenitor Cells

Due to the significant medical potential of hematopoietic stem and progenitor cells, substantial work has been done to try to improve methods for the differentiation of hematopoietic progenitor cells from embryonic stem cells. In the human adult, hematopoietic stem cells present primarily in bone marrow produce heterogeneous populations of hematopoietic (CD34+) progenitor cells that differentiate into all the cells of the blood system. In an adult human, hematopoietic progenitors proliferate and differentiate resulting in the generation of hundreds of billions of mature blood cells daily. Hematopoietic progenitor cells are also present in cord blood. In vitro, human embryonic stem cells may be differentiated into hematopoietic progenitor cells. Hematopoietic progenitor cells may also be expanded or enriched from a sample of peripheral blood as described below. The hematopoietic cells can be of human origin, murine origin or any other mammalian species.

Isolation of hematopoietic progenitor cells include any selection methods, including cell sorters, magnetic separation using antibody-coated magnetic beads, packed columns; affinity chromatography; cytotoxic agents joined to a monoclonal antibody or used in conjunction with a monoclonal antibody, including but not limited to, complement and cytotoxins; and “panning” with antibody attached to a solid matrix, e.g., plate, or any other convenient technique.

The use of separation or isolation techniques include, but are not limited to, those based on differences in physical (density gradient centrifugation and counter-flow centrifugal elutriation), cell surface (lectin and antibody affinity), and vital staining properties (mitochondria-binding dye rho123 and DNA-binding dye Hoechst 33342). Techniques providing accurate separation include but are not limited to, FACS (Fluorescence-activated cell sorting) or MACS (Magnetic-activated cell sorting), which can have varying degrees of sophistication, e.g., a plurality of color channels, low angle and obtuse light scattering detecting channels, impedance channels, etc.

The antibodies utilized in the preceding techniques or techniques used to assess cell type purity (such as flow cytometry) can be conjugated to identifiable agents including, but not limited to, enzymes, magnetic beads, colloidal magnetic beads, haptens, fluorochromes, metal compounds, radioactive compounds, drugs or haptens. The enzymes that can be conjugated to the antibodies include, but are not limited to, alkaline phosphatase, peroxidase, urease and β-galactosidase. The fluorochromes that can be conjugated to the antibodies include, but are not limited to, fluorescein isothiocyanate, tetramethylrhodamine isothiocyanate, phycoerythrin, allophycocyanins and Texas Red. For additional fluorochromes that can be conjugated to antibodies, see Haugland, Molecular Probes: Handbook of Fluorescent Probes and Research Chemicals (1992-1994). The metal compounds that can be conjugated to the antibodies include, but are not limited to, ferritin, colloidal gold, and particularly, colloidal superparamagnetic beads. The haptens that can be conjugated to the antibodies include, but are not limited to, biotin, digoxygenin, oxazalone, and nitrophenol. The radioactive compounds that can be conjugated or incorporated into the antibodies are known to the art, and include but are not limited to technetium 99m (99TC), 125I and amino acids comprising any radionuclides, including, but not limited to, 14C, 3H and 35S.

Other techniques for positive selection may be employed, which permit accurate separation, such as affinity columns, and the like. The method should permit the removal to a residual amount of less than about 20%, preferably less than about 5%, of the non-target cell populations.

Cells may be selected based on light-scatter properties as well as their expression of various cell surface antigens. The purified stem cells have low side scatter and low to medium forward scatter profiles by FACS analysis. Cytospin preparations show the enriched stem cells to have a size between mature lymphoid cells and mature granulocytes.

It also is possible to enrich the inoculation population for CD34+ cells prior to culture, using for example, the method of Sutherland et al. (1992) and that described in U.S. Pat. No. 4,714,680. For example, the cells are subject to negative selection to remove those cells that express lineage specific markers. In an illustrative embodiment, a cell population may be subjected to negative selection for depletion of non-CD34+ hematopoietic cells and/or particular hematopoietic cell subsets. Negative selection can be performed on the basis of cell surface expression of a variety of molecules, including T cell markers such as CD2, CD4 and CD8; B cell markers such as CD10, CD19 and CD20; monocyte marker CD14; the NK cell marker CD2, CD16, and CD56 or any lineage specific markers. Negative selection can be performed on the basis of cell surface expression of a variety of molecules, such as a cocktail of antibodies (e.g., CD2, CD3, CD11b, CD14, CD15, CD16, CD19, CD56, CD123, and CD235a) which may be used for separation of other cell types, e.g., via MACS or column separation.

As used herein, lineage-negative (LIN−) refers to cells lacking at least one marker associated with lineage committed cells, e.g., markers associated with T cells (such as CD2, 3, 4 and 8), B cells (such as CD10, 19 and 20), myeloid cells (such as CD14, 15, 16 and 33), natural killer (“NK”) cells (such as CD2, 16 and 56), RBC (such as glycophorin A), megakaryocytes (CD41), mast cells, eosinophils or basophils or other markers such as CD38, CD71, and HLA-DR. Preferably the lineage specific markers include, but are not limited to, at least one of CD2, CD14, CD15, CD16, CD19, CD20, CD33, CD38, HLA-DR and CD71. More preferably, LIN− will include at least CD14 and CD15. Further purification can be achieved by positive selection for, e.g., c-kit+ or Thy-1+. Further enrichment can be obtained by use of the mitochondrial binding dye rhodamine 123 and selection for rhodamine+ cells, by methods known in the art. A highly enriched composition can be obtained by selective isolation of cells that are CD34+, preferably CD34+LIN−, and most preferably, CD34+ Thy-1+LIN−. Populations highly enriched in stem cells and methods for obtaining them are well known to those of skill in the art, see e.g., methods described in PCT Patent Application Nos. PCT/US94/09760; PCT/US94/08574 and PCT/US94/10501.

Various techniques may be employed to separate the cells by initially removing cells of dedicated lineage. Monoclonal antibodies are particularly useful for identifying markers associated with particular cell lineages and/or stages of differentiation. The antibodies may be attached to a solid support to allow for crude separation. The separation techniques employed should maximize the retention of viability of the fraction to be collected. Various techniques of different efficacy may be employed to obtain “relatively crude” separations. Such separations are where up to 10%, usually not more than about 5%, preferably not more than about 1%, of the total cells present are undesired cells that remain with the cell population to be retained. The particular technique employed will depend upon efficiency of separation, associated cytotoxicity, ease and speed of performance, and necessity for sophisticated equipment and/or technical skill.

Selection of the progenitor cells need not be achieved solely with a marker specific for the cells. By using a combination of negative selection and positive selection, enriched cell populations can be obtained.

D. Sources of Blood Cells

Hematopoietic stem cells (HSCs) normally reside in the bone marrow but can be forced into the blood, a process termed mobilization used clinically to harvest large numbers of HSCs in peripheral blood. One example of a mobilizing agent of choice is granulocyte colony-stimulating factor (G-CSF).

CD34+ hematopoietic stem cells or progenitors that circulate in the peripheral blood can be collected by apheresis techniques either in the unperturbed state, or after mobilization following the external administration of hematopoietic growth factors like G-CSF. The number of the stem or progenitor cells collected following mobilization is greater than that obtained after apheresis in the unperturbed state. In a particular aspect of the present invention, the source of the cell population is a subject whose cells have not been mobilized by extrinsically applied factors because there is no need to enrich hematopoietic stem cells or progenitor cells in vivo.

Populations of cells for use in the methods described herein may be mammalian cells, such as human cells, non-human primate cells, rodent cells (e.g., mouse or rat), bovine cells, ovine cells, porcine cells, equine cells, sheep cell, canine cells, and feline cells or a mixture thereof. Non-human primate cells include rhesus macaque cells. The cells may be obtained from an animal, e.g., a human patient, or they may be from cell lines. If the cells are obtained from an animal, they may be used as such, e.g., as unseparated cells (i.e., a mixed population); they may have been established in culture first, e.g., by transformation; or they may have been subjected to preliminary purification methods. For example, a cell population may be manipulated by positive or negative selection based on expression of cell surface markers; stimulated with one or more antigens in vitro or in vivo; treated with one or more biological modifiers in vitro or in vivo; or a combination of any or all of these.

Populations of cells include peripheral blood mononuclear cells (PBMC), whole blood or fractions thereof containing mixed populations, spleen cells, bone marrow cells, tumor infiltrating lymphocytes, cells obtained by leukapheresis, biopsy tissue, lymph nodes, e.g., lymph nodes draining from a tumor. Suitable donors include immunized donors, non-immunized (naive) donors, treated or untreated donors. A “treated” donor is one that has been exposed to one or more biological modifiers. An “untreated” donor has not been exposed to one or more biological modifiers.

For example, peripheral blood mononuclear cells (PBMC) can be obtained as described according to methods known in the art. Examples of such methods are discussed by Kim et al. (1992); Biswas et al. (1990); Biswas et al. (1991).

Methods of obtaining precursor cells from populations of cells are also well known in the art. Precursor cells may be expanded using various cytokines, such as hSCF, hFLT3, and/or IL-3 (Akkina et al., 1996), or CD34+ cells may be enriched using MACS or FACS. As mentioned above, negative selection techniques may also be used to enrich CD34+ cells.

It is also possible to obtain a cell sample from a subject, and then to enrich it for a desired cell type. For example, PBMCs and/or CD34+ hematopoietic cells can be isolated from blood as described herein. Cells can also be isolated from other cells using a variety of techniques, such as isolation and/or activation with an antibody binding to an epitope on the cell surface of the desired cell type. Another method that can be used includes negative selection using antibodies to cell surface markers to selectively enrich for a specific cell type without activating the cell by receptor engagement.

Bone marrow cells may be obtained from iliac crest, femora, tibiae, spine, rib or other medullary spaces. Bone marrow may be taken out of the patient and isolated through various separations and washing procedures. An exemplary procedure for isolation of bone marrow cells comprises the following steps: a) centrifugal separation of bone marrow suspension in three fractions and collecting the intermediate fraction, or buffycoat; b) the buffycoat fraction from step (a) is centrifuged one more time in a separation fluid, commonly Ficoll (a trademark of Pharmacia Fine Chemicals AB), and an intermediate fraction which contains the bone marrow cells is collected; and c) washing of the collected fraction from step (b) for recovery of re-transfusable bone marrow cells.

E. Pluripotent Stem Cells

The cells suitable for the compositions and methods described herein may be hematopoietic stem and progenitor cells may also be prepared from differentiation of pluripotent stem cells in vitro. In some embodiments, the cells used in the methods described herein are pluripotent stem cells (embryonic stem cells or induced pluripotent stem cells) directly seeded into the ATOs. In further embodiments, the cells used in the methods and compositions described herein are a derivative or progeny of the PSC such as, but not limited to mesoderm progenitors, hemato-endothelial progenitors, or hematopoietic progenitors.

The term “pluripotent stem cell” refers to a cell capable of giving rise to cells of all three germinal layers, that is, endoderm, mesoderm and ectoderm. Although in theory a pluripotent stem cell can differentiate into any cell of the body, the experimental determination of pluripotency is typically based on differentiation of a pluripotent cell into several cell types of each germinal layer. In some embodiments, a pluripotent stem cell is an embryonic stem (ES) cell derived from the inner cell mass of a blastocyst. In other embodiments, the pluripotent stem cell is an induced pluripotent stem cell derived by reprogramming somatic cells. In certain embodiments, the pluripotent stem cell is an embryonic stem cell derived by somatic cell nuclear transfer.

Embryonic stem (ES) cells are pluripotent cells derived from the inner cell mass of a blastocyst. ES cells can be isolated by removing the outer trophectoderm layer of a developing embryo, then culturing the inner mass cells on a feeder layer of non-growing cells. Under appropriate conditions, colonies of proliferating, undifferentiated ES cells are produced. The colonies can be removed, dissociated into individual cells, then replated on a fresh feeder layer. The replated cells can continue to proliferate, producing new colonies of undifferentiated ES cells. The new colonies can then be removed, dissociated, replated again and allowed to grow. This process of “subculturing” or “passaging” undifferentiated ES cells can be repeated a number of times to produce cell lines containing undifferentiated ES cells (U.S. Pat. Nos. 5,843,780; 6,200,806; 7,029,913). A “primary cell culture” is a culture of cells directly obtained from a tissue such as the inner cell mass of a blastocyst. A “subculture” is any culture derived from the primary cell culture.

Methods for obtaining mouse ES cells are well known. In one method, a preimplantation blastocyst from the 129 strain of mice is treated with mouse antiserum to remove the trophoectoderm, and the inner cell mass is cultured on a feeder cell layer of chemically inactivated mouse embryonic fibroblasts in medium containing fetal calf serum. Colonies of undifferentiated ES cells that develop are subcultured on mouse embryonic fibroblast feeder layers in the presence of fetal calf serum to produce populations of ES cells. In some methods, mouse ES cells can be grown in the absence of a feeder layer by adding the cytokine leukemia inhibitory factor (LIF) to serum-containing culture medium (Smith, 2000). In other methods, mouse ES cells can be grown in serum-free medium in the presence of bone morphogenetic protein and LIF (Ying et al., 2003).

Human ES cells can be obtained from blastocysts using previously described methods (Thomson et al., 1995; Thomson et al., 1998; Thomson and Marshall, 1998; Reubinoff et al, 2000.) In one method, day-5 human blastocysts are exposed to rabbit anti-human spleen cell antiserum, then exposed to a 1:5 dilution of Guinea pig complement to lyse trophectoderm cells. After removing the lysed trophectoderm cells from the intact inner cell mass, the inner cell mass is cultured on a feeder layer of gamma-inactivated mouse embryonic fibroblasts and in the presence of fetal bovine serum. After 9 to 15 days, clumps of cells derived from the inner cell mass can be chemically (i.e. exposed to trypsin) or mechanically dissociated and replated in fresh medium containing fetal bovine serum and a feeder layer of mouse embryonic fibroblasts. Upon further proliferation, colonies having undifferentiated morphology are selected by micropipette, mechanically dissociated into clumps, and replated (see U.S. Pat. No. 6,833,269). ES-like morphology is characterized as compact colonies with apparently high nucleus to cytoplasm ratio and prominent nucleoli. Resulting ES cells can be routinely passaged by brief trypsinization or by selection of individual colonies by micropipette. In some methods, human ES cells can be grown without serum by culturing the ES cells on a feeder layer of fibroblasts in the presence of basic fibroblast growth factor (Amit et al., 2000). In other methods, human ES cells can be grown without a feeder cell layer by culturing the cells on a protein matrix such as Matrigel™ or laminin in the presence of “conditioned” medium containing basic fibroblast growth factor (Xu et al., 2001). The medium is previously conditioned by coculturing with fibroblasts.

Methods for the isolation of rhesus monkey and common marmoset ES cells are also known (Thomson, and Marshall, 1998; Thomson et al., 1995; Thomson and Odorico, 2000).

Another source of ES cells are established ES cell lines. Various mouse cell lines and human ES cell lines are known and conditions for their growth and propagation have been defined. For example, the mouse CGR8 cell line was established from the inner cell mass of mouse strain 129 embryos, and cultures of CGR8 cells can be grown in the presence of LIF without feeder layers. As a further example, human ES cell lines H1, H7, H9, H13 and H14 were established by Thompson et al. In addition, subclones H9.1 and H9.2 of the H9 line have been developed.

The source of ES cells can be a blastocyst, cells derived from culturing the inner cell mass of a blastocyst, or cells obtained from cultures of established cell lines. Thus, as used herein, the term “ES cells” can refer to inner cell mass cells of a blastocyst, ES cells obtained from cultures of inner mass cells, and ES cells obtained from cultures of ES cell lines.

Induced pluripotent stem (iPS) cells are cells which have the characteristics of ES cells but are obtained by the reprogramming of differentiated somatic cells. Induced pluripotent stem cells have been obtained by various methods. In one method, adult human dermal fibroblasts are transfected with transcription factors Oct4, Sox2, c-Myc and Klf4 using retroviral transduction (Takahashi et al., 2007). The transfected cells are plated on SNL feeder cells (a mouse cell fibroblast cell line that produces LIF) in medium supplemented with basic fibroblast growth factor (bFGF). After approximately 25 days, colonies resembling human ES cell colonies appear in culture. The ES cell-like colonies are picked and expanded on feeder cells in the presence of bFGF.

Based on cell characteristics, cells of the ES cell-like colonies are induced pluripotent stem cells. The induced pluripotent stem cells are morphologically similar to human ES cells, and express various human ES cell markers. Also, when growing under conditions that are known to result in differentiation of human ES cells, the induced pluripotent stem cells differentiate accordingly. For example, the induced pluripotent stem cells can differentiate into cells having neuronal structures and neuronal markers.

In another method, human fetal or newborn fibroblasts are transfected with four genes, Oct4, Sox2, Nanog and Lin28 using lentivirus transduction (Yu et al., 2007). At 12-20 days post infection, colonies with human ES cell morphology become visible. The colonies are picked and expanded. The induced pluripotent stem cells making up the colonies are morphologically similar to human ES cells, express various human ES cell markers, and form teratomas having neural tissue, cartilage and gut epithelium after injection into mice.

Methods of preparing induced pluripotent stem cells from mouse are also known (Takahashi and Yamanaka, 2006). Induction of iPS cells typically require the expression of or exposure to at least one member from Sox family and at least one member from Oct family. Sox and Oct are thought to be central to the transcriptional regulatory hierarchy that specifies ES cell identity. For example, Sox may be Sox-1, Sox-2, Sox-3, Sox-15, or Sox-18; Oct may be Oct-4. Additional factors may increase the reprogramming efficiency, like Nanog, Lin28, Klf4, or c-Myc; specific sets of reprogramming factors may be a set comprising Sox-2, Oct-4, Nanog and, optionally, Lin-28; or comprising Sox-2, Oct4, Klf and, optionally, c-Myc.

IPS cells, like ES cells, have characteristic antigens that can be identified or confirmed by immunohistochemistry or flow cytometry, using antibodies for SSEA-1, SSEA-3 and SSEA-4 (Developmental Studies Hybridoma Bank, National Institute of Child Health and Human Development, Bethesda Md.), and TRA-1-60 and TRA-1-81 (Andrews et al., 1987). Pluripotency of embryonic stem cells can be confirmed by injecting approximately 0.5-10×10⁶ cells into the rear leg muscles of 8-12 week old male SCID mice. Teratomas develop that demonstrate at least one cell type of each of the three germ layers.

### XI. Methods of Using the Cells

The iNKT cells of the disclosure may or may not be utilized directly after production. In some cases they are stored for later purpose. In any event, they may be utilized in therapeutic or preventative applications for a mammalian subject (human, dog, cat, horse, etc.) such as a patient. The patient may be in need of cell therapy for a medical condition of any kind, including allogeneic cell therapy.

Methods of treating a patient with a therapeutically effective amount of iNKT cells of the disclosure comprise administering the cells or clonal populations thereof to the patient The cells or cell populations may be allogeneic with respect to the patient. The patient does not exhibit signs of depletion of the cells or cell population, in particular embodiments. The patient may or may not have cancer and/or a disease or condition involving inflammation. In specific embodiments wherein the patient has cancer, tumor cells of the cancer patient are killed after administering the cells or cell population to the patient. In specific cases wherein the patient has inflammation, the inflammation is reduced following administering the cells or cell population to the patient. In specific embodiments of the methods of treatment, the method further comprises administering to the patient a compound that initiates the suicide gene product.

For patients with cancer, once infused into patients it is expected that this cell product can employ multiple mechanisms to target and eradicate tumor cells. The infused cells can directly recognize and kill CD1d⁺ tumor cells through cytotoxicity. They can secrete cytokines such as IFN-γ to activate NK cells to kill HLA-negative tumor cells, and also activate DCs which then stimulate cytotoxic T cells to kill HLA-positive tumor cells. Accordingly, the inventors plan a series of in vitro and in vivo studies to demonstrate the pharmacological efficacy of this cell product for cancer therapy.

Because the iNKT cells can target a large range of cancers without tumor antigen- and MHC-restrictions, an off-the-shelf iNKT cellular product is useful as a general cancer immunotherapy for treating any type of cancer and a large population of cancer patients. In specific cases, the present therapy is useful for patients with cancers that have been clinically indicated to be subject to iNKT cell regulation, including multiple types of solid tumors (melanoma, colon, lung, breast, and head and neck cancers) and blood cancers (leukemia, multiple myeloma, and myelodysplastic syndromes), for example.

In some embodiments of any of the above-disclosed methods, the subject has or is at risk of having an autoimmune disease, graft versus host disease (GVHD), or graft rejection. The subject may be one diagnosed with such disease or one that has been determined to have a pre-disposition to such disease based on genetic or family history analysis. The subject may also be one that is preparing to or has undergone a transplant. In some embodiments, the method is for treating an autoimmune disease, GVHD, or graft rejection.

Individuals treated with the present cell therapy may or may not have been treated for the particular medical condition prior to receiving the iNKT cell therapy. In cases wherein the individual has cancer, the cancer may be primary, metastatic, resistant to therapy, and so forth. patients who have exhausted conventional treatment options.

In particular embodiments, the cells are provided to the patient at 10⁷-10⁹ cells per dose. In specific embodiments, the dosing regimen is a single-dose of allogeneic iNKT cells following lymphodeleting conditioning. The cells may be administered intravenously following lymphodepleting conditioning with fludarabine and cyclophosphamide, for example.

In cases wherein antitumor efficacy in vivo is characterized for subsequent in vivo therapeutic cases, in vivo pharmacological responses may be measured by treating tumor-bearing NSG mice with escalating doses (1×10⁶, 5×10⁶, 10×10⁶) of iNKT cells (n=8 per group); treatment with PBS may be included as a control. Two tumor models may be utilized, as examples. A375.CD1d (1×10⁶ s.c.) may be used as a solid tumor model and MM.1S.Luc (5×10⁶ i.v.) may be used as a hematological malignancy model. Tumor growth can be monitored by either measuring size (A375.CD1d) or bioluminescence imaging (MM.1S.Luc). Antitumor immune responses can be measured by PET imaging, periodic bleeding, and end-point tumor harvest followed by flow cytometry and qPCR. Inhibition of tumor growth in response to iNKT treatment can indicate the therapeutic efficacy of iNKT cell therapy. Correlation of tumor inhibition with iNKT doses can confirm the therapeutic role of the iNKT cells and indicate an effective therapeutic window for human therapy. Detection of iNKT cell responses to tumors can demonstrate the pharmacological antitumor activities of these cells in vivo.

Methods may be employed with respect to individuals who have tested positive for a medical condition, who have one or more symptoms of a medical condition, or who are deemed to be at risk for developing such a condition. In some embodiments, the compositions and methods described herein are used to treat an inflammatory or autoimmune component of a disorder listed herein and/or known in the art.

In some embodiments, the method is for a patient with relapsed/refractory multiple myeloma (MM). In some embodiments, the patient has received at least 1, 2, 3, 4, 5, 6, 7, 8, or more prior treatments for MM. The prior treatments may include a treatment or therapy described herein. In some embodiments, the prior treatments comprises one or more of a proteasome inhibitor, an immunomodulatory agent, and/or an anti-CD38 antibody. Proteasome inhibitors include, for example, bortezomib or carfilzomib. Immunomodulatory agents include, for example, lenalidomide or pomalidomide. In some embodiments, the patient had received the prior therapy within 10, 20, 30, 40, 50, 60, 70, 80, or 90 days or hours of administration of the current compositions and cells of the disclosure. In some embodiments, the patient is one in which at least 5, 6, 7, 8, 9, 10, 11, 12, 13, 14, 15, 16, 17, 18, 19, 20, 21, 22, 23, 24, 25, 26, 27, 28, 39, or 30% of the malignant cells or malignant plasma cells express B cell maturation antigen (BCMA). In some embodiments, the patient is one that has undergone prior autologous BCMA-targeted CAR T cell therapy and has failed the prior treatment either because the prior treatment was not effective or because the prior treatment was deemed too toxic. In some embodiments, the patient is one that has been determined to have BCMA+ malignant cells. In some embodiments, the patient is one that has been determined to have BCMA+ malignant cells in the relapsed refractory phase of MM. In some embodiments, the method is for a patient with leukemia. In some embodiments, the patient has received at least 1, 2, 3, 4, 5, 6, 7, 8, or more more prior treatments for leukemia. In some embodiments, the patient is one in which at least 5, 6, 7, 8, 9, 10, 11, 12, 13, 14, 15, 16, 17, 18, 19, 20, 21, 22, 23, 24, 25, 26, 27, 28, 39, or 30% of the malignant cells express CD19 (i.e. are CD19+). In some embodiments, the patient is one that has undergone prior autologous CD19-targeted CAR T cell therapy and has failed the prior treatment either because the prior treatment was not effective or because the prior treatment was deemed too toxic. In some embodiments, the patient is one that has been determined to have CD19+ malignant cells.

In some embodiments, the methods relate to administration of the cells or compositions described herein for the treatment of a cancer or administration to a person with a cancer. In some embodiments, the cancer is multiple myeloma. In some embodiments, the cancer is a B-cell cancer. In some embodiments the cancer is diffuse large B-cell lymphoma, follicular lymphoma, marginal zone B-cell lymphoma, mucosa-associated lymphatic tissue lymphoma, small lymphocytic lymphoma (also known as chronic lymphocytic leukemia, CLL), mantle cell lymphoma, primary mediastinal (thymic) large B cell lymphoma, T cell/histiocyte-rich large B-cell lymphoma, primary cutaneous diffuse large B-cell lymphoma, EBV positive diffuse large B-cell lymphoma, burkitt's lymphoma, lymphoplasmacytic lymphoma, nodal marginal zone B cell lymphoma, splenic marginal zone lymphoma, intravascular large B-cell lymphoma, primary effusion lymphoma, lymphomatoid granulomatosis, central nervous system lymphoma, ALK-positive large B-cell lymphoma, plasmablastic lymphoma, or large B-cell lymphoma. In some embodiments, the cancer comprises a blood cancer. In some embodiments, the blood cancer comprises myeloma, leukemia, lymphoma, Non-Hodgkin lymphoma, Hodgkin lymphoma, a myeloid neoplasm, a lymphoid neoplasm, acute lymphoblastic leukemia (ALL), acute myelogenous leukemia (AML), chronic lymphocytic leukemia (CLL), chronic myelogenous leukemia (CML), acute monocytic leukemia (AMoL), chronic myeloid leukaemia, BCR-ABL1-positive, chronic neutrophilic leukaemia, polycythaemia vera, primary myelofibrosis, essential thrombocythaemia, chronic eosinophilic leukaemia, NOS, myeloproliferative neoplasm, cutaneous mastocytosis, indolent systemic mastocytosis, systemic mastocytosis with an associated haematological neoplasm, aggressive systemic mastocytosis, mast cell leukaemia, mast cell sarcoma, myeloid/lymphoid neoplasms with PDGFRA rearrangement, myeloid/lymphoid neoplasms with PDGFRB rearrangement, myeloid/lymphoid neoplasms with FGFR1 rearrangement, myeloid/lymphoid neoplasms with PCM1-JAK2, chronic myelomonocytic leukaemia, atypical chronic myeloid leukaemia, BCR-ABL1-negative, juvenile myelomonocytic leukaemia, myelodysplastic/myeloproliferative neoplasm with ring sideroblasts and thrombocytosis, myelodysplastic/myeloproliferative neoplasm, myelodysplastic syndrome with single lineage dysplasia, myelodysplastic syndrome with ring sideroblasts and single lineage dysplasia, myelodysplastic syndrome with ring sideroblasts and multilineage dysplasia, myelodysplastic syndrome with multilineage dysplasia, myelodysplastic syndrome with excess blasts, myelodysplastic syndrome with isolated del(5q), myelodysplastic syndrome, unclassifiable, refractory cytopenia of childhood, acute myeloid leukaemia with germline CEBPA mutation, myeloid neoplasms with germline DDX41 mutation, myeloid neoplasms with germline RUNX1 mutation, myeloid neoplasms with germline ANKRD26 mutation, myeloid neoplasms with germline ETV6 mutation, myeloid neoplasms with germline GATA2 mutation, AML with t(8;21)(q22;q22.1) RUNX1-RUNX1T1; AML with inv(16)(p13.1q22) or t(16;16)(p13.1;q22) CBFB-MYH11; acute promyelocytic leukaemia with PML-RARA, AML with t(9;11)(p21.3;q23.3) KMT2A-MLLT3; AML with t(6;9)(p23;q34.1) DEK-NUP214; AML with inv(3)(q21.3q26.2) or t(3;3)(q21.3;q26.2) GATA2, MECOM; AML (megakaryoblastic) with t(1;22)(p13.3;q13.1) RBM15-MKL1; AML with BCR-ABL1; AML with mutated NPM1; AML with biallelic mutation of CEBPA; AML with mutated RUNX1; AML with myelodysplasia-related changes; Therapy-related myeloid neoplasms; AML with minimal differentiation; AML without maturation; AML with maturation; acute myelomonocytic leukaemia, acute monoblastic and monocytic leukaemia, pure erythroid leukaemia, acute megakaryoblastic leukaemia, acute basophilic leukaemia, acute panmyelosis with myelofibrosis, myeloid sarcoma, myeloid proliferations associated with Down syndrome, blastic plasmacytoid dendritic cell neoplasm, acute undifferentiated leukaemia, mixed-phenotype acute leukaemia with t(9;22)(q34.1;q11.2) BCR-ABL1; mixed-phenotype acute leukaemia with t(v;11q23.3) KMT2A-rearranged; mixed-phenotype acute leukaemia, B/myeloid; mixed-phenotype acute leukaemia, T/myeloid; mixed-phenotype acute leukaemia, rare types; acute leukaemias of ambiguous lineage, B-lymphoblastic leukaemia/lymphoma, B-lymphoblastic leukaemia/lymphoma with t(9;22)(q34.1;q11.2) BCR-ABL1; B-lymphoblastic leukaemia/lymphoma with t(v;11q23.3) KMT2A-rearranged; B-lymphoblastic leukaemia/lymphoma with t(12;21)(p13.2;q22.1) ETV6-RUNX1; B-lymphoblastic leukaemia/lymphoma with hyperdiploidy; B-lymphoblastic leukaemia/lymphoma with hypodiploidy (hypodiploid ALL); B-lymphoblastic leukaemia/lymphoma with t(5;14)(q31.1;q32.1) IGH/IL3; B-lymphoblastic leukaemia/lymphoma with t(1;19)(q23;p13.3) TCF3-PBX1; B-lymphoblastic leukaemia/lymphoma, BCR-AQL 1-like; D-lymphoblastic leukaemia/lymphoma with iAMP21; T-lymphoblastic leukaemia/lymphoma; Early T-cell precursor lymphoblastic leukaemia; NK-lymphoblastic leukaemia/lymphoma; chronic lymphocytic leukaemia (CLL)/small lymphocytic lymphoma; monoclonal B-cell lymphocytosis, CLL-type; monoclonal B-cell lymphocytosis, non-CLL-type; B-cell prolymphocytic leukaemia; splenic marginal zone lymphoma, hairy cell leukaemia, splenic diffuse red pulp small B-cell lymphoma, hairy cell leukaemia variant, Waldentrom macroglobulinemia, IgM monoclonal gammopathy, mu heavy chain disease, gamma heavy chain disease, alpha heavy chain disease, plasma cell neoplasms, extranodal marginal zone lymphoma of mucosa-associated lymphoid tissue (MALT lymphoma), nodal marginal zone lymphoma, follicular lymphoma, paediatric-type follicular lymphoma, large B-cell lymphoma with IRF4 rearrangement, primary cutaneous follicle centre lymphoma, mantle cell lymphoma, diffuse large B-cell lymphoma (DLBCL), T-cell/histiocyte-rich large B-cell lymphoma, primary DLBCL of the CNS, primary cutaneous DLBCL, EBV-positive DLBCL, EBV-positive mucocutaneous ulcer, DLBCL associated with chronic inflammation, lymphomatoid granulomatosis, grade 1,2, lymphomatoid granulomatosis, grade 3, primary mediastinal (thymic) large B-cell lymphoma, intravascular large B-cell lymphoma, ALK-positive large B-cell lymphoma, plasmablastic lymphoma, primary effusion lymphoma, multicentric Castleman disease, HHV8-positive DLBCL, HHV8-positive germinotropic lymphoproliferative disorder, Burkitt lymphoma, Burkitt-like lymphoma with 11q aberration, high-grade B-cell lymphoma, B-cell lymphoma, unclassifiable, with features intermediate between DLBCL and classic Hodgkin lymphoma, and histiocytic and dendritic cell neoplasms.

Certain aspects of the disclosure relate to the treatment of cancer and/or use of the cells and compositions of the disclosure to treat cancer. The cancer to be treated or antigen may be an antigen associated with any cancer known in the art or, for example, epithelial cancer, (e.g., breast, gastrointestinal, lung), prostate cancer, bladder cancer, lung (e.g., small cell lung) cancer, colon cancer, ovarian cancer, brain cancer, gastric cancer, renal cell carcinoma, pancreatic cancer, liver cancer, esophageal cancer, head and neck cancer, or a colorectal cancer. In some embodiments, the cancer to be treated or antigen is from one of the following cancers: adenocortical carcinoma, agnogenic myeloid metaplasia, AIDS-related cancers (e.g., AIDS-related lymphoma), anal cancer, appendix cancer, astrocytoma (e.g., cerebellar and cerebral), basal cell carcinoma, bile duct cancer (e.g., extrahepatic), bladder cancer, bone cancer, (osteosarcoma and malignant fibrous histiocytoma), brain tumor (e.g., glioma, brain stem glioma, cerebellar or cerebral astrocytoma (e.g., pilocytic astrocytoma, diffuse astrocytoma, anaplastic (malignant) astrocytoma), malignant glioma, ependymoma, oligodenglioma, meningioma, meningiosarcoma, craniopharyngioma, haemangioblastomas, medulloblastoma, supratentorial primitive neuroectodermal tumors, visual pathway and hypothalamic glioma, and glioblastoma), breast cancer, bronchial adenomas/carcinoids, carcinoid tumor (e.g., gastrointestinal carcinoid tumor), carcinoma of unknown primary, central nervous system lymphoma, cervical cancer, colon cancer, colorectal cancer, chronic myeloproliferative disorders, endometrial cancer (e.g., uterine cancer), ependymoma, esophageal cancer, Ewing's family of tumors, eye cancer (e.g., intraocular melanoma and retinoblastoma), gallbladder cancer, gastric (stomach) cancer, gastrointestinal carcinoid tumor, gastrointestinal stromal tumor (GIST), germ cell tumor, (e.g., extracranial, extragonadal, ovarian), gestational trophoblastic tumor, head and neck cancer, hepatocellular (liver) cancer (e.g., hepatic carcinoma and heptoma), hypopharyngeal cancer, islet cell carcinoma (endocrine pancreas), laryngeal cancer, laryngeal cancer, leukemia, lip and oral cavity cancer, oral cancer, liver cancer, lung cancer (e.g., small cell lung cancer, non-small cell lung cancer, adenocarcinoma of the lung, and squamous carcinoma of the lung), lymphoid neoplasm (e.g., lymphoma), medulloblastoma, ovarian cancer, mesothelioma, metastatic squamous neck cancer, mouth cancer, multiple endocrine neoplasia syndrome, myelodysplastic syndromes, myelodysplastic/myeloproliferative diseases, nasal cavity and paranasal sinus cancer, nasopharyngeal cancer, neuroblastoma, neuroendocrine cancer, oropharyngeal cancer, ovarian cancer (e.g., ovarian epithelial cancer, ovarian germ cell tumor, ovarian low malignant potential tumor), pancreatic cancer, parathyroid cancer, penile cancer, cancer of the peritoneal, pharyngeal cancer, pheochromocytoma, pineoblastoma and supratentorial primitive neuroectodermal tumors, pituitary tumor, pleuropulmonary blastoma, lymphoma, primary central nervous system lymphoma (microglioma), pulmonary lymphangiomyomatosis, rectal cancer, renal cancer, renal pelvis and ureter cancer (transitional cell cancer), rhabdomyosarcoma, salivary gland cancer, skin cancer (e.g., non-melanoma (e.g., squamous cell carcinoma), melanoma, and Merkel cell carcinoma), small intestine cancer, squamous cell cancer, testicular cancer, throat cancer, thymoma and thymic carcinoma, thyroid cancer, tuberous sclerosis, urethral cancer, vaginal cancer, vulvar cancer, Wilms' tumor, and post-transplant lymphoproliferative disorder (PTLD), abnormal vascular proliferation associated with phakomatoses, edema (such as that associated with brain tumors), or Meigs' syndrome.

Certain aspects of the disclosure relate to the treatment of an autoimmune condition and/or use of an autoimmune-associated antigen. The autoimmune disease to be treated or antigen may be an antigen associated with any autoimmune condition known in the art or, for example, diabetes, graft rejection, GVHD, arthritis (rheumatoid arthritis such as acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, and juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, atopy including atopic diseases such as hay fever and Job's syndrome, dermatitis including contact dermatitis, chronic contact dermatitis, exfoliative dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis, x-linked hyper IgM syndrome, allergic intraocular inflammatory diseases, urticaria such as chronic allergic urticaria and chronic idiopathic urticaria, including chronic autoimmune urticaria, myositis, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, ataxic sclerosis, neuromyelitis optica (NMO), inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), bowel inflammation, pyoderma gangrenosum, erythema nodosum, primary sclerosing cholangitis, respiratory distress syndrome, including adult or acute respiratory distress syndrome (ARDS), meningitis, inflammation of all or part of the uvea, iritis, choroiditis, an autoimmune hematological disorder, rheumatoid spondylitis, rheumatoid synovitis, hereditary angioedema, cranial nerve damage as in meningitis, herpes gestationis, pemphigoid gestationis, pruritis scroti, autoimmune premature ovarian failure, sudden hearing loss due to an autoimmune condition, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, proliferative nephritis, autoimmune polyglandular endocrine failure, balanitis including balanitis circumscripta plasmacellularis, balanoposthitis, erythema annulare centrifugum, erythema dyschromicum perstans, eythema multiform, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, allergic reaction, eczema including allergic or atopic eczema, asteatotic eczema, dyshidrotic eczema, and vesicular palmoplantar eczema, asthma such as asthma bronchiale, bronchial asthma, and auto-immune asthma, conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, lupus, including lupus nephritis, lupus cerebritis, pediatric lupus, non-renal lupus, extra-renal lupus, discoid lupus and discoid lupus erythematosus, alopecia lupus, systemic lupus erythematosus (SLE) such as cutaneous SLE or subacute cutaneous SLE, neonatal lupus syndrome (NLE), and lupus erythematosus disseminatus, juvenile onset (Type I) diabetes mellitus, including pediatric insulin-dependent diabetes mellitus (IDDM), and adult onset diabetes mellitus (Type II diabetes) and autoimmune diabetes. Also contemplated are immune responses associated with acute and delayed hypersensitivity mediated by cytokines and T-lymphocytes, sarcoidosis, granulomatosis including lymphomatoid granulomatosis, Wegener's granulomatosis, agranulocytosis, vasculitides, including vasculitis, large-vessel vasculitis (including polymyalgia rheumatica and gianT cell (Takayasu's) arteritis), medium-vessel vasculitis (including Kawasaki's disease and polyarteritis nodosa/periarteritis nodosa), microscopic polyarteritis, immunovasculitis, CNS vasculitis, cutaneous vasculitis, hypersensitivity vasculitis, necrotizing vasculitis such as systemic necrotizing vasculitis, and ANCA-associated vasculitis, such as Churg-Strauss vasculitis or syndrome (CSS) and ANCA-associated small-vessel vasculitis, temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), Addison's disease, autoimmune neutropenia, pancytopenia, leukopenia, diseases involving leukocyte diapedesis, CNS inflammatory disorders, Alzheimer's disease, Parkinson's disease, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Behcet's disease/syndrome, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, thermal injury, preeclampsia, an immune complex disorder such as immune complex nephritis, antibody-mediated nephritis, polyneuropathies, chronic neuropathy such as IgM polyneuropathies or IgM-mediated neuropathy, autoimmune or immune-mediated thrombocytopenia such as idiopathic thrombocytopenic purpura (ITP) including chronic or acute ITP, scleritis such as idiopathic cerato-scleritis, episcleritis, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), experimental autoimmune encephalomyelitis, myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, gianT cell hepatitis, chronic active hepatitis or autoimmune chronic active hepatitis, lymphoid interstitial pneumonitis (LIP), bronchiolitis obliterans (non-transplant) vs NSIP, Guillain-Barre syndrome, Berger's disease (IgA nephropathy), idiopathic IgA nephropathy, linear IgA dermatosis, acute febrile neutrophilic dermatosis, subcorneal pustular dermatosis, transient acantholytic dermatosis, cirrhosis such as primary biliary cirrhosis and pneumonocirrhosis, autoimmune enteropathy syndrome, Celiac or Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, polychondritis such as refractory or relapsed or relapsing polychondritis, pulmonary alveolar proteinosis, Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, which includes monoclonal B cell lymphocytosis (e.g., benign monoclonal gammopathy and monoclonal gammopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal or segmental or focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, Dressler's syndrome, alopecia greata, alopecia totalis, CREST syndrome (calcinosis, Raynaud's phenomenon, esophageal dysmotility, sclerodactyl), and telangiectasia), male and female autoimmune infertility, e.g., due to anti-spermatozoan antibodies, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, parasitic diseases such as leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis (acute or chronic), or Fuch's cyclitis, Henoch-Schonlein purpura, human immunodeficiency virus (HIV) infection, SCID, acquired immune deficiency syndrome (AIDS), echovirus infection, sepsis, endotoxemia, pancreatitis, thyroxicosis, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, gianT cell polymyalgia, chronic hypersensitivity pneumonitis, keratoconjunctivitis sicca, epidemic keratoconjunctivitis, idiopathic nephritic syndrome, minimal change nephropathy, benign familial and ischemia-reperfusion injury, transplant organ reperfusion, retinal autoimmunity, joint inflammation, bronchitis, chronic obstructive airway/pulmonary disease, silicosis, aphthae, aphthous stomatitis, arteriosclerotic disorders, asperniogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired spenic atrophy, non-malignant thymoma, vitiligo, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, autoimmune polyglandular syndrome type I, adult-onset idiopathic hypoparathyroidism (AOIH), cardiomyopathy such as dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia syndrome, angiectasis, autoimmune disorders associated with collagen disease, rheumatism, neurological disease, lymphadenitis, reduction in blood pressure response, vascular dysfunction, tissue injury, cardiovascular ischemia, hyperalgesia, renal ischemia, cerebral ischemia, and disease accompanying vascularization, allergic hypersensitivity disorders, glomerulonephritides, reperfusion injury, ischemic reperfusion disorder, reperfusion injury of myocardial or other tissues, lymphomatous tracheobronchitis, inflammatory dermatoses, dermatoses with acute inflammatory components, multiple organ failure, bullous diseases, renal cortical necrosis, acute purulent meningitis or other central nervous system inflammatory disorders, ocular and orbital inflammatory disorders, granulocyte transfusion-associated syndromes, cytokine-induced toxicity, narcolepsy, acute serious inflammation, chronic intractable inflammation, pyelitis, endarterial hyperplasia, peptic ulcer, valvulitis, graft versus host disease, contact hypersensitivity, asthmatic airway hyperreaction, and endometriosis.

Further aspects relate to the treatment or prevention microbial infection and/or use of microbial antigens. The microbial infection to be treated or prevented or antigen may be an antigen associated with any microbial infection known in the art or, for example, anthrax, cervical cancer (human papillomavirus), diphtheria, hepatitis A, hepatitis B, Haemophilus influenzae type b (Hib), human papillomavirus (HPV), influenza (Flu), japanese encephalitis (JE), lyme disease, measles, meningococcal, monkeypox, mumps, pertussis, pneumococcal, polio, rabies, rotavirus, rubella, shingles (herpes zoster), smallpox, tetanus, typhoid, tuberculosis (TB), varicella (Chickenpox), and yellow fever.

In some embodiments, the methods and compositions may be for vaccinating an individual to prevent a medical condition, such as cancer, inflammation, infection, and so forth.

### XII. Additional Therapies

A. Immunotherapy

In some embodiments, the methods comprise administration of a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immunotherapies useful in the methods of the disclosure are described below.

2. Checkpoint Inhibitors and Combination Treatment

Embodiments of the disclosure may include administration of immune checkpoint inhibitors (also referred to as checkpoint inhibitor therapy), which are further described below. The checkpoint inhibitor therapy may be a monotherapy, targeting only one cellular checkpoint proteins or may be combination therapy that targets at least two cellular checkpoint proteins. For example, the checkpoint inhibitor monotherapy may comprise one of: a PD-1, PD-L1, or PD-L2 inhibitor or may comprise one of a CTLA-4, B7-1, or B7-2 inhibitor. The checkpoint inhibitor combination therapy may comprise one of: a PD-1, PD-L1, or PD-L2 inhibitor and, in combination, may further comprise one of a CTLA-4, B7-1, or B7-2 inhibitor. The combination of inhibitors in combination therapy need not be in the same composition, but can be administered either at the same time, at substantially the same time, or in a dosing regimen that includes periodic administration of both of the inhibitors, wherein the period may be a time period described herein.

b. PD-1, PD-L1, and PD-L2 inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PD-L1 on epithelial cells and tumor cells. PD-L2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PD-L1 activity.

Alternative names for “PD-1” include CD279 and SLEB2. Alternative names for “PD-L1” include B7-H1, B7-4, CD274, and B7-H. Alternative names for “PD-L2” include B7-DC, Btdc, and CD273. In some embodiments, PD-1, PD-L1, and PD-L2 are human PD-1, PD-L1 and PD-L2.

In some embodiments, the PD-1 inhibitor is a molecule that inhibits the binding of PD-1 to its ligand binding partners. In a specific aspect, the PD-1 ligand binding partners are PD-L1 and/or PD-L2. In another embodiment, a PD-L1 inhibitor is a molecule that inhibits the binding of PD-L1 to its binding partners. In a specific aspect, PD-L1 binding partners are PD-1 and/or B7-1. In another embodiment, the PD-L2 inhibitor is a molecule that inhibits the binding of PD-L2 to its binding partners. In a specific aspect, a PD-L2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, and pidilizumab. In some embodiments, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In some embodiments, the PD-L1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PD-L2-Fc fusion soluble receptor described in WO2010/027827 and WO2011/066342. Additional PD-1 inhibitors include MEDI0680, also known as AMP-514, and REGN2810.

In some embodiments, the immune checkpoint inhibitor is a PD-L1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PD-L2 inhibitor such as rHIgM12B7.

In some embodiments, the inhibitor comprises the heavy and light chain CDRs or VRs of nivolumab, pembrolizumab, or pidilizumab. Accordingly, in one embodiment, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another embodiment, the antibody competes for binding with and/or binds to the same epitope on PD-1, PD-L1, or PD-L2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

c. CTLA-4, B7-1, and B7-2 Inhibitors

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA-4 is a member of the immunoglobulin superfamily that is expressed on the surface of Helper T cells and transmits an inhibitory signal to T cells. CTLA-4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some embodiments, the inhibitor blocks the CTLA-4 and B7-1 interaction. In some embodiments, the inhibitor blocks the CTLA-4 and B7-2 interaction.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WOO 1/14424).

In some embodiments, the inhibitor comprises the heavy and light chain CDRs or VRs of tremelimumab or ipilimumab. Accordingly, in one embodiment, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another embodiment, the antibody competes for binding with and/or binds to the same epitope on PD-1, B7-1, or B7-2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

3. Inhibition of Co-Stimulatory Molecules

In some embodiments, the immunotherapy comprises an inhibitor of a co-stimulatory molecule. In some embodiments, the inhibitor comprises an inhibitor of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Inhibitors include inhibitory antibodies, polypeptides, compounds, and nucleic acids.

4. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment, they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor.

5. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

6. Adoptive T-Cell Therapy

Adoptive T cell therapy is a form of passive immunization by the transfusion of T-cells (adoptive cell transfer). They are found in blood and tissue and usually activate when they find foreign pathogens. Specifically, they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumor death.

Multiple ways of producing and obtaining tumor targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

It is contemplated that a cancer treatment may exclude any of the cancer treatments described herein. Furthermore, embodiments of the disclosure include patients that have been previously treated for a therapy described herein, are currently being treated for a therapy described herein, or have not been treated for a therapy described herein. In some embodiments, the patient is one that has been determined to be resistant to a therapy described herein. In some embodiments, the patient is one that has been determined to be sensitive to a therapy described herein.

B. Oncolytic Virus

In some embodiments, the additional therapy comprises an oncolytic virus. An oncolytic virus is a virus that preferentially infects and kills cancer cells. As the infected cancer cells are destroyed by oncolysis, they release new infectious virus particles or virions to help destroy the remaining tumor. Oncolytic viruses are thought not only to cause direct destruction of the tumor cells, but also to stimulate host anti-tumor immune responses for long-term immunotherapy.

C. Polysaccharides

In some embodiments, the additional therapy comprises polysaccharides. Certain compounds found in mushrooms, primarily polysaccharides, can up-regulate the immune system and may have anti-cancer properties. For example, beta-glucans such as lentinan have been shown in laboratory studies to stimulate macrophage, NK cells, T cells and immune system cytokines and have been investigated in clinical trials as immunologic adjuvants.

D. Neoantigens

In some embodiments, the additional therapy comprises neoantigen administration. Many tumors express mutations. These mutations potentially create new targetable antigens (neoantigens) for use in T cell immunotherapy. The presence of CD8⁺ T cells in cancer lesions, as identified using RNA sequencing data, is higher in tumors with a high mutational burden. The level of transcripts associated with cytolytic activity of natural killer cells and T cells positively correlates with mutational load in many human tumors.

E. Chemotherapies

In some embodiments, the additional therapy comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some embodiments, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m² to about 20 mg/m² for 5 days every three weeks for a total of three courses being contemplated in certain embodiments. In some embodiments, the amount of cisplatin delivered to the cell and/or subject in conjunction with the construct comprising an Egr-1 promoter operatively linked to a polynucleotide encoding the therapeutic polypeptide is less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain embodiments, appropriate intravenous doses for an adult include about 60 mg/m² to about 75 mg/m² at about 21-day intervals or about 25 mg/m² to about 30 mg/m² on each of 2 or 3 successive days repeated at about 3 week to about 4 week intervals or about 20 mg/m² once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN₂), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. In one suitable embodiment, the chemotherapeutic agent may be administered in an amount effective to cause arrest or regression of the cancer in a host, when the chemotherapy is administered with the construct. In other embodiments, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

F. Radiotherapy

In some embodiments, the additional therapy or prior therapy comprises radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

In some embodiments, the amount of ionizing radiation is greater than 20 Gy and is administered in one dose. In some embodiments, the amount of ionizing radiation is 18 Gy and is administered in three doses. In some embodiments, the amount of ionizing radiation is at least, at most, or exactly 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, or 40 Gy (or any derivable range therein). In some embodiments, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some embodiments, the amount of IR may be presented as a total dose of IR, which is then administered in fractionated doses. For example, in some embodiments, the total dose is 50 Gy administered in 10 fractionated doses of 5 Gy each. In some embodiments, the total dose is 50-90 Gy, administered in 20-60 fractionated doses of 2-3 Gy each. In some embodiments, the total dose of IR is at least, at most, or about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 (or any derivable range therein). In some embodiments, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein. In some embodiments, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some embodiments, at least, at most, or exactly 1, 2, 3, 4, 5, 6, 7, 8, 9, 10, 11, or 12 (or any derivable range therein) fractionated doses are administered per day. In some embodiments, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

G. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

H. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

XIII. Sequences

| | | SEQ |
|---|---|---|
| | | ID |
| Description | Sequence | NO: |
| iNKT TCR-alpha chain | gtgggcgatagaggttcagccttagggaggctgcattttggagctgggactcagct | 1 |
| cloned sequence | gattgtcatacctgacatc | |
| iNKT TCR-beta chain | gccagcggtgatgctcggggggggggaaataccctctattttggaaaaggaagc | 2 |
| cloned sequence | cggctcattgttgtagaggat | |
| iNKT TCR-beta chain | gccagcggggggacagtccattctggaaatacgctctattttggagaaggaagcc | 3 |
| cloned sequence | ggctcattgttgtagaggat | |
| iNKT TCR-beta chain | gccagcggtgatacgggacaaacaaacacagaagtcttctttggtaaaggaacca | 4 |
| cloned sequence | gactcacagttgtagaggat | |
| iNKT TCR-beta chain | gccagcggtgaggggacagcaaacacagaagtcttctttggtaaaggaaccaga | 5 |
| cloned sequence | ctcacagttgtagaggat | |
| iNKT TCR-beta chain | gccagcggtgaggcagggaacacagaagtcttctttggtaaaggaaccagactc | 6 |
| cloned sequence | acagttgtagaggat | |
| iNKT TCR-alpha chain | gtgagcgacagaggctcaaccctggggaggctatactttggaagaggaactcagt | 7 |
| cloned sequence | tgactgtctggcctgatatccag | |
| iNKT TCR-beta chain | agcagtgacctccgaggacagaacacagatacgcagtattttggcccaggcacc | 8 |
| cloned sequence | cggctgacagtgctcgaggac | |
| iNKT TCR-beta chain | agcagtgaattaaaggaaacaggggttcaagagacccagtacttcgggccaggc | 9 |
| cloned sequence | acgcggctcctggtgctcgaggac | |
| iNKT TCR-beta chain | agcagtgtatctcagggcggcactgaagctttctttggacaaggcaccagactcac | 10 |
| cloned sequence | agttgtagaggac | |
| iNKT TCR-beta chain | agcagtgtatctcagggcggcactgaagctttctttggacaaggcaccagactcac | 11 |
| cloned sequence | agttgtagaggac | |
| iNKT TCR-beta chain | agcagtgaccggacaggcgtgaacactgaagctttctttggacaaggcaccagac | 12 |
| cloned sequence | tcacagttgtagaggac | |
| iNKT TCR-beta chain | agcagtgaaccggacagggggggggctgaagctttctttggacaaggcaccaga | 13 |
| cloned sequence | ctcacagttgtagaggac | |
| Human iNKT TCR- | atgaaaaagcatctgacgaccttcttggtgattttgtggctttatttttatagggggaat | 14 |
| alpha chain cDNA | ggcaaaaaccaagtggagcagagtcctcagtccctgatcatcctggagggaaag | |
| | aactgcactcttcaatgcaattatacagtgagccccttcagcaacttaaggtggtata | |
| | agcaagatactgggagaggtcctgtttccctgacaatcatgactttcagtgagaaca | |
| | caaagtcgaacggaagatatacagcaactctggatgcagacacaaagcaaagct | |
| | ctctgcacatcacagcctcccagctcagcgattcagcctcctacatctgtgtggtga | |
| | gcgacagaggctcaaccctggggaggctatactttggaagaggaactcagttgac | |
| | tgtctggcctgatatccagaaccctgaccctgccgtgtaccagctgagagactcta | |
| | aatccagtgacaagtctgtctgcctattcaccgattttgattctcaaacaaatgtgtca | |
| | caaagtaaggattctgatgtgtatatcacagacaaaactgtgctagacatgaggtct | |
| | atggacttcaagagcaacagtgctgtggcctggagcaacaaatctgactttgcatgt | |
| | gcaaacgccttcaacaacagcattattccagaagacaccttcttccccagcccaga | |
| | aagttcctgtgatgtcaagctggtcgagaaaagctttgaaacagatacgaacctaa | |
| | actttcaaaacctgtcagtgattgggttccgaatcctcctcctgaaagtggccgggtt | |
| | taatctgctcatgacgctgcggctgtggtccagctga | |
| Human iNKT TCR- | atgaaaaagcatctgacaacattcctggtcattctgtggctgtacttctaccgaggca | 15 |
| alpha chain cDNA | acggcaaaaatcaggtggagcagtccccacagtccctgatcattctggaggggaa | |
| codon-optimized | gaactgcactctgcagtgtaattacaccgtgtctccctttagtaacctgcgctggtat | |
| | aaacaggacaccggacgaggacccgtgagcctgacaatcatgactttctcagag | |
| | aacacaaagagcaatggacggtacaccgctacactggacgcagataccaaacag | |
| | agctccctgcacatcacagcatctcagctgtcagatagcgcctcctacatttgcgtg | |
| | gtctctgaccgagggagtaccctgggccgactgtattttggaagggggacccagc | |
| | tgacagtgtggcccgacatccagaacccagatcccgccgtctaccagctgcgcg | |
| | acagcaagtctagtgataaaagcgtgtgcctgttcacagactttgattctcagactaa | |
| | tgtctctcagagtaaggacagtgacgtgtacattactgacaaaaccgtcctggatat | |
| | gaggagcatggacttcaagtcaaacagcgccgtggcttggtcaaacaagagcga | |
| | cttcgcatgcgccaatgcttttaacaattcaatcattccagaggataccttctttcctag | |
| | cccagaatcaagctgtgacgtgaagctggtcgagaaaagtttcgaaactgatacca | |
| | acctgaattttcagaacctgtctgtgatcggcttcagaatcctgctgctgaaggtcgc | |
| | cggctttaatctgctgatgacactgagactgtggtcctcttga | |
| Human iNKT TCR- | atgactatcaggctcctctgctacatgggcttttattttctgggggcaggcctcatgg | 16 |
| beta chain cDNA | aagctgacatctaccagaccccaagataccttgttatagggacaggaaagaagat | |
| (before D/J/N region) | cactctggaatgttctcaaaccatgggccatgacaaaatgtactggtatcaacaaga | |
| | tccaggaatggaactacacctcatccactattcctatggagttaattccacagagaa | |
| | gggagatctttcctctgagtcaacagtctccagaataaggacggagcattttcccct | |
| | gaccctggagtctgccaggccctcacatacctctcagtacctctgtgccagc | |
| Human iNKT TCR- | atgaccatccggctgctgtgctacatgggcttctattttctgggggcaggcctgatg | 17 |
| beta chain cDNA | gaagccgacatctaccagactcccagatacctggtcatcggaaccgggaagaaa | |
| codon-optimized | attacactggagtgttcccagacaatgggccacgataagatgtactggtatcagca | |
| | ggaccctgggatggaactgcacctgatccattactcctatggcgtgaactctaccg | |
| | agaagggcgacctgagcagcgaatccaccgtctctcgaattaggacagagcactt | |
| | tcctctgactctggaaagcgcccgaccaagtcatacatcacagtacctgtgcgcta | |
| | gc | |
| Human iNKT TCR | gtagcggttgggccccaagagacccagtacttcgggccaggcacgcggctcctg | 18 |
| Beta Chain Diverse | gtgctc | |
| Region (D/J/N) | | |
| Human iNKT TCR | gtggcagtcggacctcaggagacccagtacttcggacccggcacccgcctgctg | 19 |
| Beta Chain Diverse | gtgctg | |
| Region (D/J/N) | | |
| Human iNKT TCR | agtgggccagggtacgagcagtacttcgggccgggcaccaggctcacggtcac | 20 |
| Beta Chain Diverse | a | |
| Region (D/J/N) | | |
| Human iNKT TCR | tcaggacccggctacgagcagtatttcggccccggaactcggctgaccgtgacc | 21 |
| Beta Chain Diverse | | |
| Region (D/J/N) | | |
| Human iNKT TCR | agtccccaattaaacactgaagctttctttggacaaggcaccagactcacagttgta | 22 |
| Beta Chain Diverse | | |
| Region (D/J/N) | | |
| Human iNKT TCR | tctccacagctgaacaccgaggccttcttcgggcagggcacaaggcttaccgtgg | 23 |
| Beta Chain Diverse | tg | |
| Region (D/J/N) | | |
| Human iNKT TCR | agtgaattgcgggcgctcgggcccagctcctataattcacccctccactttgggaa | 24 |
| Beta Chain Diverse | cgggaccaggctcactgtgaca | |
| Region (D/J/N) | | |
| Human iNKT TCR | tccgaactccgagccctggggcctagctcctacaatagccccctgcactttggcaa | 25 |
| Beta Chain Diverse | cggaaccaggctgacggtcacc | |
| Region (D/J/N) | | |
| Human iNKT TCR | agtgaacaggggactactgcgggagctttctttggacaaggcaccagactcacag | 26 |
| Beta Chain Diverse | ttgta | |
| Region (D/J/N) | | |
| Human iNKT TCR | tccgaacagggaaccacagcaggagccttcttcggtcagggaacaagactgaca | 27 |
| Beta Chain Diverse | gtcgtg | |
| Region (D/J/N) | | |
| Human iNKT TCR | agtgagtcacgacatgcgacaggaaacaccatatattttggagagggaagttggct | 28 |
| Beta Chain Diverse | cactgttgta | |
| Region (D/J/N) | | |
| Human iNKT TCR | agcgagagcaggcacgcaaccgggaacaccatatactttggcgagggctcctgg | 29 |
| Beta Chain Diverse | ctgactgtggtg | |
| Region (D/J/N) | | |
| Human iNKT TCR | agtgtacccgggaacgacaggggcaatgaaaaactgttttttggcagtggaaccc | 30 |
| Beta Chain Diverse | agctctctgtcttg | |
| Region (D/J/N) | | |
| Human iNKT TCR | tccgtgcctggcaacgatagaggtaacgagaagctgatttcggatccggcacaca | 31 |
| Beta Chain Diverse | gctgtctgtcctg | |
| Region (D/J/N) | | |
| Human iNKT TCR | agtgaaggggggggccttaagctagccaaaaacattcagtacttcggcgccggg | 32 |
| Beta Chain Diverse | acccggctctcagtgctg | |
| Region (D/J/N) | | |
| Human iNKT TCR | agtgagggagggggactgaagctggctaagaatattcagtacttcggcgccggc | 33 |
| Beta Chain Diverse | actagactgtctgtgctg | |
| Region (D/J/N) | | |
| Human iNKT TCR | agtgaattcgcctcttcggtacgtggaaacaccatatattttggagagggaagttgg | 34 |
| Beta Chain Diverse | ctcactgttgta | |
| Region (D/J/N) | | |
| Human iNKT TCR | tctgagttcgcgagcagcgtccggggtaataccatttacttcggggaaggcagctg | 35 |
| Beta Chain Diverse | gctgaccgtggtg | |
| Region (D/J/N) | | |
| Human iNKT TCR | agtgcggcattaggccgggagacccagtacttcgggccaggcacgcggctcctg | 36 |
| Beta Chain Diverse | gtgctc | |
| Region (D/J/N) | | |
| Human iNKT TCR | tctgcagcccttggccgagagactcagtacttcggccctggcacaagactgctcgt | 37 |
| Beta Chain Diverse | gctc | |
| Region (D/J/N) | | |
| Human iNKT TCR | agtgcctccgggggtgaatcctacgagcagtacttcgggccgggcaccaggctc | 38 |
| Beta Chain Diverse | acggtcaca | |
| Region (D/J/N) | | |
| Human iNKT TCR | agcgcctccggaggagagtcatacgaacagtatttcggccctggcacacgcctca | 39 |
| Beta Chain Diverse | ctgtgacc | |
| Region (D/J/N) | | |
| Human iNKT TCR | agcggtcgggtctcggggggcgattccctcatagcgtttctaggccaagagaccc | 40 |
| Beta Chain Diverse | agtacttcgggccaggcacgcggctcctggtgctc | |
| Region (D/J/N) | | |
| Human iNKT TCR | tcaggacgagtgtccggaggggatagcctcatcgcatttctggggcaggaaactc | 41 |
| Beta Chain Diverse | agtacttcggacccggaacacgcctcctggtgctg | |
| Region (D/J/N) | | |
| Human iNKT TCR | agtgtacccgggaacgacaggggcaatgaaaaactgttttttggcagtggaaccc | 42 |
| Beta Chain Diverse | agctctctgtcttg | |
| Region (D/J/N) | | |
| Human iNKT TCR | tccgtgcctggcaacgatagaggtaacgagaagctgatttcggatccggcacaca | 43 |
| Beta Chain Diverse | gctgtctgtcctg | |
| Region (D/J/N) | | |
| Human iNKT TCR- | gaggacctgaacaaggtgttcccacccgaggtcgctgtgtttgagccatcagaag | 44 |
| beta chain cDNA (after | cagagatctcccacacccaaaaggccacactggtgtgcctggccacaggcttctt | |
| D/J/N region) | ccctgaccacgtggagctgagctggtgggtgaatgggaaggaggtgcacagtgg | |
| | ggtcagcacggacccgcagcccctcaaggagcagcccgccctcaatgactcca | |
| | gatactgcctgagcagccgcctgagggtctcggccaccttctggcagaacccccg | |
| | caaccacttccgctgccaagtccagttctacgggctctcggagaatgacgagtgg | |
| | acccaggatagggccaaacccgtcacccagatcgtcagcgccgaggcctgggg | |
| | tagagcagactgtggctttacctcggtgtcctaccagcaaggggtcctgtctgcca | |
| | ccatcctctatgagatcctgctagggaaggccaccctgtatgctgtgctggtcagc | |
| | gcccttgtgttgatggccatggtcaagagaaaggatttctga | |
| Human iNKT TCR- | gaggacctgaataaggtgttcccccctgaggtggctgtctttgaaccaagtgaggc | 45 |
| beta chain cDNA | agaaatttcacatacacagaaagccaccctggtgtgcctggctaccggcttctttcc | |
| codon-optimized (after | cgatcacgtggagctgagctggtgggtcaacggcaaggaagtgcatagcggagt | |
| D/J/N region) | ctccacagacccacagcccctgaaagagcagcctgctctgaatgattccagatact | |
| | gcctgtctagtagactgcgggtgtctgccaccttctggcagaacccaaggaatcatt | |
| | tcagatgtcaggtgcagttttatggcctgagcgagaacgatgaatggactcaggac | |
| | agggctaagccagtgacccagatcgtcagcgcagaggcctggggaagagcaga | |
| | ctgcgggtttacaagcgtgagctatcagcagggcgtcctgagcgccacaatcctgt | |
| | acgaaattctgctgggaaaggccactctgtatgctgtgctggtctccgctctggtgc | |
| | tgatggcaatggtcaagcggaaagatttctga | |
| Human iNKT TCR- | MKKHLTTFLVILWLYFYRGNGKNQVEQSPQSLIILE | 46 |
| alpha chain | GKNCTLQCNYTVSPFSNLRWYKQDTGRGPVSLTIM | |
| | TFSENTKSNGRYTATLDADTKQSSLHITASQLSDSAS | |
| | YICVVSDRGSTLGRLYFGRGTQLTVWPDIQNPDPAV | |
| | YQLRDSKSSDKSVCLFTDFDSQTNVSQSKDSDVYIT | |
| | DKTVLDMRSMDFKSNSAVAWSNKSDFACANAFNN | |
| | SIIPEDTFFPSPESSCDVKLVEKSFETDTNLNFQNLSVI | |
| | GFRILLLKVAGFNLLMTLRLWSS | |
| Human iNKT TCR- | MTIRLLCYMGFYFLGAGLMEADIYQTPRYLVIGTGK | 47 |
| beta chain | KITLECSQTMGHDKMYWYQQDPGMELHLIHYSYG | |
| | VNSTEKGDLSSESTVSRIRTEHFPLTLESARPSHTSQY | |
| | LCAS | |
| Human iNKT TCR | VAVGPQETQYFGPGTRLLVL | 48 |
| Beta Chain Diverse | | |
| Region (D/J/N) | | |
| Human iNKT TCR | SGPGYEQYFGPGTRLTVT | 49 |
| Beta Chain Diverse | | |
| Region (D/J/N) | | |
| Human iNKT TCR | SPQLNTEAFFGQGTRLTVV | 50 |
| Beta Chain Diverse | | |
| Region (D/J/N) | | |
| Human iNKT TCR | SELRALGPSSYNSPLHFGNGTRLTVT | 51 |
| Beta Chain Diverse | | |
| Region (D/J/N) | | |
| Human iNKT TCR | SEQGTTAGAFFGQGTRLTVV | 52 |
| Beta Chain Diverse | | |
| Region (D/J/N) | | |
| Human iNKT TCR | SESRHATGNTIYFGEGSWLTVV | 53 |
| Beta Chain Diverse | | |
| Region (D/J/N) | | |
| Human iNKT TCR | SVPGNDRGNEKLFFGSGTQLSVL | 54 |
| Beta Chain Diverse | | |
| Region (D/J/N) | | |
| Human iNKT TCR | SEGGGLKLAKNIQYFGAGTRLSVL | 55 |
| Beta Chain Diverse | | |
| Region (D/J/N) | | |
| Human iNKT TCR | SEFASSVRGNTIYFGEGSWLTVV | 56 |
| Beta Chain Diverse | | |
| Region (D/J/N) | | |
| Human iNKT TCR | SAALGRETQYFGPGTRLLVL | 57 |
| Beta Chain Diverse | | |
| Region (D/J/N) | | |
| Human iNKT TCR | SASGGESYEQYFGPGTRLTVT | 58 |
| Beta Chain Diverse | | |
| Region (D/J/N) | | |
| Human iNKT TCR | SGRVSGGDSLIAFLGQETQYFGPGTRLLVL | 59 |
| Beta Chain Diverse | | |
| Region (D/J/N) | | |
| Human iNKT TCR | SVPGNDRGNEKLFFGSGTQLSVL | 60 |
| Beta Chain Diverse | | |
| Region (D/J/N) | | |
| Human iNKT TCR- | EDLNKVFPPEVAVFEPSEAEISHTQKATLVCLATGFF | 61 |
| beta chain (after D/J/N | PDHVELSWWVNGKEVHSGVSTDPQPLKEQPALNDS | |
| region) | RYCLSSRLRVSATFWQNPRNHFRCQVQFYGLSENDE | |
| | WTQDRAKPVTQIVSAEAWGRADCGFTSVSYQQGVL | |
| | SATILYEILLGKATLYAVLVSALVLMAMVKRKDF | |
| B-2 microglobin | agtggaggcgtcgcgctggcgggcattcctgaagctgacagcattcgggccgag | 62 |
| (B2M) | atgtctcgctccgtggccttagctgtgctcgcgctactctctctttctggcctggagg | |
| | ctatccagcgtactccaaagattcaggtttactcacgtcatccagcagagaatggaa | |
| | agtcaaatttcctgaattgctatgtgtctgggtttcatccatccgacattgaagttgact | |
| | tactgaagaatggagagagaattgaaaaagtggagcattcagacttgtctttcagca | |
| | aggactggtctttctatctcttgtactacactgaattcacccccactgaaaaagatga | |
| | gtatgcctgccgtgtgaaccatgtgactttgtcacagcccaagatagttaagtgggg | |
| | taagtcttacattcttttgtaagctgctgaaagttgtgtatgagtagtcatatcataaag | |
| | ctgctttgatataaaaaaggtctatggccatactaccctgaatgagtcccatcccatc | |
| | tgatataaacaatctgcatattgggattgtcagggaatgttcttaaagatcagattagt | |
| | ggcacctgctgagatactgatgcacagcatggtttctgaaccagtagtttccctgca | |
| | gttgagcagggagcagcagcagcacttgcacaaatacatatacactcttaacactt | |
| | cttacctactggcttcctctagcttttgtggcagcttcaggtatatttagcactgaacga | |
| | acatctcaagaaggtataggcctttgtttgtaagtcctgctgtcctagcatcctataat | |
| | cctggacttctccagtactttctggctggattggtatctgaggctagtaggaagggct | |
| | tgttcctgctgggtagctctaaacaatgtattcatgggtaggaacagcagcctattct | |
| | gccagccttatttctaaccattttagacatttgttagtacatggtattttaaaagtaaaac | |
| | ttaatgtcttccttttttttctccactgtctttttcatagatcgagacatgtaagcagcatc | |
| | atggaggtaagtattgaccttgagaaaatgatttgtttcactgtcctgaggactattta | |
| | tagacagctctaacatgataaccctcactatgtggagaacattgacagagtaacattt | |
| | tagcagggaaagaagaatcctacagggtcatgttcccttctcctgtggagtggcat | |
| | gaagaaggtgtatggccccaggtatggccatattactgaccctctacagagaggg | |
| | caaaggaactgccagtatggtattgcaggataaaggcaggtggttacccacattac | |
| | ctgcaaggctttgatctttcttctgccatttccacattggacatctctgctgaggagag | |
| | aaaatgaaccactcttttcctttgtataatgttgttttattcttcagacagaagagagga | |
| | gttatacagctctgcagacatcccattcctgtatggggactgtgtttgcctcttagag | |
| | gttcccaggccactagaggagataaagggaaacagattgttataacttgatataatg | |
| | atactataatagatgtaactacaaggagctccagaagcaagagagagggaggaa | |
| | cttggacttctctgcatctttagttggagtccaaaggcttttcaatgaaattctactgcc | |
| | cagggtacattgatgctgaaaccccattcaaatctcctgttatattctagaacaggga | |
| | attgatttgggagagcatcaggaaggtggatgatctgcccagtcacactgttagtaa | |
| | attgtagagccaggacctgaactctaatatagtcatgtgttacttaatgacggggac | |
| | atgttctgagaaatgcttacacaaacctaggtgttgtagcctactacacgcataggct | |
| | acatggtatagcctattgctcctagactacaaacctgtacagcctgttactgtactga | |
| | atactgtgggcagttgtaacacaatggtaagtatttgtgtatctaaacatagaagttgc | |
| | agtaaaaatatgctattttaatcttatgagaccactgtcatatatacagtccatcattga | |
| | ccaaaacatcatatcagcattttttcttctaagattttgggagcaccaaagggataca | |
| | ctaacaggatatactctttataatgggtttggagaactgtctgcagctacttcttttaaa | |
| | aaggtgatctacacagtagaaattagacaagtttggtaatgagatctgcaatccaaa | |
| | taaaataaattcattgctaacattttatttcttttcaggtttgaagatgccgcatttggat | |
| | tggatgaattccaaattctgcttgcttgctttttaatattgatatgcttatacacttacactt | |
| | tatgcacaaaatgtagggttataataatgttaacatggacatgatcttctttataattcta | |
| | ctttgagtgctgtctccatgtttgatgtatctgagcaggttgctccacaggtagctcta | |
| | ggagggctggcaacttagaggtggggagcagagaattctcttatccaacatcaac | |
| | atcttggtcagatttgaactcttcaatctcttgcactcaaagcttgttaagatagttaag | |
| | cgtgcataagttaacttccaatttacatactctgcttagaatttgggggaaaatttaga | |
| | aatataattgacaggattattggaaatttgttataatgaatgaaacattttgtcatataag | |
| | attcatatttacttcttatacatttgataaagtaaggcatggttgtggttaatctggtttatt | |
| | tttgttccacaagttaaataaatcataaaacttga | |
| Human class II major | ggttagtgatgaggctagtgatgaggctgtgtgcttctgagctgggcatccgaagg | 63 |
| histocompatibility | catccttggggaagctgagggcacgaggaggggctgccagactccgggagctg | |
| complex transactivator | ctgcctggctgggattcctacacaatgcgttgcctggctccacgccctgctgggtc | |
| (CIITA) | ctacctgtcagagccccaaggcagctcacagtgtgccaccatggagttggggccc | |
| | ctagaaggtggctacctggagcttcttaacagcgatgctgaccccctgtgcctctac | |
| | cacttctatgaccagatggacctggctggagaagaagagattgagctctactcaga | |
| | acccgacacagacaccatcaactgcgaccagttcagcaggctgttgtgtgacatg | |
| | gaaggtgatgaagagaccagggaggcttatgccaatatcgcggaactggaccag | |
| | tatgtcttccaggactcccagctggagggcctgagcaaggacattttcaagcacat | |
| | aggaccagatgaagtgatcggtgagagtatggagatgccagcagaagttgggca | |
| | gaaaagtcagaaaagacccttcccagaggagcttccggcagacctgaagcactg | |
| | gaagccagctgagccccccactgtggtgactggcagtctcctagtgggaccagtg | |
| | agcgactgctccaccctgccctgcctgccactgcctgcgctgttcaaccaggagc | |
| | cagcctccggccagatgcgcctggagaaaaccgaccagattcccatgcctttctc | |
| | cagttcctcgttgagctgcctgaatctccctgagggacccatccagtttgtccccac | |
| | catctccactctgccccatgggctctggcaaatctctgaggctggaacaggggtct | |
| | ccagtatattcatctaccatggtgaggtgccccaggccagccaagtaccccctccc | |
| | agtggattcactgtccacggcctcccaacatctccagaccggccaggctccacca | |
| | gccccttcgctccatcagccactgacctgcccagcatgcctgaacctgccctgacc | |
| | tcccgagcaaacatgacagagcacaagacgtcccccacccaatgcccggcagct | |
| | ggagaggtctccaacaagcttccaaaatggcctgagccggtggagcagttctacc | |
| | gctcactgcaggacacgtatggtgccgagcccgcaggcccggatggcatcctag | |
| | tggaggtggatctggtgcaggccaggctggagaggagcagcagcaagagcctg | |
| | gagcgggaactggccaccccggactgggcagaacggcagctggcccaaggag | |
| | gcctggctgaggtgctgttggctgccaaggagcaccggcggccgcgtgagaca | |
| | cgagtgattgctgtgctgggcaaagctggtcagggcaagagctattgggctgggg | |
| | cagtgagccgggcctgggcttgtggccggcttccccagtacgactttgtcttctctg | |
| | tcccctgccattgcttgaaccgtccgggggatgcctatggcctgcaggatctgctct | |
| | tctccctgggcccacagccactcgtggcggccgatgaggttttcagccacatcttg | |
| | aagagacctgaccgcgttctgctcatcctagacggcttcgaggagctggaagcgc | |
| | aagatggcttcctgcacagcacgtgcggaccggcaccggcggagccctgctccc | |
| | tccgggggctgctggccggccttttccagaagaagctgctccgaggttgcaccct | |
| | cctcctcacagcccggccccggggccgcctggtccagagcctgagcaaggccg | |
| | acgccctatttgagctgtccggcttctccatggagcaggcccaggcatacgtgatg | |
| | cgctactttgagagctcagggatgacagagcaccaagacagagccctgacgctc | |
| | ctccgggaccggccacttcttctcagtcacagccacagccctactttgtgccgggc | |
| | agtgtgccagctctcagaggccctgctggagcttggggaggacgccaagctgcc | |
| | ctccacgctcacgggactctatgtcggcctgctgggccgtgcagccctcgacagc | |
| | ccccccggggccctggcagagctggccaagctggcctgggagctgggccgca | |
| | gacatcaaagtaccctacaggaggaccagttcccatccgcagacgtgaggacct | |
| | gggcgatggccaaaggcttagtccaacacccaccgcgggccgcagagtccgag | |
| | ctggccttccccagcttcctcctgcaatgcttcctgggggccctgtggctggctctg | |
| | agtggcgaaatcaaggacaaggagctcccgcagtacctagcattgaccccaagg | |
| | aagaagaggccctatgacaactggctggagggcgtgccacgctttctggctggg | |
| | ctgatcttccagcctcccgcccgctgcctgggagccctactcgggccatcggcgg | |
| | ctgcctcggtggacaggaagcagaaggtgcttgcgaggtacctgaagcggctgc | |
| | agccggggacactgcgggcgcggcagctgctggagctgctgcactgcgcccac | |
| | gaggccgaggaggctggaatttggcagcacgtggtacaggagctccccggccg | |
| | cctctcttttctgggcacccgcctcacgcctcctgatgcacatgtactgggcaaggc | |
| | cttggaggcggcgggccaagacttctccctggacctccgcagcactggcatttgc | |
| | ccctctggattggggagcctcgtgggactcagctgtgtcacccgtttcagggctgc | |
| | cttgagcgacacggtggcgctgtgggagtccctgcagcagcatggggagaccaa | |
| | gctacttcaggcagcagaggagaagttcaccatcgagcctttcaaagccaagtcc | |
| | ctgaaggatgtggaagacctgggaaagcttgtgcagactcagaggacgagaagt | |
| | tcctcggaagacacagctggggagctccctgctgacgggacctaaagaaactgg | |
| | agtagcgctgggccctgtctcaggcccccaggctaccccaaactggtgcggatc | |
| | ctcacggccattcctccctgcagcatctggacctggatgcgctgagtgagaacaa | |
| | gatcggggacgagggtgtctcgcagctctcagccaccttcccccagctgaagtcc | |
| | ttggaaaccctcaatctgtcccagaacaacatcactgacctgggtgcctacaaactc | |
| | gccgaggccctgccacgctcgctgcatccctgctcaggctaagcagtacaataa | |
| | ctgcatctgcgacgtgggagccgagagcaggctcgtgtgcaccggacatggtgt | |
| | ccctccgggtgatggacgtccagtacaacaagacacggctgccggggcccagc | |
| | agctcgctgccagccacggaggtgtcctcatgtggagacgctggcgatgtggac | |
| | gcccaccatcccattcagtgtccaggaacacctgcaacaacaggattcacggatc | |
| | agcctgagatgatcccagctgtgctctggacaggcatgactctgaggacactaac | |
| | cacgctggaccagaactgggtacttgtggacacagctcactccaggctgtatccc | |
| | atgagcctcagcatcctggcacccggcccctgctggacagggaggcccctgcc | |
| | cggctgcggaatgaaccacatcagctctgctgacagacacaggcccggctccag | |
| | gctccatagcgcccagagggtggatgcctggtggcagctgcggtccacccagg | |
| | agccccgaggccactctgaaggacattgcggacagccacggccaggccagag | |
| | ggagtgacagaggcagccccattctgcctgcccaggcccctgccaccctgggga | |
| | gaaagtacactattattatattagacagagtctcactgagcccaggctggcgtgca | |
| | gtggtgcgatctgggacactgcaacctccgcctcagggacaagcgattcactgc | |
| | ttcagcctcccgagtagctgggactacaggcacccaccatcatgtctggctaattat | |
| | cattatagtagagacagggattgccatgaggccaggctggtctcaaactcagac | |
| | ctcaggtgatccacccacctcagcctcccaaagtgctgggattacaagcgtgagc | |
| | cactgcaccgggccacagagaaagtacactccaccctgctctccgaccagacac | |
| | cttgacagggcacaccgggcactcagaagacactgatgggcaacccccagcctg | |
| | ctaattccccagattgcaacaggctgggcttcagtggcagctgcattgtctatggga | |
| | ctcaatgcactgacattgaggccaaagccaaagctaggcctggccagatgcacc | |
| | agcccttagcagggaaacagctaatgggacactaatggggcggtgagagggga | |
| | acagactggaagcacagcttcatacctgtgtcattacactacattataaatgtctcat | |
| | aatgtcacaggcaggtccagggatgagacataccctgaaccattaggggtaccc | |
| | actgctctggttatctaatatgtaacaagccaccccaaatcatagtggcttaaaacaa | |
| | cactcacattta | |
| Human T cell receptor | tatgaaacccacaaaggcagagacttgtccagcctaacctgcctgctgctcctag | 64 |
| alpha chain (TRAC) | ctcctgaggctcagggcccaggcactgtccgctctgctcagggccctccagcgt | |
| | ggccactgctcagccatgctcctgctgctcgtcccagtgctcgaggtgattatacc | |
| | ctgggaggaaccagagcccagtcggtgacccagcaggcagccacgtctctgtct | |
| | ctgaaggagccctggactgctgaggtgcaactactcatcgtctgaccaccatatct | |
| | cttctggtatgtgcaataccccaaccaaggactccagcttctcctgaagtacacatc | |
| | agcggccaccctggttaaaggcatcaacggattgaggctgaatttaagaagagtg | |
| | aaacctccaccacctgacgaaaccctcagcccatatgagcgacgcggctgagta | |
| | cactgtgctgtgagtgatctcgaaccgaacagcagtgatccaagataatctagga | |
| | tcagggaccagactcagcatccggccaaatatccagaaccctgaccctgccgtgt | |
| | accagctgagagactctaaatccagtgacaagtctgtctgcctattcaccgattttga | |
| | ttctcaaacaaatgtgtcacaaagtaaggattctgatgtgtatatcacagacaaaact | |
| | gtgctagacatgaggtctatggacttcaagagcaacagtgctgtggcctggagca | |
| | acaaatctgactttgcatgtgcaaacgccttcaacaacagcattattccagaagaca | |
| | ccttcttccccagcccagaaagttcctgtgatgtcaagctggtcgagaaaagctttg | |
| | aaacagatacgaacctaaactttcaaaacctgtcagtgattgggttccgaatcctcct | |
| | cctgaaagtggccgggtttaatctgctcatgacgctgcggctgtggtccagctgag | |
| | atctgcaagattgtaagacagcctgtgctccctcgctccttcctctgcattgcccctct | |
| | tctccctctccaaacagagggaactctcctacccccaaggaggtgaaagctgctac | |
| | cacctctgtgcccccccggtaatgccaccaactggatcctacccgaatttatgatta | |
| | agattgctgaagagctgccaaacactgctgccaccccctctgttcccttattgctgct | |
| | tgtcactgcctgacattcacggcagaggcaaggctgctgcagcctcccctggctgt | |
| | gcacattccctcctgctccccagagactgcctccgccatcccacagatgatggatc | |
| | ttcagtgggttctcttgggctctaggtcctggagaatgttgtgaggggtttattattttt | |
| | aatagtgttcataaagaaatacatagtattcttcttctcaagacgtggggggaaattat | |
| | ctcattatcgaggccctgctatgctgtgtgtctgggcgtgttgtatgtcctgctgccg | |
| | atgccttcattaaaatgatttggaa | |
| Human T cell receptor | tgcatcctagggacagcatagaaaggaggggcaaagtggagagagagcaacag | 65 |
| beta chain (TRBC1) | acactgggatggtgaccccaaaacaatgagggcctagaatgacatagttgtgcttc | |
| | attacggcccattcccagggctctctctcacacacacagagcccctaccagaacca | |
| | gacagctctcagagcaaccctggctccaacccctcttccctttccagaggacctga | |
| | acaaggtgttcccacccgaggtcgctgtgtttgagccatcagaagcagagatctcc | |
| | cacacccaaaaggccacactggtgtgcctggccacaggcttcttccccgaccacg | |
| | tggagctgagctggtgggtgaatgggaaggaggtgcacagtggggtcagcacg | |
| | gacccgcagcccctcaaggagcagcccgccctcaatgactccagatactgcctg | |
| | agcagccgcctgagggtctcggccaccttctggcagaacccccgcaaccacttcc | |
| | gctgtcaagtccagttctacgggctctcggagaatgacgagtggacccaggatag | |
| | ggccaaacccgtcacccagatcgtcagcgccgaggcctggggtagagcaggtg | |
| | agtggggcctggggagatgcctggaggagattaggtgagaccagctaccaggg | |
| | aaaatggaaagatccaggtagcagacaagactagatccaaaaagaaaggaacca | |
| | gcgcacaccatgaaggagaattgggcacctgtggttcattcttctcccagattctca | |
| | gcccaacagagccaagcagctgggtcccctttctatgtggcctgtgtaactctcatc | |
| | tgggtggtgccccccatccccctcagtgctgccacatgccatggattgcaaggac | |
| | aatgtggctgacatctgcatggcagaagaaaggaggtgctgggctgtcagagga | |
| | agctggtctgggcctgggagtctgtgccaactgcaaatctgactttacttttaattgc | |
| | ctatgaaaataaggtctctcatttattttcctctccctgctttctttcagactgtggcttta | |
| | cctcgggtaagtaagcccttccttttcctctccctctctcatggttcttgacctagaacc | |
| | aaggcatgaagaactcacagacactggagggtggagggtgggagagaccaga | |
| | gctacctgtgcacaggtacccacctgtccttcctccgtgccaacagtgtcctaccag | |
| | caaggggtcctgtctgccaccatcctctatgagatcctgctagggaaggccaccct | |
| | gtatgctgtgctggtcagcgcccttgtgttgatggccatggtaagcaggagggcag | |
| | gatggggccagcaggctggaggtgacacactgacaccaagcacccagaagtat | |
| | agagtccctgccaggattggagctgggcagtagggagggaagagatttcattcag | |
| | gtgcctcagaagataacttgcacctctgtaggatcacagtggaagggtcatgctgg | |
| | gaaggagaagctggagtcaccagaaaacccaatggatgttgtgatgagccttact | |
| | atttgtgtggtcaatgggccctactactttctctcaatcctcacaactcctggctcttaa | |
| | taacccccaaaactttctcttctgcaggtcaagagaaaggatttctgaaggcagccc | |
| | tggaagtggagttaggagcttctaacccgtcatggtttcaatacacattcttcttttgc | |
| | cagcgcttctgaagagctgctctcacctctctgcatcccaatagatatccccctatgt | |
| | gcatgcacacctgcacactcacggctgaaatctccctaacccagggggaccttag | |
| | catgcctaagtgactaaaccaataaaaatgttctggtctggcctgactctgacttgtg | |
| | aatgtctggatagctccttggctgtctctgaactccctgtgactctccccattcagtca | |
| | ggatagaaacaagaggtattcaaggaaaatgcagactcttcacgtaagagggatg | |
| | aggggcccaccttgagatcaatagcag | |
| Human TRBC2 T cell | atggcgtagtccccaaagaacgaggacctagtaacataattgtgcttcattatggtc | 66 |
| receptor beta constant 2 | ctttcccggccttctctctcacacatacacagagcccctaccaggaccagacagct | |
| (TCRB2) | ctcagagcaaccctagccccattacctcttccctttccagaggacctgaaaaacgtg | |
| | ttcccacccgaggtcgctgtgtttgagccatcagaagcagagatctcccacaccca | |
| | aaaggccacactggtgtgcctggccacaggcttctaccccgaccacgtggagctg | |
| | agctggtgggtgaatgggaaggaggtgcacagtggggtcagcacagacccgca | |
| | gcccctcaaggagcagcccgccctcaatgactccagatactgcctgagcagccg | |
| | cctgagggtctcggccaccttctggcagaacccccgcaaccacttccgctgtcaa | |
| | gtccagttctacgggctctcggagaatgacgagtggacccaggatagggccaaa | |
| | cctgtcacccagatcgtcagcgccgaggcctggggtagagcaggtgagtgggg | |
| | cctggggagatgcctggaggagattaggtgagaccagctaccagggaaaatgga | |
| | aagatccaggtagcggacaagactagatccagaagaaagccagagtggacaag | |
| | gtgggatgatcaaggttcacagggtcagcaaagcacggtgtgcacttcccccacc | |
| | aagaagcatagaggctgaatggagcacctcaagctcattcttccttcagatcctgac | |
| | accttagagctaagctttcaagtctccctgaggaccagccatacagctcagcatctg | |
| | agtggtgtgcatcccattctcttctggggtcctggtttcctaagatcatagtgaccact | |
| | tcgctggcactggagcagcatgagggagacagaaccagggctatcaaaggagg | |
| | ctgactttgtactatctgatatgcatgtgtttgtggcctgtgagtctgtgatgtaaggct | |
| | caatgtccttacaaagcagcattctctcatccatttttcttcccctgttttctttcagactg | |
| | tggcttcacctccggtaagtgagtctctcctttttctctctatctttcgccgtctctgctct | |
| | cgaaccagggcatggagaatccacggacacaggggcgtgagggaggccagag | |
| | ccacctgtgcacaggtacctacatgctctgttcttgtcaacagagtcttaccagcaa | |
| | ggggtcctgtctgccaccatcctctatgagatcttgctagggaaggccaccttgtat | |
| | gccgtgctggtcagtgccctcgtgctgatggccatggtaaggaggagggtgggat | |
| | agggcagatgatgggggcaggggatggaacatcacacatgggcataaaggaat | |
| | ctcagagccagagcacagcctaatatatcctatcacctcaatgaaaccataatgaa | |
| | gccagactggggagaaaatgcagggaatatcacagaatgcatcatgggaggatg | |
| | gagacaaccagcgagccctactcaaattaggcctcagagcccgcctcccctgcc | |
| | ctactcctgctgtgccatagcccctgaaaccctgaaaatgttctctcttccacaggtc | |
| | aagagaaaggattccagaggctagctccaaaaccatcccaggtcattcttcatcct | |
| | cacccaggattctcctgtacctgctcccaatctgtgttcctaaaagtgattctcactct | |
| | gcttctcatctcctacttacatgaatacttctctcttttttctgtttccctgaagattgagct | |
| | cccaacccccaagtacgaaataggctaaaccaataaaaaattgtgtgttgggcctg | |
| | gttgcatttcaggagtgtctgtggagttctgctcatcactgacctatcttctgatttagg | |
| | gaaagcagcattcgcttggacatctgaagtgacagccctctttctctccacccaatg | |
| | ctgctttctcctgttcatcctgatggaagtctcaacaca | |
| synthetic primer | cgcgagcacagcuaaggcca | 67 |
| synthetic primer | gauauuggcauaagccuccc | 68 |
| Human T cell receptor | ttttgaaacccttcaaaggcagagacttgtccagcctaacctgcctgctgctcctag | 70 |
| alpha chain (TRAC) | ctcctgaggctcagggcccttggcttctgtccgctctgctcagggccctccagcgt | |
| mRNA sequence | ggccactgctcagccatgctcctgctgctcgtcccagtgctcgaggtgatttttacc | |
| | ctgggaggaaccagagcccagtcggtgacccagcttggcagccacgtctctgtct | |
| | ctgaaggagccctggttctgctgaggtgcaactactcatcgtctgttccaccatatct | |
| | cttctggtatgtgcaataccccaaccaaggactccagcttctcctgaagtacacatc | |
| | agcggccaccctggttaaaggcatcaacggttttgaggctgaatttaagaagagtg | |
| | aaacctccttccacctgacgaaaccctcagcccatatgagcgacgcggctgagta | |
| | cttctgtgctgtgagtgatctcgaaccgaacagcagtgcttccaagataatctttgga | |
| | tcagggaccagactcagcatccggccaaatatccagaaccctgaccctgccgtgt | |
| | accagctgagagactctaaatccagtgacaagtctgtctgcctattcaccgattttga | |
| | ttctcaaacaaatgtgtcacaaagtaaggattctgatgtgtatatcacagacaaaact | |
| | gtgctagacatgaggtctatggacttcaagagcaacagtgctgtggcctggagca | |
| | acaaatctgactttgcatgtgcaaacgccttcaacaacagcattattccagaagaca | |
| | ccttcttccccagcccagaaagttcctgtgatgtcaagctggtcgagaaaagctttg | |
| | aaacagatacgaacctaaactttcaaaacctgtcagtgattgggttccgaatcctcct | |
| | cctgaaagtggccgggtttaatctgctcatgacgctgcggctgtggtccagctgag | |
| | atctgcaagattgtaagacagcctgtgctccctcgctccttcctctgcattgcccctct | |
| | tctccctctccaaacagagggaactctcctacccccaaggaggtgaaagctgctac | |
| | cacctctgtgcccccccggtaatgccaccaactggatcctacccgaatttatgatta | |
| | agattgctgaagagctgccaaacactgctgccaccccctctgttcccttattgctgct | |
| | tgtcactgcctgacattcacggcagaggcaaggctgctgcagcctcccctggctgt | |
| | gcacattccctcctgctccccagagactgcctccgccatcccacagatgatggatc | |
| | ttcagtgggttctcttgggctctaggtcctggagaatgttgtgaggggtttattattttt | |
| | aatagtgttcataaagaaatacatagtattcttcttctcaagacgtggggggaaattat | |
| | ctcattatcgaggccctgctatgctgtgtgtctgggcgtgttgtatgtcctgctgccg | |
| | atgccttcattaaaatgatttggaa | |
| BCMA CAR with | atggctctgcctgtgaccgccctgctgctgcctctggctctgctgctgcacgccgct | 71 |
| truncated EGFR | cggcctGacatcgttttgacacaatctcctgcgtcattggccatgagtctcgggaa | |
| | gcgcgcaacaatatcctgtcgcgccagtgaatctgtgtctgtgataggagcgcact | |
| | tgatccattggtatcagcagaaacctggacaacctcccaagctgctcatctacctcg | |
| | ccagtaaccttgaaacaggagtacctgctcggttttcaggttccgggtcagggacg | |
| | gatttcactttgactatcgacccagttgaggaagacgacgtagccatatatagctgc | |
| | ctgcagtctcggatcttcccgcgcacgttcgggggaggaactaagctggagatta | |
| | agggcggcgggggttctggtggcggcggcagcggcggtggaggatcacaaat | |
| | ccaactggttcagtccggtccagaactgaaaaagccgggggagacggtgaaaat | |
| | ctcctgtaaggcctcaggttataccttcaccgattacagcatcaattgggtaaagcg | |
| | ggctccagggaaaggtctgaaatggatgggttggatcaacacagaaacccgaga | |
| | accagcctatgcttacgactttcgaggtcgattcgctttttccttggaaacttccgcaa | |
| | gcacagcctatctgcaaatcaacaatctcaagtacgaagatacggccacgtattttt | |
| | gtgccctggattacagctatgcaatggattactggggtcaggggacgtctgttaca | |
| | gtttctagtActacaactccagcacccagaccccctacacctgctccaactatcgc | |
| | aagtcagcccctgtcactgcgccctgaagcctgtcgccctgctgccgggggagct | |
| | gtgcatactcggggactggactttgcctgtgatatctacAtctgggcgcccttggc | |
| | cgggacttgtggggtccttctcctgtcactggttatcaccctttactgcAggttcagt | |
| | gtcgtgaagagaggccggaagaagctgctgtacatcttcaagcagcctttcatgag | |
| | gcccgtgcagactacccaggaggaagatggatgcagctgtagattccctgaaga | |
| | ggaggaaggaggctgtgagctgagagtgaagttctcccgaagcgcagatgcccc | |
| | agcctatcagcagggacagaatcagctgtacaacgagctgaacctgggaagacg | |
| | ggaggaatacgatgtgctggacaaaaggcggggcagagatcctgagatgggcg | |
| | gcaaaccaagacggaagaacccccaggaaggtctgtataatgagctgcagaaag | |
| | acaagatggctgaggcctactcagaaatcgggatgaagggcgaaagaaggaga | |
| | ggaaaaggccacgacggactgtaccaggggctgagtacagcaacaaaagacac | |
| | ctatgacgctctgcacatgcaggctctgccaccaagaCgagctaaacgaggctc | |
| | aggcgcgacgaactttagtttgctgaagcaagctggggatgtagaggaaaatccg | |
| | ggtcccatgttgctccttgtgacgagcctcctgctctgcgagctgccccatccagcc | |
| | ttcctcctcatcccgcggaaggtgtgcaatggcataggcattggcgagtttaaagat | |
| | tctctgagcataaatgctacgaatattaagcatttcaagaattgtacttctattagtggc | |
| | gacctccatattcttccggttgccttcaggggtgactctttcacccacacacctccatt | |
| | ggatccacaagaacttgacatcctgaagacggttaaagagattacaggcttcctcct | |
| | tatccaagcgtggcccgagaacagaacggacttgcacgcctttgagaacctcgaa | |
| | ataatacggggtcggacgaagcaacacggccaatttagccttgcggttgttagtct | |
| | gaacattacttctctcggccttcgctctttgaaagaaatcagcgacggagatgtcatc | |
| | attagtggaaacaagaacctgtgctacgcgaacacaatcaactggaagaagctctt | |
| | cggtacttcaggccaaaagacaaagattattagtaacagaggagagaatagctgta | |
| | aggctaccggacaagtttgtcacgccttgtgtagtccagagggttgctggggaccg | |
| | gaaccaagggattgcgtcagttgccggaacgtgagtcgcggacgcgagtgtgtg | |
| | gataagtgcaatcttctggaaggggaaccgcgagagtttgtagaaaattccgaatg | |
| | tatacagtgtcatcccgagtgtcttccacaagcaatgaatatcacatgtacagggag | |
| | gggtcctgataactgtatccaatgtgcacactacatagatggtcctcactgtgtaaag | |
| | acgtgccccgccggagtaatgggtgaaaacaacaccctcgtgtggaagtacgcc | |
| | gatgccgggcatgtctgtcatttgtgtcatcccaactgcacatatggctgtaccggtc | |
| | ctggattggagggctgtccaacaaacgggccgaaaataccgagtatcgcaacag | |
| | gcatggtgggagcacttttgcttctcctcgttgtcgccctgggcatcggcttgttcat | |
| | g | |
| BCMA CAR with | MALPVTALLLPLALLLHAARPDIVLTQSPASLAMSL | 72 |
| truncated EGFR | GKRATISCRASESVSVIGAHLIHWYQQKPGQPPKLLI | |
| | YLASNLETGVPARFSGSGSGTDFTLTIDPVEEDDVAI | |
| | YSCLQSRIFPRTFGGGTKLEIKGGGGSGGGGSGGGG | |
| | SQIQLVQSGPELKKPGETVKISCKASGYTFTDYSINW | |
| | VKRAPGKGLKWMGWINTETREPAYAYDFRGRFAFS | |
| | LETSASTAYLQINNLKYEDTATYFCALDYSYAMDY | |
| | WGQGTSVTVSSTTTPAPRPPTPAPTIASQPLSLRPEAC | |
| | RPAAGGAVHTRGLDFACDIYIWAPLAGTCGVLLLSL | |
| | VITLYCRFSVVKRGRKKLLYIFKQPFMRPVQTTQEE | |
| | DGCSCRFPEEEEGGCELRVKFSRSADAPAYQQGQNQ | |
| | LYNELNLGRREEYDVLDKRRGRDPEMGGKPRRKNP | |
| | QEGLYNELQKDKMAEAYSEIGMKGERRRGKGHDG | |
| | LYQGLSTATKDTYDALHMQALPPRRAKRGSGATNF | |
| | SLLKQAGDVEENPGPMLLLVTSLLLCELPHPAFLLIP | |
| | RKVCNGIGIGEFKDSLSINATNIKHFKNCTSISGDLHI | |
| | LPVAFRGDSFTHTPPLDPQELDILKTVKEITGFLLIQA | |
| | WPENRTDLHAFENLEIIRGRTKQHGQFSLAVVSLNIT | |
| | SLGLRSLKEISDGDVIISGNKNLCYANTINWKKLFGT | |
| | SGQKTKIISNRGENSCKATGQVCHALCSPEGCWGPE | |
| | PRDCVSCRNVSRGRECVDKCNLLEGEPREFVENSEC | |
| | IQCHPECLPQAMNITCTGRGPDNCIQCAHYIDGPHCV | |
| | KTCPAGVMGENNTLVWKYADAGHVCHLCHPNCTY | |
| | GCTGPGLEGCPTNGPKIPSIATGMVGALLLLLVVAL | |
| | GIGLFM | |
| Leader | atggctctgcctgtgaccgccctgctgctgcctctggctctgctgctgcacgccgct | 73 |
| | cggcct | |
| BCMA scFv | gacatcgttttgacacaatctcctgcgtcattggccatgagtctcgggaagcgcgca | 74 |
| | acaatatcctgtcgcgccagtgaatctgtgtctgtgataggagcgcacttgatccatt | |
| | ggtatcagcagaaacctggacaacctcccaagctgctcatctacctcgccagtaac | |
| | cttgaaacaggagtacctgctcggttttcaggttccgggtcagggacggatttcact | |
| | ttgactatcgacccagttgaggaagacgacgtagccatatatagctgcctgcagtct | |
| | cggatcttcccgcgcacgttcgggggaggaactaagctggagattaagggcggc | |
| | gggggttctggtggcggcggcagcggcggtggaggatcacaaatccaactggtt | |
| | cagtccggtccagaactgaaaaagccgggggagacggtgaaaatctcctgtaag | |
| | gcctcaggttataccttcaccgattacagcatcaattgggtaaagcgggctccagg | |
| | gaaaggtctgaaatggatgggttggatcaacacagaaacccgagaaccagcctat | |
| | gcttacgactttcgaggtcgattcgctttttccttggaaacttccgcaagcacagcct | |
| | atctgcaaatcaacaatctcaagtacgaagatacggccacgtatttttgtgccctgg | |
| | attacagctatgcaatggattactggggtcaggggacgtctgttacagtttctagt | |
| | actacaactccagcacccagaccccctacacctgctccaactatcgcaagtcagc | |
| CD8 hinge | ccctgtcactgcgccctgaagcctgtcgccctgctgccgggggagctgtgcatact | 75 |
| | cggggactggactttgcctgtgatatctac | |
| CD8 transmembrane | atctgggcgcccttggccgggacttgtggggtccttctcctgtcactggttatcacc | 76 |
| | ctttactgc | |
| 4-1BB costimulatory | aggttcagtgtcgtgaagagaggccggaagaagctgctgtacatcttcaagcagc | 77 |
| domain | ctttcatgaggcccgtgcagactacccaggaggaagatggatgcagctgtagattc | |
| | cctgaagaggaggaaggaggctgtgagctgaga | |
| CD3 zeta intracellular | gtgaagttctcccgaagcgcagatgccccagcctatcagcagggacagaatcag | 78 |
| signaling domain | ctgtacaacgagctgaacctgggaagacgggaggaatacgatgtgctggacaaa | |
| | aggcggggcagagatcctgagatgggcggcaaaccaagacggaagaaccccc | |
| | aggaaggtctgtataatgagctgcagaaagacaagatggctgaggcctactcaga | |
| | aatcgggatgaagggcgaaagaaggagaggaaaaggccacgacggactgtac | |
| | caggggctgagtacagcaacaaaagacacctatgacgctctgcacatgcaggct | |
| | ctgccaccaaga | |
| P2A peptide | cgagctaaacgaggctcaggcgcgacgaactttagtttgctgaagcaagctgggg | 79 |
| | atgtagaggaaaatccgggtccc | |
| Truncated EGFR | atgttgctccttgtgacgagcctcctgctctgcgagctgccccatccagccttcctcc | 80 |
| | tcatcccgcggaaggtgtgcaatggcataggcattggcgagtttaaagattctctga | |
| | gcataaatgctacgaatattaagcatttcaagaattgtacttctattagtggcgacctc | |
| | catattcttccggttgccttcaggggtgactctttcacccacacacctccattggatcc | |
| | acaagaacttgacatcctgaagacggttaaagagattacaggcttcctccttatcca | |
| | agcgtggcccgagaacagaacggacttgcacgcctttgagaacctcgaaataata | |
| | cggggtcggacgaagcaacacggccaatttagccttgcggttgttagtctgaacat | |
| | tacttctctcggccttcgctctttgaaagaaatcagcgacggagatgtcatcattagt | |
| | ggaaacaagaacctgtgctacgcgaacacaatcaactggaagaagctcttcggta | |
| | cttcaggccaaaagacaaagattattagtaacagaggagagaatagctgtaaggct | |
| | accggacaagtttgtcacgccttgtgtagtccagagggttgctggggaccggaac | |
| | caagggattgcgtcagttgccggaacgtgagtcgcggacgcgagtgtgtggataa | |
| | gtgcaatcttctggaaggggaaccgcgagagtttgtagaaaattccgaatgtatac | |
| | agtgtcatcccgagtgtcttccacaagcaatgaatatcacatgtacagggaggggt | |
| | cctgataactgtatccaatgtgcacactacatagatggtcctcactgtgtaaagacgt | |
| | gccccgccggagtaatgggtgaaaacaacaccctcgtgtggaagtacgccgatg | |
| | ccgggcatgtctgtcatttgtgtcatcccaactgcacatatggctgtaccggtcctgg | |
| | attggagggctgtccaacaaacgggccgaaaataccgagtatcgcaacaggcat | |
| | ggtgggagcacttttgcttctcctcgttgtcgccctgggcatcggcttgttcatg | |
| Leader | MALPVTALLLPLALLLHAARP | 81 |
| BCMA scFv | DIVLTQSPASLAMSLGKRATISCRASESVSVIGAHLIH | 82 |
| | WYQQKPGQPPKLLIYLASNLETGVPARFSGSGSGTD | |
| | FTLTIDPVEEDDVAIYSCLQSRIFPRTFGGGTKLEIKG | |
| | GGGSGGGGSGGGGSQIQLVQSGPELKKPGETVKISC | |
| | KASGYTFTDYSINWVKRAPGKGLKWMGWINTETRE | |
| | PAYAYDFRGRFAFSLETSASTAYLQINNLKYEDTAT | |
| | YFCALDYSYAMDYWGQGTSVTVSS | |
| CD8 hinge | TTTPAPRPPTPAPTIASQPLSLRPEACRPAAGGAVHT | 83 |
| | RGLDFACDIY | |
| CD8 transmembrane | IWAPLAGTCGVLLLSLVITLYC | 84 |
| 4-1BB costimulatory | RFSVVKRGRKKLLYIFKQPFMRPVQTTQEEDGCSCR | 85 |
| domain | FPEEEEGGCELR | |
| CD3 zeta intracellular | VKFSRSADAPAYQQGQNQLYNELNLGRREEYDVLD | 86 |
| signaling domain | KRRGRDPEMGGKPRRKNPQEGLYNELQKDKMAEA | |
| | YSEIGMKGERRRGKGHDGLYQGLSTATKDTYDALH | |
| | MQALPPR | |
| P2A peptide | RAKRGSGATNFSLLKQAGDVEENPGP | 87 |
| Truncated EGFR | MLLLVTSLLLCELPHPAFLLIPRKVCNGIGIGEFKDSL | 88 |
| | SINATNIKHFKNCTSISGDLHILPVAFRGDSFTHTPPL | |
| | DPQELDILKTVKEITGFLLIQAWPENRTDLHAFENLEI | |
| | IRGRTKQHGQFSLAVVSLNITSLGLRSLKEISDGDVII | |
| | SGNKNLCYANTINWKKLFGTSGQKTKIISNRGENSC | |
| | KATGQVCHALCSPEGCWGPEPRDCVSCRNVSRGRE | |
| | CVDKCNLLEGEPREFVENSECIQCHPECLPQAMNITC | |
| | TGRGPDNCIQCAHYIDGPHCVKTCPAGVMGENNTL | |
| | VWKYADAGHVCHLCHPNCTYGCTGPGLEGCPTNG | |
| | PKIPSIATGMVGALLLLLVVALGIGLFM | |
| iNKT TCR-apha chain | gggagatactcagcaactctggataaagatgc | 89 |
| forward primer | | |
| iNKT TCR-apha chain | ccagattccatggttttcggcacattg | 90 |
| reverse primer | | |
| iNKT TCR-beta chain | ggagatatccctgatggatacaaggcctcc | 91 |
| forward primer | | |
| iNKT TCR-beta chain | gggtagccttttgtttgtttgcaatctctg | 92 |
| reverse primer | | |

### XIV. Examples

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1: Hematopoietic Stem Cell (HSC) Approach to Engineer Off-the-Shelf iNKT Cells

The present example concerns generation of off-the-shelf iNKT cells that comprise lack of or down-regulated surface expression of one or more HLA-I and/or HLA-II molecules. In a specific embodiment, iNKT cells are expanded from healthy donor peripheral blood mononuclear cells (PBMCs), followed by CRISPR-Cas9 engineering to knockout B2M and CIITA genes. Because of the high-variability and low-frequency of iNKT cells in human population (˜0.001-0.1% in blood), it is beneficial to produce methods that allow alternative means to obtaining iNKT cells.

The present disclosure provides a powerful method to generate iNKT cells from hematopoietic stem cells (HSCs) through genetically engineering HSCs with an iNKT TCR gene and programming these HSCs to develop into iNKT cells (Smith et al., 2015). This method takes advantage of two molecular mechanisms governing iNKT cell development: 1) an Allelic Exclusion mechanism that blocks the rearrangement of endogenous TCR genes in the presence of a transgenic iNKT TCR gene, and 2) a TCR Instruction Mechanism that guides the developing T cells down an iNKT lineage path (Smith et al., 2015). The resulting HSC-engineered iNKT (HSC-iNKT) cells are a homogenous “clonal” population that do not express endogenous TCRs. Mouse HSC-iNKT cells have been generated with a potent anti-cancer efficacy of these iNKT cells in a mouse bone marrow transfer and melanoma lung metastasis model (Smith et al., 2015).

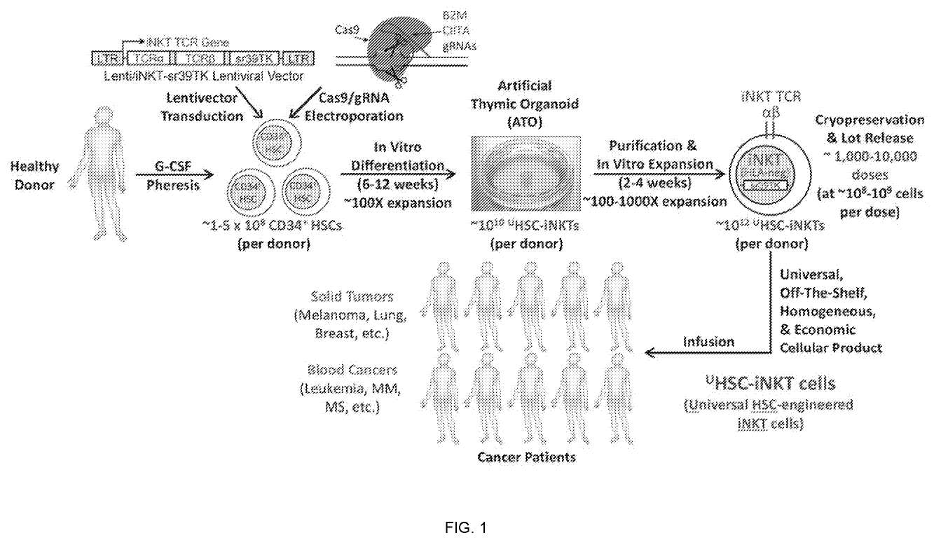

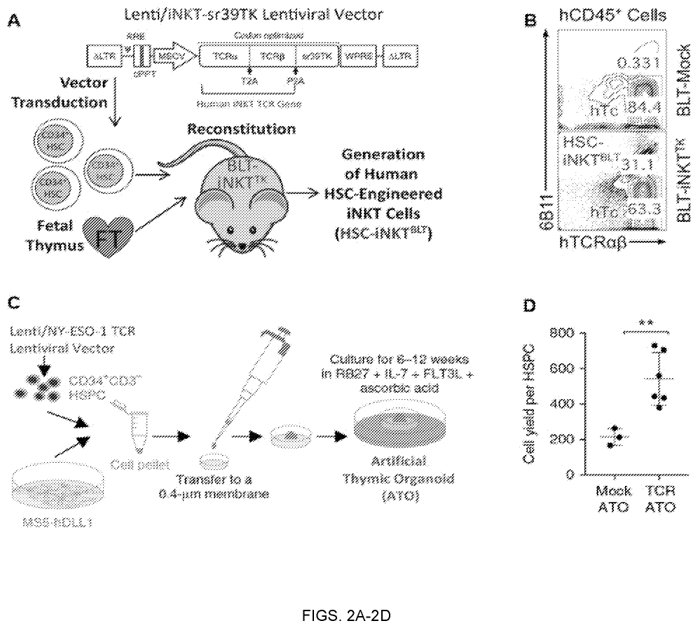

HSC-engineered human iNKT cells are produced by genetically engineering human CD34+ peripheral blood stem cells (PBSCs) with a human iNKT TCR gene followed by transferring the engineered PBSCs into a BLT humanized mouse model (FIGS. 2A and 2B). However, such an in vivo approach can only be translated as an autologous HSC adoptive therapy. In particular embodiments, a serum-free, “Artificial Thymic Organoid (ATO)” in vitro culture system that supports the differentiation of TCR-engineered human CD34+ HSCs into clonal T cells at high-efficiency and high yield (FIGS. 2C and 2D) (Seet et al., 2017) is utilized. This ATO culture system allows one to move the HSC-iNKT production to an in vitro system, and based on this, an off-the-shelf universal HSC-engineered iNKT (UHSC-iNKT) cell adoptive therapy may be utilized (FIG. 1). Because iNKT cells can target multiple types of cancer without tumor antigen- and major histocompatibility complex (MHC)-restrictions, the ^{U}HSC-iNKT therapy is useful as a universal cancer therapy for treating multiple cancers and a large population of cancer patients, thus addressing the unmet medical need (FIG. 1) (Vivier et al., 2012; Berzins et al., 2011). Particularly, the disclosed HSC-iNKT therapy is useful to treat the many types of cancer that have been clinically implicated to be subject to iNKT cell regulation, including blood cancers (leukemia, multiple myeloma, and myelodysplastic syndromes), and solid tumors (melanoma, colon, lung, breast, and head and neck cancers) (Berzins et al., 2011).

Allogeneic HLA-negative human iNKT cells cultured in vitro from gene-engineered healthy donor HSCs are encompassed herein. Examples of their production are provided below.

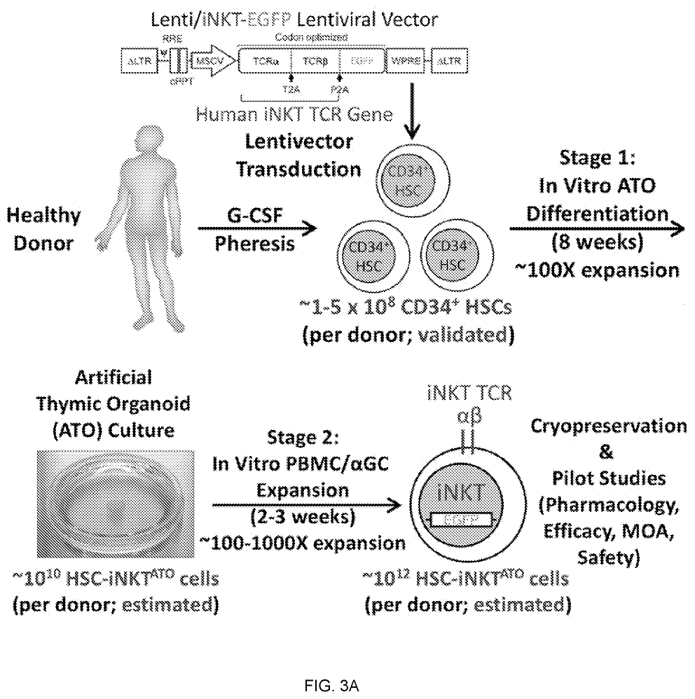

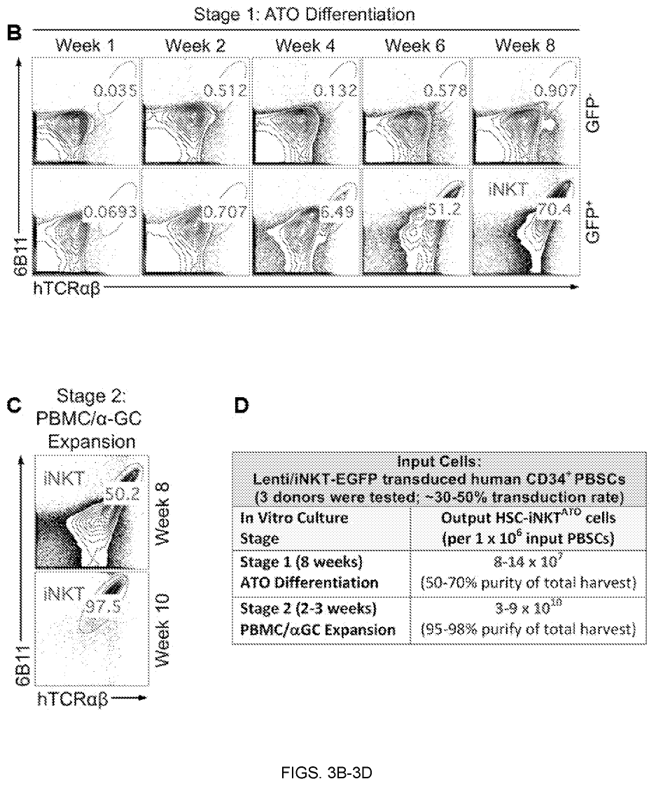

A. Initial CMC Study (FIG. 3)

Unless otherwise noted, human G-CSF-mobilized peripheral blood CD34+ cells contain both hematopoietic stem and progenitor cells. Herein, these CD34+ cells are referred to as HSCs.

An initial chemistry, manufacturing, and controls (CMC) study is conducted to test the in vitro manufacture of human HSC-engineered iNKT cells. In specific cases, HSC-iNKTATO cells are produced, which are HSC-engineered human iNKT cells generated in vitro in a two-stage ATO-αGC culture system.

G-CSF-mobilized human CD34+ HSCs were collected from three different healthy donors, transduced with an analog lentiviral vector Lenti/iNKT-EGFP, followed by culturing in vitro in a two-stage ATO-αGC culture system (FIG. 3A). Gene-engineered HSCs (labeled as GFP+) efficiently differentiated into human iNKT cells in the Artificial Thymic Organoid (ATO) culture stage over 8 weeks (FIG. 3B), then further expanded in the PBMC/αGC stimulation stage for another 2-3 weeks (FIG. 3C). This manufacturing process was robust and of high yield and high purity for all three donors tested (FIG. 3D). Based on the results, it was estimated that from 1×10⁶ input HSCs (˜30-50% lentivector transduction rate), about 3-9×1010 HSC-iNKTATO cells (>95% purity) could be produced, giving a theoretical yield of over 1012 therapeutic iNKT cells from a single random donor (FIG. 3D).

B. Initial Pharmacology Study (FIG. 4)

An initial pharmacology study was performed to study the phenotype and functionality of human HSC-engineered iNKT cells. The phenotype and functionality of the human HSC-engineered iNKT cells were studied using flow cytometry. Both HSC-iNKTATO cells (HSC-engineered human iNKT cells generated in vitro in an ATO culture system) and HSC-iNKT^{BLT} cells (HSC-engineered human iNKT cells generated in vivo in a BLT (human bone marrow-liver-thymus engrafted NOD/SCID/γc−/−) humanized mouse model displayed typical iNKT cell phenotype and functionality similar to that of the endogenous PBMC-iNKT cells: they expressed high levels of memory T cell marker CD45RO and NK cell marker CD161 (FIG. 4A); they expressed the CD4 and CD8 co-receptors at a mixed pattern (CD4 single-positive, CD8 single-positive, and CD4/CD8 double-negative) (FIG. 4A); and they produced exceedingly high levels of effector cytokine like IFN-γ and cytotoxic molecules like Perforin and Granzyme B, compared to that of the conventional PBMC-Tc cells (FIG. 4B).

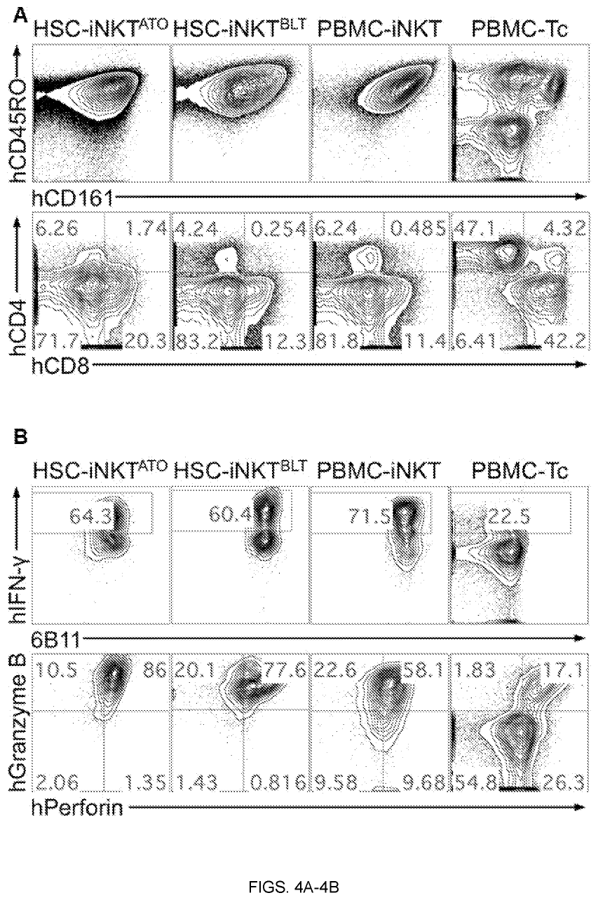

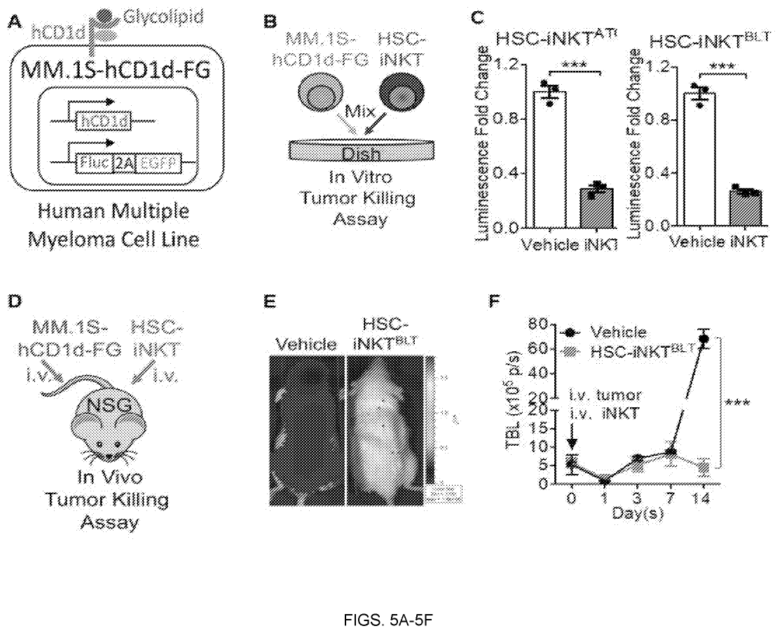

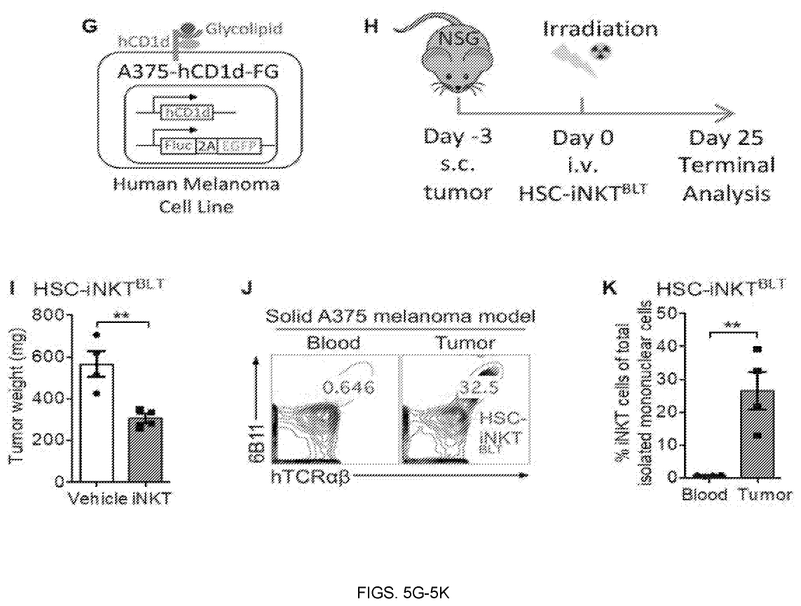

C. Initial Efficacy Study (FIG. 5)

An initial efficacy study was performed to study the tumor killing efficacy of human HSC-engineered iNKT cells. Human multiple myeloma (MM) cell line MM.1S was engineered to overexpress the human CD1d gene, as well as a firefly luciferase (Fluc) reporter gene and an enhanced green fluorescence protein (EGFP) reporter gene (FIG. 5A). The resulting MM.1S-hCD1d-FG cell line was then used to study iNKT cell-targeted tumor killing in vitro in a mixed culture assay (FIG. 5B) and in vivo in an NSG (NOD/SCID/γc^{-/-}) mouse human multiple myeloma (MM) metastasis model (FIG. 5D). Both HSC-iNKT^{ATO} and HSC-iNKT^{BLT} cells showed efficient and comparable tumor killing in vitro (FIG. 5C). HSC-iNKT^{BLT} cells were also tested in vivo and they mediated robust tumor killing (FIGS. 5E and 5F). To study tumor killing efficacy for solid tumors, an A375-hCD1d-FG human melanoma cell line was generated (FIG. 5G). When tested in an NSG mice A375-hCD1d-FG xenograft solid tumor model (FIG. 5H), HSC-iNKT^{BLT} cells efficiently suppressed solid melanoma tumor growth (FIG. 5I). Importantly, HSC-iNKT^{BLT} cells showed targeted infiltration into the tumor sites, presumably due to the potent tumor-trafficking capacity of these cells (FIGS. 5J and 5K).

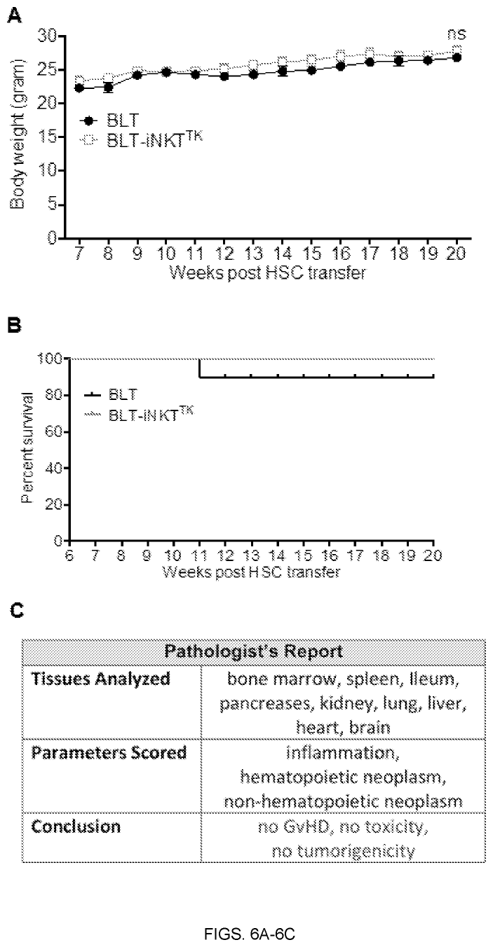

D. Initial Safety Study—GvHD/Toxicology/Tumorigenicity (FIG. 6)

To access the in vivo long-term GvHD, toxicology, and tumorigenicity of human HSC-engineered iNKT cells, the BLT humanized mice that harbored HSC-iNKT^{BLT} cells were monitored over a period of 5 months post HSC transfer, followed by tissue collection and pathological analysis (FIG. 6). Monitoring of mouse body weight (FIG. 6A), survival (FIG. 6B), and tissue pathology (FIG. 6C) revealed no GvHD, no toxicity, and no tumorigenicity in the BLT-iNKT^{TK} mice (FIG. 2A) compared to the control BLT mice.

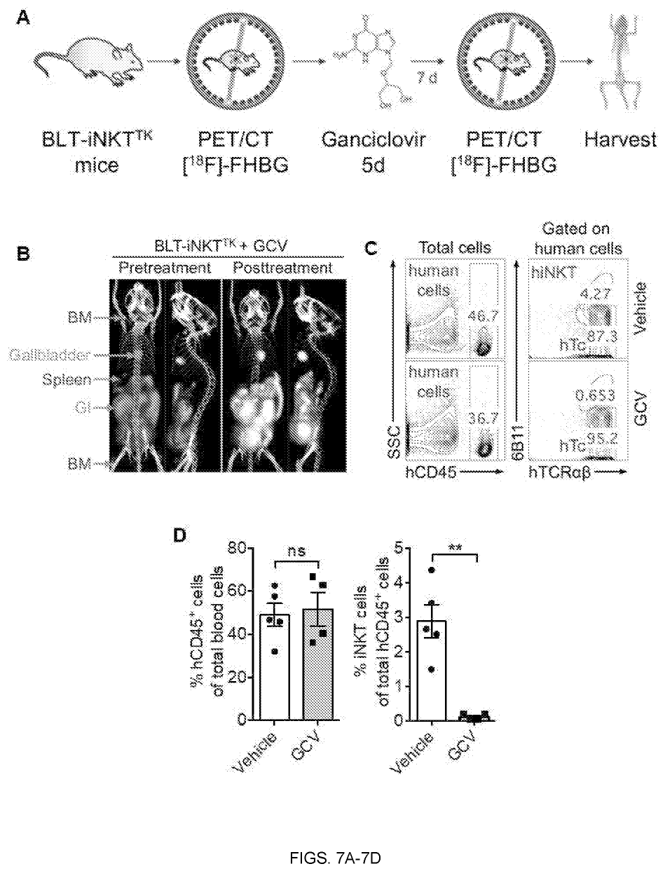

E. Initial Safety Study—sr39TK Gene for PET Imaging and Safety Control (FIG. 7)

BLT-iNKT^{TK} humanized mice harboring human HSC-engineered iNKT (HSC-iNKT^{BLT}) cells were studied (FIG. 7A). The HSC-iNKT^{BLT} cells were engineered from human HSCs transduced with a Lenti/iNKT-sr39TK lentiviral vector (FIG. 13). Using PET imaging combined with CT scan, the inventors detected the distribution of gene-engineered human cells across the lymphoid tissues of BLT-iNKT^{TK} mice, particularly in bone marrow (BM) and spleen (FIG. 7B). Treating BLT-iNKT^{TK} mice with GCV effectively depleted gene-engineered human cells across the body (FIG. 7B). Importantly, the GCV-induced depletion was specific, evidenced by the selective depletion of the HSC-engineered human iNKT cells but not other human immune cells in BLT-iNKT^{TK} mice as measured by flow cytometry (FIGS. 7C and 7D).

F. Production of Universal HSC-Engineered iNKT Cells

In specific embodiments, a stem cell-based therapeutic composition is produced that comprises allogeneic HSC-engineered HLA-I/II-negative human iNKT cells (denoted as the Universal HSC-Engineered iNKT cells, ^{U}HSC-iNKT cells).

Generate a Lenti-iNKT-sr39TK vector In certain embodiments, a clinical lentiviral vector Lenti/iNKT-sr39TK is utilized (FIG. 8A).

Generate a CRISPR-Cas9/B2M-CIITA-gRNAs complex In specific embodiments, the powerful CRISPR-Cas9/gRNA gene-editing tool is used to disrupt the B2M and CIITA genes in human HSCs (Ren et al., 2017; Liu et al., 2017). iNKT cells derived from such gene-edited HSCs will lack the HLA-I/II expression, thereby avoiding rejection by the host T cells. In an initial study, a CRISPR-Cas9/B2M-CIITA-gRNAs complex was successfully generated and tested (Cas9 from the UC Berkeley MacroLab Facility; gRNAs from the Synthego; B2M-gRNA sequence 5′-CGCGAGCACAGCUAAGGCCA-3′ (SEQ ID NO:68) (Ren et al., 2017); CIITA-gRNA sequence 5′-GAUAUUGGCAUAAGCCUCCC-3′ (SEQ ID NO:69) (Abrahimi et al., 2015)). To minimize an “off-target” effect, one can utilize the high-fidelity Cas9 protein from IDT (Kohn et al., 2016; Slaymaker et al., 2016; Tsai and Joung, 2016). One can start with the pre-tested single dominant B2M-gRNA and CIITA-gRNA, but in specific embodiments multiple gRNAs are incorporated to further improve gene-editing efficiency.

Collect G-CSF-mobilized CD34⁺ HSCs One can obtain G-CSF-mobilized leukopaks of at least two different healthy donors from a commercial vendor, followed by isolating the CD34⁺ HSCs using a CliniMACS system. After isolation, G-CSF-mobilized CD34⁺ HSCs may be cryopreserved and used later.

Gene-engineer HSCs HSCs may be engineered with both the Lenti-iNKT-sr39TK vector and the CRISPR-Cas9/B2M-CIITA-gRNAs complex. Cryopreserved CD34⁺ HSCs may be thawed and cultured in X-Vivo-15 serum-free medium supplemented with 1% HAS and TPO/FLT3L/SCF for 12 hours in flasks coated with retronectin, followed by addition of the Lenti/iNKT-sr39TK vector for an additional 8 hours (Gschweng et al., 2014). 24 hours post the lentivector transduction, cells may be mixed with pre-formed CIRSPR-Cas9/B2M-CIITA-gRNAs complex and subjected to electroporation using a Lonza Nucleofector. In initial studies, high lentivector transduction rate (>50% transduction rate with VCN=1-3 per cell; FIG. 8B) and high HLA-I/II expression deficiency (˜60% HLA-I/II double-negative cells post a single round of electroporation; FIG. 8C) was achieved using CD34⁺ HSCs from a random donor. One can further optimize the gene-editing procedure to improve efficiency. Evaluation parameters may include cell viability, deletion (indel) frequency (on-target efficiency) measured by a T7E1 assay and next-generation sequencing (NGS) targeting the B2M and CIITA sites (Tsai et al., 2015), HLA-I/II expression by flow cytometry, and hematopoietic function of edited HSCs measured by the colony formation unit (CFU) assay. One can achieve 30-50% triple-gene editing efficiency of HSCs, which in initial studies could give rise to ˜100 iNKT cells per input HSC post ATO culture (FIG. 3).

Produce ^{U}HSC-iNKT cells One can culture the lentivector and CRISPR-Cas9/gRNA double-engineered HSCs in a 2-stage ATO-αGC in vitro system to produce ^{U}HSC-iNKT cells. At Stage 1, the gene-engineered HSCs will be differentiated into iNKT cells via the Artificial Thymic Organoid (ATO) culture following a standard protocol (FIG. 8A) (Seet et al., 2017). ATO involves pipetting a cell slurry (5 μl) containing a mixture of HSCs (1×10⁴) and irradiated (80 Gy) MS5-hDLL1 stromal cells (1.5×10⁵) as a drop format onto a 0.4-μm Millicell transwell insert, followed by placing the insert into a 6-well plate containing 1 ml RB27 medium (Seet et al., 2017); medium will be changed every 4 days for 8 weeks (Seet et al., 2017). The total harvest from the Stage 1 are expected to contain a mixture of cells. One can perform a purification step to purify the ^{U}HSC-iNKT cells through MACS sorting (2M2/Tü39 mAb-mediated negative selection followed by 6B11 mAb-mediated positive selection) (FIG. 8D). Initial studies showing the effectiveness of this MACS sorting strategy (FIGS. 8E and 8F) are completed. The purified ^{U}HSC-iNKT cells then enter the Stage 2 culture, stimulated with αGC loaded onto irradiated matched-donor CD34-PBMCs (as APCs) and with the supplement of IL-7 and IL-15 (FIG. 8A). Based on initial studies (FIG. 3), ˜10¹⁰ scale of ^{U}HSC-iNKT cells (>99% purity) may be produced from every 1×10⁶ starting HSCs, that will give ˜10¹² pure and homogenous ^{U}HSC-iNKT cellular product from HSCs of a single random donor (FIG. 8A). The resulting ^{U}HSC-iNKT cells may then be cryopreserved and ready for preclinical characterizations.

G. Characterization of the ^{U}HSC-iNKT Cells

Identity/activity/purity One can study the purity, phenotype, and functionality of the ^{U}HSC-iNKT cell product using pre-established flow cytometry assays (FIG. 4). In specific cases, >99% purity of ^{U}HSC-iNKT cells (gated as hTCRαβ⁺6B11⁺HLA-I/II^{neg}) is achieved. In specific embodiments, these ^{U}HSC-iNKT cells display a typical iNKT cell phenotype (hCD45RO^{hi}hCD161^{hi}hCD4^{+/-}hCD8^{+/-}), express no detectable endogenous TCRs due to allelic exclusion (Seet et al., 2017; Smith et al., 2015; Giannoni et al., 2013), and respond to PBMC/αGC stimulation by producing excess amount of effector cytokines (IFN-γ) and cytotoxic molecules (Granzyme B, perforin) (FIG. 4) (Watarai et al., 2008).

Pharmacokinetics/pharmacodynamics (PK/PD) One can study the bio-distribution and in vivo dynamics of the ^{U}HSC-iNKT cells by adoptively transferring these cells into tumor-bearing NSG mice. A pre-established A375 human melanoma solid tumor xenograft model may be used (FIG. 5H), for example. Flow cytometry analysis may be performed to study the presence of ^{U}HSC-iNKT cells in tissues. PET imaging may be performed to study the whole-body distribution of ^{U}HSC-iNKT cells, following established protocols (FIG. 7). Based on initial studies, in specific embodiments the ^{U}HSC-iNKT cells can persist in tumor-bearing animals for some time post adoptive transfer, can home to the lymphoid organs (spleen and bone marrow), and most importantly, and can traffic to and infiltrate into solid tumors (FIGS. 5I-5K).

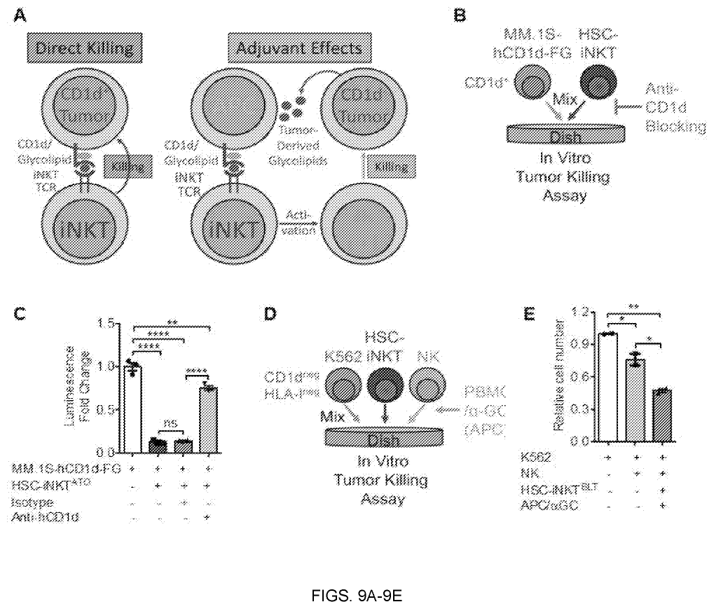

Mechanism of action (MOA) iNKT cells can target tumor through multiple mechanisms: 1) they can directly kill CD1d⁺ tumor cells through iNKT TCR stimulation, and 2) they can indirectly target CD1d⁻ tumor cells through recognizing tumor-derived glycolipids presented by tumor-associated antigen-presenting cells (which constantly express CD1d), then activating the downstream effector cells, like NK cells and CTLs, to kill these CD1d⁻ tumor cells (FIG. 9A) (Vivier et al., 2012). Many cancer cells produce glycolipids that can stimulate iNKT cells, albeit the nature of such “altered” glycolipids remain to be elucidated (Bendelac et al., 2007). Using an in vitro direct tumor killing assay (FIG. 9B), the therapeutic surrogates HSC-iNKT^{ATO} and HSC-iNKT^{BLT} cells directly killed tumor cells in an CD1d/TCR-dependent manner (FIG. 9C). Using an in vitro mixed culture assay (FIG. 9D), it was further shown that HSC-iNKT^{BLT} cells stimulated by APCs could activate NK cells to kill CD1d⁻HLA-I^{-/-} K562 human myeloid leukemia cells (FIG. 9E). These pre-established assays may be utilized to study ^{U}HSC-iNKT cell targeting of tumor cells. In particular embodiments, the ^{U}HSC-iNKT cells can target tumor through both direct killing and adjuvant effects.

Efficacy One can study the tumor killing efficacy of ^{U}HSC-iNKT cells using the pre-established in vitro and in vivo assays (FIG. 5). Both a human blood cancer model (MM1.S multiple myeloma) and a human solid tumor model (A375 melanoma) may be used (FIG. 5), for example. In certain embodiments, the ^{U}HSC-iNKT cells can effectively kill both MM1.S and A375 tumor cells in vitro and in vivo, similar to what has been observed for the therapeutic surrogates HSC-iNKT^{ATO} and HSC-iNKT^{BLT} cells (FIG. 5).

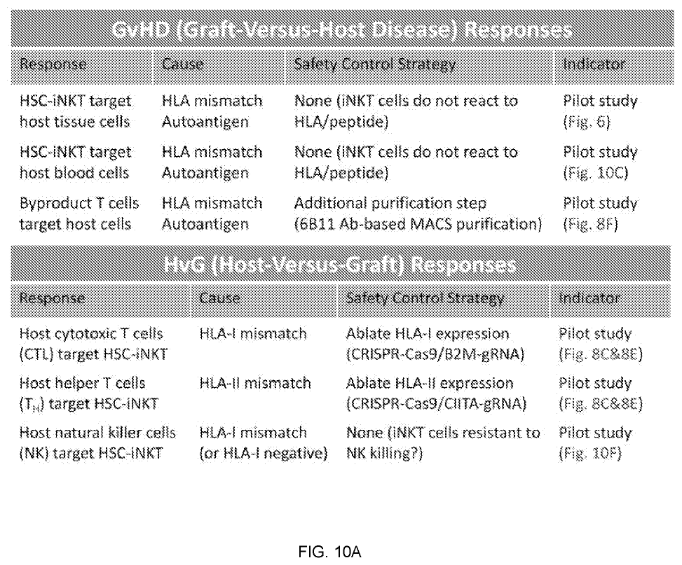

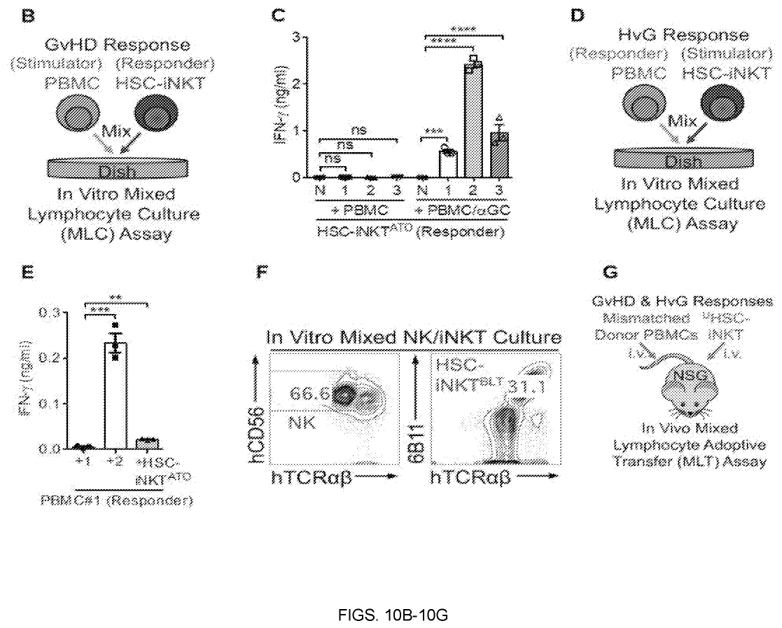

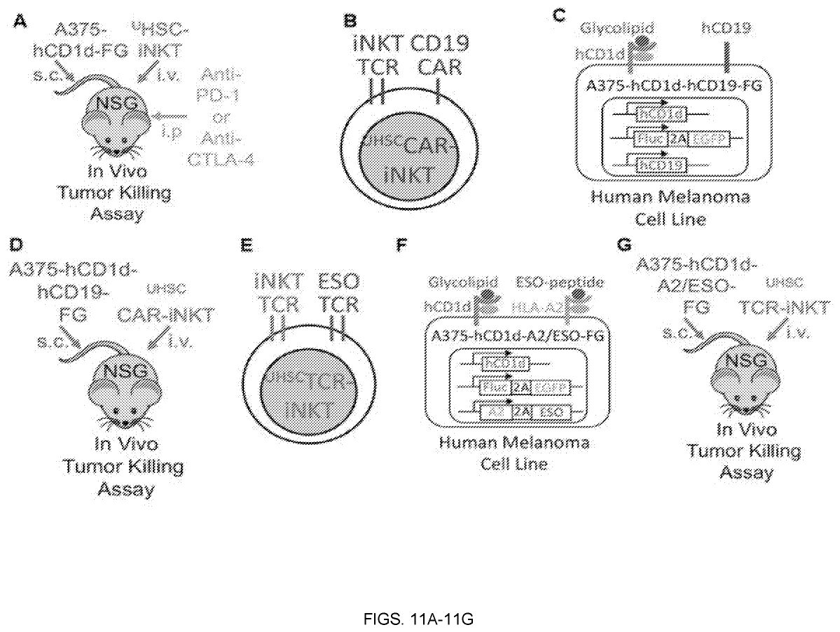

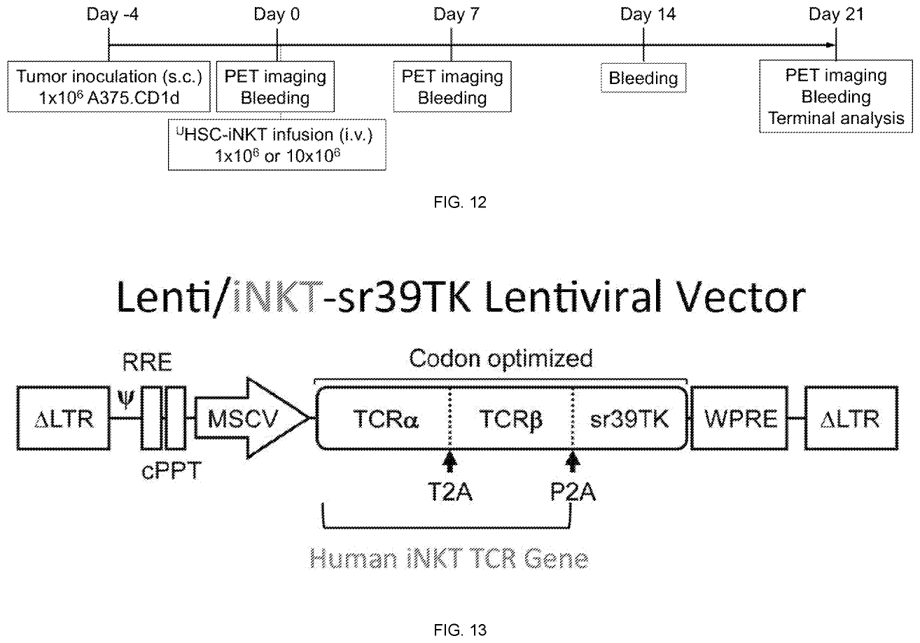

Safety One can study the safety of ^{U}HSC-iNKT adoptive therapy on three aspects, as example: a) general toxicity/tumorigenicity, b) immunogenicity, and c) suicide gene “kill switch”. 1) The long-term GvHD (against recipient animal tissues), toxicology, and tumorigenicity of ^{U}HSC-iNKT cells may be studied through adoptively transferring these cells into NSG mice and monitoring the recipient mice over a period of 20 weeks ended by terminal pathology analysis, following an established protocol (FIG. 6). No GvHD, no toxicity, and no tumorigenicity are expected (FIG. 6). 2) For immune cell-based adoptive therapies, there are always two immunogenicity concerns: a) Graft-Versus-Host Disease (GvHD) responses, and b) Host-Versus-Graft (HvG) responses. Engineered safety control strategies mitigate the possible GvHD and HvG risks for the ^{U}HSC-iNKT cellular product (FIG. 10A). Possible GvHD and HvG responses are studied using an established in vitro Mixed Lymphocyte Culture (MLC) assay (FIGS. 10B and 10D) and an in vivo Mixed Lymphocyte Adoptive Transfer (MLT) Assay (FIG. 10G). The readouts of the in vitro MLC assays may be IFN-γ production analyzed by ELISA, while the readouts of the in vivo MLT assays may be the elimination of targeted cells analyzed by bleeding and flow cytometry (either the killing of mismatched-donor PBMCs as a measurement of GvHD response, or the killing of ^{U}HSC-iNKT cells as a measurement of HvG response). Based on initial studies, in specific embodiments the ^{U}HSC-iNKT cells do not induce GvHD response against host animal tissues (FIG. 6), and do not induce GvHD response against mismatched-donor PBMCs (FIG. 10C). In specific embodiments, ^{U}HSC-iNKT cells are resistant to HvG-induced elimination. Initial studies showed that even with HLA-I/II expression, HSC-iNKT^{ATO} cells were already weak targets for mismatched-donor PBMC T cells (FIG. 10E). In specific cases there is a total lack of T cell-mediated HvG response against the ^{U}HSC-iNKT cells. Interestingly, initial studies showed that the surrogate HSC-iNKT^{BLT} cells were resistant to killing by mismatched-donor NK cells (FIG. 10F). In some cases, lack of HLA-I expression on ^{U}HSC-iNKT cells may make these cells more susceptible to NK killing. Therefore the final ^{U}HSC-iNKT cellular product may be tested. 3) One can study the elimination of ^{U}HSC-iNKT cells in recipient NSG mice through GCV administration, following an established protocol (FIG. 7). Based on initial studies, the sr39TK suicide gene can function as a potent “kill switch” to eliminate ^{U}HSC-iNKT cells in case of a safety need.

Combination therapy One can examine ^{U}HSC-iNKT cells for combination immunotherapy. In particular, there are synergistic therapeutic effects combining the ^{U}HSC-iNKT adoptive therapy with the checkpoint blockade therapy (e.g., PD-1 and CTLA-4 blockade) (Pilones et al., 2012; Durgan et al., 2011). A pre-established human melanoma solid tumor model (A375-hCD1d-FG) may be used (FIG. 11A). One can further engineer the ^{U}HSC-iNKT cells to express cancer-targeting CARs (chimeric antigen receptors) or TCRs (T cell receptors) for next-generation universal CAR-iNKT and TCR-iNKT therapies (denoted as ^{UHSC}CAR-iNKT and ^{UHSC}TCR-iNKT therapies) (Oberschmidt et al., 2017; Bollino and Webb, 2017; Heczey et al., 2014; Chodon et al., 2014). For the study of ^{UHSC}CAR-iNKT therapy, ^{U}HSC-iNKT cells may be transduced with a lentivector encoding a CD19-CAR gene (FIG. 11B). Meanwhile, the human melanoma cell line A375-hCD1d-FG, as an example, may be further engineered to overexpress the human CD19 antigen (FIG. 11C). The anti-tumor efficacy of the ^{UHSC}CAR-iNKT cells may be studied using the A375-hCD1d-hCD19-FG tumor xenograft model (FIG. 11D). For the study of ^{UHSC}TCR-iNKT therapy, ^{U}HSC-iNKT cells may be transduced with a lentivector encoding an NY-ESO-1 TCR gene (FIG. 11E). The A375-hCD1d-FG cell line may be further engineered to overexpress the human HLA-A2 molecule and the NY-ESO-1 antigen (FIG. 11F). The anti-tumor efficacy of the ^{UHSC}TCR-iNKT cells may be studied using the A375-hCD1d-A2/ESO-FG tumor xenograft model (FIG. 11G).

H. Pharmacology Embodiments

Drug mechanism for ^{U}HSC-iNKT therapy ^{U}HSC-iNKT is a cellular product that at least in some cases is generated by 1) genetic modification of donor HSCs to express iNKT TCRs via lentiviral vectors and to knockout HLAs via CRISPR/Cas9-based gene editing, 2) in vitro differentiation into iNKT cells via an ATO culture, 3) in vitro iNKT cell expansion, and 4) formulation and cryopreservation. Once infused into patients, this cell product can employ multiple mechanisms to target and eradicate tumor cells, in at least some embodiments. The infused cells can directly recognize and kill CD1d⁺ tumor cells through cytotoxicity. They can secrete cytokines such as IFN-γ to activate NK cells to kill HLA-negative tumor cells, and also activate DCs which then stimulate cytotoxic T cells to kill HLA-positive tumor cells. Accordingly, a series of in vitro and in vivo studies may be utilized to demonstrate the pharmacological efficacy of this cell product for cancer therapy.

In vitro surface and functional characterization An efficient protocol to generate ^{U}HSC-iNKT cells is provided herein. An efficient gene editing of HSCs to ablate the expression of class I HLA via knockout of B2M is also demonstrated. Taking advantage of the multiplex editing CRISPR/Cas9, one can also simultaneously disrupt class II HLA expression via knockout of the gene for the class II transactivator (CIITA), a key regulator of HLA-II expression (Steimle et al., 1994), using a validated gRNA sequence (Abrahimi et al., 2015). Thus, incorporating this gene editing step to disrupt HLA-I and HLA-II expression and the microbeads purification step, one can generate ^{U}HSC-iNKT cells (details provided elsewhere herein). Flow cytometric analysis may be used to measure the purity and the surface phenotypes of these engineered iNKT cells. The cell purity may be characterized by TCR Vα24-Jα18(6B11)⁺HLA-I/II^{neg}. In at least some cases, this iNKT cell population should be CD45RO⁺CD161⁺, indicative of memory and NK phenotypes, and contain CD4⁺CD8⁻ (CD4 single-positive), CD4⁻CD8⁺ (CD8 single-positive), and CD4⁻CD8⁻ (double-genative, DN)(Kronenberg and Gapin, 2002). One can analyze CD62L expression, as a recent study indicated that its expression is associated with in vivo persistence of iNKT cells and their antitumor activity (Tian et al., 2016). One can compare these phenotypes of ^{U}HSC-iNKT with that iNKT from PBMCs. RNAseq may be employed to perform comparative gene expression analysis on ^{U}HSC-iNKT and PBMC iNKT cells.

IFN-γ production and cytotoxicity assays may be used to assess the functional properties of ^{U}HSC-iNKT, using PBMC iNKT as the benchmark control. ^{U}HSC-iNKT cells may be simulated with irradiated PBMCs that have been pulsed with αGalCer and supernatants harvested from one day stimulation will be subjected to IFN-γ ELISA (Smith et al., 2015). Intracellular cytokine staining (ICCS) of IFN-γ may be performed as well on iNKT cells after 6-hour stimulation. The cytotoxicity assay may be conducted by incubating effector ^{U}HSC-iNKT cells with αGC-loaded A375.CD1d target cells engineered to expression luciferase and GFP for 4 hours and cytotoxicity may be measured by a plate reader for its luminescence intensity. Because sr39TK is introduced as a PET/suicide gene, one can verify its function by incubating ^{U}HSC-iNKT with ganciclovir (GCV) and cell survival rate may be measured by a MTT assay and an Annexin V-based flow cytometric assay.

Pharmacokinetics/Pharmacodynamics (PK/PD) studies The PK/PD studies may determine in vivo in animal models: 1) expansion kinetics and persistence of infused ^{U}HSC-iNKT; 2) biodistribution of ^{U}HSC-iNKT in various tissues/organs; 3) ability of ^{U}HSC-iNKT to traffic to tumors and how this filtration relates to tumor growth. Immunodeficient NSG mice bearing A375.CD1d (A375.CD1d) tumors may be utilized as the solid tumor animal model. The study design is outlined in FIG. 11. Two examples of cell dose groups (1×10⁶ and 10×10⁶; n=8) may be investigated. The tumors are inoculated (s.c.) on day −4 and the baseline PET imaging and bleeding is conducted on day 0. Subsequently, ^{U}HSC-iNKT cells is infused intravenously (i.v.) and monitored by 1) PET imaging in live animals on days 7 and 21; 2) periodic bleeding on days 7, 14 and 21; 3) end-point tissue collection after animal termination on day 21. Cell collected from various bleedings may be analyzed by flow cytometry; iNKT cells are TCRαβ⁺6B11⁺, in specific embodiments. One can examine the expression of other markers such as CD45RO, CD161, CD62L, and CD4/CD8 to see how iNKT subsets vary over the time. PET imaging via sr39TK will allow tracking of the presence of iNKT cells in tumors and other tissues/organs such as bone, liver, spleen, thymus, etc. At the end of the study, tumors and mouse tissues including spleen, liver, brain, heart, kidney, lung, stomach, bone marrow, ovary, intestine, etc., are harvested for qPCR analysis to examine the distribution of ^{U}HSC-iNKT cells.

Antitumor efficacy in vivo In vivo pharmacological responses are measured by treating tumor-bearing NSG mice with escalating doses (1×10⁶, 5×10⁶, 10×10⁶) of ^{U}HSC-iNKT cells (n=8 per group); treatment with PBS is included as a control. Two tumor models may be utilized as examples. A375.CD1d (1×10⁶ s.c.) may be used as a solid tumor model and MM.1S.Luc (5×10⁶ i.v.) may be used as a hematological malignancy model. Tumor growth is monitored by either measuring size (A375.CD1d) or bioluminescence imaging (MM.1S.Luc). Antitumor immune responses are measured by PET imaging, periodic bleeding, and end-point tumor harvest followed by flow cytometry and qPCR. Inhibition of tumor growth in response to ^{U}HSC-iNKT treatment indicates the therapeutic efficacy of proposed ^{U}HSC-iNKT cell therapy. Correlation of tumor inhibition with iNKT doses confirms the therapeutic role of the iNKT cells and can indicate an effective therapeutic window for human therapy. Detection of iNKT cell responses to tumors demonstrates the pharmacological antitumor activities of these cells in vivo.

Mechanism of action (MOA) iNKT cells are known to target tumor cells through either direct killing, or through the massive release of IFN-γ to direct NK and CD8 T cells to eradicate tumors (Fujii et al., 2013). An in vitro pharmacological study provides evidence of direct cytotoxicity. Here one can investigate the possible roles of NK and CD8 T cells in assisting antitumor reactivity in vivo. Tumor-bearing NSG mice (A375.CD1d or MM.1S.Luc) may be infused with either ^{U}HSC-iNKT alone (a dose chosen based on above in vivo study) or in combination with PBMCs (mismatched donor, 5×10⁶); owing to the MHC negativity of ^{U}HSC-iNKT, no allogenic immune response is expected between ^{U}HSC-iNKT and unrelated PBMCs. Tumor growth may be monitored and compared between with and without PBMC groups (n=8 per group). If a greater antitumor response is observed from the combination group, it will indicate that at least in specific embodiments components in PBMCs, presumably NK and/or CD8 T cells, play a role to boost therapeutic efficacy. To further determine their individual roles, PBMCs with depletion of NK (via CD56 beads), CD8 T cells (via CD8 beads), or myeloid (via CD14 beads) cells, are co-infused along with ^{U}HSC-iNKT cells into tumor-bearing mice. Immune checkpoint inhibitors such as PD-1 and CTLA-4 have been suggested to regulate iNKT cell function (Pilones et al., 2012; Durgan et al., 2011). Through adding anti-PD-1 or anti-CTLA-4 treatment to the ^{U}HSC-iNKT therapy, one can understand how these molecules modulate ^{U}HSC-iNKT therapy and provide valuable guidance on the design of combination cancer therapy, for example.

I. Embodiments of Chemistry, Manufacturing and Controls

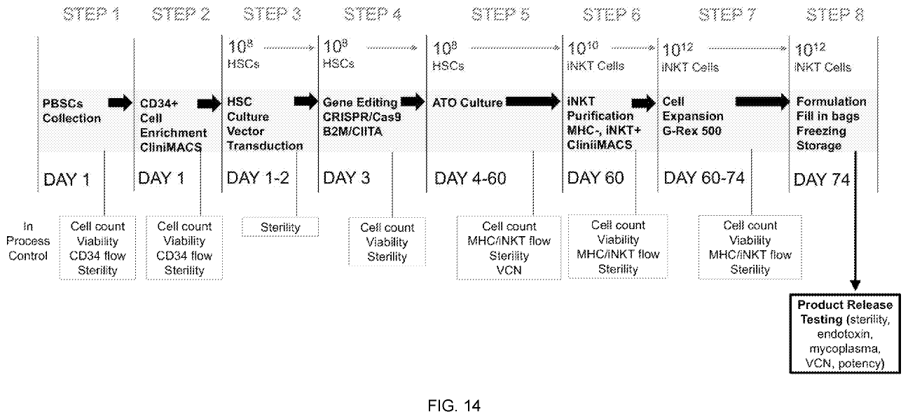

CMC overview In certain embodiments, the manufacturing of ^{U}HSC-iNKT involves: 1) collection of G-CSF-mobilized leukopak; 2) purification of GCSF-leukopak into CD34⁺ HSCs; 3) transduction of HSCs with lentiviral vector Lenti/iNKT-sr39TK; 4) gene editing of B2M and CIITA via CRISPR/Cas9; 5) in vitro differentiation into iNKT cells via ATO; 6) purification of iNKT cells; 7) in vitro cell expansion; 8) cell collection, formulation and cryopreservation (FIG. 14). As examples, there are two drug substances (Lenti/iNKT-sr39TK vector and ^{U}HSC-iNKT cells), and the final drug product is the formulated and cryopreserved ^{U}HSC-iNKT in infusion bags, in at least some cases.

1. Vector Manufacturing

Vector structure One vector for genetic engineering of HSCs into iNKT cells is an HIV-1 derived lentiviral vector Lenti/iNKT-sr39TK encoding a human iNKT TCR gene along with an sr39TK PET imaging/suicide gene (FIG. 13). The key components of this third generation self-inactivating (SIN) vector are: 1) 3′ self-inactivating long-term repeats (ΔLTR); 2) Ψ region vector genome packaging signal; 3) Rev Responsive Element (RRE) to enhance nuclear export of unspliced vector RNA; 4) central PolyPurine Tract (cPPT) to facilitate unclear import of vector genomes; 5) expression cassette of the α chain gene (TCRα) and β chain gene (TCRβ) of a human iNKT TCR, as well as the PET/suicide gene sr39TK (Gschweng et al., 2014) driven by internal promoter from the murine stem cell virus (MSCV). The iNKT TCRα and TCRβ and sr39TK genes are all codon-optimized and linked by 2A self-cleaving sequences (T2A and P2A) to achieve their optimal co-expression (Gschweng et al., 2014).

Quality control of vector A series of QC assays may be performed to ensure that the vector product is of high quality. Those standard assays such as vector identity, vector physical titer, and vector purity (sterility, mycoplasma, viral contaminants, replication-competent lentivirus (RCL) testing, endotoxin, residual DNA and benzonase) is conducted at IU VPF and provided in the Certificate of Analysis (COA). Additional QC assays one can perform include 1) the transduction/biological titer (by transducing HT29 cells with serial dilutions and performing ddPCR, ≥1×10⁶ TU/ml); 2) the vector provirus integrity (by sequencing the vector-integrated portion of genomic DNA of transduced HT29 cells, same to original vector plasmid sequence); 3) the vector function. The vector function maybe measured by transducing human PBMC T cells (Chodon et al., 2014). The expression of iNKT TCR gene may be detected by staining with the 6B11 specific for iNKT TCR (Montoya et al., 2007). The functionality of expressed iNKT TCRs may be analyzed by IFN-γ production in response to αGalCer stimulation (Watarai et al., 2008). The expression and functionality of sr39TK gene may be analyzed by penciclovir update assay and GCV killing assay (Gschweng et al., 2014). The stability of the vector stock (stored in −80 freezer) may be tested every 3 months by measuring its transduction titer. These QC assays may be validated.

2. Cell Manufacturing and Product Formulation

Overview of manufacturing ^{U}HSC-iNKT cells ^{U}HSC-iNKT cells are one embodiment of a drug substance that will function as “living drug” to target and fight tumor cells. They are generated by in vitro differentiation and expansion of genetically modified donor HSCs. Initial data demonstrate a novel and efficient protocol to produce them in a laboratory scale. In order to make them as an “off-the-shelf” cell product, one can develop and validate a GMP-comparable manufacturing process. As an example, target of production scale is 10¹² cells per batch, which is estimated to treat 1000-10,000 patients.

Cell manufacturing process One embodiment of a cell manufacturing process is outlined in FIG. 13, with defined timelines and key “In-Process-Control (IPC)” measurements for each process step. Step 1 is to harvest donor G-CSF-mobilized PBSCs in blood collection facilities, which has become a routine procedure in many hospitals (Deotare et al., 2015). One can obtain fresh PBSCs in Leukopaks from the HemaCare for this project; HemaCare has IRB-approved collection protocols and donor consents and can support clinical trials and commercial product manufacturing (A Support Letter from Hemacare is included in the Application). Step 2 is to enrich CD34⁺ HSCs from PBSCs using a CliniMACS system; one can use such a system located at the UCLA GMP facility to complete this step and expect to yield at least 10⁸ CD34⁺ cells. CD34⁻ cells are collected and stored as well (may be used as PBMC feeder in Step 7).

Step 3 involves the HSC culture and vector transduction. CD34⁺ cells are cultured in X-VIVO15 medium supplemented with 1% HAS (USP) and growth factor cocktails (c-kit ligand, fit-3 ligand and tpo; 50 ng/ml each) for 12 hrs in flasks coated with retronectin, followed by addition of the Lenti/iNKT-sr39TK vector for additional 8 hrs (Gschweng et al., 2014). Vector integration copies (VCN) are measured by sampling ˜50 colonies formed in the methylcellulose assay for transduced cells and one can determine the average vector copy number per cell using ddPCR (Nolta et al., 1994). One can routinely achieved >50% transduction with VCN=1-3 per cell, in at least some cases.

Step 4 is to utilize the powerful CRISPR/Cas9 multiplex gene editing method to target the genomic loci of both B2M and CIITA in HSCs and disrupt their gene expression (Ren et al., 2017; Liu et al., 2017), and iNKT cells derived from edited HSCs will lack the MHC/HLA expression, thereby avoiding the rejection by the host immune system. Initial data has demonstrated the success of the B2M disruption for CD34⁺ HSCs with high efficiency (˜75% by flow analysis) via electroporation of Cas9/B2M-gRNA. B2M/CIITA double knockout may be achieved by electroporation of a mixture of RNPs (Cas9/B2M-gRNA and Cas9/CIITA-gRNA (Abrahimi et al., 2015)). One can optimize and validate this process (Gundry et al., 2016) by varying electroporation parameters, ratios of two RNPs, stem cell culture time (24, 48, or 72 hrs post-transduction) prior to electroporation, etc; one can use the high fidelity Cas9 protein (Slaymaker et al., 2016; Tsai and Joung, 2016) from IDT to minimize the “off-target” effect. Evaluation parameters may be viability, deletion (indel) frequency (on-target efficiency) measured by a T7E1 assay and next-generation sequencing (NGS) targeting the B2M and CIITA sites, MHC expression by flow cytometry, and hematopoietic function of edited HSCs measured by the colony formation unit (CFU) assay, for example.

Step 5 is to in vitro differentiate modified CD34⁺ HSCs into iNKT cells via the artificial thymic organoid (ATO) culture (Seet et al., 2017). Initial studies have shown that functional iNKT cells can be efficiently generated from HSCs engineered to express iNKT TCRs. Building upon this data, one can test and validate an 8-week, GMP-compatible ATO culture process to produce 10¹⁰ iNKT cells from 10⁸ modified CD34⁺ HSCs. ATO involves pipetting a cell slurry (5 μl) containing mixture of HSCs (5×10⁴) and irradiated (80 Gy) MS5-hDLL1 stromal cells (10⁶) as a drop format onto a 0.4-μm Millicell transwell insert, followed by placing the insert into a 6-well plate containing 1 ml RB27 medium (Seet et al., 2017); medium can be changed every 4 days for 8 weeks. Considering 3 ATOs per insert, one may need approximately 170 six-well plates for each batch production. An automated programmable pipetting/dispensing system (epMontion 5070f from Eppendorf) placed in biosafety cabinet for plating ATO droplets and medium exchange may be used; a 2-hr operation may be needed for completing 170 plates each round. At the end of ATO culture, iNKT cells are harvested and characterized. As one example, a component of ATO is the MS5-hDLL1 stromal cell line that is constructed by lentiviral transduction to express human DLL1 followed by cell sorting. In preparation for one embodiment of the GMP process, one can perform a single cell clonal selection process on this polyclonal cell population to establish several clonal MS5-hDLL1 cell lines, from which one can choose an efficient one (evaluated by ATO culture) and use it to generate a master cell bank. Once certified, this bank may be used to supply irradiated stromal cells for future clinical grade ATO culture.

Step 6 is to purify ATO-derived iNKT cells using the CliniMACS system. This step purification is to deplete MHCI⁺ and MHCII⁺ cells and enrich iNKT⁺ cells. Anti-MHCI and anti-MHCII beads may be prepared by incubating Miltenyi anti-Biotin beads with commercially available biotinylated anti-B2M (clone 2M2), anti-MHCI (clone W6/32, HLA-A, B, C), anti-MHCII (clone Tu39, HLA-DR, DP, DQ), and anti-TCR Vα24-Jα18 (clone 6B11) antibodies; microbeads directly coated with 6B11 antibodies are also are available from Miltenyi Biotec. Harvested iNKT cells are labeled by anti-MHC bead mixtures and washed twice and MHCI⁺ and/or MHCII⁺ cells are depleted using the CliniMACS depletion program; if necessary, this depletion step can be repeated to further remove residual MHC⁺ cells. Subsequently, iNKT cells are further purified using the standard anti-iNKT beads and the CliniMACS enrichment program. The cell purity may be measured by flow cytometry.

Step 7 is to expand purified iNKT cells in vitro. Starting from 10¹⁰ cells, one can expand into 10¹² iNKT cells using an already validated PBMC feeder-based in vitro expansion protocol (Yamasaki et al., 2011; Heczey et al., 2014). One can evaluate a G-Rex-based bioprocess for this cell expansion. G-Rex is a cell growth flask with a gas-permeable membrane at the bottom allowing more efficient gas exchange; A G-Rex500M flask has the capacity to support a 100-fold cell expansion in 10 days (Vera et al., 2010; Bajgain et al., 2014; Jin et al., 2012). The stored CD34⁻ cells (used as feeder cells) from the Step 1 are thawed, pulsed with αGalCer (100 ng/ml), and irradiated (40 Gy). iNKT cells will be mixed with irradiated feeder cells (1:4 ratio), seeded into G-Rex flasks (1.25×10⁸ iNKT each, 80 flasks), and allowed to expand for 2 weeks. IL-2 (200 U/ml) will be added every 2-3 days and one medium exchange will occur at day 7; all medium manipulation may be achieved by peristaltic pumps. This expansion process should be GMP-compatible because a similar PBMC feeder-based expansion procedure (termed rapid expansion protocol) has been already utilized to produce therapeutic T cells for many clinical trials Dudley et al., 2008; Rosenberg et al., 2008).

Step 8 is to formulate the harvested iNKT cells from Step 7 (the active drug component) into cell suspension for direct infusion. After at least 3 rounds of extensive washing, cells from Step 7 may be counted and suspended into an infusion/cold storage-compatible solution (10⁷-10⁸ cells/ml), which is composed of Plasma-Lyte A Injection (31.25% v/v), Dextrose and Sodium Chloride Injection (31.25% v/v), Human Albumin (20% v/v), Dextran 40 in Dextrose Inject (10%, v/v) and Cryoserv DMSO (7.5%, v/v); this solution has been used to formulate tisagenlecleucel, an approved T cell product from Novartis (Grupp et al., 2013). Once filled into FDA-approved freezing bags (such as CryoMACS freezing bags from Miltenyi Biotec), the product may be frozen in a controlled rate freezer and stored in a liquid nitrogen freezer. One can perform validation and/or optimization studies by measuring viability and recovery to ensure that this formulation is appropriate for the ^{U}HSC-iNKT cell product.

Quality control for bioprocessing and product Various IPC assays such as cell counting, viability, sterility, mycoplasma, identity, purity, VCN, etc.) may be incorporated into the proposed bioprocess to ensure a high-quality production. The proposed product releasing testing include 1) appearance (color, opacity); 2) cell viability and count; 3) identity and VCN by qPCR for iNKT TCR; 4) purity by iNKT positivity and B2M negativity; 5) endotoxins; 6) sterility; 7) mycoplasma; 8) potency measured by IFN-γ release in response to αGalCer stimulation; 9) RCL (replication-competent lentivirus) (Cornetta et al., 2011). Most of these assays are either standard biological assays or specific assays unique to this product that may be validated. Product stability testing may be performed by periodically thawing LN-stored bags and measuring their cell viability, purity, recovery, potency (IFN-γ release) and sterility. In particular embodiments, the product is stable for at least one year.

3. Safety Embodiments

Tumorigenecity in vitro and in vivo and acute toxicity in vivo One can evaluate the potential of ^{U}HSC-iNKT cells for transformation or autonomous proliferation. The in vitro assays include 1) G-banded karyotyping, which may be conducted on αGalCer-restimuated, actively dividing ^{U}HSC-iNKT cells to determine whether a normal karyotype is maintained; 2) homeostatic proliferation (without stimulation) of the cell product, which may be measured by flow cytometric analysis of the dilution of cell-labeled PKH dyes (the αGalCer-stimulated cell group will be used as a proliferation-positive control)(Hurton et al., 2016); 3) the soft agar colony formation assay (Horibata et al., 2015), which may be employed to evaluate the anchorage-independent growth capacity of the iNKT cell product. NSG naïve mice infused with 10⁷ iNKT cells may be used to examine the in vivo tumorigenecity and long-term toxicity (4-6 months, n=6) by analyzing various harvested tissues/organs for any abnormality and by measuring the presence of iNKT cells in blood, spleen, bone marrow and liver for any aberrant proliferation (Hurton et al., 2016); the control group may be mice transferred with PBMC-purified iNKT cells. The pilot in vivo acute toxicity may be carried out by infusing naïve NSG mice with a low (10⁶) or a high (10⁷) dose iNKT cells. Mice (n=8) may then be observed 2 weeks for any alterations in body weight and food consumption, as well as any abnormal behaviors. After 2 weeks, mice may be euthanized and blood may be collected for blood hematology and blood serum chemistry analysis (UCSD murine hematology and coagulation core lab); various mouse tissues may be harvested and submitted to UCLA core for pathological analysis.

Allogeneic transplant-associated safety testing in vitro and in vivo The ^{U}HSC-iNKT therapy is of allogeneic transplant nature and thus its related safety may be evaluated. The potential of allogeneic reaction may be first determined by a standard two-way in vitro mixed lymphocyte reactions (MLR) assay (Bromelow et al., 2001). ^{U}HSC-iNKT cells may be mixed with mismatched donor PBMCs (at least three different donor batches) and T cell proliferation may be measured by the BrdU incorporation assay. For the study of GvHD, ^{U}HSC-iNKT may be the responder cells and PBMCs may be the stimulator cells; a reverse setting may be used to investigate HvG reactivity; stimulator cells will be irradiated prior to the incubation. One can also exploit an in vivo NSG mouse model to assess the in vivo GvHD and HvG reaction. Mice may be infused with ^{U}HSC-iNKT (5×10⁶, Group 1), human PBMCs (5×10⁶, Group 2), or combination (5×10⁶ each, Group 3). Mice may be observed for 2 months for any signs of toxicity (weight loss, behaviors, etc.). Mononuclear cells from bi-weekly mouse bleeding may be analyzed for human T cell activation markers (upregulation of hCD69 and hCD44, downregulation of hCD62L); ^{U}HSC-iNKT, human PBMC-derived CD8⁺ T, and human PBMC-derived CD4⁺ T cells may be identified by hCD45⁺6B11⁺, hCD45⁺6B11⁻TCRαβ⁺CD8⁺, and hCD45⁺6B11⁻TCRαβ⁺CD4⁺, respectively. Compared to Groups 1 and 2, lack of activation of iNKT cells and lack of depletion of PBMCs in the Group 3 mice may indicate the lack of GvHD reactions, whereas lack of the activation of PBMC CD8/CD4 T cells and lack of depletion of ^{U}HSC-iNKT cells in the Group 3 mice may indicate the lack of HvG reactions.

Lentiviral vector safety and gene editing-related off-target analysis As a product releasing testing, the RCL assay may be measured to ensure patients not to be inadvertently exposed to replicating virus. One can also extract the genomic DNA from ^{U}HSC-iNKT cells and submit it for lentivirus integration site sequencing (Applied Biological Materials Inc.) to detect any unusual integrations other than the known lentiviral integration patterns. To analyze the gene editing-related off-target effect, one can use the CRISPR design tool from MIT to predict potential off-target sites and assess/confirm them by targeted re-sequencing of the genomic DNA of ^{U}HSC-iNKT cells. Additionally, one can perform unbiased genome-wide scans for off-target sites using GUILDE-seq in K562 cells electroporated with the Cas9/B2M-gRNA and Cas9/CIITA-gRNA RNPs and a dsODN tag (Tsai et al., 2015); these off-target sites may then be analyzed by NGS in ^{U}HSC-iNKT cells to detect the frequencies of off-target activity.

### Example 2: A Hematopoietic Stem Cell (HSC) Approach to Engineer Off-the-Shelf INKT Cells

Multiple myeloma (MM) is a malignant monoclonal plasma cell disorder characterized by osteolytic bone lesions, anemia, hypercalcemia, and renal failure. It is the second most common hematological malignancy, affecting millions of people worldwide. Although novel agents such as proteasome inhibitors, immunomodulatory drugs, and autologous hematopoietic stem cell transplantation have improved the treatment, MM remains an incurable disease with a high relapse rate. In 2019 alone, it is estimated that over 3000 Californians will be diagnosed with MM and more than 1320 Californians will die from this disease. Therefore, novel therapies with curative potential are urgently desired in order to address this unmet medical need. Autologous transfer of chimeric antigen receptor-engineered T cells (CAR-T) targeting B-cell maturation antigen (BCMA) has shown impressive clinical responses for treating relapsed/refractory MM in ongoing clinical trials and is expected to get regulatory approval in 2020 as a fourth-line treatment for MM. However, such a treatment procedure requires the collection and manufacturing of T cells from each individual patient, making this type of autologous therapy costly, labor intensive, and difficult to broadly deliver to all MM patients in need. Allogeneic cell therapies that can be manufactured at large scale and distributed readily to treat a broad base of MM patients therefore are in great demand.

Invariant natural killer T (iNKT) cells are a small subpopulation of αβ T lymphocytes. These immune cells have several unique features that make them ideal cellular carriers for developing off-the-shelf cellular therapy for cancer: 1) they have roles in cancer immunosurveillance; 2) they have the remarkable capacity to target tumors independent of tumor antigen- and major histocompatibility complex (MHC)-restrictions; 3) they can deploy multiple mechanisms to attack tumor cells through direct killing and adjuvant effects; 4) and most attractively, they do not cause graft-versus-host disease (GvHD). However, the development of an allogeneic off-the-shelf iNKT cellular product is greatly hindered by their availability—these cells are of extremely low number and high variability in humans (˜0.001-1% in human blood), making it very difficult to produce therapeutic numbers of iNKT cells from blood cells of allogeneic human donors. A novel method that can reliably generate a homogenous population of iNKT cells at large quantities is thus pivotal to developing an off-the-shelf iNKT cell therapy.

To overcome the critical limitation of iNKT cell numbers, the inventors have previously developed a powerful method to generate iNKT cells from hematopoietic stem cells (HSCs) through iNKT T cell receptor (TCR) gene engineering. This innovative technology allowed the inventors to develop an autologous gene-engineered HSC adoptive therapy for cancer. Recently, researchers another technology breakthrough on establishing an Artificial Thymic Organoid (ATO) culture system that supports the in vitro differentiation of human HSCs into T cells at high efficiency and high yield. The inventors demonstrated that the ATO in vitro culture system can be used to produce human HSC-engineered iNKT (HSC-iNKT) cells which can be further engineered into BCMA CAR-iNKT cells with a remarkable yield: from a single random healthy donor, the inventors can harvest G-CSF-mobilized CD34⁺ HSCs and utilize these HSCs to produce 10¹² scale of homogenous BCMA CAR-iNKT cells of potent tumor killing capacity, which can potentially be formulated into 1,000-10,000 doses of therapeutic cellular product.

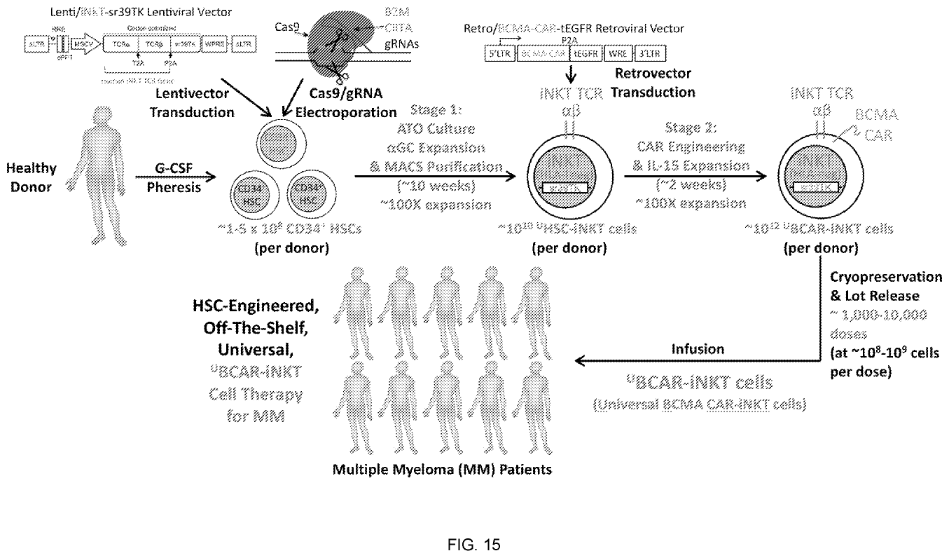

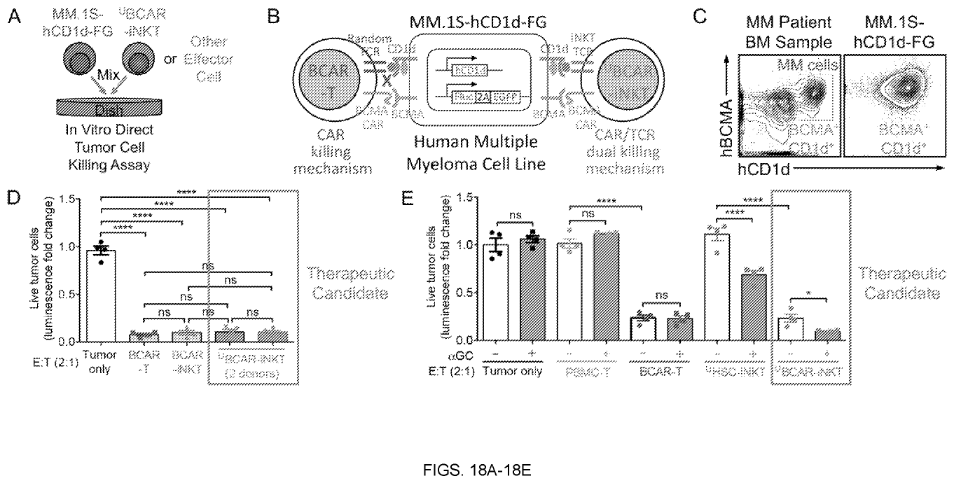

Efficacy of the therapeutic candidate. In this example, the inventors propose the HSC-Engineered Universal BCMA CAR-iNKT (^{U}BCAR-iNKT) cells as a therapeutic candidate (FIG. 15). With the incorporation of chimeric antigen receptor (CAR) targeting B-cell maturation antigen (BCMA), studies demonstrate potent and direct killing of MM tumor cells in vitro (FIG. 18) and complete eradication of tumor cells in vivo in a preclinical animal model (FIG. 19). The inventors also observed the synergistic effect of both BCMA CAR- and iNKT TCR-mediated killing of MM cells (FIG. 18E). The data indicate that the ^{U}BCAR-iNKT product 1) is at least as potent as conventional BCMA CAR-T cells; 2) can deploy multiple mechanisms to target tumors, thereby mitigating tumor antigen escape; 3) have a strong safety profile (no GvHD), and 4) can be reliably manufactured with high yield. Thus, this allogeneic ^{U}BCAR-iNKT cell product may be useful for treating MM.

Status of stromal cell line MS5-hDLL1 for manufacturing. The inventors have tested many cGMP-compliant conditions for this cell line. This cell line has already been authenticated with regard to species and strain of origin by STR analysis. Through Charles River Animal Diagnostic Service, the cell line has tested negative for mycoplasma and negative for infectious diseases by a Mouse Essential CLEAR panel. It has also tested negative for interspecies contamination for rat, Chinese hamster, Golden Syrian hamster, human, and non-human primate. These testing results are consistent with the FDA's statement regarding the xenogeneic feeder cells for GMP manufacturing.

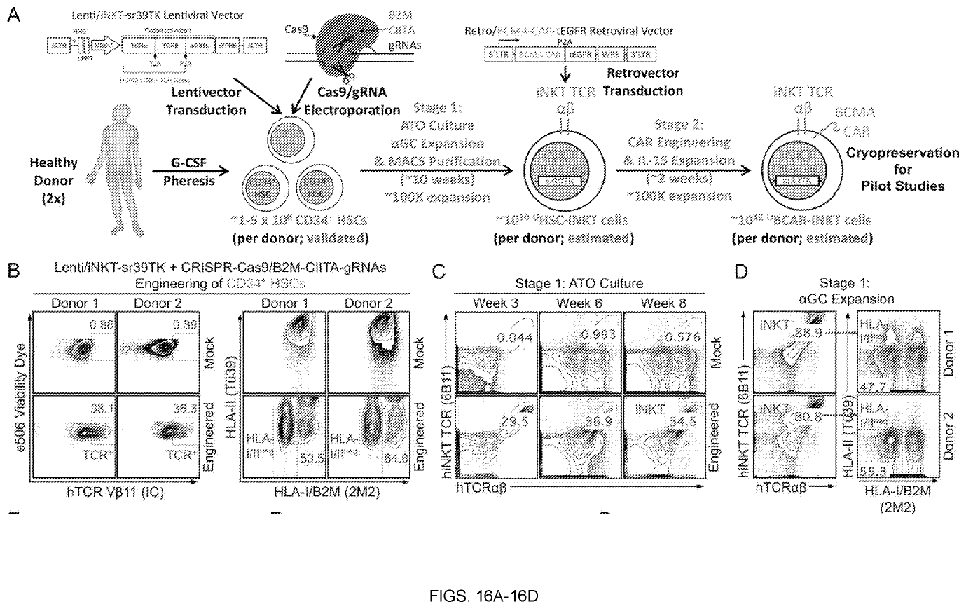

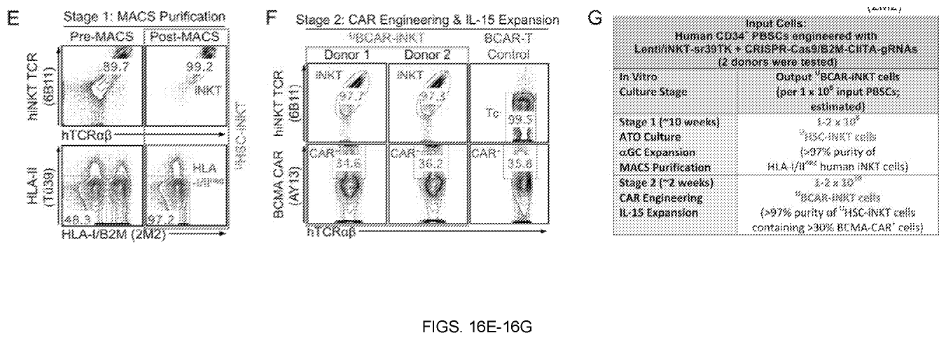

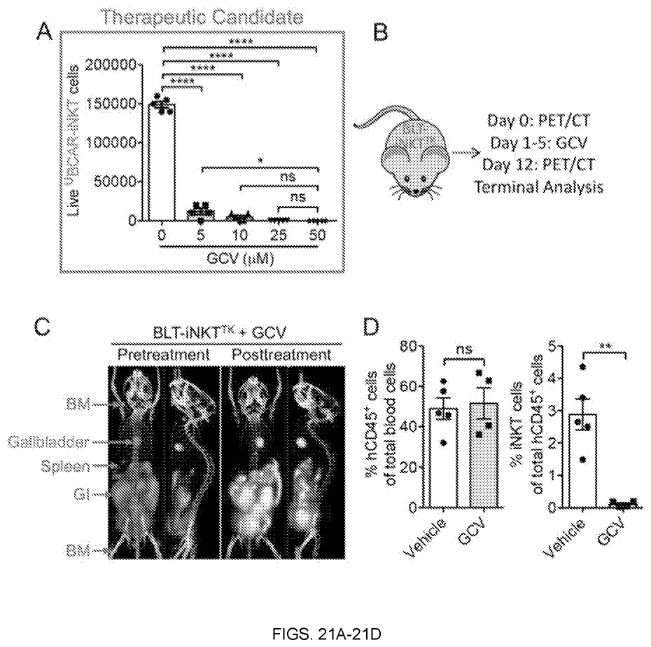

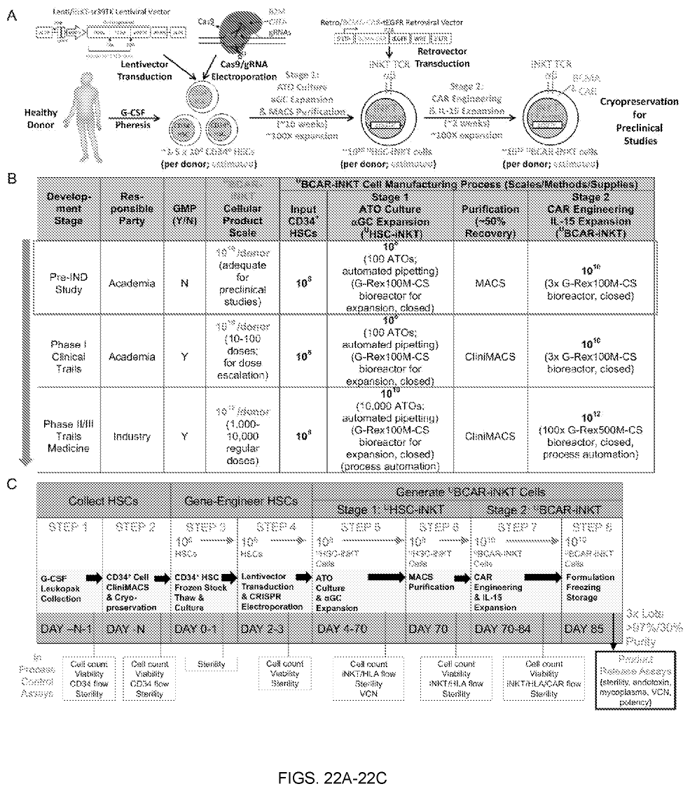

Manufacturing and process development. The inventors have tested G-Rex bioreactors for the expansion of iNKT and CAR-iNKT cells, and current data suggest that they are compatible for the process and could enhance both the yield of expansion and the quality of cells (FIG. 16). With the GatheRex Liquid Handling system, the G-Rex bioreactors can be operated as a closed system for cell manufacturing (FIG. 22). The inventors will also test the automated pipetting system (epMotion from Eppendorf) to simplify the ATO culture. Overall, it is contemplated that most process steps can be easily automated for commercial-scale production.

Biosafety evaluation of cytokine release syndrome (CRS) and neurotoxicity. Recent findings suggest that monocytes and macrophages are two major cell sources for eliciting these reactions and triple transgenic (human SCF, GM-CSF, and IL-3) NSG mice reconstituted with human CD34⁺ cells can model CRS and neurotoxicity induced by CAR-T treatment. The inventors will therefore propose to use this animal model to investigate these events in the setting of MM treated by ^{U}BCAR-iNKT cell therapy; the conventional BCMA CAR-T treatment will be included as a control. If these toxicities are observed, the inventors contemplate the use of combination therapy with tocilizumab (anti-IL-6R antibody) or anakinra (IL-1R antagonist) to ameliorate these side-effects.

A. Patient Populations

Group 1A: Adults with relapsed/refractory multiple myeloma (MM) who have received three or more prior treatments including a proteasome inhibitor (e.g., bortezomib or carfilzomib), an immunomodulatory agent (IMiD; e.g., lenalidomide or pomalidomide), and an anti-CD38 antibody, defined as disease progression within 60 days of the most recent regimen. More than 15% of patients' malignant plasma cells express B cell maturation antigen (BCMA).

Group 2A: Relapsed/refractory MM patients meeting the above criteria who have also failed prior autologous BCMA-targeted CAR-T cell therapy and whose malignant cells remain BCMA positive.

Group 1B: Adults with relapsed/refractory multiple myeloma (MM) who have received at least 3 prior lines of therapy including a proteasome inhibitor (e.g., bortezomib or carfilzomib), an immunomodulatory agent (IMiD; e.g., lenalidomide or pomalidomide), and an anti-CD38 antibody, defined as disease progression within 60 days of the most recent regimen. Expression of B cell maturation antigen (BCMA) is detectable on patients' malignant plasma cells.

Group 2B: Relapsed/refractory MM patients meeting the above criteria who have also failed prior autologous BCMA-directed CAR-T cell therapy.

B. Contemplated Biological Activity Outcomes

The optimal biological activity of the ^{U}BCAR-iNKT cell product is to achieve safe allogenic engraftment without causing GvHD and engrafting at sufficient levels and time durations to mediate potent anti-tumor immune responses and eliminate cancer cells.

Allogeneic ^{U}BCAR-iNKT cells do not express endogenous TCRs and do not cause GvHD.

Allogeneic ^{U}BCAR-iNKT cells do not express HLA-I/II and resist host CD8⁺ and CD4⁺ T cell-mediated allograft depletion and sr39TK immunogen-targeted depletion.

BCMA CAR expressed on allogeneic ^{U}BCAR-iNKT cells can exhibit potent functions to recognize and kill malignant plasma cells.

Expression of sr39TK gene in allogeneic ^{U}BCAR-iNKT cells allows for sensitive tracking of these genetically modified cells with PET imaging and elimination of these cells through the sr39TK suicide gene function in case of a safety need.

The minimally acceptable biological activity of the ^{U}BCAR-iNKT cell product is to achieve safe allogeneic engraftment without causing GvHD and engrafting at detectable levels and certain duration with measurable anti-tumor immune responses.

Allogeneic ^{U}BCAR-iNKT cells do not express alloreactive endogenous TCRs and do not cause GvHD.

Allogeneic ^{U}BCAR-iNKT cells do not express adequate HLA-I/II and resist host CD8⁺ and CD4⁺ T cell-mediated allograft depletion and sr39TK immunogen-targeted depletion.

BCMA CAR expressed on allogeneic ^{U}BCAR-iNKT cells can exhibit adequate functions to mediate the recognition and killing of malignant plasma cells.

Expression of sr39TK gene in allogeneic ^{U}BCAR-iNKT cells allows for measurable tracking of these genetically modified cells with PET imaging and elimination of these cells through the sr39TK suicide gene function in case of a safety need.

C. Contemplated Efficacy Outcomes

It is contemplated that the compositions of the disclosure can achieve one or more of the following outcomes: succeeded in manufacturing of final cell product that meets all release criteria for all healthy donors; from one healthy donor, produce a minimum of 1,000 doses of allogeneic ^{U}BCAR-iNKT cell product (10⁸-10⁹ cells per dose); efficient engraftment of allogeneic ^{U}BCAR-iNKT cells at therapeutic effective levels and time durations following lymphodepleting conditioning and infusion; clinical response rate similar to current autologous BCMA CAR-T cell therapy for Group 1 patients, namely ORR≥70% with ≥50% CR; median PFS 10 months. ORR≥30% observed for Group 2 patients; succeeded in manufacturing of final cell product that meets all release criteria for at least 50% of healthy donors; from one healthy donor, produce a minimum of 100 doses of allogeneic ^{U}BCAR-iNKT cell product (10⁸-10⁹ cells per dose); detectable engraftment of allogeneic ^{U}BCAR-iNKT cells following lymphodepleting conditioning and infusion; and clinical response rate observed with ORR≥30% for Group 1 patients. Objective responses observed for Group 2 patients.

D. Safety Embodiments

It is contemplated that the compositions of the disclosure can achieve one or more of the following outcomes: absence of any grade nonhematological SAEs related to the cell product (NCI CTCAE v4); absence of replication-competent lentivirus (RCL); absence of monoclonal expansion or lymphoproliferative disorder from vector insertional events; absence of GvHD; absence of higher than grade 2 cytokine release syndrome; absence of higher than grade 2 neurologic toxicity; all CRS and neurotoxicity events reversible; absence of grade 3-4 nonhematological SAEs related to the cell product (NCI CTCAE v4); absence of grade 3 or higher GvHD; absence of grade 4 or higher cytokine release syndrome; and absence of grade 4 or higher neurologic toxicity.

E. Dose/Regimen Embodiments

It is contemplated that the following dosing and regimen embodiments may be used in the methods of the disclosure

The dosing regimen is a single dose of allogeneic ^{U}BCAR-iNKT cells administered intravenously following lymphodepleting conditioning with fludarabine and cyclophosphamide. The dosing regimen may be redefined based on safety and efficacy data from the Phase I study.

Based on previous clinical experiences on autologous BCMA CAR-T cell therapy, the dose range is 10⁷-10⁹ cells per patient per injection. However, the dosing of the allogeneic ^{U}BCAR-iNKT cell product may differ from that of autologous cells.

An open-label phase I dose escalation study will be performed to determine the safety and clinical activity of the allogeneic ^{U}BCAR-iNKT cell product. This will enroll relapsed/refractory MM patients in three dosing cohorts (1×10⁸, 3×10⁸, and 6×10⁸ cells per patient) with 6 patients per cohort, following a 3+3 design. Within each cohort, patients will be assigned to receive one of two different lots of ^{U}BCAR-iNKT cell products. The primary outcome measure will be dose-limiting toxicity.

An open-label phase I dose escalation study will be performed to determine the safety and clinical activity of the allogeneic ^{U}BCAR-iNKT cell product. This will enroll relapsed/refractory MM patients in three dosing cohorts (1×10⁸, 3×10⁸, and 6×10⁸ cells per patient) with 3 patients per cohort, following a 3+3 design. Patients will receive cells from a single lot of ^{U}BCAR-iNKT cell product. The primary outcome measure will be dose-limiting toxicity.

Dose escalation stops at the lowest dose that shows efficacy.

The product, ^{U}BCAR-iNKT cells, should be formulated as a cell suspension in a single dose form and compatible with cryopreservation in 5% DMSO and 2.5% human albumin, and intravenous administration over less than one hour.

The formulated cell suspension should be stable at room temperature for 4 hours or more from time of thawing.

The formulated cell suspension should be stable at room temperature for 1 hour from time of thawing.

F. Value Proposition for the Proposed Stem Cell-Based Therapeutic Product

The treatment costs for a single cancer patient managed by standard treatments vary depending on the type/stage of the cancer and the medical care that the patient receives. The Agency for Healthcare Research and Quality (AHRQ) estimates that the direct medical costs (the total of all health care costs) for cancer in the US are projected to rise to $157.7 billion by 2020. Newly approved cancer drugs cost up to $30 k per month, according to the American Society of Clinical Oncology (ASCO).

Autologous gene-modified cellular therapy, like the newly FDA-approved Kymriah and Yescarta (CAR-T therapy), has a market price of ˜$300-500 k per patient per treatment. It is so costly because a personalized cellular product needs to be manufactured for each patient and can only be utilized to treat that single patient. An off-the-shelf product, like the ^{U}BCAR-iNKT cells proposed in this application, could greatly reduce cost. The cost of manufacturing one batch of ^{U}BCAR-iNKT cells may be higher than that of manufacturing one batch of autologous BCMA CAR-T cells, but it is unlikely to exceed a 10-fold increase. Even assuming a 10-fold higher manufacturing cost, the proposed off-the-shelf ^{U}BCAR-iNKT cell therapy will still only cost ˜$3-5 k per dose, making the therapy much more affordable.

Cell-Based Immunotherapy for MM—Autologous vs. Allogeneic Approaches: Autologous transfer of BCMA-targeted CAR-engineered T cells has shown remarkable efficacy for treating relapsed/refractory MM in ongoing clinical studies and will likely obtain regulatory approval as a fourth-line treatment for MM in 2020. However, such a protocol requires that source T cells collected from a patient will be manufactured and used to treat that single patient, making this type of autologous therapy costly, labor intensive, and difficult to efficiently deliver to all MM patients in need. Therefore, allogeneic cell therapy that can be manufactured on a large scale and distributed readily to treat a broad base of MM patients is in great demand.

G. Therapeutic Candidate Description: Allogeneic HSC-Engineered Off-the-Shelf Universal BCMA CAR-INKT (^{U}BCAR-INKT) Cells

The therapeutic candidate, ^{U}BCAR-iNKT cells, were used for all pilot studies; exempt for the in vivo efficacy and safety study, which was performed using a therapeutic surrogate, BCAR-iNKT cells; ^{U}BCAR-iNKT (HLA-I/II-negative) and BCAR-iNKT (HLA-I/II-positive) cells were generated following the same manufacturing process (+/− CRISPR), and displayed comparable iNKT phenotype and functionality; 3. Conventional BCMA CAR-T (BCAR-T) cells were generated using the same Retro/BCMA-CAR-tEGFR retrovector transduction approach, and were included as a control in all relevant pilot studies; 4. When applicable, pilot study data were presented as the mean±SEM. N numbers were indicated. Statistical analyses were performed using either the Student's t test or one-way ANOVA, as appropriate. ns, not significant; *P<0.05; **P<0.01; ***P<0.001; ****P<0.0001.)

H. Pilot CMC Study (FIG. 16)

G-CSF-mobilized human CD34⁺ HSCs were collected from two different healthy donors (˜3-5×10⁸ HSCs per donor), transduced with a Lenti/iNKT-sr39TK vector and electroporated with a CRISPR-Cas9/B2M-CIITA-gRNAs complex, followed by culturing in vitro in a 2-Stage culture system FIG. 16A). CRISPR-Cas9/B2M-CIITA-gRNAs complex (Cas9 from the UC Berkeley MacroLab Facility; gRNAs from Synthego; B2M-gRNA sequence 5′-CGCGAGCACAGCUAAGGCCA-3′ (SEQ ID NO:68); CIITA-gRNA sequence 5′-GAUAUUGGCAUAAGCCUCCC-3—SEQ ID NO:69′) was utilized to disrupt the B2M and CIITA genes in human HSCs to generate HLA-I/II-negative iNKT cells (FIG. 16A, upper middle). Co-engineering of HSCs with Lenti/iNKT-sr39TK and CRISPR-Cas9/B2M-CIITA-gRNAs was highly efficient, resulting in ˜30-40% TCR gene delivery rate and ˜50-70% HLA-I/II double-deficiency rate (FIG. 16B). In Stage 1 culture, gene-engineered HSCs were efficiently differentiated into human iNKT cells in the Artificial Thymic Organoid (ATO) culture over a period of 3-8 weeks with peak production at week 8 (FIG. 16C). At week 8, ATO iNKT cells were collected and expanded with αGC-loaded irradiated PBMCs (as antigen presenting cells) for 2 weeks, followed by isolating HLA-I/II-negative universal HSC-engineered human iNKT cells (denoted as ^{U}HSC-iNKT cells) through a 2-Step MACS purification strategy: 1) a MACS negative selection step selecting against surface HLA-I/B2M (by 2M2 mAb recognizing B2M) and HLA-II (by Tü39 mAb recognizing HLA-DR, DP, DQ) molecules and 2) a MACS positive selection step selecting for surface iNKT TCR molecules (by 6B11 mAb recognizing human iNKT TCR) (FIG. 16E). Post-MACS purification, the Stage 1 culture yielded a highly homogenous HLA-I/II-Negative Universal HSC-Engineered iNKT (^{U}HSC-iNKT) cellular product of over 97% purity (>99% iNKT cells, of which >97% are HLA-I/II-negative), that expanded ˜100-fold compared to the input HSCs (FIG. 16E). In Stage 2 culture, ^{U}HSC-iNKT cells were further engineered by transducing them with a Retro/BCMA-CAR-tEGFR retroviral vector followed by IL-15 expansion for 2 weeks, leading to BCMA-CAR expression in ^{U}HSC-iNKT cells and another ˜100-fold expansion of the engineered cells (FIG. 16A, upper right). The Retro/BCMA-CAR-tEGFR retroviral vector has been successfully utilized to manufacture autologous BCMA CAR-T for ongoing Phase I clinical trials treating MM. In the experiments, the inventors routinely obtained >30% BCMA-CAR engineering rate of ^{U}HSC-iNKT cells, comparable to engineering peripheral blood T cells (FIG. 16F). This manufacturing process was robust and of high yield and high purity for both donors tested. Based on these results, it was estimated that from 1×10⁶ input HSCs, about 1-2×10¹⁰ HLA-I/II-negative universal BCMA CAR-engineered iNKT (^{U}BCAR-iNKT) cells could be produced, giving a theoretical yield of over 10¹² therapeutic candidate ^{U}BCAR-iNKT cells from a single healthy donor (FIG. 16G).

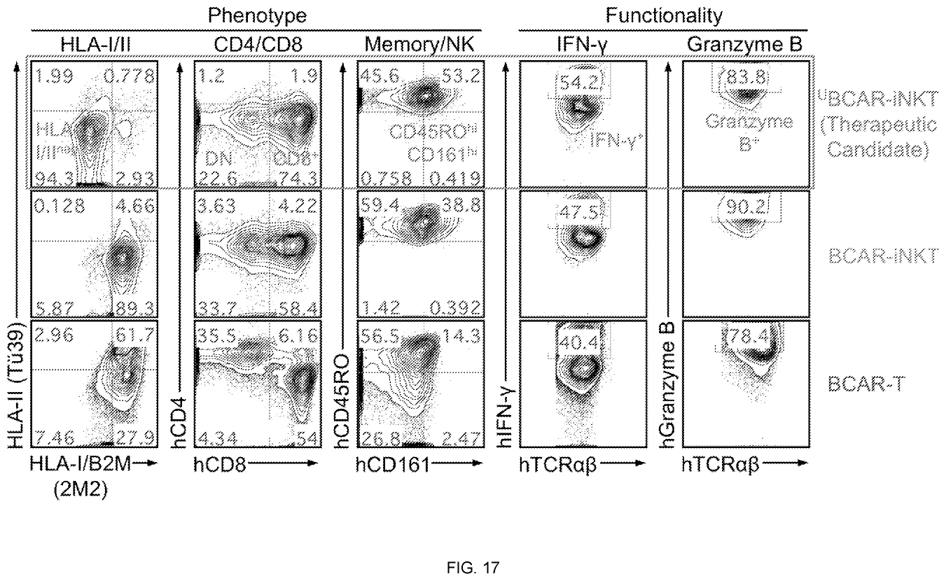

I. Pilot Pharmacology Study (FIG. 17)

The phenotype and functionality of ^{U}BCAR-iNKT cells (FIG. 16F) were studied using flow cytometry. Two controls were included: 1) BCAR-iNKT cells that were manufactured in parallel with ^{U}BCAR-iNKT cells but without the CRISPR-Cas9/B2M-CIITA-gRNA engineering step, and 2) BCAR-T cells, that were generated by transducing healthy donor peripheral blood T cells with the Retro/BCMA-CAR retroviral vector (FIG. 16F). As expected, control BCAR-T cells expressed high levels of HLA-I and HLA-II molecules, while ^{U}BCAR-iNKT cells were double-negative, confirming their suitability for allogeneic therapy (FIG. 17, left panels). Interestingly, even without CRISPR engineering, BCAR-iNKT cells already expressed low levels of HLA-II molecules, suggesting that these cells are naturally of low immunogenicity compared to conventional T cells (FIG. 17, left panels). Nonetheless, HLA-II expression could be further reduced by CRISPR engineering (in ^{U}BCAR-iNKT cells). Both ^{U}BCAR-iNKT and BCAR-iNKT cells displayed typical iNKT cell phenotype and functionality: they expressed the CD4 and CD8 co-receptors with a mixed pattern (CD4/CD8 double-negative and CD8 single-positive); they expressed high levels of memory T cell marker CD45RO and NK cell marker CD161; and they produced high levels of effector cytokines like IFN-γ and cytotoxic molecules like perforin and granzyme B comparable to or better than their counterpart conventional BCAR-T cells development or phenotype/functionality of the therapeutic candidate ^{U}BCAR-iNKT cells, making the manufacturing of this off-the-shelf cellular product possible.

J. Pilot In Vitro Efficacy and MOA Study (FIG. 18)

The inventors established an in vitro MM tumor cell killing assay for this study (FIG. 18A). A human MM cell line, MM.1S, was engineered to overexpress the human CD1d gene as well as a firefly luciferase (Fluc) reporter gene and an enhanced green fluorescent protein (EGFP) reporter gene, resulting in an MM.1S-hCD1d-FG cell line that was used for this assay (FIG. 18B). Of note, a large portion of primary MM tumor cells express both BCMA and CD1d, making these cells subject to both BCMA-CAR- and iNKT-TCR-mediated targeting (FIGS. 18B & 18C). Although the parental MM.1S cells express BCMA, they have lost CD1d expression like most existing MM cell lines; therefore, the inventors engineered MM.1S cells to express CD1d mimicking primary MM tumor cells (FIGS. 18B & 18C). ^{U}BCAR-iNKT cells effectively killed MM tumor cells, at an efficacy comparable to that of BCAR-iNKT and conventional BCAR-T cells, for two different CD34⁺ HSC donors (FIG. 18D). Importantly, in the presence of a cognate lipid antigen (αGC), ^{U}BCAR-iNKT cells, but not conventional BCAR-T cells, demonstrated enhanced tumor-killing efficacy, likely because ^{U}BCAR-iNKT cells could deploy a CAR/TCR dual tumor killing mechanism (FIGS. 18B & 18E). This unique CAR/TCR-mediated dual targeting capacity of ^{U}BCAR-iNKT cells is attractive, because it can potentially circumvent antigen escape, a phenomenon that has been reported in autologous BCMA CAR-T therapy clinical trials wherein MM tumor cells down-regulated their expression of BCMA antigen to escape attack from CAR-T cells.

K. Pilot In Vivo Efficacy and Safety Study (FIG. 19)

An NSG (NOD/SCID/γc^{-/-}) mouse MM.1S-hCD1d-FG tumor xenograft model was used for this study (FIG. 19A). BCAR-iNKT cells were studied as a therapeutic surrogate, and based on the in vitro characterization (phenotype/function/efficacy), were expected to resemble ^{U}BCAR-iNKT cells regarding in vivo efficacy and safety; conventional BCAR-T cells were included as a control. Both BCAR-iNKT and BCAR-T cells effectively eradicated pre-established metastatic MM tumor cells (FIGS. 19B & 19C). However, mice receiving the conventional BCAR-T cells, despite being tumor-free, eventually died of graft-versus-host disease (GvHD) (FIGS. 19D & 19E). On the contrary, mice receiving BCAR-iNKT cells remained tumor-free and survived long-term without GvHD (FIGS. 19D & 19E). These results validated the therapeutic potential of BCAR-iNKT therapy and highlighted the remarkable safety profile of the proposed off-the-shelf cellular therapy.

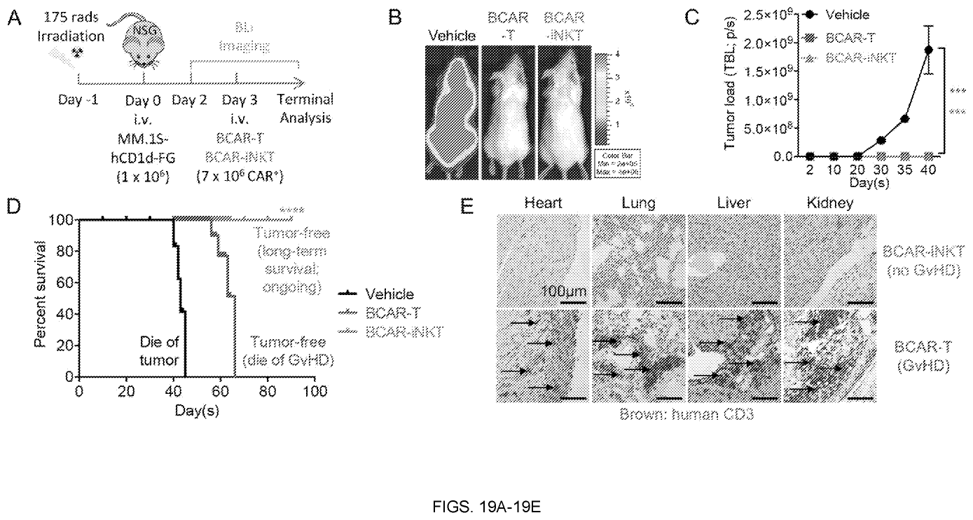

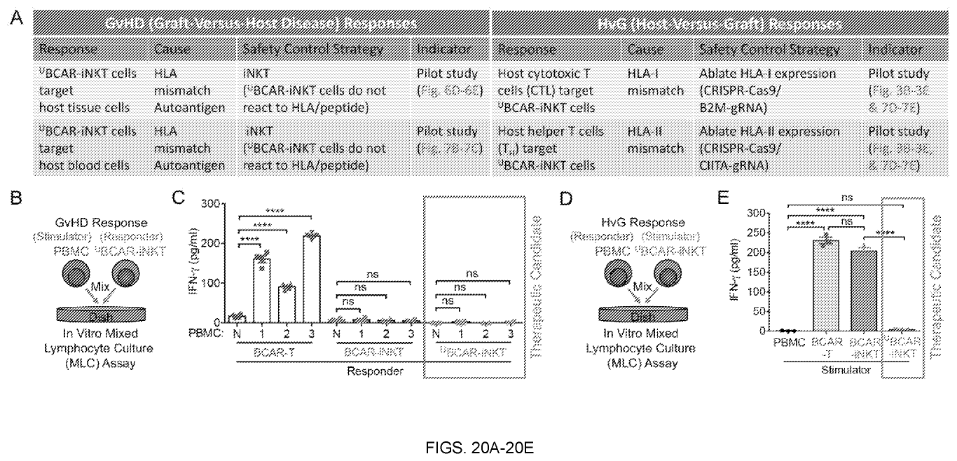

L. Pilot Immunogenicity Study (FIG. 20)

For allogeneic cell therapies, there are two immunogenicity concerns: a) GvHD responses, and b) host-versus-graft (HvG) responses. The inventors have considered the possible GvHD and HvG risks for the proposed ^{U}BCAR-iNKT cellular product, and evaluated the engineered mitigation and safety control strategies (FIG. 20A). GvHD is the major safety concern. However, because iNKT cells do not react to mismatched HLA molecules and protein autoantigens, they are not expected to induce GvHD. This notion is evidenced by the lack of GvHD in human clinical experiences in allogeneic HSC transfer and autologous iNKT transfer, and is supported by the pilot in vivo safety study (FIGS. 19D & 19E) and in vitro mixed lymphocyte culture (MLC) assay (FIGS. 20B & 20C). On the other hand, HvG risk is largely an efficacy concern, mediated through elimination of allogeneic therapeutic cells by host immune cells, mainly by conventional CD8 and CD4 T cells which recognize mismatched HLA-I and HLA-II molecules. ^{U}BCAR-iNKT cells are engineered with CRISPR to ablate their surface display of HLA-I/II molecules and therefore are expected not to induce host T cell-mediated responses (FIG. 17 and FIG. 20A). Indeed, in an In Vitro MLC assay, in sharp contrast to the conventional BCAR-T cells and the HLA-I/II-positive BCAR-iNKT cells, ^{U}BCAR-iNKT cells triggered no responses from PBMC T cells from multiple mismatched donors (FIGS. 20D & 20E). These results strongly support ^{U}BCAR-iNKT cells as an ideal candidate for off-the-shelf cellular therapy that are GvHD-free and HvG-resistant.

M. Pilot Safety Study—SR39TK Gene for Pet Imaging and Safety Control (FIG. 21)

To further enhance the safety profile of ^{U}BCAR-iNKT cell product, the inventors have engineered an sr39TK PET imaging/suicide gene in ^{U}BCAR-iNKT cells, which allows for the in vivo monitoring of these cells using PET imaging and the elimination of these cells through GCV-induced depletion in case of a serious adverse event (FIG. 16A). In cell culture, GCV induced effective killing of ^{U}BCAR-iNKT cells (FIG. 21A). A pilot in vivo study was performed using BLT-iNKT^{TK} humanized mice harboring human HSC-engineered iNKT (HSC-iNKT^{BLT}) cells (FIG. 2A-2B & FIG. 21B). The HSC-iNKT^{BLT} cells were engineered from human HSCs transduced with a Lenti/iNKT-sr39TK lentiviral vector, the same vector used for engineering the ^{U}BCAR-iNKT cellular product in this proposal (FIG. 15 & FIG. 2A). Using PET imaging combined with CT scan, the inventors detected the distribution of gene-engineered human cells across the lymphoid tissues of BLT-iNKT^{TK} mice, particularly in bone marrow (BM) and spleen (FIG. 21C). Treating BLT-iNKT^{TK} mice with GCV effectively depleted gene-engineered human cells across the body (FIG. 21C). Importantly, the GCV-induced depletion was specific, as evidenced by the selective depletion of the HSC-engineered human iNKT cells but not other human immune cells in BLT-iNKT^{TK} mice as measured by flow cytometry (FIG. 21D). Therefore, the ^{U}BCAR-iNKT cellular product is equipped with a powerful “kill switch”, further enhancing its safety profile.

The current data demonstrates the feasibility and potential of the proposed off-the-shelf ^{U}BCAR-iNKT cell therapy for MM, covering all important aspects of pre-IND development. In vitro and in vivo assays have been established to support a comprehensive characterization of the ^{U}BCAR-iNKT therapeutic candidate. Tumor-killing activity has been demonstrated for ^{U}BCAR-iNKT cells generated from HSCs of two different donors, suggesting the robustness of the proposed cellular therapy. Importantly, ^{U}BCAR-iNKT cells showed a tumor-killing efficacy comparable to or better than that of the conventional BCMA CAR-T cells, in addition to a remarkable safety profile (no GvHD), highlighting the promise of ^{U}BCAR-iNKT cell therapy as a next-generation off-the-shelf therapy for MM.

N. Further Contemplated Embodiments

1. Pharmacology, Biodistribution, Pharmacokinetics

Task A1: Identity/activity/purity The inventors will study the purity, phenotype, and functionality of the ^{U}BCAR-iNKT cell product using pre-established flow cytometry assays and ELISA (FIG. 17). The inventors expect >97%/30% purity of ^{U}BCAR-iNKT cells (>97% ^{U}HSC-iNKT cells, gated as hTCRαβ⁺6B11⁺HLA-I/II^{neg}; and >30% BCMA-CAR-positive cells, gated as tEGFR⁺). The inventors expect that these ^{U}BCAR-iNKT cells display a typical human iNKT cell phenotype (hCD45RO^{hi}hCD161^{hi}hCD4⁻hCD8^{+/-}), express no detectable endogenous TCRs due to allelic exclusion, and respond to both BCMA/CAR and αGC-CD1d/TCR mediated stimulation upon co-culturing with the MM.1S-hCD1d-FG target cells (FIG. 17 & FIG. 18). Anti-tumor activities of ^{U}BCAR-iNKT cells will be studied through measuring their proliferation and production of effector cytokines (IFN-γ) and cytotoxic molecules (Granzyme B, perforin) (FIG. 17).

Task A2: Pharmacokinetics/pharmacodynamics (PK/PD) The inventors plan to study the bio-distribution and in vivo dynamics of the ^{U}BCAR-iNKT cells by adoptively transferring these cells into tumor-bearing NSG mice (10×10⁶ cells per mouse). The pre-established human MM (MM.1S-hCD1d-FG) xenograft NSG mouse model will be used (FIG. 19A). Flow cytometry analysis will be performed to study the presence of ^{U}BCAR-iNKT cells in blood and tissues. PET imaging will be performed to study the whole-body distribution of ^{U}BCAR-iNKT cells, following established protocols (FIG. 21C). Based on preliminary studies, the inventors expect to observe that the ^{U}BCAR-iNKT cells can persist in tumor-bearing animals for some time post-adoptive transfer, can home to the lymphoid organs (spleen and bone marrow), and most importantly, can traffic to and infiltrate metastatic tumor sites.

Task A3: Dose/Regimen/Route of Administration The inventors plan to conduct dose escalation study to evaluate the in vivo antitumor efficacy/safety of the ^{U}BCAR-iNKT cells. The pre-established human MM (MM.1S-hCD1d-FG) xenograft NSG mouse model will be used (FIG. 19A). In the pilot studies, a dose of 7×10⁶ BCAR-iNKT therapeutic surrogate cells (without HLA knockout) effectively suppressed tumor growth without causing apparent toxicity (FIG. 19). The inventors therefore propose a dose escalation study for the therapeutic candidate ^{U}BCAR-iNKT cells as depicted in Table 1. Results from this task will be valuable to help design the dose escalation study for the future Phase I clinical trial. The preconditioning regimen will be lymphoablation of the recipient: for humans it will be fludarabine plus cyclophosphamide treatment; for mice it will be sub-lethal whole-body irradiation (175 rads for NSG mice) (FIG. 19A). The route of administration will be intravenous injection.

TABLE 1
Dose Escalation Study Design

| Mouse Cohort | | | | |
|---|---|---|---|---|
| (n = 8) | A | B | C | D |
| Dose of ^{U}BCAR- | 0 | 2 × 10⁶ | 5 × 10⁶ | 10 × 10⁶ |
| iNKT (CAR⁺) | | | | |

| | |
|---|---|
| Measurements | Efficacy (tumor suppression) & Safety |
| | (see Project Plan C2) |

Task A4: Efficacy The inventors plan to study the tumor killing efficacy of ^{U}BCAR-iNKT cells using the pre-established in vitro tumor cell killing assay (FIG. 18A) and in vivo tumor killing animal model (FIG. 19A). In addition to the MM.1S-hCD1d-FG model, the inventors will also test the efficacy in an L363-based MM mode; two models will increase the rigor of efficacy evaluation. For in vivo efficacy studies, tumor-bearing mice will receive escalating doses of ^{U}BCAR-iNKT cells (as indicated in Table 1). The inventors expect to observe that the ^{U}BCAR-iNKT cells can effectively kill MM.1S and L363 tumor cells in vitro and in vivo, similar to that observed in the pilot studies (FIG. 18 & FIG. 19). From the in vivo tumor killing dose escalating study, the inventors expect to identify the minimal effective dose of ^{U}BCAR-iNKT cells that can eradicate MM tumors, defined as undetectable by BLI imaging and flow cytometry as well as long-term survival.

Task A5: Mechanism of action (MOA) ^{U}BCAR-iNKT cells can target MM tumor cells through CAR/TCR dual killing mechanism, as demonstrated in the pilot MOA study (FIGS. 18B & 18E). The inventors plan to assess and validate these mechanisms for the manufactured ^{U}BCAR-iNKT cell products. The inventors expect to observe that ^{U}BCAR-iNKT cells can kill MM tumor cells through both CAR- and TCR-mediated mechanisms, with a possible synergistic effect between these two mechanisms.

2. Chemistry, Manufacturing and Controls

The pilot CMC study demonstrated the successful production of ^{U}BCAR-iNKT cells using a 2-Stage in vitro culture system (FIG. 16). The inventors plan to build on the previous success to further optimize the manufacturing process and establish critical quality control assays, in order to prepare the therapeutic candidate ^{U}BCAR-iNKT cells to enter Phase I clinical trials, and in the future, to advance to further clinical and commercial development (FIG. 22A-C). The inventors aim to 1) establish a manufacturing process that can be readily adapted to GMP production and be scaled up to supply Phase I clinical trials (FIG. 22B), 2) establish critical In Process Control (IPC) assays and product release assays to ensure the quality of the intended cellular product (FIG. 22C), and 3) demonstrate the robustness of the CMC design by completing the production and release of three lots, from three different donors, ^{U}BCAR-iNKT cells that are at the scale of 10¹⁰ and of high purity (>97% HLA-I/II-negative human iNKT cells, of which >30% are BCMA-CAR-positive cells) (FIG. 22C). The 10¹⁰ product scale is chosen because it is feasible for a research laboratory setting; it is adequate to supply the proposed preclinical studies; and importantly, this manufacturing scale is sufficient for future Phase I clinical trials (FIG. 22B). In order to accomplish these goals, the inventors proposed the following 5 tasks.

Task B1: Generate a Lenti/iNKT-sr39TK Vector The inventors propose to utilize a clinical lentiviral vector Lenti/iNKT-sr39TK that has been developed by the inventors' previous TRAN1-08533 project for the delivery of a human iNKT TCR gene together with an sr39TK PET imaging/suicide gene (FIG. 22A). The same lentivector has been utilized in the pilot CMC study (FIG. 16A), and the same lentivector backbone has already been used in two CIRM-funded clinical trials led by co-investigators Dr. Donald Kohn and Dr. Antoni Ribas (IND #16028; IND #17471). In the TRAN1-08533 project, the inventors have successfully produced research-grade Lenti/iNKT-sr39TK vector at the UCLA Vector Core (10 L; 1×10⁶ TU/ml). For the current translational project (TRAN1-11597), the inventors plan to produce another medium-scale (4-10 L) Lenti/iNKT-sr39TK vector at the UCLA Vector Core, to support the proposed preclinical studies. Notably, the Indiana University Vector Production Facility (IUVPF) has produced a GMP-compatible test lot of the Lenti/iNKT-sr39TK vector for us that was of a similar high titer and has agreed to produce clinical-grade vector for us when the project moves to the clinical development and GMP production stage (see Support Letter).

Task B2: Generate a Retro/BCMA-CAR-tEGFR Vector The inventors plan to use gammaretroviral vector Retro/BCMA-CAR-tEGFR for CAR engineering. The vector backbone is based on a modified moloney murine leukemia virus described previously. The BCMA CAR is a second-generation design consisting of an anti-BCMA single chain variable fragment, a CD8 hinge and transmembrane region, and 4-1BB and CD3, cytoplasmic regions. Through a P2A linker, the vector also encodes a truncated epidermal growth factor receptor (tEGFR) as a safety switch. The cDNA sequence encoding this CAR was codon-optimized, synthesized and cloned into the retroviral vector backbone. The inventors generated a retroviral producer line for making Retro/BCMA-CAR-tEGFR with the use of the PG13 gibbon ape leukemia virus packaging cell line. One clone with the highest titer was chosen and used to produce vectors for the described pilot study (FIG. 16-19 & FIG. 21). In this project, the inventors plan to use this clonal producer line to generate a medium-scale (5 L) Retro/BCMA-CAR-tEGFR vector in the laboratory to support the proposed preclinical studies. The inventors also plan to establish a contract service with Charles River to generate cGMP-compliant master and working cell banks for the vector producer line. The inventors plan to ask IUVPF to use these cell banks to produce clinical-grade vector when the project moves to the clinical development and GMP production stage.

Task B3: Generate a CRISPR-Cas9/B2M-CIITA-gRNAs Complex The inventors propose to utilize the powerful CRISPR-Cas9/gRNA gene-editing tool to disrupt the B2M and CIITA genes in human HSCs (FIG. 22A). BCAR-iNKT cells derived from such gene-edited HSCs will lack HLA-I/II expression, thereby avoiding rejection by the host T cells. In the pilot CMC study, the inventors have successfully generated and validated a CRISPR-Cas9/B2M-CIITA-gRNAs complex (Cas9 from the UC Berkeley MacroLab Facility; gRNAs from Synthego; B2M-gRNA sequence 5′-CGCGAGCACAGCUAAGGCCA-3′ (SEQ ID NO:68); CIITA-gRNA sequence 5′-GAUAUUGGCAUAAGCCUCCC-3′—SEQ ID NO:69), that induced HLA-I/II double-deficiency in starting HSCs and the resulting ^{U}BCAR-iNKT cells at high efficiency (˜40-60%) (FIG. 16). The inventors plan to obtain the Cas9 recombinant protein and the synthesized gRNAs from verified vendors to use in the proposed TRAN1-11597 project. In particular, to minimize the “off-target” effect, the inventors will utilize the high-fidelity Cas9 protein from IDT. The inventors will start with the pre-tested single dominant B2M-gRNA and CIITA-gRNA, but will consider incorporating multiple gRNAs to further improve the gene-editing efficiency if needed.

Task B4: Produce ^{U}BCAR-iNKT cells The proposed manufacturing process and IPC/product releasing assays are shown in a flow diagram (FIG. 22C). Eight steps are involved, which are detailed below.

Collect HSCs (Steps 1 & 2) The inventors plan to obtain G-CSF-mobilized LeukoPaks of three different healthy donors from the commercial vendor HemaCare, followed by isolating the CD34⁺ HSCs using a CliniMACS system located at the UCLA GMP Facility. HemaCare has IRB-approved collection protocols and donor consents, and is capable of supporting both preclinical research and future clinical trials and commercial product manufacturing (see Support Letter). In the inventors' previous CIRM TRAN1-08533 project, the inventors successfully obtained G-CSF LeukoPaks of multiple donors from HemaCare and isolated CD34⁺ HSCs at high yield and of high purity (1-5×10⁸ HSCs per donor; >99% purity). The inventors expect a similar yield and purity for the new collections. After isolation, G-CSF-mobilized CD34⁺ HSCs will be cryopreserved and be used for the proposed TRAN1-11597 project.

Gene-Engineer HSCs (Steps 3 & 4) The inventors plan to engineer HSCs with both the Lenti-iNKT-sr39TK vector and the CRISPR-Cas9/B2M-CIITA-gRNAs complex following a protocol well-established at the laboratories of the PI and the co-investigator, Dr. Donald Kohn. Cryopreserved CD34⁺ HSCs will be thawed and cultured in X-Vivo-15 serum-free medium supplemented with 1% HAS and TPO/FLT3L/SCF for 12 hours in flasks coated with retronectin, followed by addition of the Lenti/iNKT-sr39TK vector for an additional 8 hours. 24 hours after the lentivector transduction, cells will be mixed with pre-formed CRISPR-Cas9/B2M-CIITA-gRNAs complex and subjected to electroporation using a Lonza Nucleofector. In the pilot studies, the inventors have achieved high lentivector transduction rate (˜30-40% transduction rate with VCN=1-3 per cell; FIG. 16B) and high HLA-I/II double-deficiency (˜50-70% HLA-I/II double-negative cells of cultured HSCs after a single round of electroporation; FIG. 16B) using CD34⁺ HSCs of two random healthy donors. The inventors plan to further optimize the gene-editing procedure to improve efficiency. The evaluation parameters will be cell viability, deletion (indel) frequency (on-target efficiency) measured by a T7E1 assay and next-generation sequencing targeting the B2M and CIITA sites, HLA-I/II expression by flow cytometry, and hematopoietic function of edited HSCs measured by the Colony Formation Unit (CFU) assay. The inventors aim to achieve 20-50% triple-gene editing efficiency of HSCs, which in the preliminary studies could give rise to ˜100 ^{U}HSC-iNKT cells per input HSC after Stage 1 culture (FIG. 16G).

Generate ^{U}BCAR-iNKT Cells (Steps 5-8) The inventors propose to culture the lentivector and CRISPR-Cas9/gRNA double-engineered HSCs in a 2-Stage in vitro system to produce ^{U}BCAR-iNKT cells. At Stage 1, the gene-engineered HSCs will be differentiated into iNKT cells via ATO culture following a standard protocol developed by the laboratory of co-investigator, Dr. Gay Crooks (FIG. 2C). ATO involves pipetting a cell slurry (5 μl) containing a mixture of HSCs (1×10⁴) and irradiated (80 Gy) MS5-hDLL1 stromal cells (1.5×10⁵) as a drop format onto a 0.4-μm Millicell transwell insert, followed by placing the insert into a 6-well plate containing 1 ml RB27 medium; medium will be changed every 4 days for 8 weeks. The inventors will use the automated pipetting system (epMotion) to simplify and optimize ATO culture procedure. The harvested cells will be matured and expanded for two weeks with αGC loaded onto irradiated donor-matched CD34⁻ PBMCs (as APCs) and supplemented with IL-7 and IL-15 using G-Rex bioreactors (FIG. 22C). The resulting cells will be purified through MACS sorting (2M2/Tü39 mAb-mediated negative selection followed by 6B11 mAb-mediated positive selection) to generate pure ^{U}HSC-iNKT cells (FIG. 16E). At Stage 2, iNKT cells will be activated by anti-CD3/CD28 beads, transduced with the Retro/BCMA-CAR-tEGFR vector under RetroNectin conditions, and expanded with T cell culture medium in G-Rex bioreactors supplemented with IL-15 to yield the final ^{U}BCAR-iNKT cell product; the total duration for Stage 2 is two weeks (FIG. 22C). Based on the pilot CMC study (FIG. 16), the inventors expect to produce ˜10¹⁰ scale of ^{U}BCAR-iNKT cells from each of the 3 donors (1×10⁶ starting HSCs), that are of high purity (>97% HLA-I/II-negative human iNKT cells, of which >30% are BCMA-CAR-positive cells). The resulting ^{U}BCAR-iNKT cells will then be cryopreserved and used for preclinical characterizations. The inventors will use GatheRex liquid handling to operate G-Rex bioreactors to ensure a closed system for cell expansion. Overall, the inventors believe that most process steps can be easily automated for commercial scale production.

Quality Control for Bioprocessing and Product (Steps 1-8) As outlined in FIG. 22C, various IPC assays will be incorporated into the proposed bioprocess to ensure a high-quality production. The proposed product releasing testing include 1) appearance (color, opacity); 2) cell viability and count; 3) identity and VCN by qPCR for iNKT TCR and BCMA CAR; 4) purity by iNKT positivity, HLA-I/II negativity, and CAR positivity; 5) endotoxins; 6) sterility; 7) mycoplasma; 8) potency measured by IFN-γ release in response to MM.1S-hCD1d-FG stimulation; 9) RCL (replication-competent lentivirus). Most of these assays are either standard biological assays or specific assays unique to this product that will be validated in the PI's laboratory. Product stability testing will be performed by periodically thawing LN-stored ^{U}BCAR-iNKT cells and measuring their cell viability, purity, recovery, potency (IFN-γ release), and sterility. Although it remains to be determined the achievable shelf life, the inventors expect that the product should be stable for at least one year.

Task B5: Generate cGMP-compliant MS5-hDLL1 cell banks The stromal cell line, MS5-hDLL1, for ATO culture has already been authenticated with regard to species and strain of origin by STR analysis, and has been tested negative for mycoplasma contamination. It has also been tested by Charles River and is negative for infectious diseases by a Mouse Essential CLEAR panel, and negative for interspecies contamination for rat, Chinese hamster, Golden Syrian hamster, and non-human primate. These testing results are consistent with the FDA's position regarding xenogeneic feeder cells and thus give us confidence that this cell should meet requirements for GMP manufacturing. The inventors have banked enough cells for this preclinical study. In preparation for future GMP production, the inventors will establish a contract service with Charles River to generate cGMP-compliant MS5-hDLL1 master and working cell banks.

3. Safety Embodiments

The inventors plan to study the safety of ^{U}BCAR-iNKT cellular product on four criteria: 1) general graft-versus-host disease (GvHD), toxicity, and tumorigenicity; 2) cytokine release syndrome and neurotoxicity; 3) immunogenicity; and 4) suicide gene “kill switch”.

Task C1: General GvHD/toxicity/tumorigenicity The long-term GvHD (against recipient animal tissues), toxicology, and tumorigenicity of ^{U}BCAR-iNKT cells will be studied through adoptively transferring these cells into tumor-free NSG mice and monitoring the recipient mice over a period of 20 weeks, ended with terminal pathology analysis, following an established protocol (FIG. 19). The inventors expect no GvHD, no toxicity, and no tumorigenicity as that observed for the therapeutic surrogate BCAR-iNKT cells (FIG. 19).

Task C2: Cytokine release syndrome (CRS) and neurotoxicity The main adverse side-effects of CAR-T therapy are CRS and neurotoxicity. Accumulating evidence suggests that monocytes and macrophages are major cell sources for mediating these toxicities. The inventors will evaluate the potential of CRS and neurotoxicity after MM treatment by ^{U}BCAR-iNKT using humanized mice; the team has extensive experience in this type of mouse model. NSG-SGM3 mice (NSG mice with triple transgenics of human proteins SCF, GM-CSF and IL-3, available from JAX) will be sublethally irradiated (170 cGy) and transplanted with human CD34⁺ HSCs (10⁵, for reconstitution of human immune cells such as monocytes, macrophages, B cells) and MM.1S-hCD1d-FG cells (0.5×10⁶, MM tumor cells). Once high MM tumor burdens are established (in 4 weeks, confirmed by BLI imaging), two doses of ^{U}BCAR-iNKT cells (2×10⁶ and 10×10⁶) will be infused; two of the same doses of conventional BCMA CAR-T cells will be included as controls. Mice will be monitored for CRS occurrence by measuring daily for weight loss and body temperature (by rectal thermometry), and weekly for mouse serum amyloid A (homologous to human C-reactive protein) and human cytokines (IL-1, IL-6, GM-CSF, IFN-γ, etc.) via multiplex cytokine assays. The inventors will report CRS mortality defined as death preceded by >15% weight loss, ΔT>2° C. and serum IL-6>1,000 pg/ml, and lethal neurotoxicity defined as death in the absence of CRS observation but preceded by either paralysis or seizures. The inventors anticipate no more severe CRS and neurotoxicity generated by ^{U}BCAR-iNKT as compared to BCMA CAR-T. If these toxicities are observed, the inventors will also investigate whether administration of tocilizumab (anti-IL-6R antibody) or anakinra (IL-1R antagonist) can ameliorate these side-effects.

Task C3: Immunogenicity For immune cell-based adoptive therapies, there are always two immunogenicity concerns: a) GvHD, and b) Host-Versus-Graft (HvG) responses. The inventors have considered the possible GvHD and HvG risks for the ^{U}BCAR-iNKT cellular product and engineered safety control strategies (FIG. 20A). The HvG concern is actually an efficacy concern; but for the convenience of discussion, the inventors include it under the “Safety” section. The inventors will study the possible GvHD and HvG responses using established in vitro Mixed Lymphocyte Culture (MLC) assays FIGS. 20B & 20D) and an in vivo Mixed Lymphocyte Adoptive Transfer (MLT) Assay. The readouts of the in vitro MLC assays will be IFN-γ production analyzed by ELISA, while the readouts of the in vivo MLT assays will be the elimination of targeted cells analyzed by bleeding and flow cytometry (either the killing of mismatched-donor PBMCs as a measurement of GvHD response, or the killing of ^{U}BCAR-iNKT cells as a measurement of HvG response). Based on pilot studies, the inventors expect to observe that the ^{U}BCAR-iNKT cells do not induce GvHD response against host animal tissues (FIG. 19E), do not induce GvHD response against mismatched-donor PBMCs (FIG. 20B), and are not subject to HvG responses from mismatched-donor PBMC T cells (FIG. 20E).

Task C4: Suicide gene “kill switch” The inventors plan to study the elimination of ^{U}BCAR-iNKT cells in recipient NSG mice through GCV administration, following an established protocol (FIG. 21B). Based on pilot studies, the inventors expect to find that the sr39TK suicide gene can function as a powerful “kill switch” to eliminate ^{U}BCAR-iNKT cells in case of a safety need.

4. Risks, Mitigation Strategies

sr39TK PET imaging/suicide gene The imaging/safety control sr39TK gene engineered into the ^{U}BCAR-iNKT cell product is potentially immunogenic because of its viral origin (HSV1). However, this immunogenic concern has been mitigated greatly as 1) the cell product lacks the expression of HLA-I/II molecules so that the likelihood of T cell-related immunogenicity is reduced; 2) MM patients will be pre-conditioned with the lymphodepleting chemotherapy prior to the drug infusion. Importantly, this is likely to be the first-in-human study for infusion of allogeneic iNKT cells and thus safety will be the paramount consideration.

Purity of the cell product The manufacturing process includes a purification step (negative/positive selection using MACS) to ensure the high purity of the ^{U}BCAR-iNKT cellular product. It should be pointed out that the 6B11 antibody has superior specificity, stability and affinity (as compared to traditional tetramers) for human iNKT TCRs and thus is a robust reagent for iNKT cell purification. As shown in the pilot studies, the inventors expect to achieve >98%/95% purity (>98% iNKT cells; of which >95% are HLA-I/II-negative) (FIG. 16E). However, it remains theoretically possible that the product contains trace amounts of conventional αβ T cells, which pose the risk of GvHD. Thus, the inventors will keep the option open to further improve the product purity by increasing the rounds of MACS purification. Because of the safeguard sr39TK gene, the clinical risk of GvHD can be managed as well.

Risk of rejection by host NK cells The lack of HLA expression in the cell product can trigger the risk of rejection/killing by the host NK cells. The preliminary studies did not detect such killing/rejection during the coculture of iNKT with mismatched-donor NK cells. Nonetheless, if further studies show that NK reactivity can not only occur but also impact the therapy via reducing engraftment efficiency, the inventors can engineer ^{U}BCAR-iNKT cells to express NK inhibitors such as HLA-E to mitigate this effect.

### Example 3: Generation of Allogeneic Hematopoietic Stem Cell-Engineered Invariant Natural Killer T Cells for Off-the-Shelf Immunotherapy

A. Generation of Allogeneic HSC-Engineered iNKT (^{Allo}HSC-iNKT) Cells (FIG. 23)

The inventors used an artificial thymic organoid (ATO) system to generate allogeneic HSC-engineered human iNKT cells. This system supported efficient and reproducible differentiation and positive selection of human T cells from hematopoietic stem cells (HSCs) (Montel-Hagen et al., 2019; Seet et al., 2017). Human HSCs were collected either from granulocyte-colony stimulating factor (G-CSF)-mobilized human PBMCs, or cord blood (CB) cells. These HSCs were transduced with a Lenti/iNKT-sr39TK vector and then cultured in vitro in a two-stage ATO/α-galactosylceramid (αGC, a synthetic glycolipid ligand specific to iNKT cells) culture system (FIGS. 23A and 23B). The genetic modifications from the Lenti/iNKT-sr39TK vector efficiently differentiated the HSCs into human iNKT cells in the ATO culture system over 8 weeks with 100 times expansion (FIG. 23C). These cells then further expanded in the APC/αGC stimulation stage for another 2-3 weeks with another 100-1000 times expansion (FIG. 23D). ^{Allo}HSC-iNKT cells followed a typical iNKT cell development path defined by CD4/CD8 co-receptor expression, with the start from DN (double negative) precursor cells by week 4, followed by a predominance of DP (double positive) by week 6, and then to CD8 SP (single positive) or back to DN cells by week 8 (FIG. 23E) (Godfrey and Berzins, 2007). After APC/αGC stimulation, ^{Allo}HSC-iNKT cells expressed a CD8 SP and DP mixed pattern (FIG. 23E). Following the generation process, the cells were tested in 12 donors (4 donors for CB cells and 8 donors for PBSCs) which demonstrated how robust this process was regarding to its level of yield and purity (FIG. 23F). It was estimated that from 1×10⁶ input CB cells (˜30%-50% lentivector transduction rate), about 5-15×10^{10 Allo}HSC-iNKT cells (95%-98% purity) could be generated, and from 1×10⁶ input PBSCs, about 3-9×10^{10 Allo}HSC-iNKT cells (95%-98% purity) could be generated (FIG. 23F).

B. Analysis of TCR Vα and Vβ Sequences in ^{Allo}HSC-iNKT Cells (FIG. 23)

Next, the inventors studied the TCR repertoire ^{Allo}HSC-iNKT cells, in comparison with that of conventional αβ T cells and endogenous human iNKT cells isolated from the peripheral blood of healthy human donors (denoted as PBMC-Tc and PBMC-iNKT cells, respectively). PBMC-Tc cells displayed a highly diverse distribution of TCR Vα and Vβ gene usage (FIG. 23F). While PBMC-iNKT cells showed a ubiquitous and highly conserved TCR Vα sequence TRAV10/TRAJ18 (Vα24-Jα18), and a more diverse TCR V3 sequence but predominantly TRBV25-1⁺ (Vβ11) (FIG. 23F). In sharp contrast, the ^{Allo}HSC-iNKT cells showed markedly reduced sequence diversity, with nearly undetectable endogenous TCR Vα and Vβ sequences (FIG. 23F), which is due to allelic exclusion (Giannoni et al., 2013; Vatakis et al., 2013).

C. Phenotype and Functionality of ^{Allo}HSC-iNKT Cells (FIG. 24)

^{Allo}HSC-iNKT cells displayed typical iNKT cell phenotype similar to that of PBMC-iNKT cells, but distinct from that of PBMC-Tc cells: ^{Allo}HSC-iNKT cells expressed CD4 and CD8 co-receptors with a mixed pattern (CD4/CD8 DN and CD8 SP) and they expressed high levels of memory T cell marker CD45RO and NK cell marker CD161. In addition, they also upregulated peripheral tissue and inflammatory site homing markers (CCR4, CCR5 and CXCR3) (FIG. 24A) and produced exceedingly high levels of effector cytokines such as IFN-γ, TNF-α and IL-2, and cytotoxic molecules like perforin and granzyme B in comparison to those of PBMC-Tc cells (FIG. 24B).

To test the functionality of ^{Allo}HSC-iNKT cells, the inventors first stimulated them with αGC. This antigen caused ^{Allo}HSC-iNKT cells to proliferate at a much higher rate (FIG. 24C) and secrete higher levels of Th0/Th1 cytokines, including IFN-γ, TNF-α and IL-2 (FIG. 24D). Upon stimulation, ^{Allo}HSC-iNKT cells secreted negligible amounts of Th2 cytokines such as IL-4 and Th17 cytokines such as IL-17 (FIG. 24D), indicating that these iNKT cells had a Th0/Th1-biased profile.

D. Transcriptional Analysis of ^{Allo}HSC-iNKT Cells (FIG. 24)

The inventors analyzed the global gene expression profiles of ^{Allo}HSC-iNKT cells, and other lymphoid cell subsets, including healthy donor PBMC-derived conventional CD8⁺ αβ T (PBMC-αβTc), γδ T (PBMC-γδT), NK (PBMC-NK), and CD8⁺PBMC-iNKT cells. PBMC-αβTc, −iNKT and −γδT cells were all expanded in vitro by antigen/TCR stimulation, and PBMC-TC and −iNKT cells were flow sorted out CD8⁺ population in order to be consistent with ^{Allo}HSC-iNKT cells. Principal component analysis using global expression profiles for all populations demonstrated that both CB-derived and PBSC-derived ^{Allo}HSC-iNKT cells were closest to PBMC-iNKT cells and next closest to PBMC-Tc and PBMC-γδT cells, while farthest to PBMC-NK cells (FIG. 24E).

The signature transcription factors of innate type T cells ZBTB16 (PLZF), Th1 type T cells TBX21 (T-bet), and TCR signaling NFKB1 and JUN, were highly expressed in ^{Allo}HSC-iNKT cells. Those transcription factors were required for the generation and effector function of iNKT cells (Kovalovsky et al., 2008; Matsuda et al., 2006; Park et al., 2019). However, these cells displayed low Th2 and TH17 type transcription factors (FIG. 24F), showing a Th1-prone effector function of ^{Allo}HSC-iNKT cells, which was consistent with the cytokines profiling results (FIG. 24D).

To examine the immunogenicity of ^{Allo}HSC-iNKT cells, the inventors compared HLA gene expression in the six cell types. HLA compatibility is a main criterion for donor selection in stem cell transplantation, and HLA mismatches increase the risk of mortality caused by alloreactivity (Furst et al., 2019). Interestingly, both CB and PBSC derived ^{Allo}HSC-iNKT cells displayed a universal low expression of HLA molecules, including HLA-I, HLA-II, B2M and HLA-II transactivators (FIG. 24G), suggesting that the HSC-engineered cells were naturally of low immunogenicity compared to conventional PBMC cells. The low HLA-I and HLA-II molecules on ^{Allo}HSC-iNKT cells might ameliorate recognition of host CD8 and CD4 T cells, thus largely reducing host-versus-graft (HvG) responses. These results strongly support ^{Allo}HSC-iNKT cells are an ideal candidate for allogeneic cellular therapy which have low immunogenicity.

As to immune checkpoint inhibitors, ^{Allo}HSC-iNKT cells displayed a lower expression of PD-1, CTLA-4, TIGIT, LAG3, PD-L1 and PD-L2, in comparison of PBMC-iNKT, PBMC-αβTc, and PBMC-γδT cells (FIG. 24H). These immune checkpoint inhibitors expressed on effector cells lead to inhibition of cell activation upon binding to their ligands on tumor cells or antigen-presenting cells (Darvin et al., 2018). The low expression of immune checkpoint inhibitors on ^{Allo}HSC-iNKT cells might sustain iNKT cell activation when they target tumor cells. Of note, recent clinical data showed the cancer patients with low PD-1 or PD-L1 expression in T cells were more likely to experience treatment benefit with checkpoint blockade therapy and show prolonged progression-free survival (Brody et al., 2017; Mazzaschi et al., 2018), indicating the potential clinical benefit of ^{Allo}HSC-iNKT cells-based checkpoint blockade combination therapy.

Reflecting NK-like cytotoxicity of ^{Allo}HSC-iNKT cells, the NK-activating receptor genes, including NCAM1, NCR1, NCR2, KLR2, KLR3, etc. were highly expressed in ^{Allo}HSC-iNKT cells compared to other cell types (FIG. 24I). Interestingly, the NK inhibitory receptor genes, including KIR3DL1, KIR3DL2, KIR2DL1, KIR2DL2, etc. had lower expressions compared to PBMC-NK cells (FIG. 24I). Taken together, these observations indicated ^{Allo}HSC-iNKT cells might exhibited a stronger killing capacity to tumor cells through NK pathway in comparison to PBMC-NK cells.

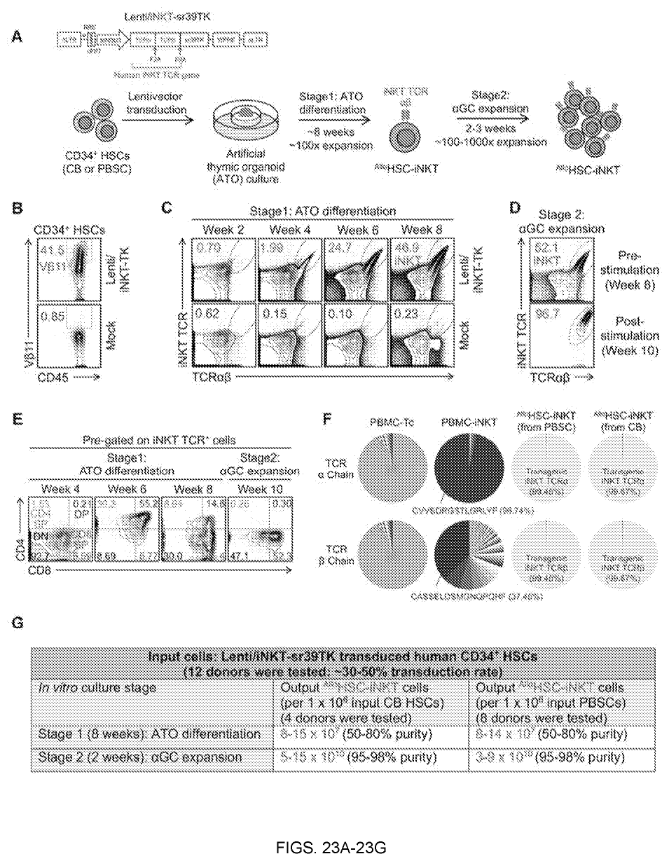

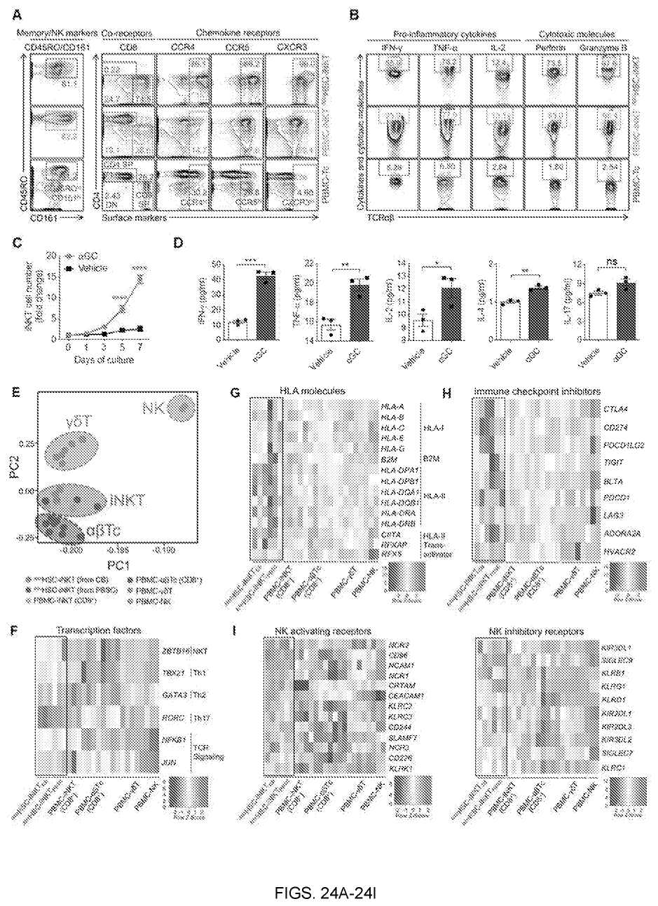

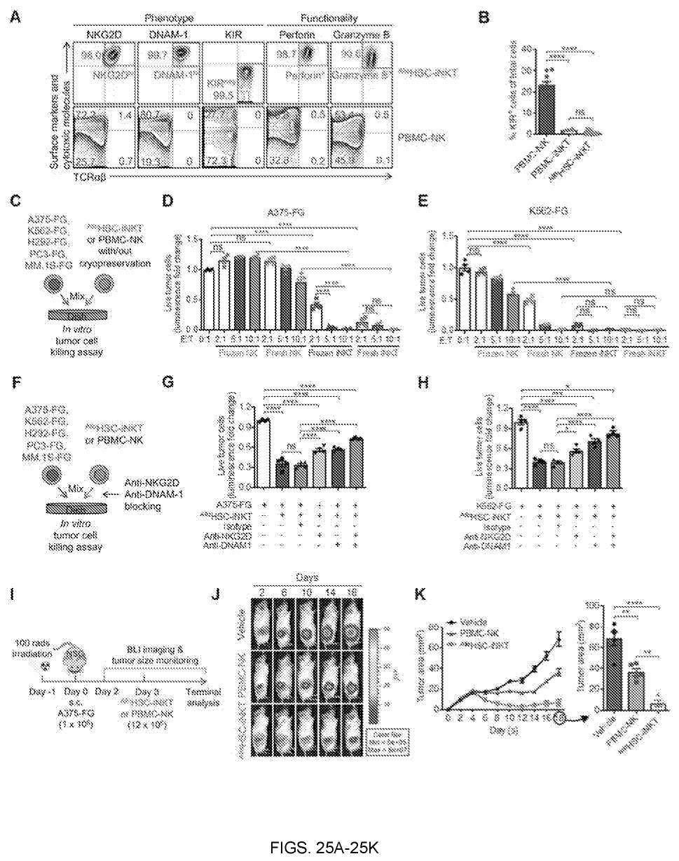

E. Tumor Targeting of ^{Allo}HSC-iNKT Cells Through NK Pathway (FIG. 25)

iNKT cells are narrowly defined as a T cell lineage expressing NK lineage receptors (Bendelac et al., 2007), therefore the inventors studied the NK phenotype and functionality of ^{Allo}HSC-iNKT cells in comparison with endogenous PBMC-NK cells. ^{Allo}HSC-iNKT cells expressed higher levels of NK activating receptors NKG2D and DNAM-1 and produced higher levels of cytotoxic molecules perforin and granzyme B compared to PBMC-NK cells (FIG. 25A). Interestingly, the ^{Allo}HSC-iNKT cells did not express killer cell immunoglobulin-like receptor (KIR), which acted as an inhibitory receptor for NK cell activation and prevented those MHC matched ‘self-cells’ from NK killing (FIGS. 25A AND 25B) (Ewen et al., 2018; Del Zotto et al., 2017).

In order to test the direct killing capabilities of iNKT cells through the NK pathway (Fujii et al., 2013; Vivier et al., 2012), the inventors utilized an in vitro tumor cell killing assay with CD1d negative tumor cells. The inventors tested five CD1d-negative tumor cell lines, including a human melanoma cell line A375, a human myelogenous leukemia cell line K562, a human mucoepidermoid pulmonary carcinoma cell line H292, a human adenocarcinoma cell line PC3, and a human multiple myeloma cell line MM.1S. All five tumor cell lines were engineered to overexpress the firefly luciferase (Fluc) and EGFP reporters (FIG. 30A). In the absence of CD1d expression on tumor cells and αGC supplementation, ^{Allo}HSC-iNKT exhibited a stronger and more aggressive killing capacity across all five tumor cell lines in comparison to the PBMC-NK cells (FIGS. 25C-25E, and FIGS. 30B-30D). In addition, ^{Allo}HSC-iNKT cells displayed strong anti-tumor killing after cryopreservation, while PBMC-NK cells were sensitive to freeze-thaw cycles and had diminished anti-tumor capability following cryopreservation (FIGS. 25C-25E, and FIGS. 30B-30D). Using anti-NKG2D and anti-DNAM-1 blocking antibodies, the inventors revealed that ^{Allo}HSC-iNKT cells mediated cell lysis on A375, K562, PC3 and H292 cells were NKG2D- and DNAM-1-dependent (FIGS. 25F-25H, and FIGS. 30E-30F), while cell lysis on MM.1S cells was mainly mediated by DNAM-1 (FIG. 30G). This suggested that ^{Allo}HSC-iNKT cells could kill CD1d negative tumor cells via NKG2D- and DNAM-1-dependent mechanisms.

F. In Vivo Antitumor Efficacy of ^{Allo}HSC-iNKT Cells Against Solid Tumors Through NK Pathway in a Human Melanoma Xenograft Mouse Model (FIG. 25)

In vivo antitumor efficacy of ^{Allo}HSC-iNKT cells against solid tumors through NK pathway was studied using human melanoma xenograft NSG (NOD.Cg-Prkdc^{scid}Il2rg^{tm1Wj1}/SzJ) mouse model. A375-IL-15-FG tumor cells were subcutaneously inoculated into NSG mice to form solid tumors, which was followed by a paratumoral injection of ^{Allo}HSC-iNKT and PBMC-NK cells (FIG. 25I). Compared with PBMC-NK cells, the ^{Allo}HSC-iNKT cells treated mice displayed a more significant suppression of tumor growth, detected by time-course bioluminescence (BLI) imaging (FIG. 25J and FIG. 30H), tumor size measurement (FIG. 25K), and terminal tumor weight assessment FIG. 30I). The NK pathway dependent dramatic enhancement of anti-tumor effect of ^{Allo}HSC-iNKT cells from in vivo demonstrated the promising therapeutic potential of ^{Allo}HSC-iNKT cells for treating solid tumors.

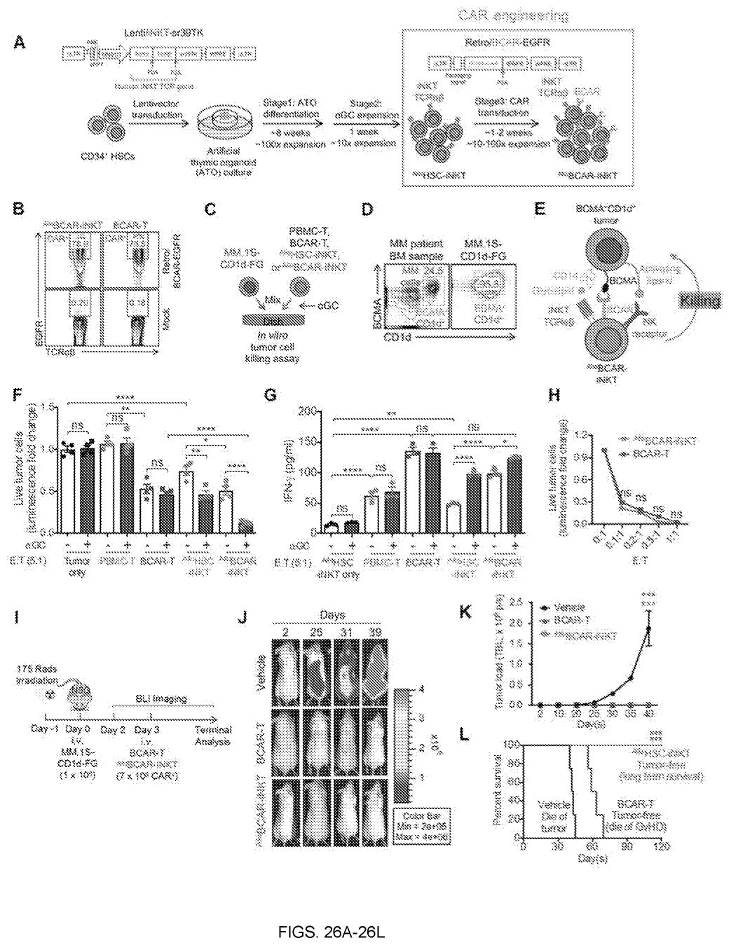

G. Engineering of BCMA-CAR (BCAR) on ^{Allo}HSC-iNKT Cells (FIG. 26)

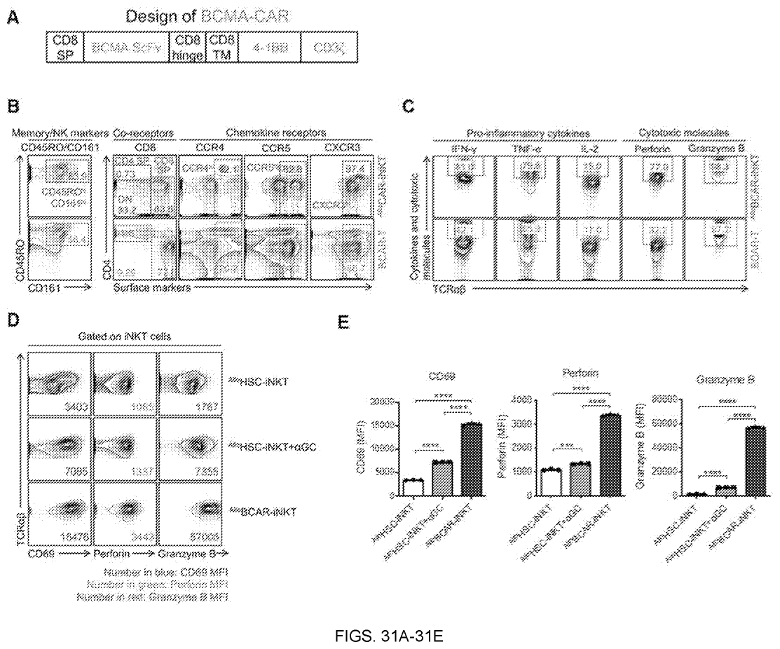

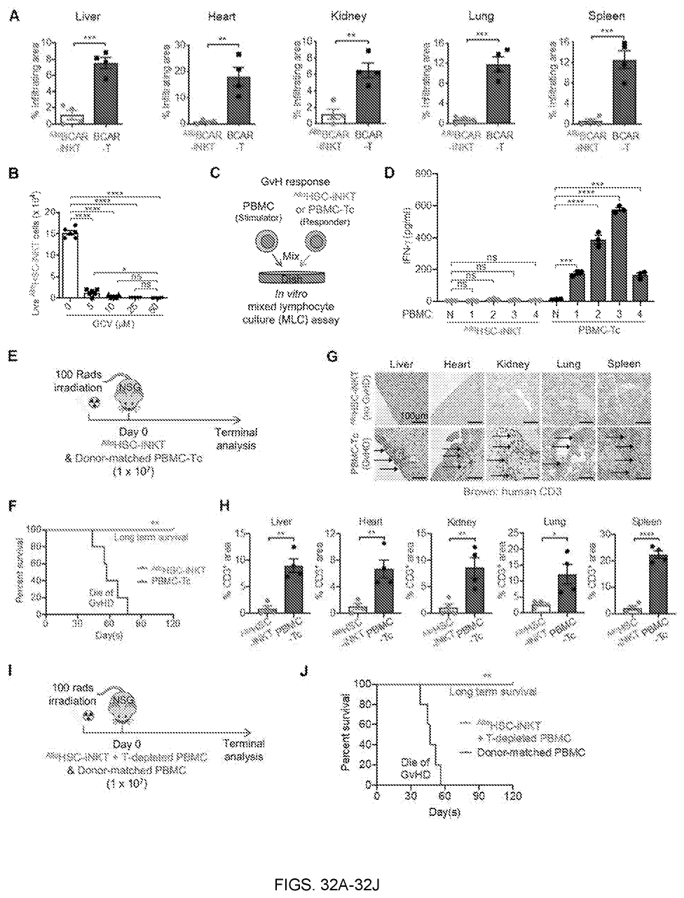

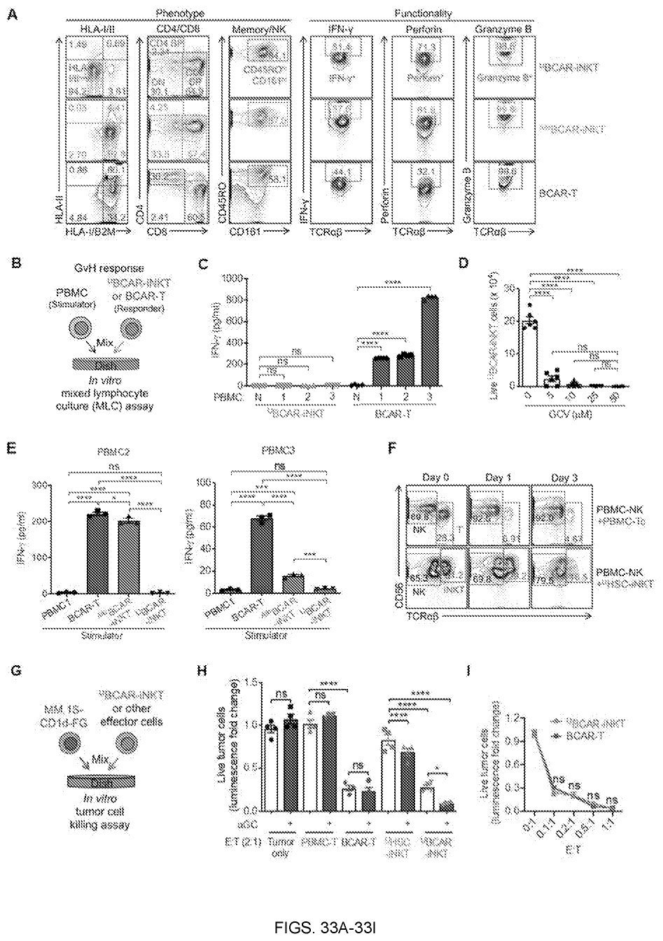

The inventors further engineered a BCAR on ^{Allo}HSC-iNKT cells, which were armed with a single-chain variable fragment (scFv) specific to BCMA plus 4-1BB endodomains. Truncated EGFR was also included and utilized as a surface marker tag to track transduced cells (FIG. 31A). The ^{Allo}HSC-iNKT cells were transduced with the Retro/BCMA-CAR-tEGFR retroviral vector followed by IL-7/IL-15 expansion for 1-2 weeks, leading to BCMA-CAR expression (denoted as ^{Allo}BCAR-iNKT cells) (FIG. 26A). The Retro/BCMA-CAR-tEGFR retroviral vector has been successfully utilized to manufacture autologous BCMA CAR-T cells (denoted as BCAR-T cells) for ongoing Phase I clinical trials treating MM (Timmers et al., 2019). The inventors successfully generated viable and highly transduced (˜30%-80% BCAR engineering rate) ^{Allo}BCAR-iNKT cells, comparable to engineering conventional T cells (FIG. 26B).

The phenotype and functionality of ^{Allo}BCAR-iNKT cells were studied using flow cytometry, in comparison to two controls: 1) PBMC-Tc cells from healthy donor peripheral T cells, and 2) BCAR-T cells generated by transducing healthy donor peripheral T cells with Retro/BCMA-CAR retroviral vector. ^{Allo}BCAR-iNKT cells displayed a distinct surface phenotype and functionality. They expressed CD4 and CD8 co-receptors in a mixed pattern (CD4/CD8 double-negative and CD8 single-positive) and expressed high levels of memory T cell marker CD45RO and NK cell marker CD161. In addition, they also upregulated peripheral tissue and inflammatory site homing markers (CCR4, CCR5 and CXCR3) (FIG. 31B) and produced high levels of effector cytokines such as INF-γ, TNF-α and IL-2, as well as cytotoxic molecules like perforin and granzyme B on levels comparable to or better than BCAR-T and PBMC-Tc cells (FIG. 31C).

H. Tumor-Attacking Mechanisms of ^{Allo}BCAR-iNKT cells (FIG. 26)

The inventors established an in vitro multiple myeloma (MM) tumor cell killing assay to study the tumor-attacking capacity of ^{Allo}BCAR-iNKT cells. A human MM cell line, MM.1S, was engineered to overexpress the human CD1d, Flue and EGFP reporter genes, resulting in an MM-CD1d-FG cell line that was used for this assay (FIG. 26C). Importantly, a large portion of primary MM tumor cells express both BCMA and CD1d, making these cells subject to both BCAR- and iNKT-TCR-mediated targeting (FIG. 26D). However, although the parental MM.1S cells express BCMA, they lose CD1d expression. Therefore, the inventors overexpressed CD1d in MM.1S cells to mimic primary MM tumor cells. As a result, a triple tumor killing mechanism was deployed by BCAR-iNKT (FIG. 26E). The ^{Allo}HSC-iNKT cells were able to kill the MM tumor cells through NK pathway on their own (FIG. 26F) and in the presence of αGC, the cells were able to activate a TCR-mediated killing pathway to facilitate tumor killing. In addition, engineered BCMA-CAR further enhanced the tumor killing efficacy of ^{Allo}BCAR-iNKT cells, as their efficacy was shown to be correlated with IFN-γ levels (FIG. 26F-26H). Importantly, upon stimulated by tumor antigen, ^{Allo}BCAR-iNKT cells displayed a more activated phenotype than ^{Allo}HSC-iNKT cells, as evidenced by upregulation of CD69, perforin and granzyme B (FIGS. 31D and 31E). The unique CAR/TCR/NK-mediated triple tumor killing mechanism made the inventors' ^{Allo}BCAR-iNKT cells powerful and compelling resources for MM cancer cell targeting. One additional benefit is that these cells can potentially avoid antigen escape, a phenomenon in autologous BCAR-T therapy clinical trials wherein MM cells were able to escape BCAR targeting. Furthermore, by using ^{Allo}HSC-iNKT cells as a platform, products can be easily armed with other CARs by replacing BCMA specificity to benefit other types of cancer treatment.

I. In Vivo Antitumor Efficacy of ^{Allo}BCAR-iNKT Cells Against Hematologic Malignancies in A Human MM Xenograft Mouse Model (FIG. 26)

In vivo antitumor efficacy of ^{Allo}BCAR-iNKT cells was studied using a human MM xenograft NSG mouse model with the MM.1S-CD1d-FG cell line. The experimental mice were pre-conditioned with 175 rads of total body irradiation, followed by intravenously (i.v.) inoculation of MM.1S-CD1d-FG. After 3 days, effector cells, including ^{Allo}BCAR-iNKT and BCAR-T, were i.v. injected into the mice (FIG. 26I). Both ^{Allo}BCAR-iNKT and BCAR-T cells effectively eradicated pre-established metastatic MM tumor cells (FIGS. 26J and 26K). However, mice receiving the conventional BCAR-T cells, eventually died because of graft-versus-host disease (GvHD) (FIG. 26L). In contrast, mice receiving ^{Allo}BCAR-iNKT cells survived long-term without GvHD in addition to being tumor free (FIG. 26L). These results validated the safety profile and therapeutic potential of the off-the-shelf ^{Allo}BCAR-iNKT-based immunotherapy.

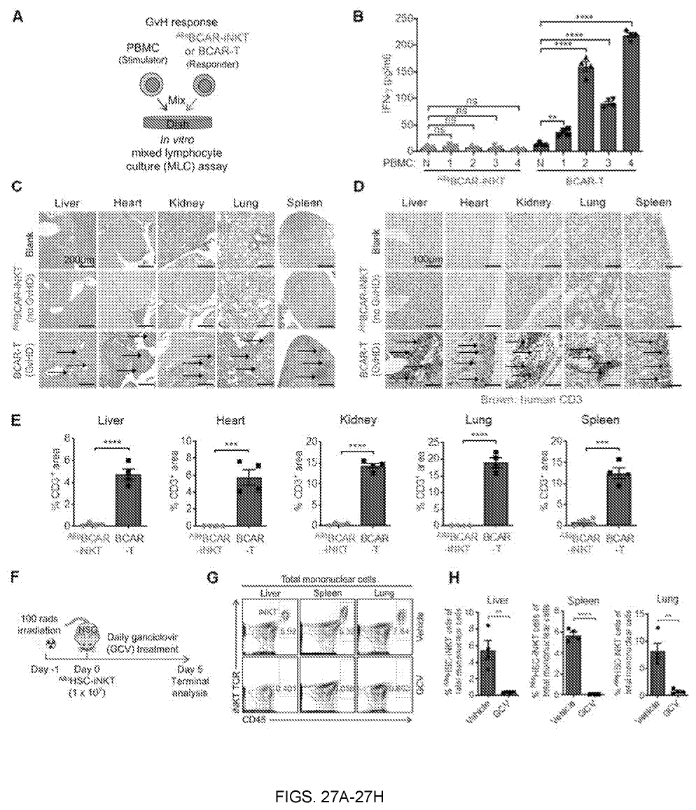

J. Lack of GvH Responses of ^{Allo}HSC-iNKT Cells (FIG. 27)

Since iNKT cells do not react with mismatched HLA molecules, they are not expected to cause GvHD (Haraguchi et al., 2004; de Lalla et al., 2011). The inventors studied the GvH responses using an established in vitro mixed lymphocyte culture (MLC) assay, which can be readout by IFN-γ production (FIG. 27A and FIG. 32C). As a result, both ^{Allo}HSC-iNKT and ^{Allo}BCAR-iNKT cells did not induce GvH response against multiple mismatched-donor PBMCs in contrast to conventional PBMC-Tc and BCAR-T cells, respectively (FIG. 27B and FIG. 32D).

In human MM xenograft NSG mice, although both ^{Allo}BCAR-iNKT and BCAR-T cells efficiently eradicated tumor, only ^{Allo}BCAR-iNKT treated mice showed long term survival (FIGS. 26K and 26L). Tissue analysis from tumor-bearing mice receiving ^{Allo}BCAR-iNKT cells, compared with those receiving BCAR-T cells, showed significantly less mononuclear cell infiltration into the tissues including the liver, heart, kidney, lung and spleen (FIGS. 27C and 27E). The infiltrates primarily consisted of human CD3⁺ T cells (FIG. 27D and FIG. 32A), indicating GvHD occurrence.

Pre-conditioned NSG mice were transplanted with ^{Allo}HSC-iNKT cells or donor-matched PBMC-Tc cells (FIG. 32E). Administration of ^{Allo}HSC-iNKT cells achieved long term survival (FIG. 32F) and lack of GvHD (FIGS. 32G and 32H) in comparison to mice transplanted with human PBMC-Tc cells. In previous work involving CAR19-iNKT anti-lymphoma activity, the lack of GvHD in iNKT-treated mice might be due to the absence of human myeloid cells and highly purified iNKT cells (Rotolo et al., 2018; Schroeder and DiPersio, 2011). Therefore, the inventors further tested the GvHD by transplanting pre-conditioned NSG mice with ^{Allo}HSC-iNKT cells mixed with T cell-depleted PBMC or donor-matched PBMC (FIG. 32I). As note, there was still no GvHD occurring in the mice injected with ^{Allo}HSC-iNKT mixed with myeloid cells (FIG. 32J). These results validated the therapeutic potential of ^{Allo}HSC-iNKT therapy and highlighted the remarkable safety profile of the proposed off-the-shelf cellular therapy.

K. Controlled Depletion of ^{Allo}HSC-iNKT Cells Via Ganciclovir (GCV) Treatment (FIG. 27)

To further enhance the safety profile of ^{Allo}HSC-iNKT cell products, the inventors incorporated a sr39TK suicide gene in the human iNKT TCR gene delivery vector, which allowed for the elimination of these cells through GCV-induced depletion. GCV, the guanosine analog, has been used in clinic as a prodrug to obtain a suicide effect in cellular products as a safety control in immunotherapy (Candolfi et al., 2009). In cell culture, GCV induced effective killing of ^{Allo}HSC-iNKT cells (FIG. 32B). In addition, an in vivo study was performed in NSG mice with i.v. injection of ^{Allo}HSC-iNKT and intraperitoneal (i.p.) injection of GCV for five consecutive days (FIG. 27F). The ^{Allo}HSC-iNKT cells were completely depleted by GCV treatment in liver, spleen and lung, as measured by flow cytometry (FIGS. 27G and 27H). Therefore, the ^{Allo}HSC-iNKT cellular product is equipped with a powerful “kill switch”, further elevating its safety profile.

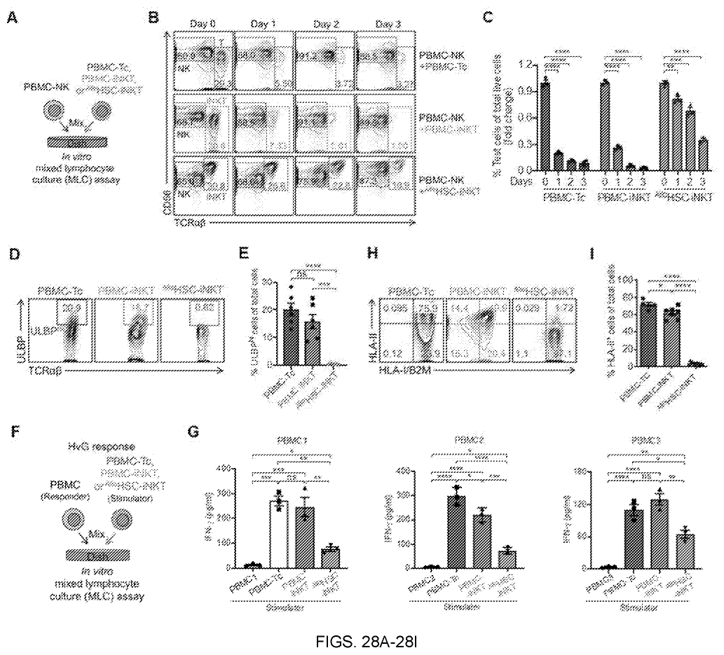

L. Naturally Low Immunogenicity of ^{Allo}HSC-iNKT Cells (FIG. 28)

For allogeneic cell therapies, one immunogenicity concern is host NK cell-mediated cytotoxicity (Braud et al., 1998; Torikai et al., 2013). The inventors utilized an in vitro MLC assay to study the NK cell killing to ^{Allo}HSC-iNKT cells (FIG. 28A). Interestingly, NK cells showed a strong resistance to allogeneic PBMC-Tc and PBMC-iNKT cells, but less killing to ^{Allo}HSC-iNKT cells (FIGS. 28B and 28C), which was likely due to the low expression of ULBP, a ligand for NK activating receptor NKG2D (Cosman et al., 2001), on ^{Allo}HSC-iNKT cells (FIGS. 28D and 28E).

HvG response is another huge immunogenicity concern for allogeneic cell therapy, mediated through elimination of allogeneic cells from host immune cells, mainly by conventional CD8 and CD4 T cells which recognize mismatched HLA-I and HLA-II molecules correspondingly (Ren et al., 2017; Steimle et al., 1994). In an in vitro MLC assay, in contrast to PBMC-Tc and PBMC-iNKT cells, ^{Allo}HSC-iNKT cells triggered less responses from PBMC from multiple mismatched donors (FIG. 28F, 28G, 28I). The low HvG response of ^{Allo}HSC-iNKT cells might be caused by their low MHC-I and MHC-II molecules expression (FIG. 28H-28J), which are in accordance to their RNAseq results (FIG. 24G).

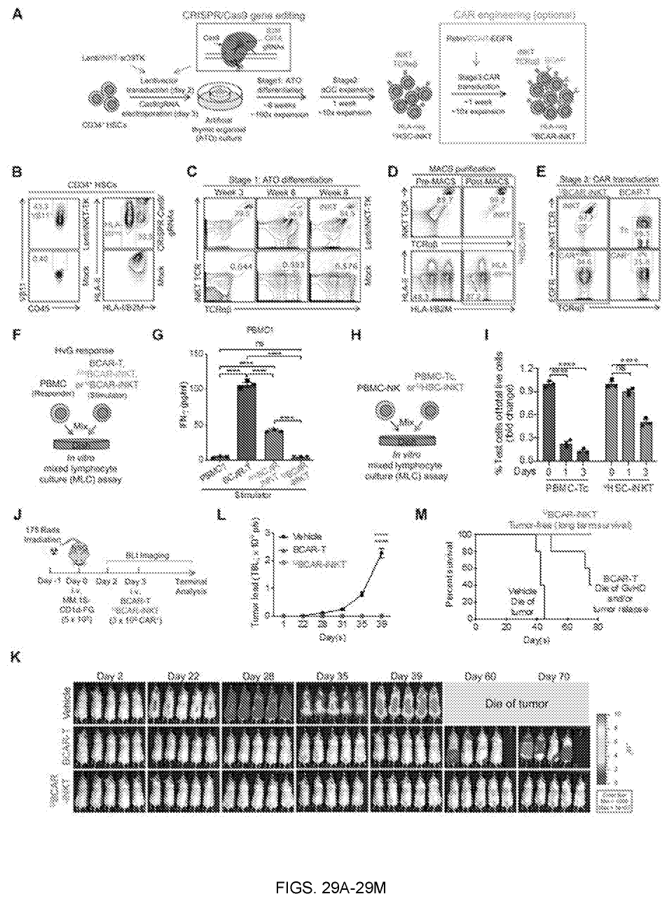

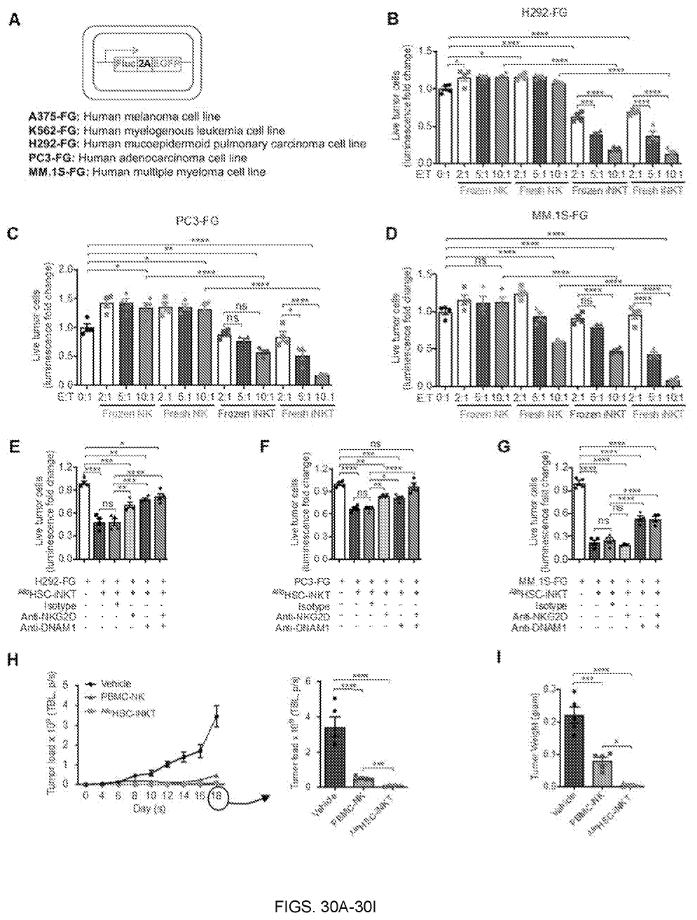

M. Generation of HLA-I/II-Negative Universal HSC-Engineered iNKT (^{U}HSC-iNKT) Cells (FIG. 29)

The availability of powerful gene-editing tools like CRISPR-Cas9/gRNA system enabled the genetically engineering of iNKT cells to make them resistant to host immune cell targeted depletion. The inventors knocked out the beta 2-microglobulin (B2M) gene to ablate HLA-I molecule expression on iNKT cells to avoid host CD8⁺ T cell-mediated killing (Ren et al., 2017); and the inventors knocked out CIITA gene to ablate HLA-II molecule to avoid host CD4⁺ T cell-mediated killing (Steimle et al., 1994). Both B2M and CIITA genes have been demonstrated as efficient and feasible targets for CRISPR-Cas9 system in human primary cells (Abrahimi et al., 2015).

CD34⁺ CB cells or G-CSF-mobilized human PBSCs transduced with lentiviral vector Lenti/iNKT-srTK was further engineered with CRISPR-Cas9/B2M-CIITA-gRNAs complex, which achieved ˜50-70% HLA-I/II double-deficiency rate (FIG. 29A). In stage 1 culture, gene-engineered HSCs were efficiently differentiated into human iNKT cells in ATO culture over 8 weeks with 100 times expansion (FIGS. 29B and 29C). In stage 2, iNKT cells were collected and expanded with αGC-loaded irradiated PBMCs (as APCs) for 1 week with 10 times expansion. A two-step MACS purification strategy was applied here to isolate HLA-I/II-negative universal HSC-engineered human iNKT cells (denoted as ^{U}HSC-iNKT cells) with over 97% purity (>99% iNKT cells, of which >97% are HLA-I/II-negative cells) FIG. 29D). The first step used MACS negative selection selecting against surface HLA-I/B2M and HLA-II molecules and the second step was a MACS positive selection selecting for surface iNKT TCR molecules. Additionally, ^{U}HSC-iNKT cells could be further engineered by transducing them with Retro/BCMA-CAR-tEGFR retroviral vector followed by IL-15 expansion for 1 weeks with 10 fold expansion, leading to HLA-I/II-negative universal BCMA CAR-engineered iNKT (denoted as ^{U}BCAR-iNKT cells) (FIGS. 29A and 29E).

N. The Phenotype, Functionality and Tumor Killing Efficacy of ^{U}HSC-iNKT and ^{U}BCAR-iNKT Cells

Flow cytometry analysis showed that ^{U}BCAR-iNKT displayed a typical iNKT cell phenotype similar to ^{Allo}HSC-iNKT and ^{Allo}BCAR-iNKT but distinct from BCAR-T cells. As expected, control BCAR-T cells expressed high levels of HLA-I and HLA-II molecules, while ^{U}BCAR-iNKT cells were double-negative, confirming their suitability for allogeneic therapy (FIG. 33A). Both ^{U}BCAR-iNKT and ^{Allo}BCAR-iNKT expressed mixed pattern of CD4 and CD8 co-receptors (CD4−CD8− and CD4−CD8+), expressed high levels of memory T cell marker CD45RO and NK cell marker CD161, and produced high levels of cytokines such as IFN-γ and cytotoxic molecules like perforin and granzyme B (FIG. 33A). In the in vitro tumor killing model of MM.1S-CD1d-FG, ^{U}BCAR-iNKT cells effectively killed MM tumor cells, at an efficacy comparable to that of conventional BCAR-T cells (FIG. 33G-33I). Importantly, in the presence of αGC, ^{U}BCAR-iNKT cells could deploy a stronger tumor killing through both CAR- and TCR-mediated targeting capacity (FIG. 33H). Therefore, HLA-I/II-depletion does not affect the development, phenotype and functionality of ^{U}HSC-iNKT and ^{U}BCAR-iNKT, making the manufacturing of the off-the-shelf cellular products possible. Meanwhile, the sr39TK suicide gene in the iNKT TCR gene delivery vector allowed the elimination of ^{U}BCAR-iNKT cells through GCV-induced depletion (FIG. 33D), ensuring safety profile of the cellular product.

O. Immunogenicity of ^{U}HSC-iNKT Cells (FIG. 29)

Next, the inventors tested the immunogenicity of ^{U}HSC-iNKT cells. For GvH response, the same as ^{Allo}HSC-iNKT cells, ^{U}HSC-iNKT cells did not induce GvH response, as supported by in vitro MLC assay (FIG. 33B-33D). For HvG response, As ^{U}HSC-iNKT cells engineered with CRISPR lack of surface HLA-I/II molecules, they are not expected to cause HvG responses, which the inventors verified in the in vitro MLC assay (FIG. 29F). In contrast to conventional BCAR-T and ^{Allo}BCAR-iNKT cells, ^{U}BCAR-iNKT cells triggered no response from responder PBMC T cells from multiple mismatched donors (FIG. 29G and FIG. 33E). These results strongly support ^{U}BCAR-iNKT cells to be the ideal candidate for off-the-shelf cellular therapy which are resistant to HvG response. For allogeneic NK response, the lack of HLA expression in the cell product may trigger the risk of rejection by the host NK cells (Braud et al., 1998; Torikai et al., 2013). However, the inventors did not detect such rejection during the co-culture of ^{U}HSC-iNKT cells with mismatched-donor NK cells (FIG. 29H, 29I and FIG. 33F), indicating the NK killing resistance of the inventors' cellular products.

P. In Vivo Antitumor Efficacy of ^{U}BCAR-iNKT Cells Against Hematologic Malignancies in a Human MM Xenograft Mouse Model

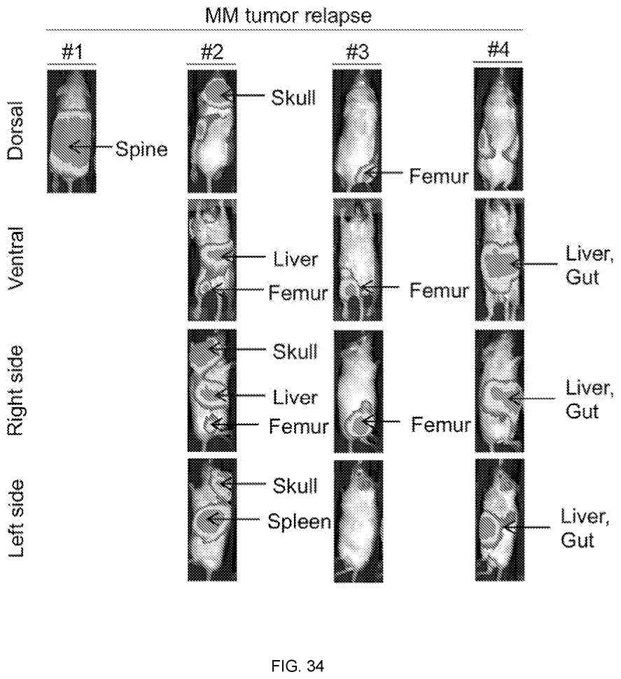

In vivo antitumor efficacy of ^{U}BCAR-iNKT cells was studied using a human MM xenograft NSG mouse model with the MM.1S-CD1d-FG cell line. The pre-conditioned mice were i.v. inoculated of MM.1S-CD1d-FG cells. After 3 days, effector cells, including ^{U}BCAR-iNKT and BCAR-T, were i.v. injected into the mice (FIG. 29J). Both ^{U}BCAR-iNKT and BCAR-T cells effectively eradicated pre-established metastatic MM tumor cells at the first 6 weeks (FIGS. 29L and 29K). However, mice receiving the conventional BCAR-T cells, eventually died because of either GvHD or tumor relapse (FIGS. 29K and 29M). The MM tumor relapse occurred at multiple organs, including spine, skull, femur, spleen, liver, and gut (FIG. 34). In contrast, mice receiving ^{U}BCAR-iNKT cells survived long-term without GvHD and tumor relapse in addition to being tumor free FIG. 29K-29M). These results demonstrated the safety profile and therapeutic potential of the ^{U}BCAR-iNKT-based cancer therapy.

Q. Experimental Model and Subject Details

1. Mice

NOD.Cg-Prkdc^{SCID}Il2rg^{tm1Wj1}/SzJ (NOD/SCID/IL-2Rγ−/−, NSG) mice were maintained in the animal facilities of the University of California, Los Angeles (UCLA). Six- to ten-week-old mice were used for all experiments unless otherwise indicated. All animal experiments were approved by the Institutional Animal Care and Use Committee of UCLA.

2. Cell Lines

The MS5-DLL4 murine bone marrow derived stromal cell line was obtained from Dr. Gay Crooks' lab in UCLA. Human multiple myeloma cancer cell line MM.1S, chronic myelogenous leukemia cancer cell line K562, melanoma cell line A375, lung carcinoma cell line H292, and prostate cancer cell line PC3 were purchased from American Type Culture Collection (ATCC). MM.1S cells were cultured in RPMI1640 supplemented with 10% (vol/vol) FBS and 1% (vol/vol) penicillin/streptomycin/glutamine (R10 medium). K562 cells were cultured in RPMI1640 supplemented with 10% (vol/vol) FBS, 1% (vol/vol) penicillin/streptomycin/glutamine, 1% (vol/vol) MEM NEAA, 10 mM HEPES, 1 mM sodium pyruvate and 50 uM β-ME (C10 medium). A375, H292 and PC3 were cultured in DMEM supplemented with 10% (vol/vol) FBS and 1% (vol/vol) penicillin/streptomycin/glutamine (D10 medium). Stable tumor cell lines for in vitro and in vivo analysis were made by transducing parental cell lines with lentiviral vector overexpressing human CD1d, human HLA-A2.1, human NY-ESO-1, and/or firefly luciferase and enhanced green fluorescence protein (see Star Methods).

3. Human CD34⁺ HSC and PBMC Cells

Cord blood cells were purchased from HemaCare (Los Angeles, USA). G-CSF-mobilized healthy donor peripheral blood cells were purchased from HemaCare or Cincinnati Children's Hospital Medical Center (CCHMC) (Los Angeles, USA). Human CD34⁺ HSCs were isolated through magnetic-activated cell sorting using ClinMACs CD34⁺ microbeads (Miltenyi Biotech, USA). Cells were cryopreserved in Cryostor CS10 (BioLife Solution, Seattle, Wash.) using CoolCell (BioCision, San Diego, Calif.), and were frozen in liquid nitrogen for all experiments and long-term storage. Healthy donor human peripheral blood mononuclear cells (PBMCs) were obtained from UCLA/CFAR Virology Core Laboratory.

4. Lentiviral/Retroviral Vectors and Transduction

The Lenti/iNKT vector and lentivirus was constructed and packaged as previously described (Zhu et al, 2019).

The Retro/BCAR-EGFR vector was constructed by inserting into the parental MP71 vector a synthetic gene encoding human BCMA scFV-41BB-CD3ζ-P2A-tEGFR. The synthetic gene fragments were obtained from IDT. Vsv-g-pseudotyped Retro/BCAR-EGFR retroviruses were generated by transfecting HEK 293T cells following a standard calcium precipitation protocol and an ultracentrifugation concentration protocol (Smith et al., 2016); the viruses were then used to transduce PG13 cells to generate a stable retroviral packaging cell line producing Retro/BCAR-EGFR retroviruses (denoted as PG13-BCAR-EGFR cell line). For retrovirus production, the PG13-BCAR-EGFR cells were seeded at a density of 0.8×10⁶ cells per ml in D10 medium, and cultured in a 15 cm-dish (30 ml per dish) for 2 days; virus supernatants were then harvested and stored at −80° C. for future use.

Healthy donor PBMCs or ^{Allo}HSC-iNKT cells were stimulated with CD3/CD28 T-activator beads (ThermoFisher Scientific) as instructed in the presence of recombinant human IL-2 (300 U/mL). On day 2, cells were spin-infected with frozen-thawed Retro/BCAR-EGFR retroviral supernatants supplemented with polybrene (10 μg/ml, Sigma-Aldrich) at 660 g at 30° C. for 90 min following an established protocol (Zhu et al., 2019). Retronectin (Takara) could be coated on plate one day before transduction to promote transduction efficiency. Transduced human T or ^{Allo}HSC-iNKT cells were expanded for another 7-10 days, and then were cryopreserved for future use. Mock-transduced human T or ^{Allo}HSC-iNKT cells were generated as controls. Transduction rate was determined by flow cytometry as percentage of EGFR⁺ cells.

5. Antibodies and Flow Cytometry

All flow cytometry stains were performed in PBS for 15 min at 4° C. The samples were stained with Fixable Viability Dye eFluor506 (e506) mixed with Mouse Fc Block (anti-mouse CD16/32) or Human Fc Receptor Blocking Solution (TrueStain FcX) prior to antibody staining. Antibody staining was performed at a dilution according to the manufacturer's instructions. Fluorochrome-conjugated antibodies specific for human CD45 (Clone H130), TCRaP (Clone I26), CD4 (Clone OKT4), CD8 (Clone SK1), CD45RO (Clone UCHL1), CD45RA (Clone HI100), CD161 (Clone HP-3G10), CD69 (Clone FN50), CD56 (Clone HCD56), CD62L (Clone DREG-56), CD14 (Clone HCD14), CD11b (Clone ICRF44), CD11c (Clone N418), CD1d (Clone 51.1), CCR4 (Clone L291H4), CCR5 (Clone HEK/1/85a), CXCR3 (Clone G025H7), NKG2D (Clone 1D11), DNAM-1 (Clone 11A8), CD158 (KIR2DL1/S1/S3/S5) (Clone HP-MA4), IFN-γ (Clone B27), granzyme B (Clone QA16A02), perforin (Clone dG9), TNF-α (Clone Mab11), IL-2 (Clone MQ1-17H12), HLAE (Clone 3D12), 02-microglobulin (B2M) (Clone 2M2), HLA-DR (Clone L243) were purchased from BioLegend; Fluorochrome-conjugated antibodies specific for human CD34 (Clone 581) and TCR Vα24-J18 (Clone 6B11) were purchased from BD Biosciences; Fluorochrome-conjugated antibodies specific for human Vβ11 was purchased from Beckman-Coulter. Human Fc Receptor Blocking Solution (TrueStain FcX) was purchased from Biolegend, and Mouse Fc Block (anti-mouse CD16/32) was purchased from BD Biosciences. Fixable Viability Dye e506 were purchased from Affymetrix eBioscience. Intracellular cytokines were stained using a Cell Fixation/Permeabilization Kit (BD Biosciences). Flow cytometry were performed using a MACSQuant Analyzer 10 flow cytometer (Miltenyi Biotech) and data analyzed with FlowJo software version 9.

6. ^{Allo}HSC-iNKT Cell Culture in Artificial Thymic Organoid

CD34⁺ HSC cells were transduced with lentivirus carrying iNKT-TCR vector in X-VIVO 15 Serum-free Hematopoietic Cell Medium supplemented with SCF (50 ng/ml), FLT3-L (50 ng/ml), TPO (50 ng/ml) and IL-3 (10 ng/ml) as described previously (Zhu et al., 2019). Artificial thymic organoid (ATO) was generated following previous established protocol (Montel-Hagen et al., 2019; Seet et al., 2017). MS5-DLL4 cells were harvest and resuspended in serum-free ATO culture medium, which was composed of RPMI 1640 (Corning), 1% penicillin/streptomycin (Gemini Bio-Products), 1% Glutamax (ThermoFisher Scientific), 4% B27 supplement (ThermoFisher Scientific), and 30 μM L-ascorbic acid 2-phosphate sesquimagnesium salt hydrate (Sigma-Aldrich) reconstituted in PBS. 1.5×10⁵ to 6×10⁵ MS5-DLL4 cells were mixed with 3×10³ to 1×10⁵ transduced HSCs per ATO aggregate in 1.5-ml microcentrifuge tubes and centrifuged at 300 g for 5 min at 4° C. Supernatants were carefully removed, and the cell pellet was resuspended in 6 μl ATO media and plated on a 0.4 μm Millicell transwell insert (EMD Millipore). ATO culture medium was supplemented with FLT3-L (Peprotech) and IL-7 (Peprotech) at a final concentration of 5 ng/ml, and was changed twice per week. ATO aggregates were harvested and homogenized by passage through a 50-μm nylon strainer (ThermoFisher Scientific) for further staining or expansion.

7. ^{Allo}HSC-iNKT Cell In Vitro Expansion

^{Allo}HSC-iNKT cells were harvested from ATO aggregates, processed into single mononuclear cells, and pooled together for in vitro culture. Healthy donor-derived PBMCs were loaded with αGC by culturing 1×10⁷ to 1×10⁸ PBMCs in 5 ml C10 medium containing 5 μg/ml αGC for 1 hour. αGC-loaded PBMCs were irradiated at 6,000 rads, and then mix with^{Allo}HSC-iNKT cells at ratio 1:1. These cells were cultured in C10 medium supplemented with human IL-7 (10 ng/ml) and IL-15 (10 ng/ml) for 10-14 days. ^{Allo}HSC-iNKT cells were expanded further with αGC-loaded PBMCs and IL-7/IL-15 for another 10-14 days, then were cryopreserved for future use.

8. PBMC-Derived Lymphoid Cell In Vitro Expansion

Healthy donor PBMCs were purchased from UCLA/CFAR Virology Core Laboratory, and were used to expand PBMC-Tc, PBMC-iNKT and PBMC-γδT cells. For PBMC-Tc cells, PBMCs were stimulated with CD3/CD28 T-activator beads (ThermoFisher Scientific) as instructed, cultured in C10 medium supplemented with human IL-2 (20 ng/mL) for 2-3 weeks. For PBMC-iNKT cells, iNKT cells were MACS-sorted from PBMCs using anti-iNKT microbeads (Miltenyi Biotech), then were co-cultured with donor matched irradiated αGC-loaded PBMCs at the ratio of 1:1 in C10 medium supplemented with human IL-7 (10 ng/ml) and IL-15 (10 ng/ml) for 2 weeks. For PBMC-γδT cells, PBMCs were cultured in C10 media supplemented with IL-2 (20 ng/ml) and Zoledronate (5 uM) (Sigma-Aldrich) for 2 weeks, and then were MACS-sorted using human TCRγ/δ T Cell Isolation Kit (Miltenyi Biotech).

9. TCR Repertoire Deep Sequencing

^{Allo}HSC-iNKT cells (6B11⁺TCRαβ⁺), PBMC-iNKT cells (6B11⁺TCRαβ⁺) and PBMC-Tc cells (6B11⁻TCRαβ⁺) were FACS-sorted. RNAs were directly extracted from sorted cells. cDNA library and deep sequencing was performed by UCLA TCGB (Technology Center for Genomics and Bioinformatics). Analysis of TCR a and R CDR3 regions was performed using 2×150 cycle setting with 5,000 reads/cell by 10× Genomics Chromium™ Controller Single Cell Sequencing System (10× Genomics).

10. Cell Phenotype and Functional Study

Phenotype and functionality of multiple types of cells were analyzed, including ^{Allo}HSC-iNKT, ^{Allo}BCAR-iNKT, and ^{U}BCAR-iNKT cells. Phenotype of these cells was studied using flow cytometry, by analyzing cell surface markers including co-receptors (CD4 and CD8), NK cell markers (CD161, NKG2D, DNAM-1, and KIR), memory T cell markers (CD45RO), and homing markers (CCR4, CCR5, and CXCR3). Capacity of cells to produce cytokines (IFN-γ, TNF-α and IL-2) and cytotoxic factors (perforin and granzyme B) were studied using Cell Fixation/Permeabilization Kit (BD Biosciences). PBMC-Tc, PBMC-NK, PBMC-iNKT or BCAR-T cells were included as FACS analysis controls.

Response of ^{Allo}HSC-iNKT cells to antigen stimulation was studied by culturing ^{Allo}HSC-iNKT cells in vitro in C10 medium for 7 days, in the presence or absence of αGC (100 ng/ml). Proliferation of ^{Allo}HSC-iNKT cells was measured by cell counting and flow cytometry (identified as 6B11⁺TCRαβ⁺) over time. Cytokine production was assessed by ELISA analysis of cell culture supernatants collected on day 3 (for human IFN-γ. TNF-α, IL-2, IL-4, IL-10 and IL-17).

11. Enzyme-Linked Immunosorbent Cytokine Assays (ELISA)

The ELISAs for detecting human cytokines were performed following a standard protocol from BD Biosciences. Supernatants from co-culture assays were collected and assayed to quantify IFN-γ, TNF-α, IL-2, IL-4, IL-10 and IL-17. The capture and biotinylated pairs for detecting cytokines were purchased from BD Biosciences. The streptavidin-HRP conjugate was purchased from Invitrogen. Human cytokine standards were purchased from eBioscience. Tetramethylbenzidine (TMB) substrate was purchased from KPL. The samples were analyzed for absorbance at 450 nm using an Infinite M1000 microplate reader (Tecan).

12. RNA Sequencing (RNA-seq) and Data Analysis

PBSC-derived ^{Allo}HSC-iNKT, CB-derived ^{Allo}HSC-iNKT, PBMC-iNKT (CD8⁺), PBMC-αβTc (CD8⁺), PBMC-NK, and PBMC-Tγδ cells were FACS-sorted. All the samples were chosen from 2-8 independent experiments from different donors. Total RNA was isolated from these cells by using miRNeasy Mini Kit (QIAGEN). RNA concentration was measured using Nanodrop 2000 spectrophotometer (Thermal Scientific).

| Name of Cell | Number of | | |
|---|---|---|---|
| Population | replicates (n) | Phenotype | Description |
| ^{Allo}HSC-iNKT | 3 | 6B11⁺TCRαβ⁺ | Allogeneic PBSC-engineered human |
| (from PBSC) | | | iNKT cells |
| ^{Allo}HSC-iNKT | 3 | 6B11⁺TCRαβ⁺ | Allogeneic CB cell-engineered human |
| (from CB) | | | iNKT cells |
| PBMC-iNKT | 3 | 6B11⁺TCRαβ⁺CD8⁺ | Cells isolated from healthy donor |
| (CD8⁺) | | | PBMCs, stimulated by αGC-pulsed |
| | | | APCs, and sorted CD8⁺ by flow |
| PBMC-αβTc | 8 | 6B11⁻TCRαβ⁺CD8⁺ | Cells isolated from healthy donor |
| (CD8⁺) | | | PBMCs, stimulated by CD3/CD28 T- |
| | | | Activator beads, and sorted CD8⁺ by |
| | | | flow |
| PBMC-NK | 2 | CD56⁺TCRαβ⁻ | Cells collected from healthy donoe |
| | | | PBMCs, sorted CD56⁺ by flow |
| PBMC-γδT | 6 | TCRγδ⁺TCRαβ⁻ | Cells isolated from healthy donor |
| | | | PBMCs, stimulated by Zoledronate, |
| | | | and sorted TCRγδ⁺ by flow |

cDNA library construction and deep sequencing were performed by UCLA TCGB (Technology Center for Genomics and Bioinformatics). Single-Read 50 bp sequencing was performed on Illumina Hiseq 3000. A total of 25 libraries were multiplexed and sequenced in 3 lanes. Raw sequence files were obtained, and quality checked using Illumina's proprietary software, and are available at NCBI's Gene Expression Omnibus.

13. In Vitro Tumor Killing Assay

A375-FG, K562-FG, PC3-FG, MM.1S-FG, or H292-FG tumor cells (lx 10⁴ cells per well) were co-cultured with ^{Allo}HSC-iNKT cells at certain ratios (indicated in figure legends) in Corning 96-well clear bottom black plates in C10 medium for 24 hours. Freshly sorted or cryopreserved PBMC-NK cells were included as controls. MM.1S-CD1d-FG tumor cells (lx 10⁴ cells per well) were co-cultured with ^{Allo}BCAR-iNKT or ^{U}BCAR-iNKT cells at certain ratios (indicated in figure legends) in Corning 96-well clear bottom black plates for 8-24 hours, in C10 medium with or without αGC (100 ng/ml). PBMC-T and BCAR-T cells were included as controls. At the end of culture, live tumor cells were detected by adding D-luciferin (150 μg/ml) (Caliper Life Science) to cell cultures and reading out luciferase activities using an Infinite M1000 microplate reader (Tecan). In the antibody blocking assay, 10 ug/ml of LEAF™ purified anti-human NKG2D (Clone 1D11, Biolegend), anti-human DNAM-1 antibody (Clone 11A8, Biolegend), or LEAF™ purified mouse lgG2bk isotype control antibody (Clone MG2B-57, Biolegend) was added to tumor cell cultures one hour prior to adding effector cells.

14. ^{Allo}HSC-iNKT Cell In Vivo Anti-tumor Efficacy Study in Human Melanoma Xenograft NSG Mouse Model

NSG mice (6-10 weeks of age) were pre-conditioned with 100 rads of total body irradiation (day −1), and then inoculated with 1×10⁶ A375-FG cells subcutaneously (day 0). On day 2, mice were imaged by BLI and randomized into different groups. Three days post-tumor inoculation (day 3), the mice were i.v. injected vehicle (PBS), 1.2×10^{7 Allo}HSC-iNKT cells, or 1.2×10⁷ PBMC-NK cells. Over time, tumor loads were monitored by total body luminescence using BLI and tumor size measurement using a Fisherbrand™ Traceable™ digital caliper (Thermo Fisher Scientific). The tumor size was calculated as W×L mm². At approximately week 3, mice were terminated for analysis, and solid tumors were retrieved and weighed using a PA84 precision balance (Ohaus).

15. Bioluminescence Live Animal Imaging (BLI)

Before imaging, mice were anesthetized with 2% isoflurane (Zoetis UK)/medical oxygen. All mice received a single intraperitoneal injection of D-luciferin (1 mg per mouse) in PBS for 5 min before scanning. BLI was performed using an IVIS 100 imaging system (Xenogen/PerkinElmer). Imaging results were analyzed using a Living Imaging 2.50 software (Xenogen/PerkinElme).

16. ^{Allo}BCAR-iNKT Cell In Vivo Anti-Tumor Efficacy Study in Human MM Xenograft NSG Mouse Model

NSG mice were pre-conditioned with 175 rads of total body irradiation (day −1), and then inoculated with 1×10⁶ MM-CD1d-FGFP cells intravenously (day 0). On day 2, mice were imaged by BLI and randomized into different groups. Three days post-tumor inoculation (day 3), mice received i.v. injection of vehicle (PBS), 7×10^{6 Allo}BCAR-iNKT cells, or 7×10⁶ conventional BCAR-T cells. Tumor were monitored by BLI. Survival curve was recorded when the mice died of tumor or GvHD.

17. Ganciclovir (GCV) In Vitro and In Vivo Killing Assay

^{Allo}HSC-iNKT cells were cultured in C10 medium. Titrated amount of GCV (0-50 μM) were added into the cell culture. After 4 days, live ^{Allo}HSC-iNKT cells were counted. GCV in vivo killing assay were performed on NSG mice. Experimental mice were i.v. injected with 10×10^{6 Allo}HSC-iNKT cells and received i.p. injection of GCV for 5 consecutive days (50 mg/kg per injection per day) before humanely euthanization. Spleen, liver, and lung were collected, homogenized and processed into single mononuclear cell suspension by filtering through 70 uM cell strainer (Fisher Scientific). Cells from liver and lung were resuspended in 33% Percoll in PBS at room temperature (RT), and spun at 800 g for 30 min with no brake at RT. Then the pellet cells were resuspended in TAC buffer at RT for 15-20 min to lysis of the red blood cells. Cells from spleen were directly resuspended in TAC buffer. After that, the cells were spun and resuspended in C10 and ready for staining. ^{Allo}HSC-iNKT cells were detected by flow cytometry (identified as CD45⁺6B11⁺ cells).

18. Histologic Analysis

Heart, liver, kidney, lung and spleen tissues collected from the experimental mice were fixed in 10% Neutral Buffered Formalin for up to 36 hours and embedded in paraffin for sectioning (5 μm thickness). Tissue sections were stained either with Hematoxylin and Eosin or anti-human CD3 primary antibodies following standard procedures by UCLA Translational Pathology Core Laboratory. Stained sections were imaged using an Olympus BX51 upright microscope equipped with an Optronics Macrofire CCD camera (AU Optronics) at 20× and 40× magnifications. The images were analyzed using Optronics PictureFrame software (AU Optronics).

19. Electroporation

CD34⁺ HSCs were spun at 90×g for 10 minutes and then resuspended in 20 μl P3 solution (Lonza, Basel, Switzerland). 1 μl gRNA (100 μM) and 4 μl Cas9 (6.5 mg/ml) were added to each sample per reaction. Cells were added in the cuvette and electroporated using the Amaxa 4D Nucleofector X Unit (Lonza, Basel, Switzerland) under ER-100 program. Cells were rested at RM for 10 minutes after electroporation and then transferred to a 24-well tissue culture treated plate overnight before ATO culture.

20. In Vitro Mixed Lymphocyte Culture (MLC) Assay

To test GvH response, PBMCs (as stimulators) from different donors were irradiated with 2500 rads, seeded in 96-well plate (5×10⁵ cells/well) in C10 medium, and co-cultured with ^{Allo}BCAR-iNKT or ^{U}BCAR-iNKT cells (2×10⁴ cells/well) (as responders). BCAR-T cells were included as a responder control. After 4 days, cell culture supernatants were collected, and IFN-γ was measured using ELISA.

To test HvG response, PBMCs (as responders) from different donors were seeded in 96-well plates (2×10⁴ cells/well) in C10 medium, and co-cultured with 2500-rad irradiated ^{Allo}BCAR-iNKT or ^{U}BCAR-iNKT cells (5×10⁵ cells/well) (as stimulators). PBMC-Tc, PBMC-iNKT and BCAR-T were included as stimulator control. After 4 days, cell culture supernatants were collected, and IFN-γ was measured using ELISA.

To test allogeneic NK cytotoxicity, donor-mismatched PBMC-NK were collected and seeded in 96-well plate (2×10⁴ cells/well) in C10 medium, and co-cultured with ^{Allo}HSC-iNKT or ^{U}HSC-iNKT (2×10⁴ cells/well) cells. PBMC-Tc and PBMC-iNKT cells were included as controls. Flow cytometry was used to detect the cell numbers at indicated days.

21. Statistical Analysis

GraphPad Prism 6 (Graphpad Software) was used for statistical data analysis. Student's two-tailed t test was used for pairwise comparisons. Ordinary 1-way ANOVA followed by Tukey's multiple comparisons test was used for multiple comparisons. Log rank (Mantel-Cox) test adjusted for multiple comparisons was used for Meier survival curves analysis. Data are presented as mean±SEM, unless otherwise indicated. In all figures and figure legends, “n” represents the number of samples or animals utilized in the indicated experiments. A P value of less than 0.05 was considered significant. ns, not significant; *P<0.05; **P<0.01; ***P<0.001; ****P<0.0001.

### Example 4: A Feeder-Free Ex Vivo Differentiation Culture Method to Generate Off-the-Shelf Monoclonal iNKT TCR-Armed Gene-Engineered T (iTARGET) Cells

Invariant natural killer T (iNKT) cells are a small subpopulation of αβ T lymphocytes with the ability to bridge innate and adaptive immunity. Unlike the conventional αβ T cells, the T cell receptor (TCR) of iNKT cells recognizes lipid antigens presented by CD1d, a major histocompatibility complex (MHC)-like molecule, instead of MHC itself. Because of this unique property, iNKT cells do not cause graft-versus-host disease (GvHD) when transplanted allogeneically. Additionally, iNKT cells have several other unique features that make them ideal cellular carriers for developing off-the-shelf cellular therapy for cancer: 1) they have roles in cancer immune surveillance; 2) they have the remarkable capacity to target tumors independent of tumor antigen- and major histocompatibility complex (MHC)-restrictions; 3) they can employ multiple mechanisms to attack tumor cells through direct killing and adjuvant effects. However, the development of an allogeneic off-the-shelf iNKT cellular product is greatly hindered by their availability—these cells are of extremely low number and high variability in humans (˜0.001-1% in human blood), making it very difficult to produce therapeutic numbers of iNKT cells from blood cells of allogeneic human donors.

Two prior methods have been used to generate enough iNKT cells for therapeutic uses. One method is to screen large numbers of donors and find “super donors” who naturally have high percentage of iNKT cells in peripheral blood. iNKT cells are enriched by the magnetic bead-based purification procedure and then expanded by either anti-CD3/CD28 bead stimulation or co-culture with antigen-presenting cells loaded with alpha-galactosylceramide (αGC). Although expansion can be achieved by this method, the expansion fold is limited, and the expansion is unreliable. Another method is based on the genetic modification of hematopoietic stem cells (HSCs) with iNKT TCRs followed by an artificial thymic organoid (ATO) culture system that supports the in vitro differentiation of human HSCs into iNKT cells. Although this method can generate iNKT cells with high yield, the production requires the use of feeder cells of mouse origin, which poses significant challenges to develop a reliable process for GMP-compatible manufacturing.

A novel method that can reliably generate a homogenous monoclonal population of iNKT cells at large quantities with a feeder-free differentiation system is thus pivotal to developing an off-the-shelf iNKT cell therapy.

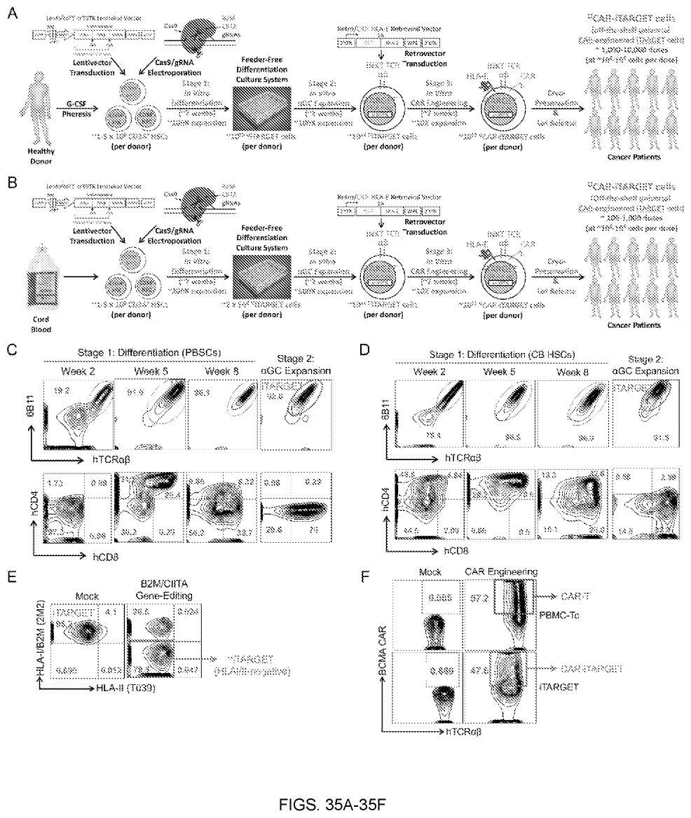

A. CMC Study—iTARGET, UiTARGET, and CAR-iTARGET Cells (FIG. 35)

HSCs from G-CSF-mobilized peripheral blood HSCs (PBSCs) or cord blood (CB HSCs) were transduced with a Lenti/iNKT-sr39TK vector that encoded a human iNKT TCR gene as well as a suicide/PET imaging gene, then put into the feeder-free ex vivo TARGET cell culture to generate iNKT TCR-Armed Gene-Engineered T (iTARGET) cells (FIGS. 35A and 35B). Both PBSCs and CB HSCs can effectively differentiate into and expand as monoclonal iTARGET cells (FIGS. 35C and 35D), that could be further engineered to be deficient of both HLA-I/II resulting in Universal iTARGET (UiTARGET) cells (FIG. 35E), and could be further engineered to express CAR resulting in CAR-iTARGET cells (FIG. 35F). It is estimated that ˜10¹² scale of UCAR-iTARGET cells can be produced from PBSCs of a healthy donor, which can be formulated into 1,000-10,000 doses (at ˜10⁸-10⁹ cells per dose); and that ˜10¹¹ scale of UCAR-iTARGET cells can be produced from HSCs of a CB sample, which can be formulated into 100-1,000 doses (FIGS. 35A and 35B). Despite the difference in cell yields, iTARGET cells and their derivatives generated from PBSCs and CB HSCs displayed similar phenotype and functionality. Unless otherwise indicated, CB HSC-derived iTARGET cells and their derivatives were utilized for the proof-of-principle studies described below.

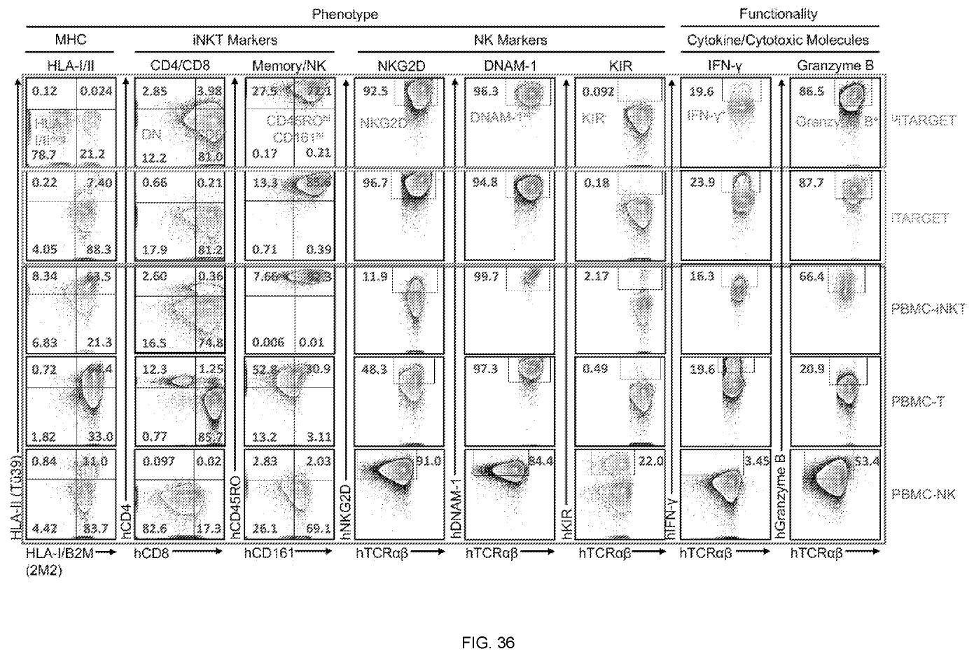

B. Pharmacology Study—iTARGET and ^{U}iTARGET Cells (FIG. 36)

The phenotype and functionality of iTARGET and ^{U}iTARGET (HLA-I/II-negative iTARGET) cells were studied using flow cytometry (FIG. 36). Three controls were included: 1) native human iNKT cells that were isolated from healthy donor peripheral blood and expanded in vitro with αGC stimulation, identified as hTCRαβ⁺6B11⁺ and denoted as PBMC-iNKT cells; 2) native human conventional αβ T cells that were isolated from healthy donor peripheral blood and expanded in vitro with anti-CD3/CD28 stimulation, identified as hTCRαβ⁺6B11⁻ and denoted as PBMC-T cells; and 3) native human NK cells that were isolated from healthy donor peripheral blood, identified as hTCRαβ⁻hCD56⁺ and denoted as PBMC-NK cells.

As expected, all three types of native human immune cells (PBMC-iNKT, PBMC-T, and PBMC-NK cells) expressed homogenously high levels of HLA-I molecules and mixed high/low levels of HLA-II molecules, while ^{U}iTARGET cells were dominantly double-negative (>70%), confirming their suitability for allogeneic therapy (FIG. 36, left panels). Interestingly, even without B2M/CIITA gene-editing, iTARGET cells already expressed low levels of HLA-II molecules, suggesting that these cells are naturally of low immunogenicity compared to native human iNKT/T/NK cells (FIG. 36, left panels). Nonetheless, HLA-II expression could be further reduced by CIITA gene-editing (in ^{U}iTARGET cells).

Both ^{U}iTARGET and iTARGET cells displayed typical human iNKT cell phenotype and functionality: they expressed the CD4 and CD8 co-receptors with a mixed pattern (CD4/CD8 double-negative and CD8 single-positive); they expressed high levels of memory T cell marker CD45RO and NK cell marker CD161; and they produced exceedingly high levels of multiple effector cytokines (like IFN-7) and cytotoxic molecules (like perforin and Granzyme B), resembling that of native iNKT cells (FIG. 36). Interestingly, ^{U}iTARGET and iTARGET cells expressed some NK activation receptors (NKG2D) at levels higher than that of native iNKT and NK cells; meanwhile, these cells did not express inhibitory NK receptors (KIR), very different from native iNKT and NK cells (FIG. 36). These results suggest that ^{U}iTARGET and iTARGET cells may have enhanced NK-path tumor killing capacity stronger than that of native iNKT and even native NK cells. Importantly, HLA-I/II-deficiency does not interfere with either the development or phenotype/functionality of ^{U}iTARGET cells, making the manufacturing of this off-the-shelf cellular product possible.

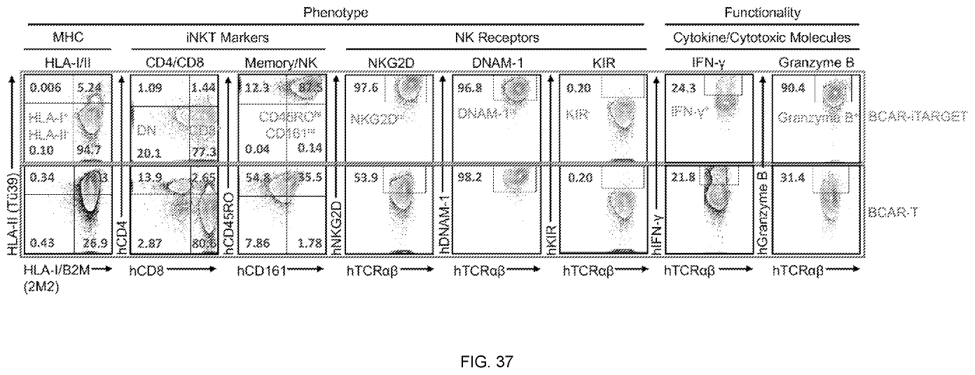

C. Pharmacology Study—CAR-iTARGET Cells (FIG. 37)

The phenotype and functionality of BCMA CAR-engineered iTARGET (BCAR-iTARGET) cells were studied using flow cytometry (FIG. 37). BCMA CAR-engineered conventional αβ T (BCAR-T) cells generated through BCMA CAR-engineering of healthy donor peripheral blood T cells were included as a control.

As expected, control BCAR-T cells expressed high levels of HLA-I and HLA-II molecules. Interestingly, BCAR-iTARGET cells expressed low levels of HLA-II molecules, suggesting that these cells are naturally of low immunogenicity compared to conventional BCAR-T cells (FIG. 37, left panels). BCAR-iTARGET cells displayed typical human iNKT cell phenotype and functionality: they expressed the CD4 and CD8 co-receptors with a mixed pattern (CD4/CD8 double-negative and CD8 single-positive); they expressed high levels of memory T cell marker CD45RO and NK cell marker CD161; and they produced high levels of effector cytokines like IFN-γ and cytotoxic molecules like Granzyme B comparable to or better than their counterpart conventional BCAR-T cells.

Interestingly, BCAR-iTARGET cells expressed exceedingly high levels of certain NK activation receptors like NKG2D, suggesting that BCAR-iTARGET cells may kill tumor cells through both CAR-mediated and NK receptor-mediated pathways.

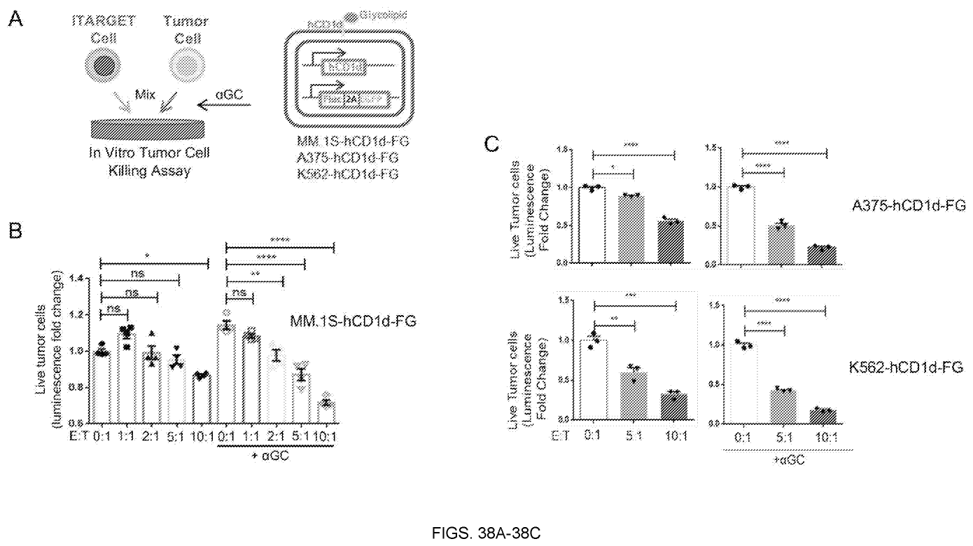

D. In Vitro Efficacy and MOA Study—iTARGET Cells (FIG. 38)

Even without being engineered to express additional tumor-targeting molecules like Chimeric Antigen Receptors (CARs) and T Cell Receptors (TCRs), iTARGET cells should be able to target tumor cells through iNKT TCR-mediated and NK receptor-mediated pathways. The inventors established an in vitro tumor cell killing assay to study such tumor killing capacities (FIG. 38A). Various human tumor cell lines were engineered to overexpress human CD1d as well as the firefly luciferase (Fluc) and enhanced green fluorescence protein (EGFP) dual reporters. Expression of human CD1d is to enable the tumor cells to present iNKT TCR cognate glycolipid antigens, such as endogenous tumor lipid antigens or synthetic lipid antigens like αGC. Expression of Flue and EGFP facilitate the detection of tumor cell killing using sensitive luciferase activity assay and flow cytometry assay. Three engineered human tumor cell lines were used in this study, including a human multiple myeloma (MM) cell line MM.1S-hCD1d-FG, a human melanoma cell line A375-hCD1d-FG, and a human chronic myelogenous leukemia cancer cell line K562-hCD1d-FG (FIG. 38A). iTARGET cells effectively killed MM, A375, and K562 tumor cells in the absence of αGC stimulation; tumor killing efficacy was further enhanced in the presence of αGC stimulation (FIGS. 38B and 38C).

These results proved the tumor killing capacity of iTARGET cells through an iNKT TCR/CD1d/lipid antigen-dependent mechanism, or through an antigen-independent NK path-mediated mechanism.

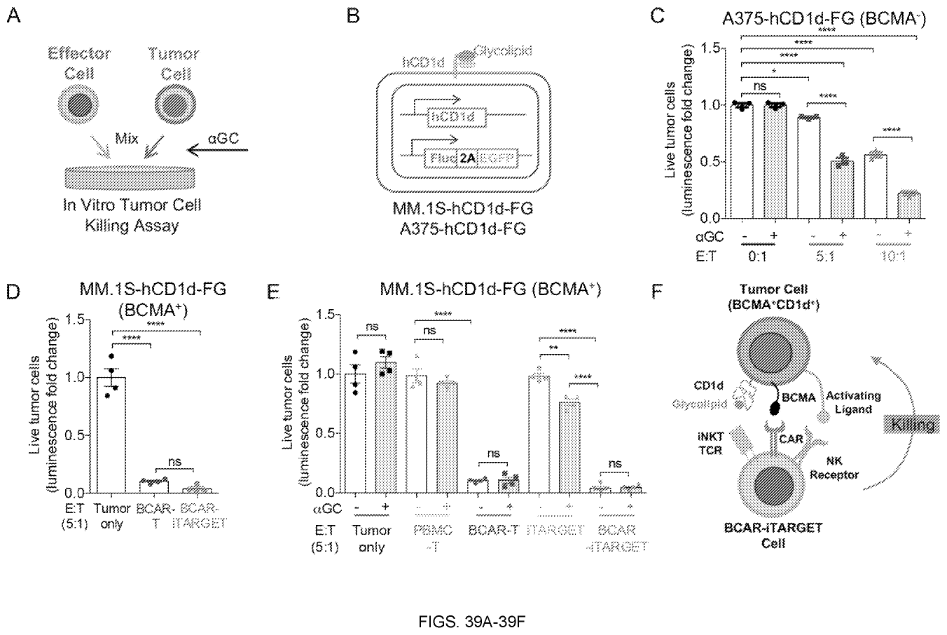

E. In Vitro Efficacy and MOA Study—CAR-iTARGET Cells (FIG. 39)

The inventors established an in vitro tumor cell killing assay for this study (FIG. 39A). BCMA CAR-engineered iTARGET (BCAR-iTARGET) cells were studied as the effector cells. Two human tumor cell lines were included in this study: 1) a human MM cell line, MM.1S, that were BCMA+ and served as a target of CAR-mediated killing; and 2) a human melanoma cell line, A375, that were BCMA- and served as a negative control target of CAR-mediated killing. Both human tumor cell lines were engineered to overexpress human CD1d as well as the firefly luciferase (Fluc) and enhanced green fluorescence protein (EGFP) dual reporters (FIG. 39B). Expression of human CD1d enabled the tumor cells to present iNKT TCR cognate glycolipid antigens, such as endogenous tumor lipid antigens or synthetic lipid antigens like αGC, making the CD1d+ tumor cells susceptible to iNKT TCR/CD1d/glycoantigen-mediated tumor killing pathway. Expression of Flue and EGFP facilitate the detection of tumor cell killing using sensitive luciferase activity assay and flow cytometry assay. The resulting MM.1S-hCD1d-FG and A375-hCD1d-FG cell lines were then utilized in the BCAR-iTARGET cells killed A375-hCD1d-FG tumor cells at certain efficacy, presumably through an NK killing path; tumor killing efficacy was further enhanced in the presence of αGC, likely through the addition of a TCR/CD1d/αGC killing path (FIG. 39C). Therefore, CAR-iTARGET cells can target tumor through CAR-independent mechanisms.

BCAR-iTARGET cells effectively killed MM.1S-hCD1d-FG tumor cells, at an efficacy comparable to or better than that of conventional BCAR-T cells (FIG. 39D). Importantly, in the presence of a cognate lipid antigen ((αGC), iTARGET cells, but not conventional PBMC-T cells, demonstrated enhanced tumor-killing efficacy, likely because of the activation of a TCR/CD1d/αGC tumor killing path (FIG. 39E). Note that in this study, BCAR-iTARGET cells already exhibited maximal tumor killing in the absence of αGC, making it difficult to study possible tumor killing enhancement after αGC addition (FIG. 39E). The synergistic tumor killing effects can be studied under conditions wherein CAR-mediated tumor killing is suboptimal.

Taken together, these results indicate that CAR-iTARGET cells can target tumor using three mechanisms: 1) CAR-dependent path, 2) iNKT TCR-dependent path, and 3) NK path (FIG. 39F). This unique triple-targeting capacity of CAR-iTARGET cells is attractive, because it can potentially circumvent antigen escape, a phenomenon that has been reported in autologous CAR-T therapy clinical trials wherein tumor cells down-regulated their expression of CAR-targeting antigen to escape attack from CAR-T cells.

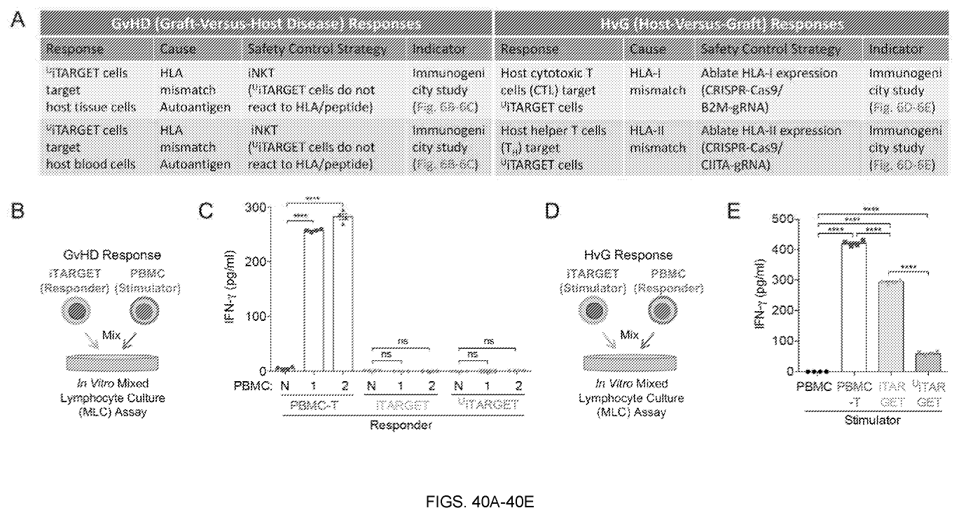

F. Immunogenicity Study—iTARGET and ^{U}iTARGET Cells (FIG. 40)

For allogeneic cell therapies, there are two immunogenicity concerns: a) GvHD responses, and b) host-versus-graft (HvG) responses. The inventors have considered the possible GvHD and HvG risks for the intended ^{U}iTARGET cellular product, and evaluated the engineered mitigation and safety control strategies (FIG. 40A). iTARGET cells were also included in the study.

GvHD is the major safety concern. However, because iNKT cells do not react to mismatched HLA molecules and protein autoantigens, they are not expected to induce GvHD¹² This notion is evidenced by the lack of GvHD in human clinical experiences in allogeneic HSC transfer and autologous iNKT transfer^{10,11}, and is supported by the inventors' in vitro mixed lymphocyte culture (MLC) assay (FIGS. 40B and 40C). Note that neither iTARGET nor ^{U}iTARGET cells responded to allogenic PBMCs, in sharp contrast to that of the conventional PBMC-T cells (FIGS. 40B and 40C).

On the other hand, HvG risk is largely an efficacy concern, mediated through elimination of allogeneic therapeutic cells by host immune cells, mainly by conventional CD8 and CD4 T cells which recognize mismatched HLA-I and HLA-II molecules. ^{U}iTARGET cells are engineered with B2M/CIITA gene-editing to ablate their surface display of HLA-I/II molecules and therefore are expected not to induce host T cell-mediated responses (FIG. 36 and FIG. 40A). Indeed, in an In Vitro MLC assay, in contrast to the conventional PBMC-T cells and the iTARGET cells, ^{U}iTARGET cells triggered significantly reduced responses from PBMC T cells from multiple mismatched donors (FIGS. 40D and 40E). Note that compared to conventional PBMC-T cells, iTARGET cells already showed reduced immunogenicity, likely because of their expression of very low levels of HLA-II molecules (FIG. 36). Also note that the ^{U}iTARGET cell product used in this study did not go through a purification step and therefore still contained ˜20% HLA-I⁺HLA-II^{lo} cell population (FIG. 36). The purity of HLA-I/II-negative ^{U}iTARGET cells can be conveniently enriched through MACS negative selection against cell surface HLA-I/B2M (by a 2M2 monoclonal antibody recognizing B2M) and HLA-II (by a Tü39 monoclonal antibody recognizing HLA-DR, DP, DQ) molecules, resulting in a highly pure and homogeneous cell product (>95% hTCRαβ⁺6B11⁺HLA-I/II⁻ cells). The purified ^{U}iTARGET cell product are expected to fully resist host T cell (both CD4⁺ and CD8⁺ conventional T cell)-mediated depletion in allogenic recipients. Lack of surface HLA-I expression may make ^{U}iTARGET cells susceptible to host NK cell-mediated depletion, that can be mitigated by further engineering the ^{U}iTARGET cells to overexpress HLA-E (FIGS. 35A and 35B).

Taken together, these results strongly support ^{U}iTARGET cells as an ideal candidate for off-the-shelf cellular therapy that are GvHD-free and HvG-resistant.

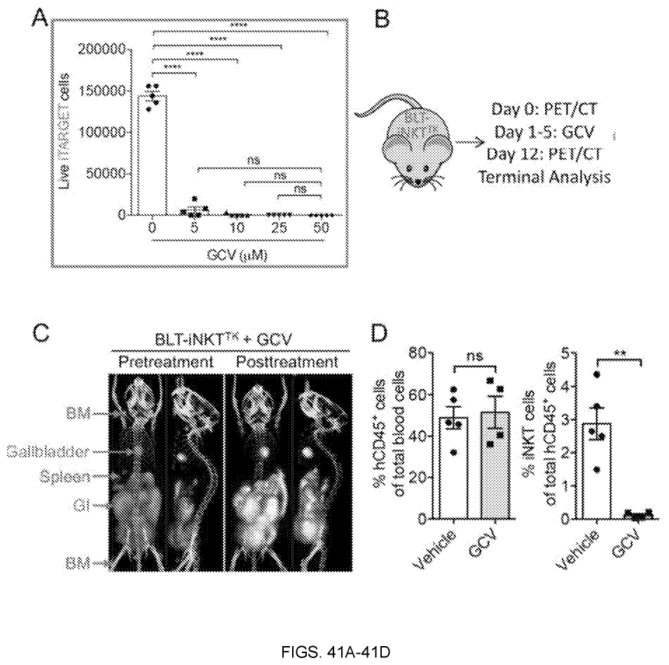

G. Safety Study—sr39TK Gene for PET Imaging and Safety Control (iTARGET Cells) (FIG. 41)

To further enhance the safety profile of iTARGET cellular products, the inventors have engineered an sr39TK PET imaging/suicide gene in iTARGET cells, which allows for the in vivo monitoring of these cells using PET imaging and the elimination of these cells through GCV-induced depletion in case of a serious adverse event (FIGS. 35A and 35B). In cell culture, GCV induced effective killing of iTARGET cells (FIG. 41A). A pilot in vivo study was performed using BLT-iNKT^{TK} humanized mice harboring human HSC-engineered iNKT (HSC-iNKT^{BLT}) cells (FIG. 41B). The HSC-iNKT^{BLT} cells were engineered from human HSCs transduced with a Lenti/iNKT-sr39TK lentiviral vector, the same vector used for engineering the iTARGET cellular products in the proof-of-principle study. Using PET imaging combined with CT scan, the inventors detected the distribution of gene-engineered human cells across the lymphoid tissues of BLT-iNKT^{TK} mice, particularly in bone marrow (BM) and spleen (FIG. 41C). Treating BLT-iNKT^{TK}mice with GCV effectively depleted gene-engineered human cells across the body (FIG. 41C). Importantly, the GCV-induced depletion was specific, as evidenced by the selective depletion of the HSC-engineered human iNKT cells but not other human immune cells in BLT-iNKT^{TK} mice as measured by flow cytometry (FIG. 41D). Therefore, the iTARGET cellular products are equipped with a powerful “kill switch”, further enhancing their safety profiles.

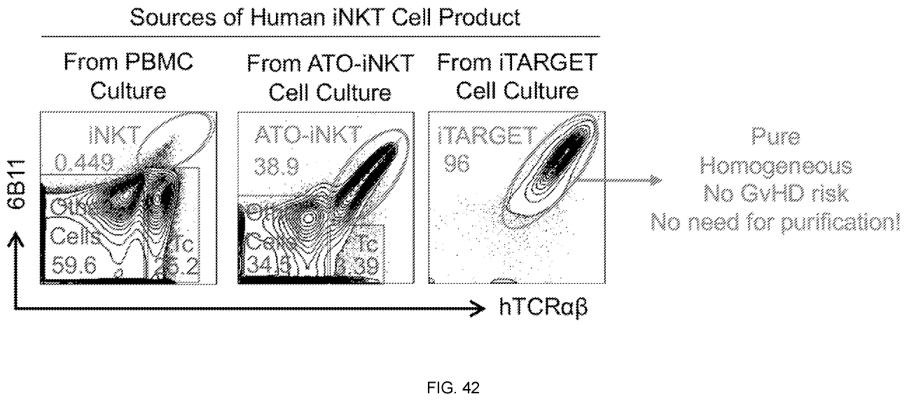

H. Comparison Study—Unique Properties of iTARGET Cell Product (FIG. 42)

Existing methods generating human iNKT cell products include expanding human iNKT cells from human PBMC cell cultures, from Artificial Thymic Organoid (ATO) cultures, and from other sources (FIG. 42). All these culture methods start from a mixed cell population containing human iNKT cells as well as other cells, in particular heterogeneous conventional αβ T (Tc) cells that may cause GvHD when transferred into allogeneic recipients (FIG. 42). As a result, these pre-existing methods require a purification step to make “off-the-shelf” iNKT cell products, to avoid GvHD. The iTARGET cell culture is unique in two aspects: 1) It does not support TCR V/D/J recombination to produce randomly rearranged endogenous TCRs, thereby no GvHD risk; 2) It supports the synchronized differentiation of transgenic TARGET cells, thereby eliminating the presence of un-differentiated progenitor cells and other lineages of immune cells. As a result, the TARGET cell product is pure, homogenous, of no GvHD risk, and therefore no need for a purification step.

I. In Vivo Efficacy Study of BCAR-iTARGET Cells.

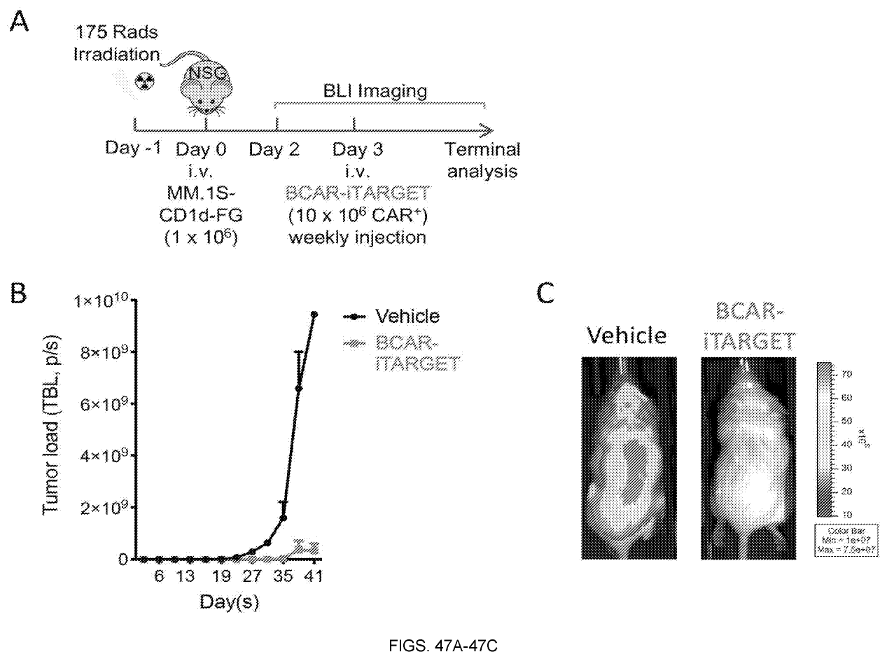

FIG. 47 demonstrates the efficient suppression of human MM growth in vivo by BCAR-iTARGET cells.

### Example 5: A Feeder-Free Ex Vivo Differentiation Culture Method to Generate Off-The-Shelf Monoclonal NY-ESO-1 Tumor Antigen Specific TCR-Armed Gene-Engineered T (esoTARGET) Cells

The αβ T cell receptor (TCR) determines the unique specificity of each nascent T cell. Upon assembly with CD3 signaling proteins on the T cell surface, the TCR surveils peptide ligands presented by MHC molecules on the surface of nucleated cells. The specificity of the TCR for a peptide-MHC complex is determined by both the presenting MHC molecule and the presented peptide. The MHC locus (also known as the HLA locus in humans) is the most multiallelic locus in the human genome, comprising >18,000 MHC class I and II alleles that vary widely in frequency across ethnic subgroups. Ligands presented by MHC class I molecules are derived primarily from proteasomal cleavage of endogenously expressed antigens. Infected and cancerous cells present peptides that are recognized by CD8+ T cells as foreign or aberrant, resulting in T cell-mediated killing of the presenting cell.

NY-ESO-1—the product of the CTAG1B gene—is an attractive target for off-the-shelf TCR gene therapy. As the prototypical cancer-testis antigen, NY-ESO-1 is not expressed in normal, nongermline tissue, but it is aberrantly expressed in many tumors. The frequency of aberrant expression ranges from 10 to 50% among solid tumors, 25-50% of melanomas, and up to 80% of synovial sarcomas with increased expression observed in higher-grade metastatic tumor tissue. Moreover, NY-ESO-1 is highly immunogenic, precipitating spontaneous and vaccine-induced T cell immune responses against multiple epitopes presented by various MHC alleles. As a result, the epitope NY-ESO-1157-165 (SLLMWITQC) presented by HLA-A*02:01 has been targeted with cognate 1G4 TCR in gene therapy trials, yielding objective responses in 55% and 61% of patients with metastatic melanoma and synovial sarcoma, respectively, and engendering no adverse events related to targeting. Targeting this same A2-restricted epitope with lentiviral-mediated TCR gene therapy in patients with multiple myeloma similarly resulted in 70% complete or near-complete responses without significant safety concerns. The majority of patients who respond to therapy relapse within months, and loss of heterozygosity at the MHCI locus has been reported as a mechanism by which tumors escape adoptive T cell therapy targeting HLA-A*02:01/NY-ESO-1157-165. Thus, NY-ESO-1 is a tumor-specific, immunogenic public antigen that is expressed across an array of tumor types and is safe to target in the clinic.

An off-the-shelf NY-ESO-1 TCR-Armed TARGET (esoTARGET) cellular product is therefore of great therapeutic potential and need.

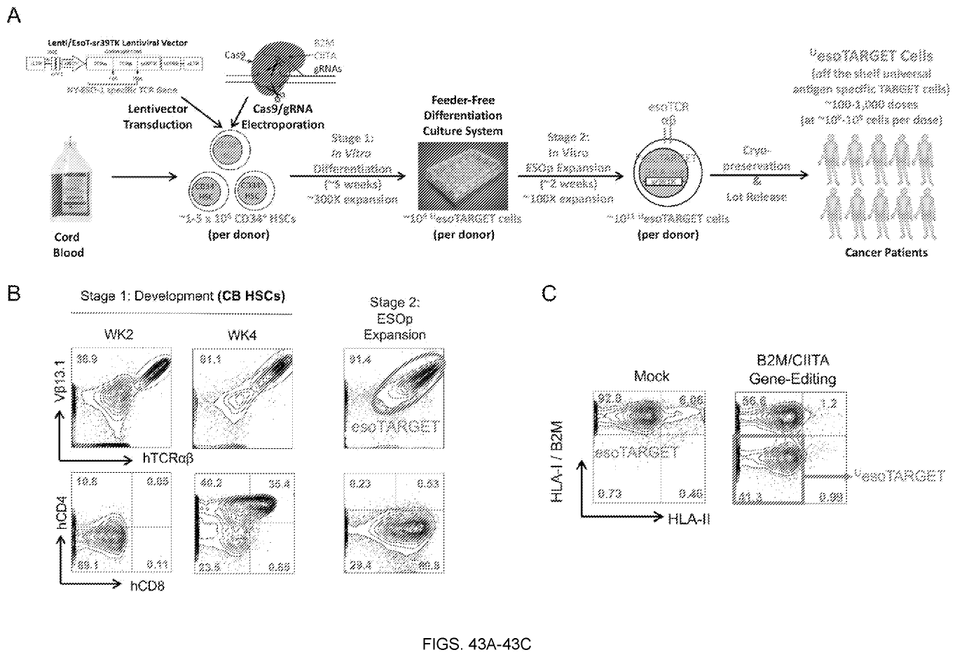

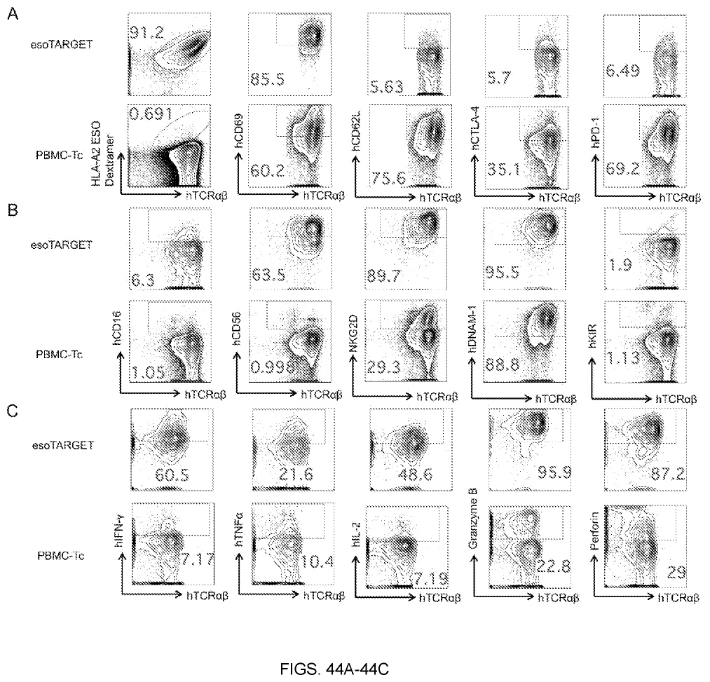

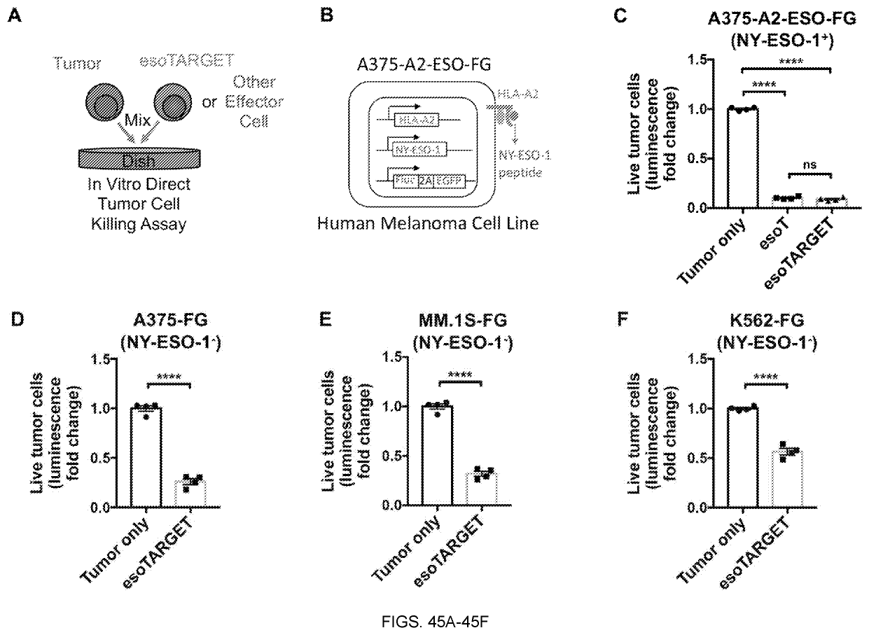

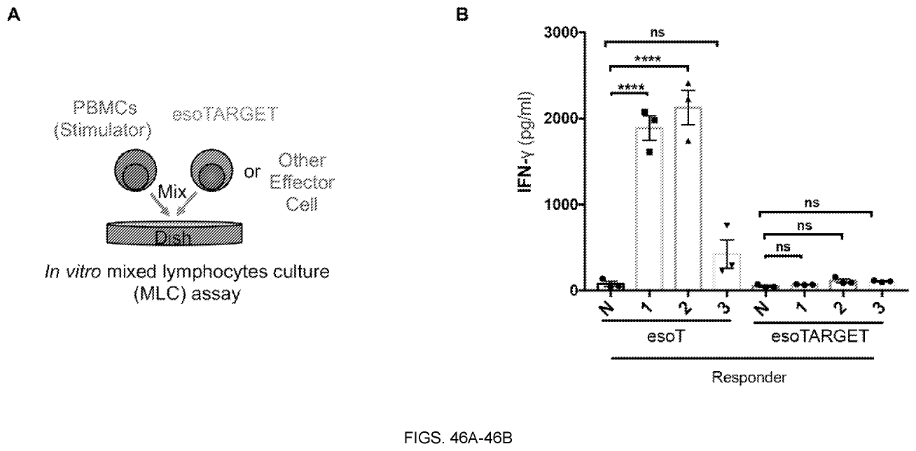

Certain embodiments relating to this example are demonstrated in FIGS. 43-46.

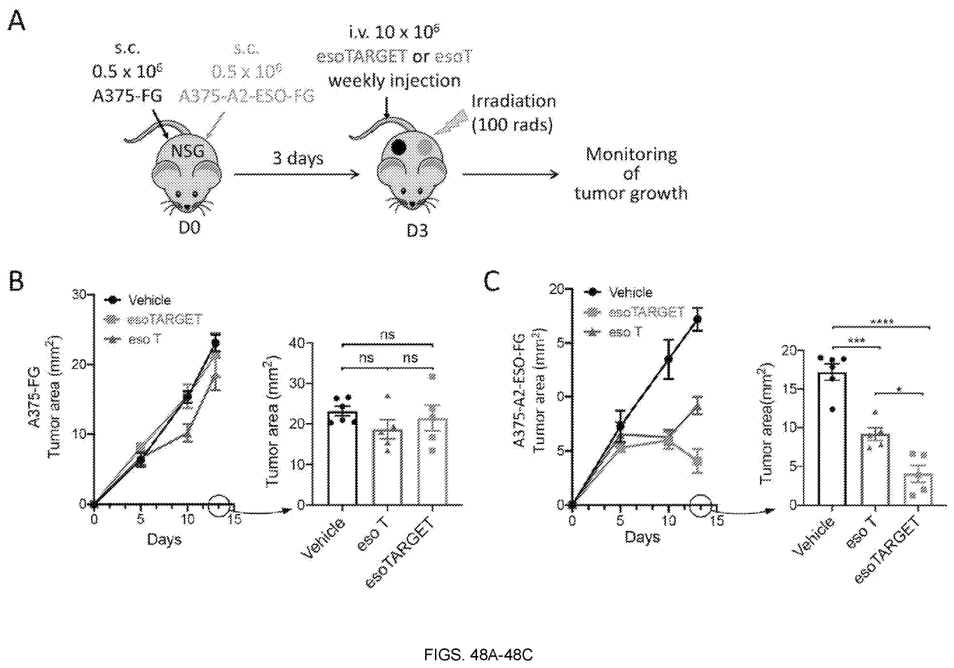

Shown in FIG. 48 is the in vivo efficacy of cells produced by the methods of the disclosure. Note the tumor antigen-specific suppression of human melanoma solid tumor growth in vivo by esoTARGET cells, at an efficacy comparable to or better than that of esoT cells (ESO TCR-engineered peripheral blood human CD8 T cells).

### Example 6: A Feeder-Free Ex Vivo Differentiation Culture Method to Generate Off-The-Shelf Monoclonal iNKT TCR-Armed Natural Killer (iTANK) Cells

Type 1 invariant natural killer T (iNKT) cells recognize glycolipid antigens presented by a non-polymorphic non-classical MHC Class I-like molecule CD1d. Consequently, iNKT cells do not cause graft-versus-host disease (GvHD) when adoptively transferred into allogeneic recipients. iNKT TCR comprises an invariant alpha chain (Vα14-Jα18 in mouse; Vα24-Jα18 in human), and a limited selection of beta chains (predominantly Vβ8/Vβ7/Vβ2 in mouse; predominantly Vβ 11 in human). Both mouse and human iNKT cells respond to a synthetic agonist glycolipid ligand, alpha-Galactosylceramide (αGC, or α-GC, or α-GalCer).

An off-the-shelf iNKT TCR-Armed TANK (iTANK) cellular product and its derivative CAR-engineered iTANK (CAR-iTANK) are novel cellular products that may be of therapeutic potential.

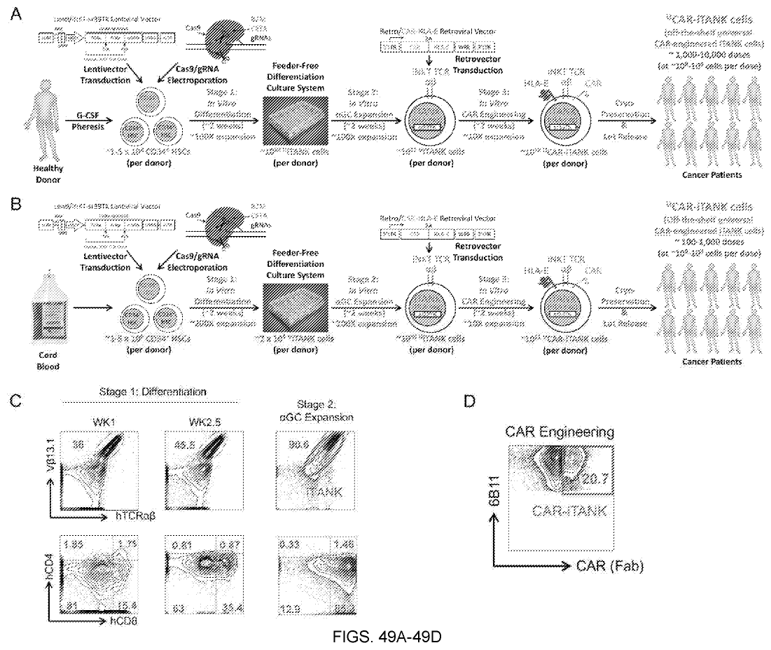

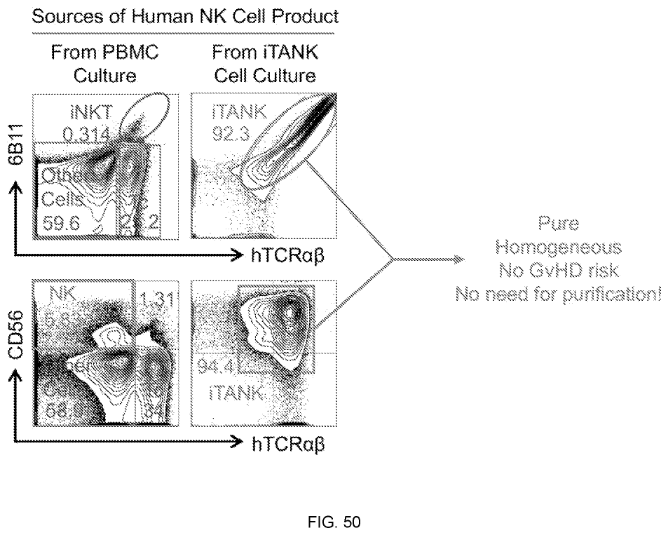

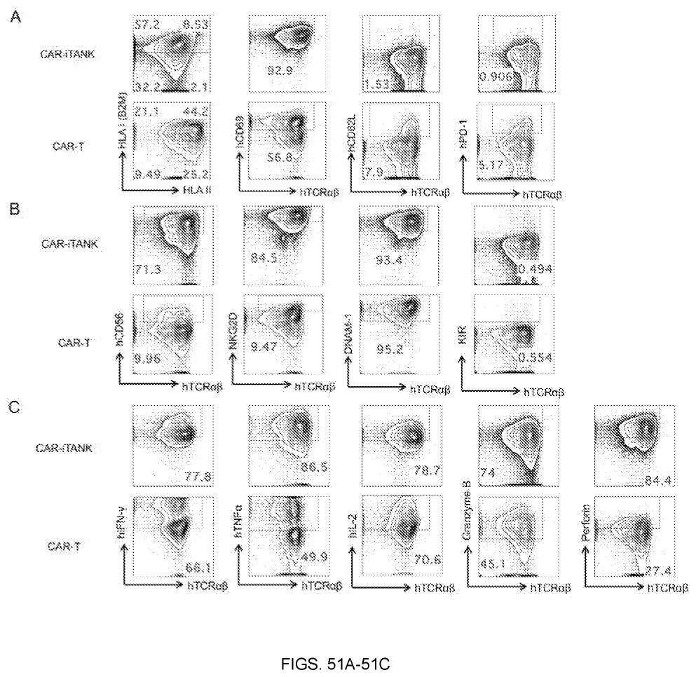

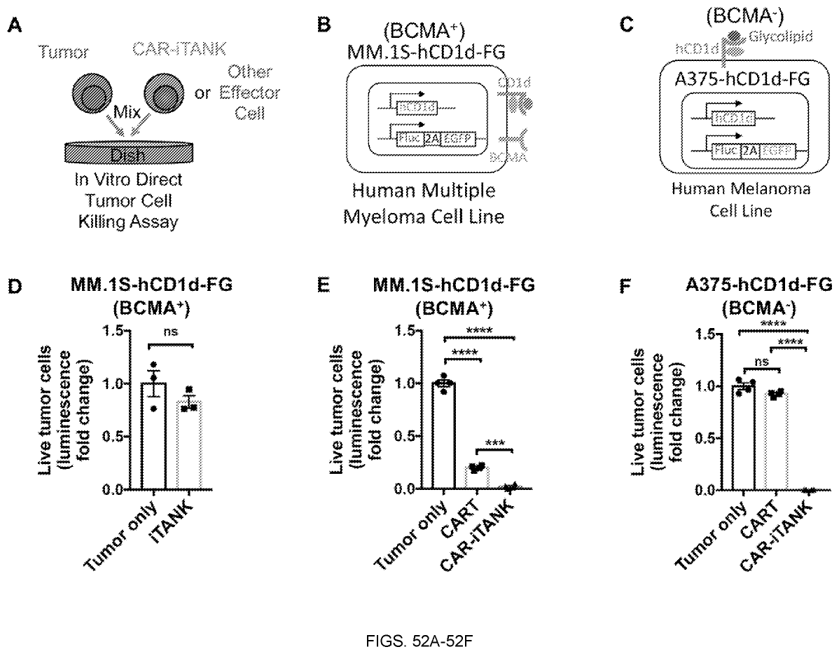

Certain embodiments relating to this example are demonstrated in FIGS. 49-52.

### Example 7: A Feeder-Free Ex Vivo Differentiation Culture Method to Generate Off-The-Shelf Monoclonal NY-ESO-1 Tumor Antigen Specific TCR-Armed Natural Killer (esoTANK) Cells

The αβ T cell receptor (TCR) determines the unique specificity of each nascent T cell. Upon assembly with CD3 signaling proteins on the T cell surface, the TCR surveils peptide ligands presented by MHC molecules on the surface of nucleated cells. The specificity of the TCR for a peptide-MHC complex is determined by both the presenting MHC molecule and the presented peptide. The MHC locus (also known as the HLA locus in humans) is the most multiallelic locus in the human genome, comprising >18,000 MHC class I and II alleles that vary widely in frequency across ethnic subgroups. Ligands presented by MHC class I molecules are derived primarily from proteasomal cleavage of endogenously expressed antigens. Infected and cancerous cells present peptides that are recognized by CD8⁺ T cells as foreign or aberrant, resulting in T cell-mediated killing of the presenting cell.

NY-ESO-1 the product of the CTAG1B gene is an attractive target for off-the-shelf TCR gene therapy. As the prototypical cancer-testis antigen, NY-ESO-1 is not expressed in normal, nongermline tissue, but it is aberrantly expressed in many tumors. The frequency of aberrant expression ranges from 10 to 50% among solid tumors, 25-50% of melanomas, and up to 80% of synovial sarcomas with increased expression observed in higher-grade metastatic tumor tissue. Moreover, NY-ESO-1 is highly immunogenic, precipitating spontaneous and vaccine-induced T cell immune responses against multiple epitopes presented by various MHC alleles. As a result, the epitope NY-ESO-1₁₅₇₋₁₆₅ (SLLMWITQC) presented by HLA-A*02:01 has been targeted with cognate 1G4 TCR in gene therapy trials, yielding objective responses in 55% and 61% of patients with metastatic melanoma and synovial sarcoma, respectively, and engendering no adverse events related to targeting. Targeting this same A2-restricted epitope with lentiviral-mediated TCR gene therapy in patients with multiple myeloma similarly resulted in 70% complete or near-complete responses without significant safety concerns. The majority of patients who respond to therapy relapse within months, and loss of heterozygosity at the MHCI locus has been reported as a mechanism by which tumors escape adoptive T cell therapy targeting HLA-A*02:01/NY-ESO-1₁₅₇₋₁₆₅. Thus, NY-ESO-1 is a tumor-specific, immunogenic public antigen that is expressed across an array of tumor types and is safe to target in the clinic.

An off-the-shelf NY-ESO-1 TCR-Armed NK (esoTANK) cellular product is therefore of great therapeutic potential and need.

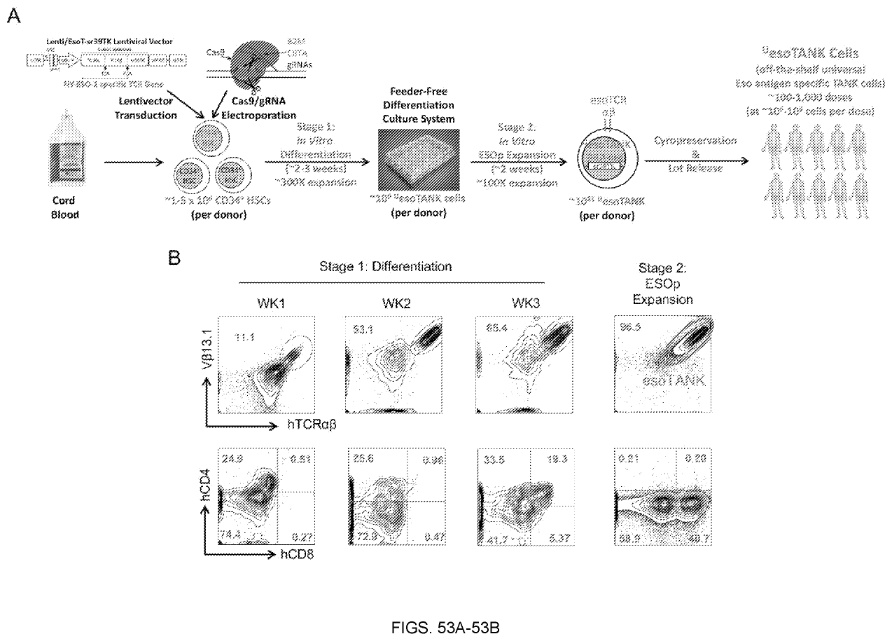

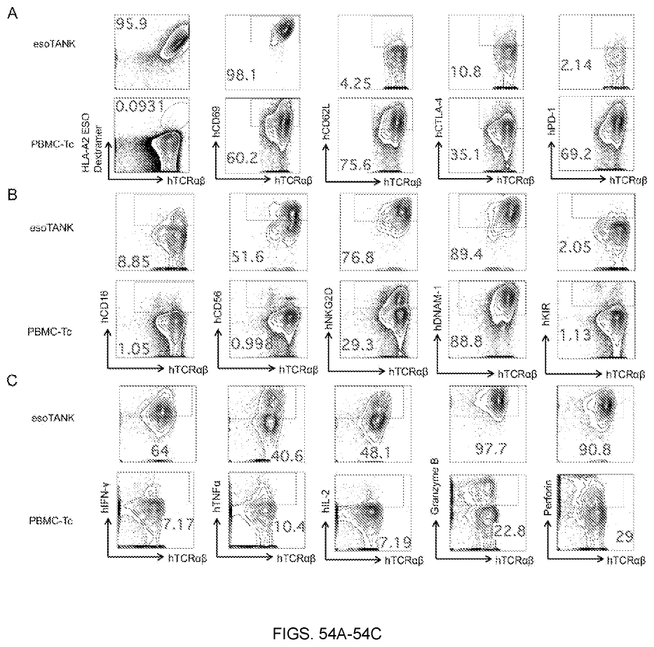

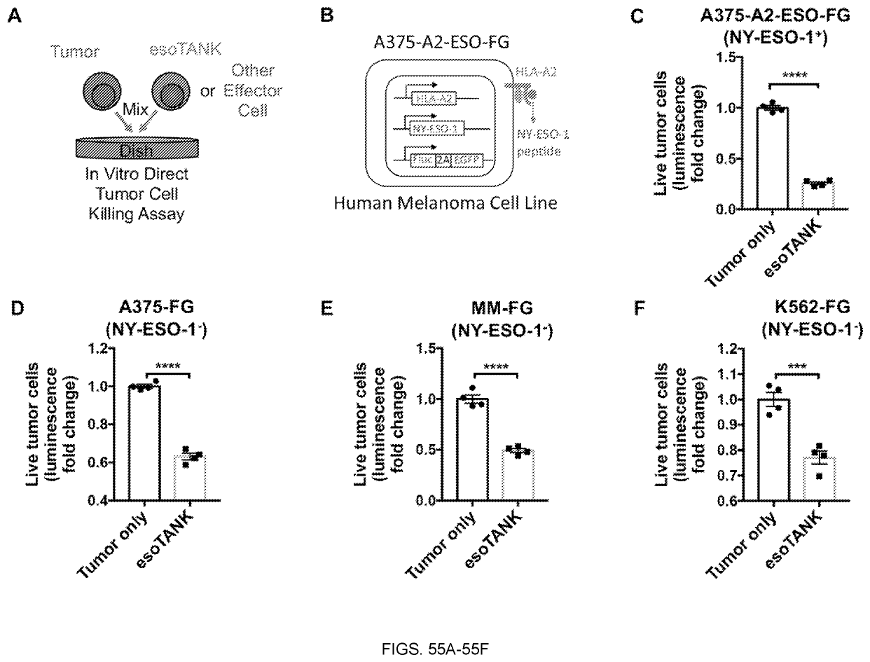

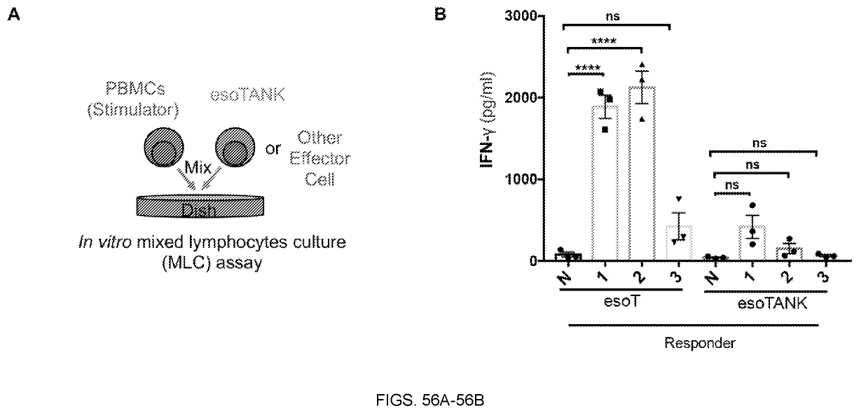

Certain embodiments relating to this example are demonstrated in FIGS. 53-56.

### Example 8: Hematopoietic Stem Cell-Engineered IL-15-Enhanced Off-The-Shelf CAR-iNKT Cells for Cancer Immunotherapy

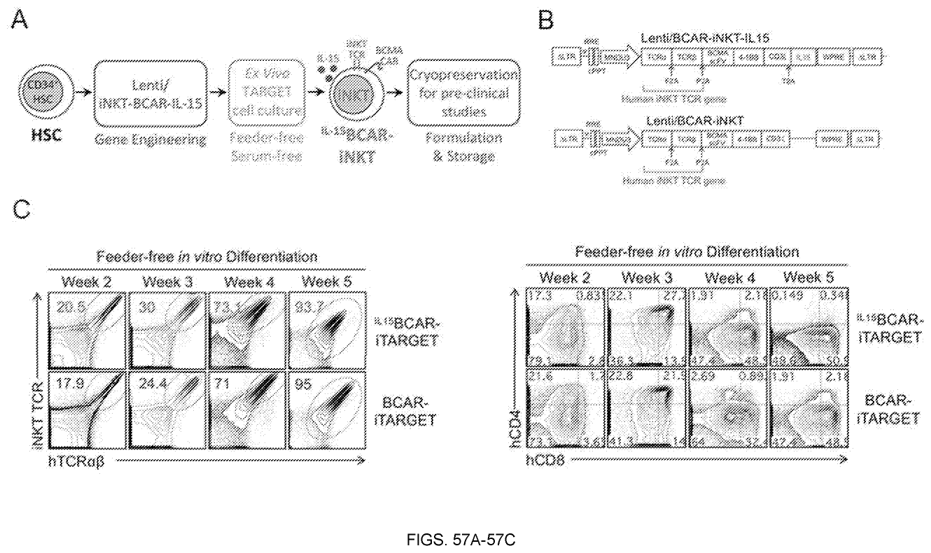

IL-15-enhanced BCAR-iTARGET (^{IL-15}BCAR-iTARGET) cells were engineered by transducing hematopoietic stem cells with a Lenti/iNKT-BCAR-IL-15 lentiviral vector. IL-15 enhancement did not interfere with the development of BCAR-iTARGET cells. FIGS. 57A-57C show embodiments and results related to these studies.

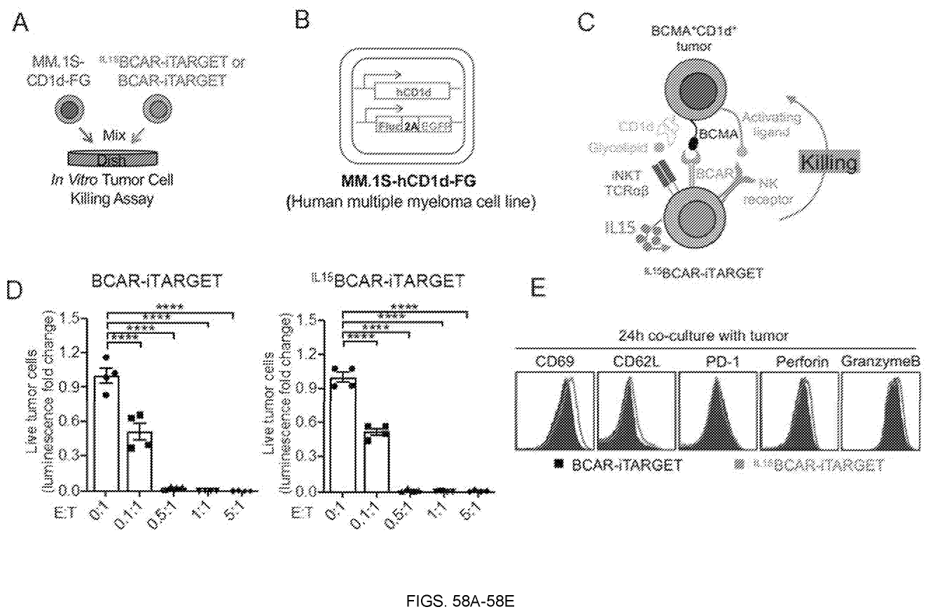

In vitro studies were performed to study the anti-cancer efficacy of IL15-CAR-iNKT cells. Compared to BCAR-iTARGET cells, ^{IL-15}BCAR-iTARGET cells showed comparable in vitro antitumor efficacy. FIGS. 58A-58E show embodiments and results related to these studies.

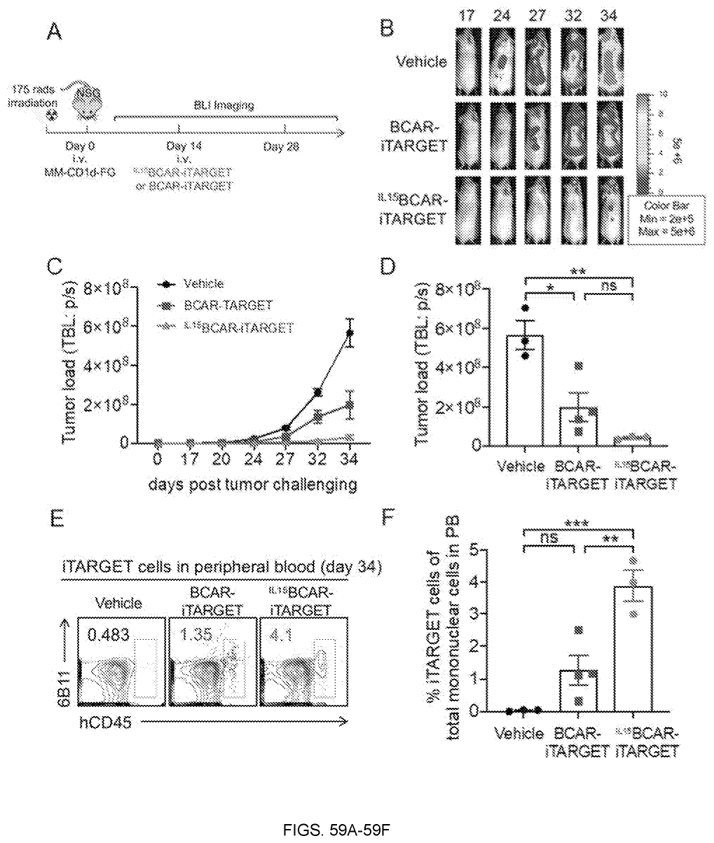

In vivo studies were performed to study the anti-cancer efficacy of IL15-CAR-iNKT cells. An MM.1S-hCD1d-FG human multiple myeloma xenograft NSG mouse model was used. Compared to BCAR-iTARGET cells, ^{IL-15}BCAR-iTARGET cells showed significantly enhanced in vivo antitumor efficacy associated with significantly improved in vivo persistency. FIGS. 59A-59F show embodiments and results related to these studies.

### Example 9: An Ex Vivo Feeder-Free Culture Method to Generate Hematopoietic Stem Cell-ENGINEERED Off-the-Shelf CAR-iNKT Cells for Cancer Immunotherapy

Cancer immunotherapy aims to harness and enhance the inherent power of the human immune system to fight cancer. After over a century of pursuit, significant breakthroughs have been achieved in the past few years1. In particular, chimeric antigen receptor-engineered T (CAR-T) cell therapy has shown unprecedented clinical efficacy and has recently been approved by the US Food and Drug Administration (FDA) for treating B cell malignancies; FDA approval for treating multiple myeloma (MM) is expected in 20202. These breakthroughs mark the beginning of a new era and are transforming cancer medicine.

CARs are synthetic receptors that redirect the specificity and function of T cells. By designing CARs to recognize corresponding antigens, CAR-T cells can target a broad range of cancers, as well as many other diseases. The potential clinical applications of CAR-T cell therapy are therefore enormous, and various CAR-T cell therapies are currently under active development.

The first two FDA-approved CAR-T therapies, Kymriah and Yescarta, are priced at $475,000 and $373,000 respectively. They are so expensive because personalized autologous CAR-T cell products need to be manufactured for each patient and can only be used to treat that single patient. Moreover, the manufacturing of autologous CAR-T cell products varies hugely from site to site and is not always successful. The steep price and manufacturing inconsistencies make it difficult to deliver the powerful CAR-T cell therapy to millions of patients in need. It is therefore of paramount importance to develop universal, standardized, off-the-shelf CAR-T cell products that can be manufactured on a large scale at centralized sites at dramatically reduced costs and that can be pre-stored for expeditious distribution to all patients in need.

Allogeneic conventional ab T cells have been utilized to develop off-the-shelf CAR-T cell products. However, these T cells have a critical limitation in that they risk inducing graft-versus-host disease (GvHD) when transferred into allogeneic hosts. Gene-editing tools have been applied to disrupt T cell receptor (TCR) expression on such CAR-T cells, aiming to alleviate GvHD risk. However, it is a significant manufacturing challenge to achieve complete elimination of TCR-expression in the cells, and GvHD has been observed in clinical trials testing these allogeneic CAR-T cell products. Utilization of alternative allogeneic cells that have no GvHD risk is therefore an attractive option to develop safe and universal off-the-shelf CAR-T cell products.

Disclosed herein are off-the-shelf cell therapies for cancers developed by generating allogenic and/or universal CAR-engineered iNKTs targeting cancer.

Gene delivery lentiviral vectors were constructed for use in these studies. FIGS. 60A-57D show embodiments and results related to construction of these vectors.

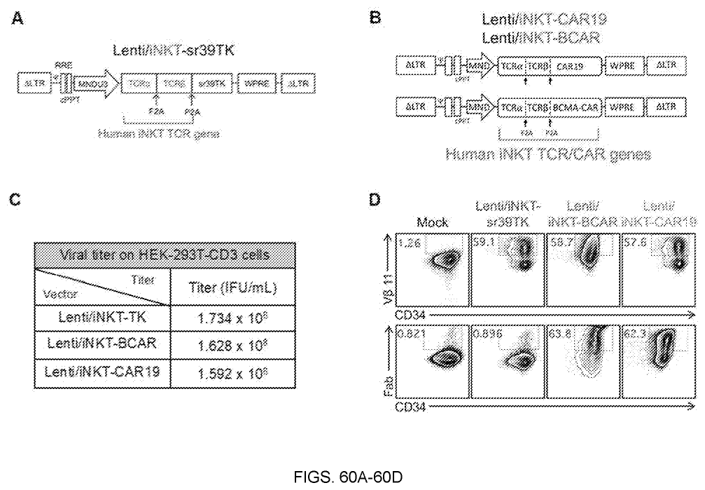

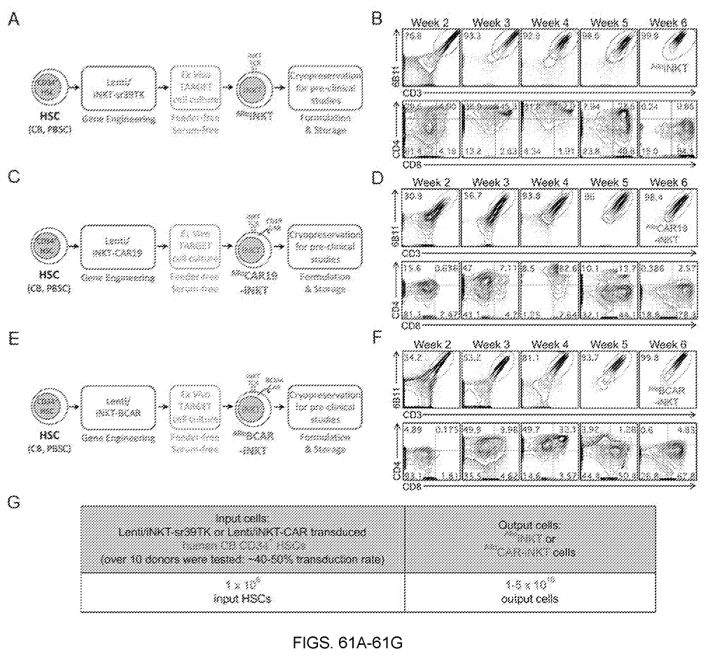

Allogeneic iNKT (^{Allo}iNKT), CAR-iNKT (^{Allo}CAR-iNKT), and ^{Allo}BCAR-iNKT cells were engineered by transducing hematopoietic stem cells with Lenti-iNKT-sr39TK, Lenti-iNKT-CAR19, and Lenti-BCAR-iNKT lentiviral vectors. FIGS. 61A-61G show embodiments and results related to these studies.

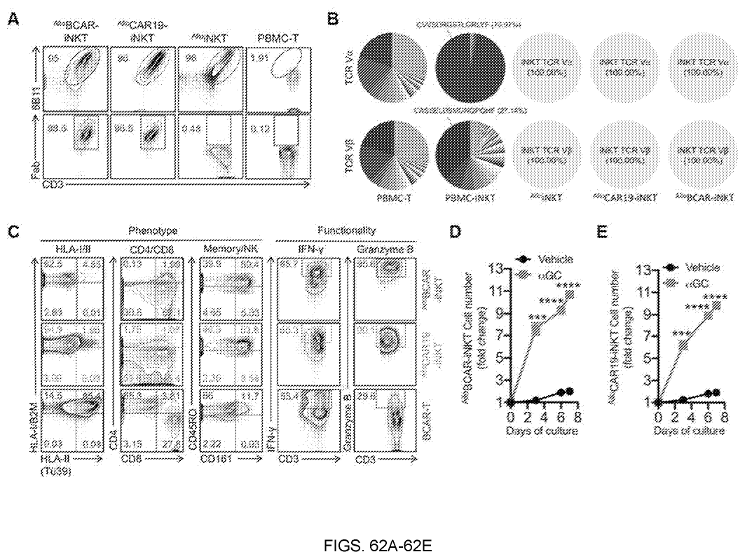

FACS analyses were conducted to characterize the phenotype of the ^{Allo}CAR-iNKT cells. In vitro studies assessing the expansion of the ^{Allo}CAR-iNKT cells in response to antigen stimulation were conducted to characterize the functionality of the ^{Allo}CAR-iNKT cells. FIGS. 62A-62E show embodiments and results related to these studies.

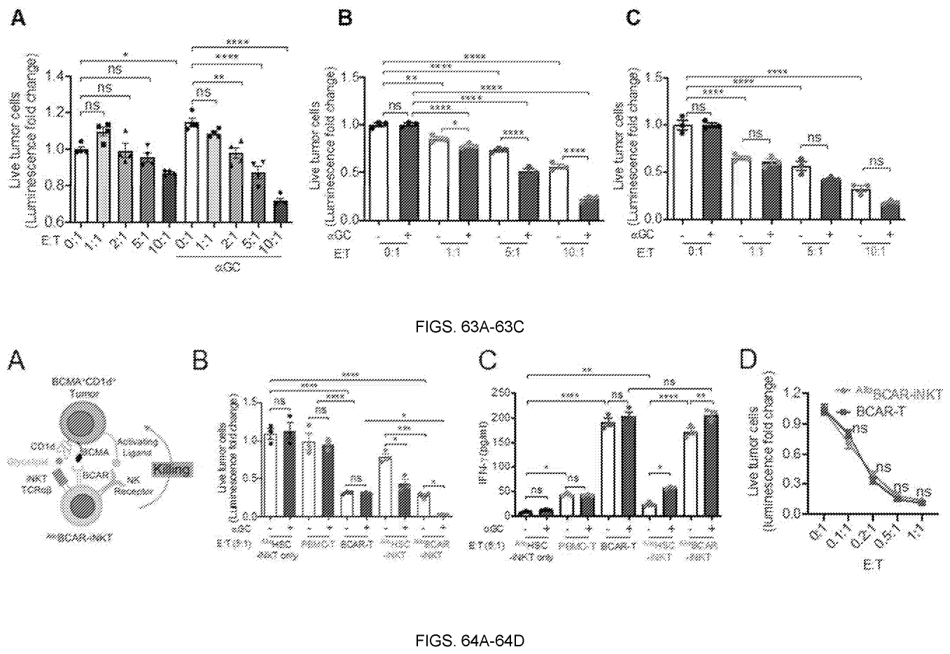

In vitro studies were performed to study the anti-cancer efficacy and mechanism of action of the ^{Allo}iNKT cells. ^{Allo}iNKT cells effectively killed multiple types of human cancer cells using both TCR-dependent and TCR-independent (i.e., via NK path) mechanisms. FIGS. 63A-63C show embodiments and results related to these studies.

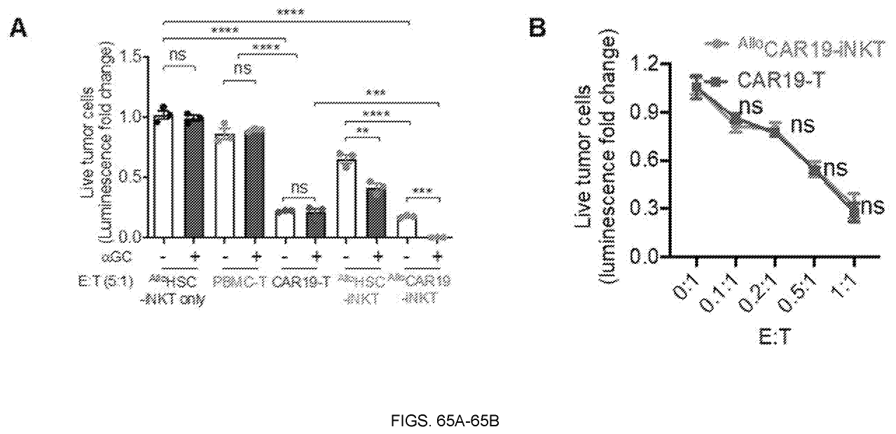

In vitro studies were performed to study the anti-cancer efficacy and mechanism of action of the ^{Allo}BCAR-iNKT cells. ^{Allo}BCAR-iNKT cells effectively killed human multiple myeloma tumor cells using the NK/TCR/CAR triple mechanisms, at an efficacy comparable to or better than that of the conventional BCAR-T cells. FIGS. 64A-64D show embodiments and results related to these studies.

In vitro studies were performed to study the anti-cancer efficacy and mechanism of action of the ^{Allo}CAR-iNKT cells. ^{Allo}CAR-iNKT cells effectively killed human B cell lymphoma cells using the NK/TCR/CAR triple mechanisms, at an efficacy comparable to or better than that of the conventional CAR19-T cells. FIGS. 65A-65B show embodiments and results related to these studies.

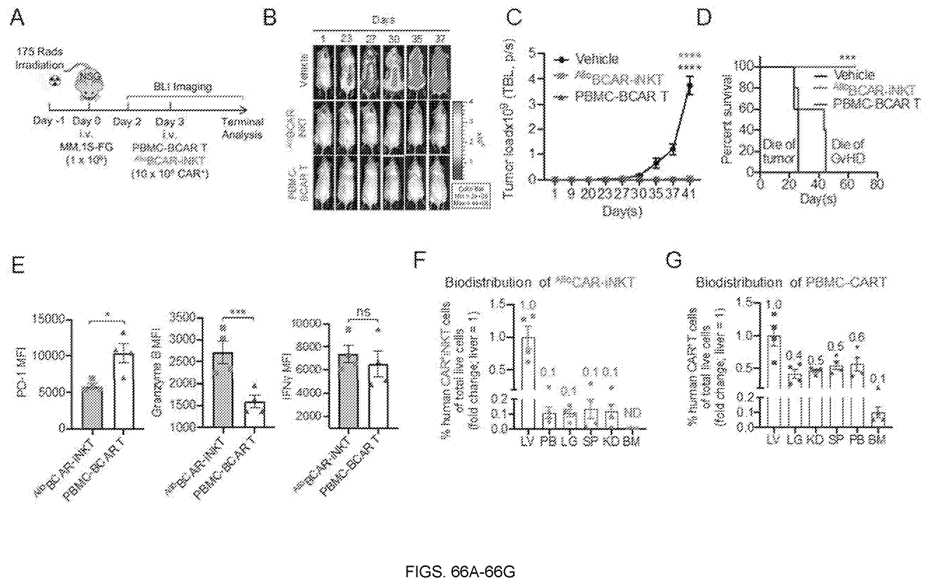

In vivo studies were performed to study the anti-cancer efficacy of the ^{Allo}BCAR-iNKT cells. A MM.1S-FG human multiple myeloma xenograft NSG mouse model was utilized. The conventional PBMC-derived BCAR-T cells were included as a control. Both ^{Allo}BCAR-iNKT cells and BCAR-T cells effectively eliminated MM cells. Although BCAR-T cells eliminated MM cells but also killed the recipient mice due to GvHD. In contrast, ^{Allo}BCAR-iNKT cells eliminated MM cells and did not cause GvHD, resulting in long-lived tumor-free recipient mice. Compared to the conventional BCAR-T cells, ^{Allo}BCAR-iNKT cells expressed significantly lower levels of surface PD-1 and produced significantly higher levels of Granzyme-B. Compared to BCAR-T cells, ^{Allo}BCAR-iNKT cells showed enhanced tumor-homing. FIGS. 66A-66G show embodiments and results related to these studies.

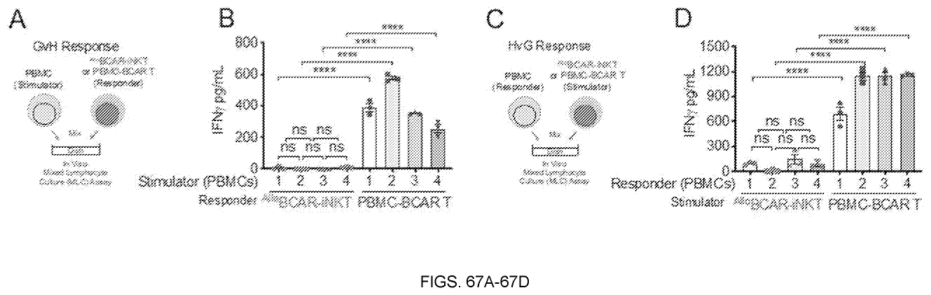

In vitro mixed lymphocyte (MLC) assays were used to study the immunogenicity of ^{Allo}BCAR-iNKT cells in comparison with conventional BCAR-T cells. Different from the conventional BCAR-T cells, ^{Allo}BCAR-iNKT cells showed no GvH response and significantly reduced HvG response. FIGS. 67A-67D show embodiments and results related to these studies.

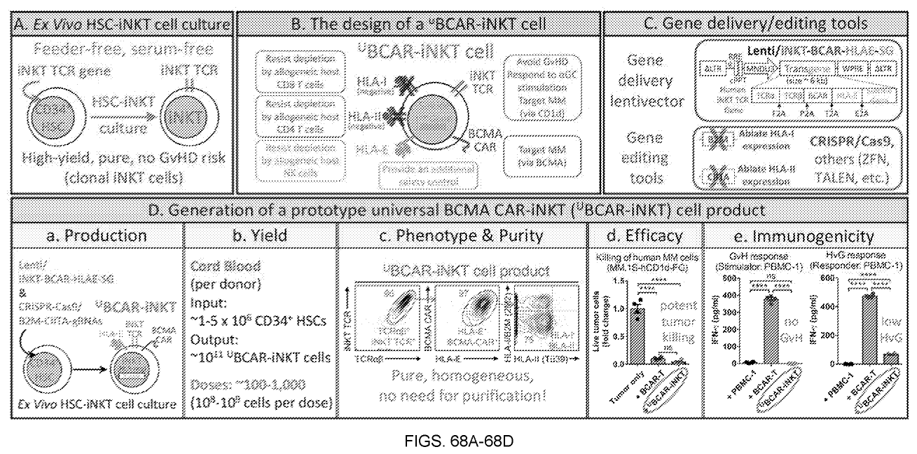

Allogeneic HLA-I/II-negative “universal” BCAR-iNKT (^{U}BCAR-iNKT) cells were also generated and characterized. An “ideal” ^{U}BCAR-iNKT cell should meet the following criteria: 1) express iNKT TCRs to avoid GvHD, as well as to respond to alpha-galactosylceramide (αGC) stimulation and target MM via recognition of CD1d, 2) express BCMA CARs to target MM via recognition of BCMA, 3) lack surface expression of HLA-I and HLA-II molecules so as to resist depletion by allogeneic host CD8 and CD4 T cells, 4) express HLA-E molecules to resist depletion by allogeneic host natural killer (NK) cells, and 5) express a suicide gene to provide an additional safety control (FIG. 68B). A neat, two-pronged strategy accomplishes these HSC gene-engineering goals: first, a Lenti/iNKT-BCAR-HLAE-SG lentiviral vector has been successfully constructed to efficiently co-deliver all 5 transgenes to CD34+ HSCs, encoding an iNKT TCR a and b chain pair (iNKT), a BCMA CAR (BCAR), an HLA-E molecule (HLAE), and a thymidine kinase suicide gene (SG); second, a CRISPR-Cas9/B2M-CIITA-gRNAs complex has been successfully generated to efficiently disrupt the beta-2 microglobulin (B2M) and Class II Major Histocompatibility Complex Transactivator (CIITA) genes in CD34+ HSCs, resulting in an absence of surface HLA-I and HLA-II molecules in engineered HSCs and their progeny iNKT cells (FIG. 68C). Other SGs and gene editing tools may be used, but in some embodiments, the thymidine kinase SG and the CRISPR/Cas9 tool are used (FIG. 68C).

Using these technological innovations, ^{U}BCAR-iNKT cells were generated. Cord blood (CB) CD34+ HSCs were gene-engineered, then placed in the Ex Vivo HSC-iNKT cell culture (FIGS. 68A and 68D). The cell yield was impressive: from one CB donor, ˜10^{11 U}BCARiNKT cells were generated-cells that can potentially be formulated into 100-1,000 doses of off-the-shelf cell product, assuming 10⁸-10⁹ cells per dose based on the FDA-approved CAR-T therapy standard (FIG. 68D). The ^{U}BCAR-iNKT cell product was pure and homogeneous, with a high surface HLA-I/II ablation rate (FIG. 68D). Functionally, these ^{U}BCAR-iNKT cells killed MM tumor cells effectively, comparable to or better than conventional BCMA CAR-T (BCAR-T) cells (FIG. 68D). Immunogenicity studies showed that these ^{U}BCAR-iNKT cells did not induce graft-versus-host (GvH) responses and were resistant to host-verse-graft (HvG) responses (FIG. 68D). Taken together, these pilot studies point to a clear path for developing a ^{U}BCAR-iNKT cell product.

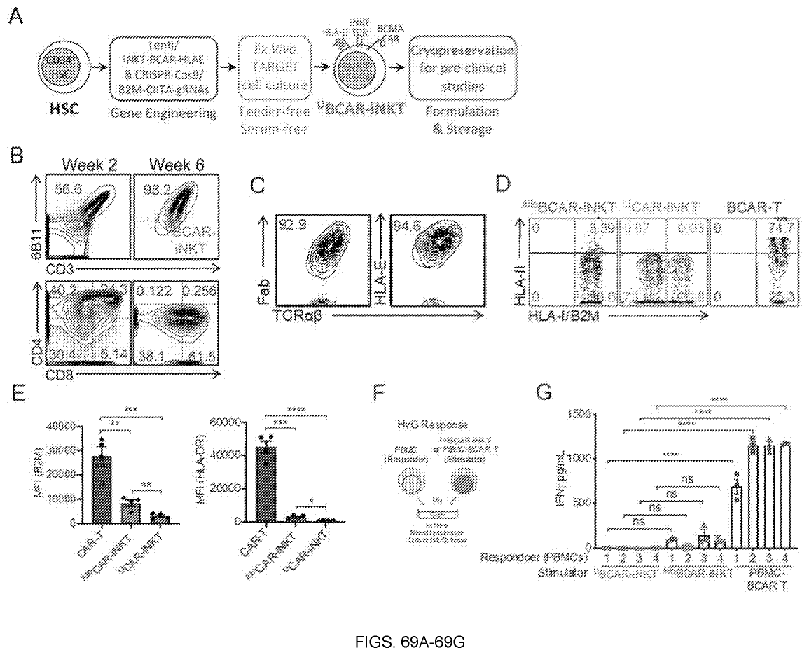

^{U}BCAR-iNKT cells' phenotype and immunogenicity were also characterized. FIGS. 69A-69G show embodiments and results related to these studies.

### Example 10: An Ex Vivo Feeder-Free Culture Method to Generate Hematopoietic Stem Cell-Engineered Off-the-Shelf Cytotoxic cd8 Cells for Cancer Immunotherapy

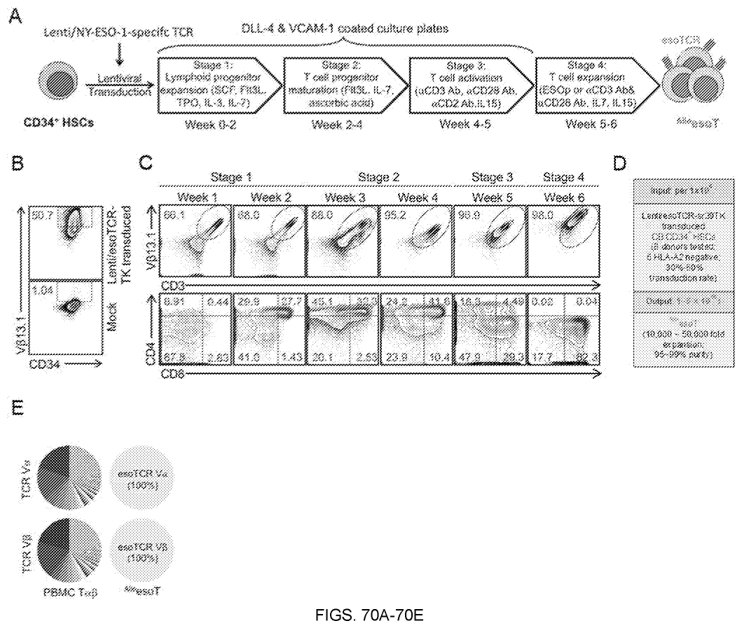

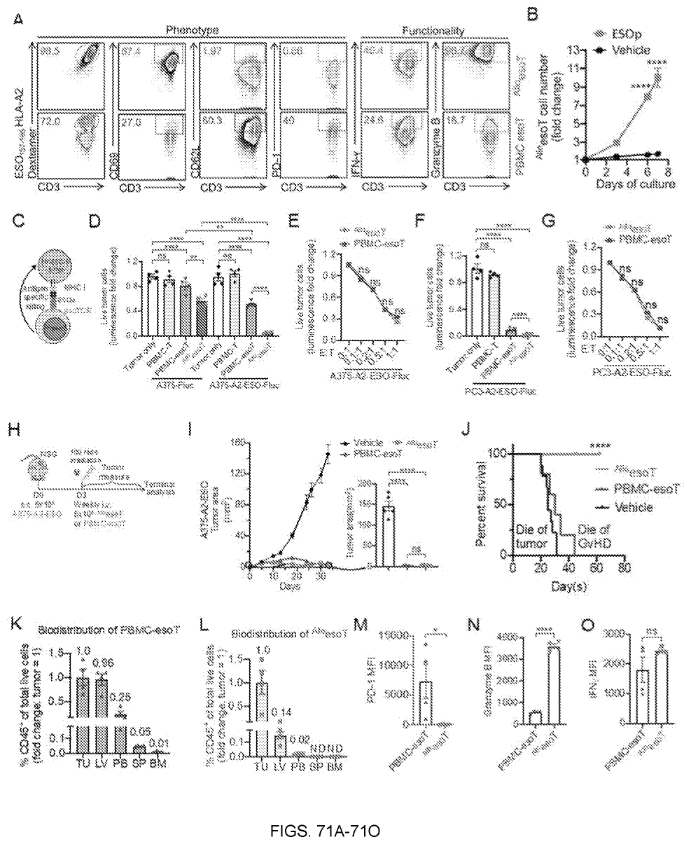

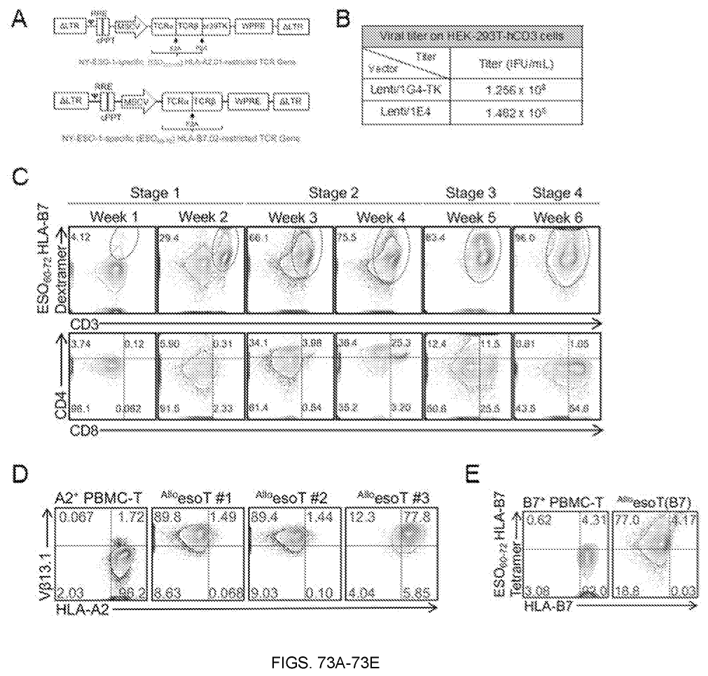

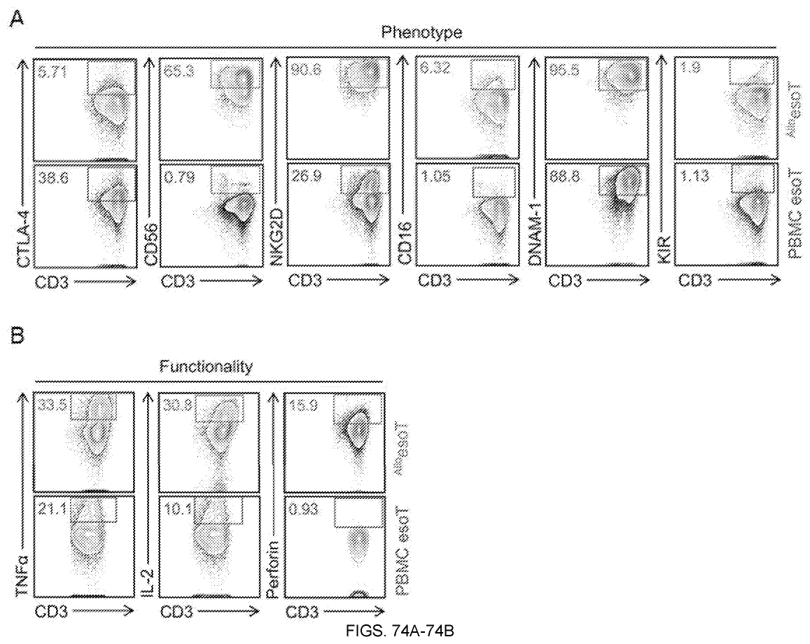

NY-ESO-1-specific T (^{Allo}esoT) cells were engineered by transducing hematopoietic stem cells with a lentiviral vector. Phenotype was characterized using FACS. FIGS. 70A-70E and FIGS. 73A-73E show embodiments and results related to these studies.

In vitro studies were performed to study the anti-cancer capacity and efficacy of ^{Allo}esoT cells. FIGS. 71A-710 show embodiments and results related to these studies.

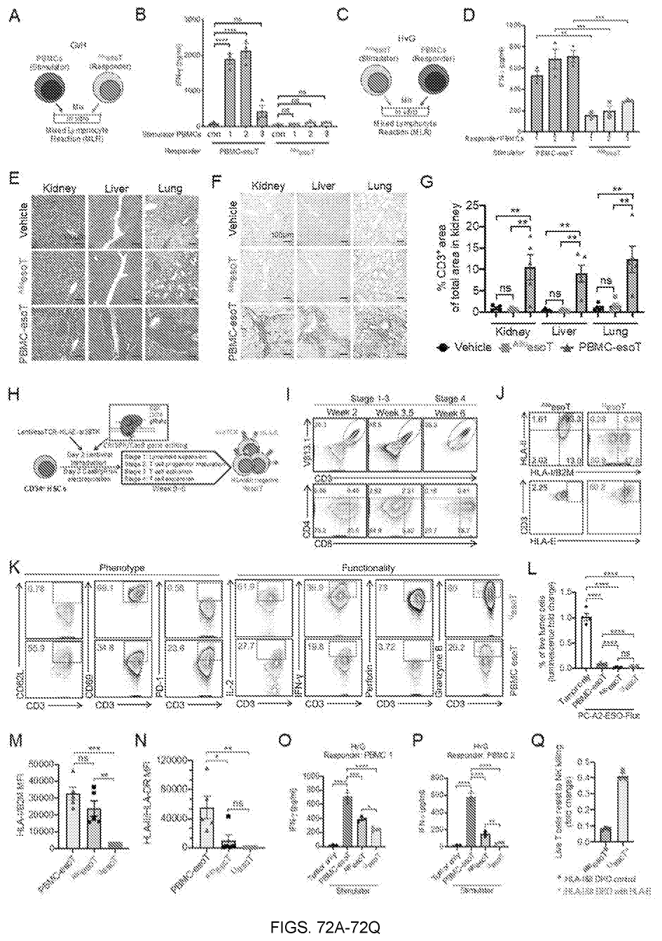

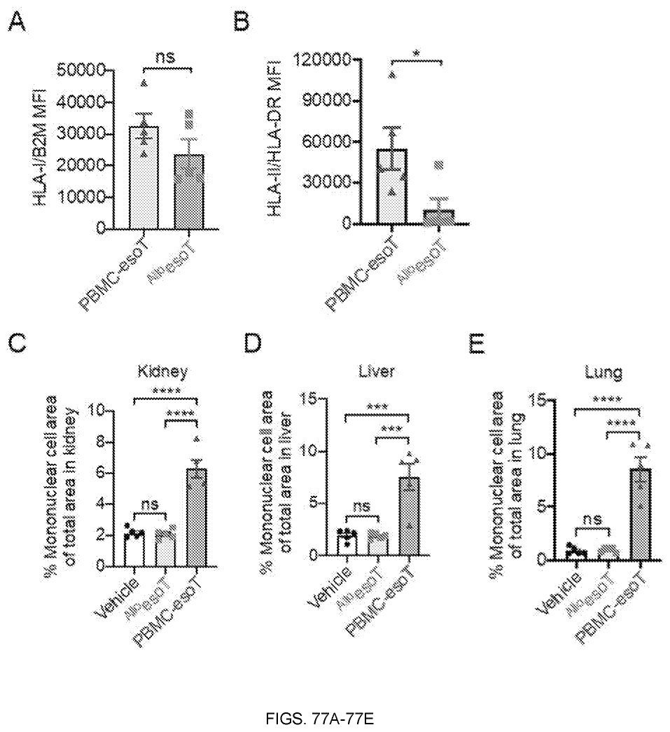

In vitro studies were performed to assess the safety of ^{Allo}esoT cells and reduce the immunogenicity of the cells using gene editing. ^{U}esoT cells were also engineered and compared to the safety and immunogenicity of ^{Allo}esoT cells. FIGS. 72A-720 show embodiments and results related to these studies. PBMC-esoT cells were also obtained and compared to the safety and immunogenicity of ^{Allo}esoT cells. FIGS. 72A-720 and FIGS. 77A-77E show embodiments and results related to these studies.

In vitro FACS analyses were performed to characterize the phenotype and functionality of ^{Allo}esoT cells. FIGS. 74A-74B show embodiments and results related to these studies.

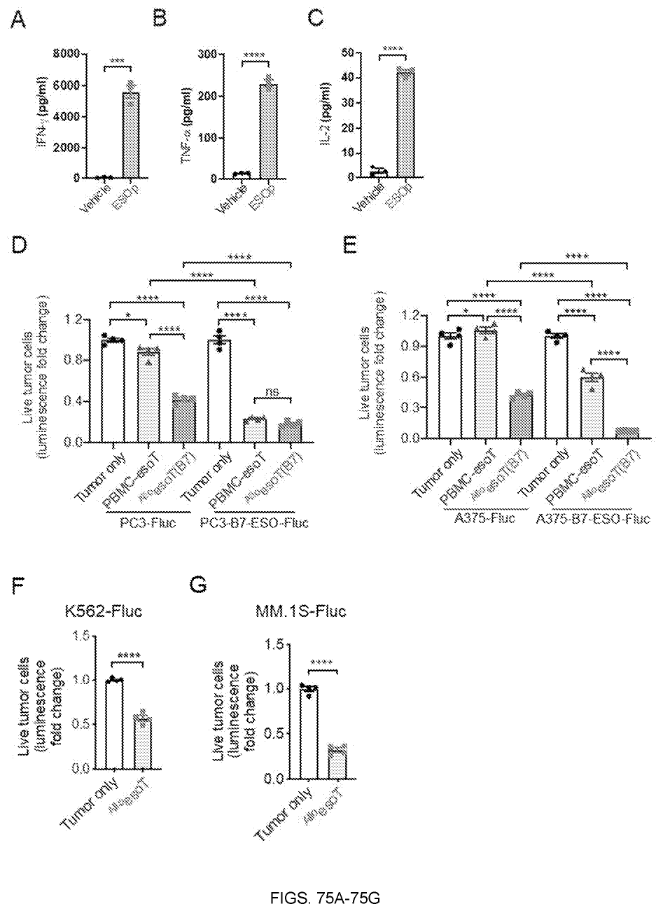

In vitro studies were performed to assess the antigen response and tumor killing capacity of ^{Allo}esoT cells. FIGS. 75A-75G show embodiments and results related to these studies.

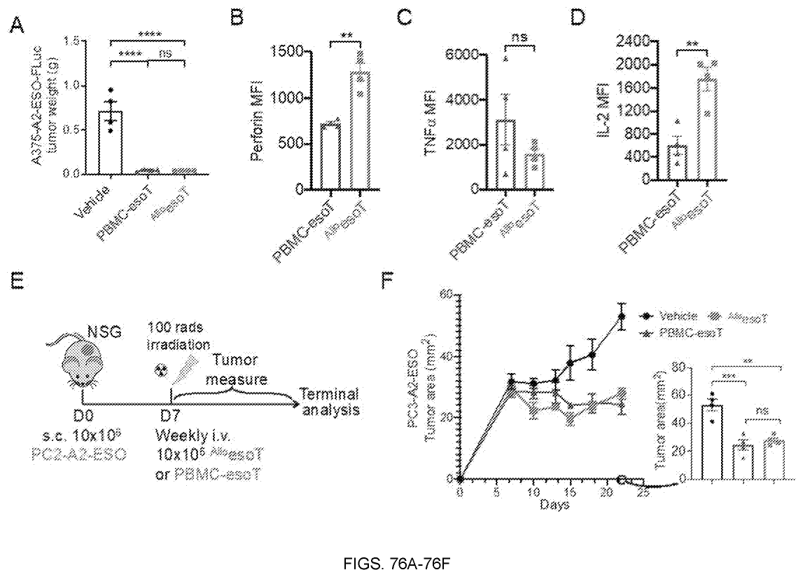

In vivo studies were performed to study the anti-cancer efficacy of ^{Allo}esoT cells. FIGS. 76A-76F show embodiments and results related to these studies.

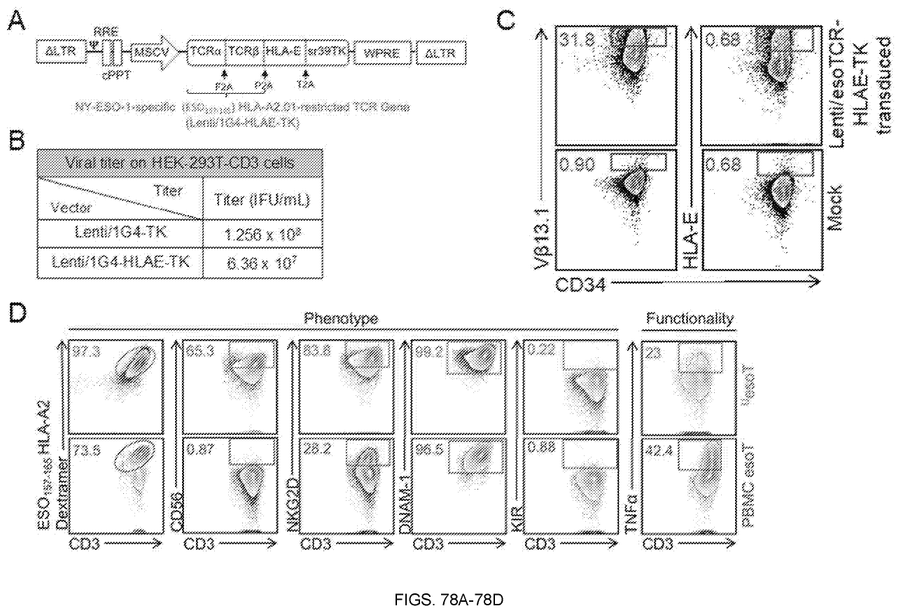

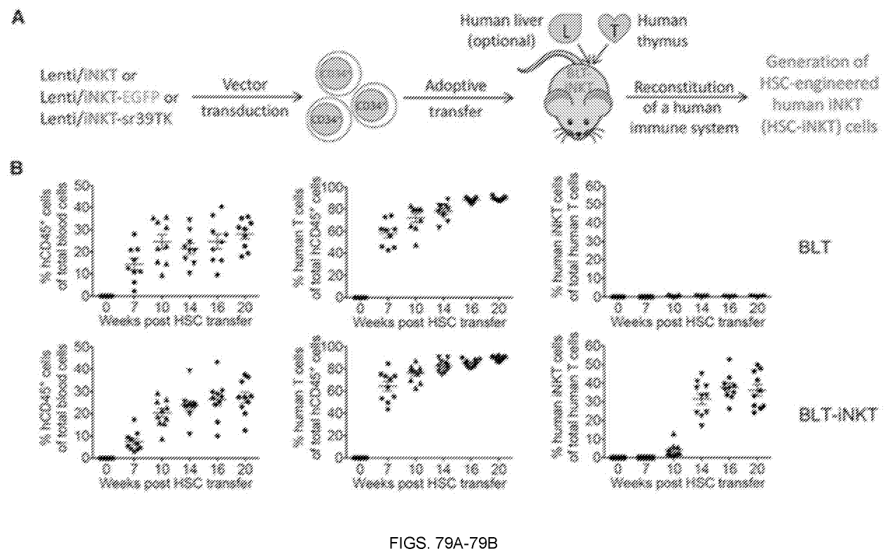

^{U}esoT cells were engineered by transducing hematopoietic stem cells with a lentiviral vector. Phenotype and functionality were characterized using FACS. FIGS. 78A-78D show embodiments and results related to these studies.

### Example 11: HSC-Engineered Off-The-Shelf iNKT Cells for the Prevention of Graft-Versus-Host Disease Associated with Allogeneic HCT

HSC-engineered human iNKT cells were engineered by transducing hematopoietic cells with a lentiviral vector and performing adoptive transfer into BLT mice. 79A-79B show embodiments and results related to these studies.

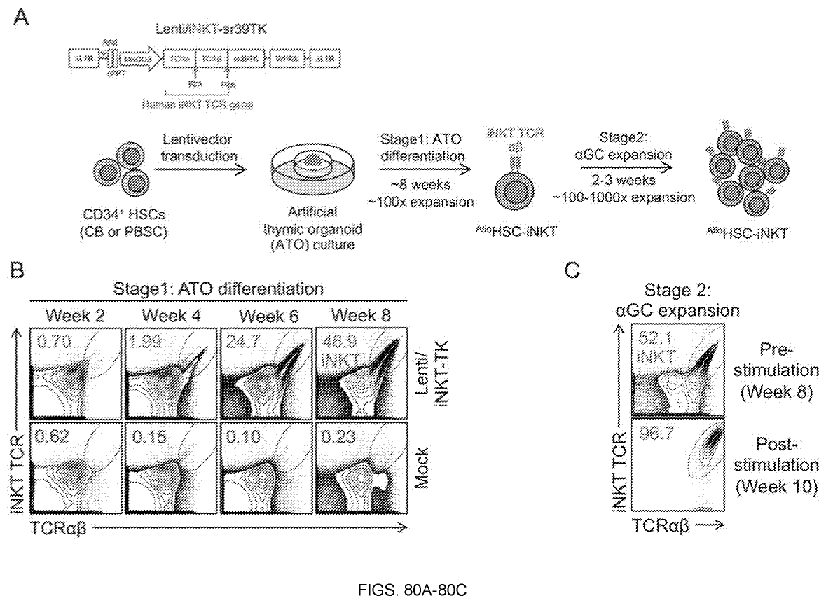

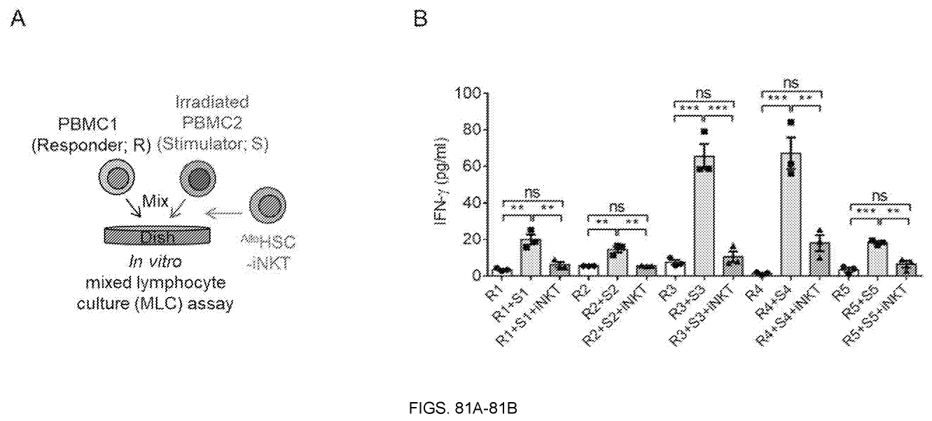

^{Allo}HSC-iNKT Cells were engineered by transducing hematopoietic stem cells with a lentiviral vector, culturing in an ATO system to differentiate the cells, and stimulating with αGC in an expansion culture. FIGS. 80A-80C show embodiments and results related to these studies.

In vitro studies including mixed lymphocyte reaction assays were performed to demonstrate that ^{Allo}HSC-iNKT cells reduce T cell alloreaction. FIGS. 81A-81B show embodiments and results related to these studies.

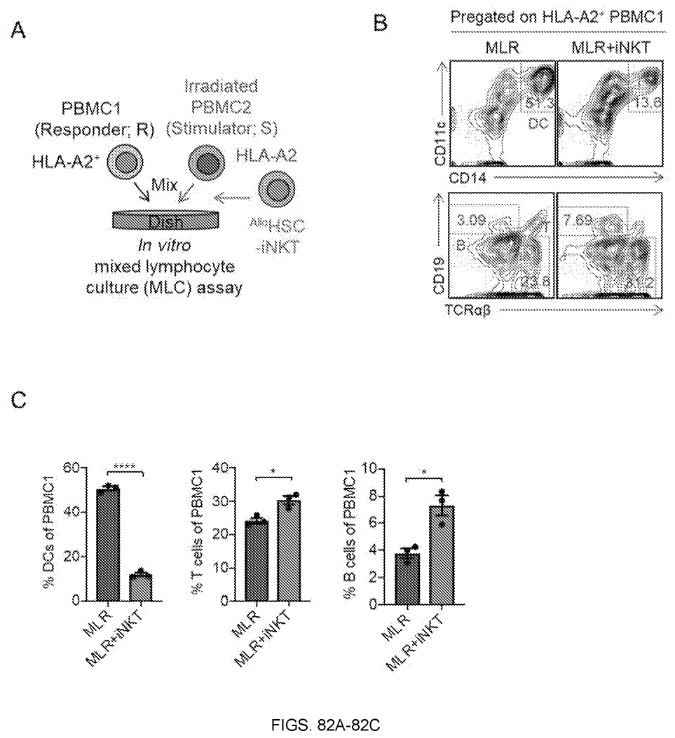

In vitro studies were performed to determine that ^{Allo}HSC-iNKT cells target allogenic myeloid APCs. FIGS. 82A-82C show embodiments and results related to these studies.

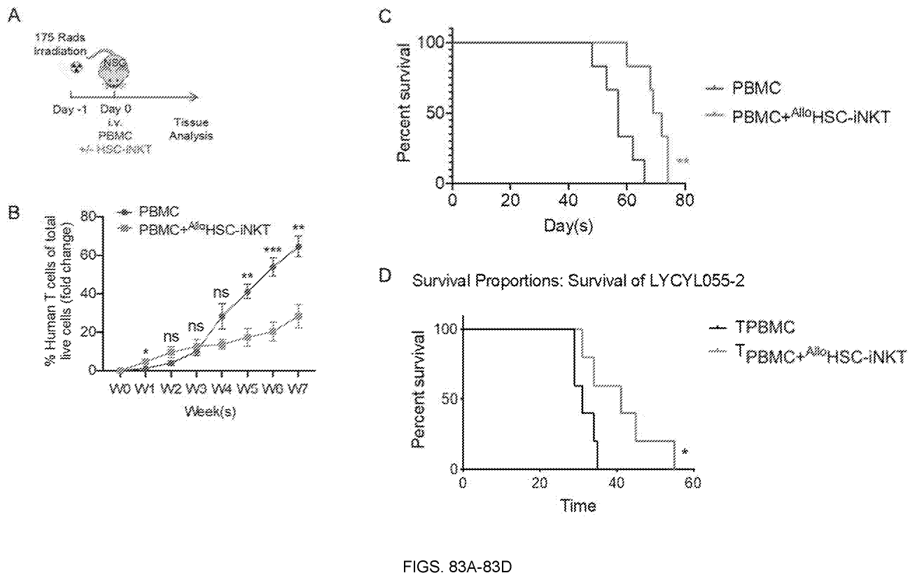

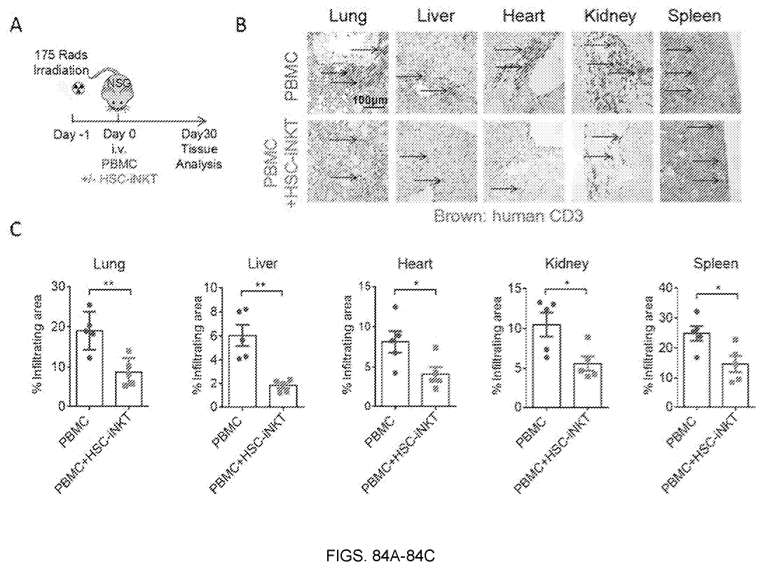

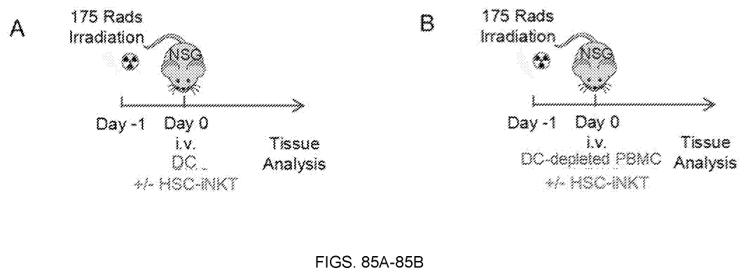

In vivo studies were performed to show that iNKT cells prevent allogenic T cell proliferation and GvHD in NSG mice. FIGS. 83A-83D, 84A-84C, and 85A-85B show embodiments and results related to these studies.

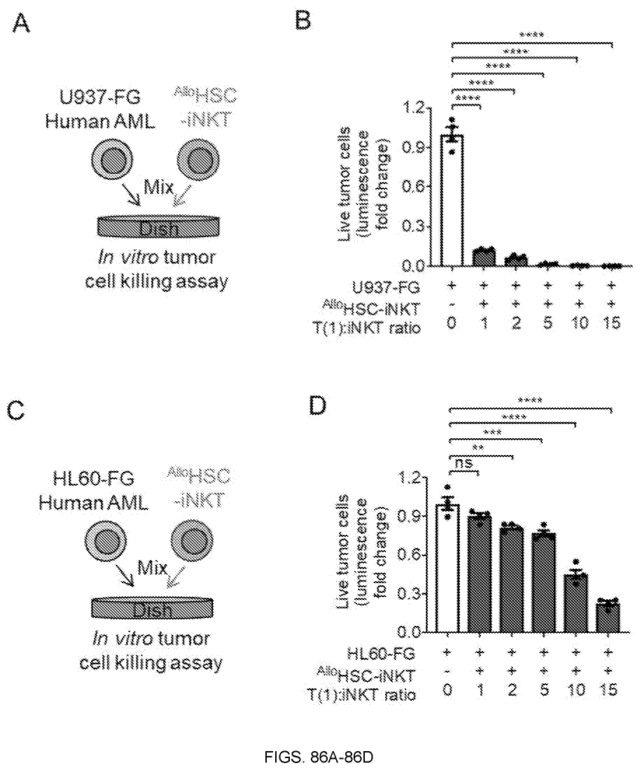

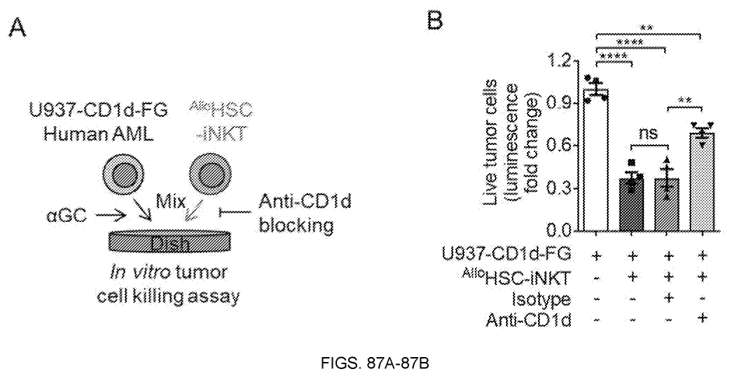

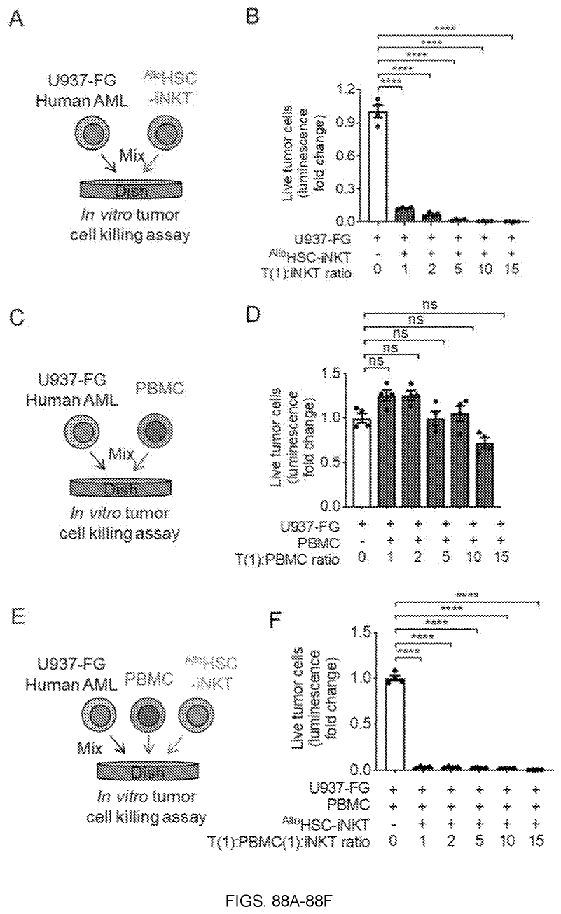

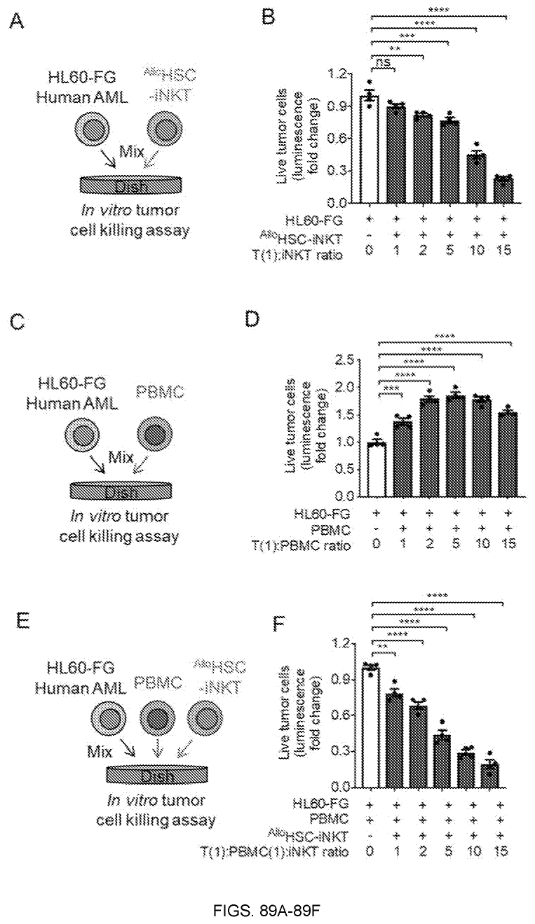

In vitro studies were performed to demonstrate that ^{Allo}HSC-iNKT cells show anti-cancer efficacy and capacity against U937 and HL60 AML tumor cells. FIGS. 86A-86D, 87A-87B, and 88A-88F, and 89A-89F show embodiments and results related to these studies.

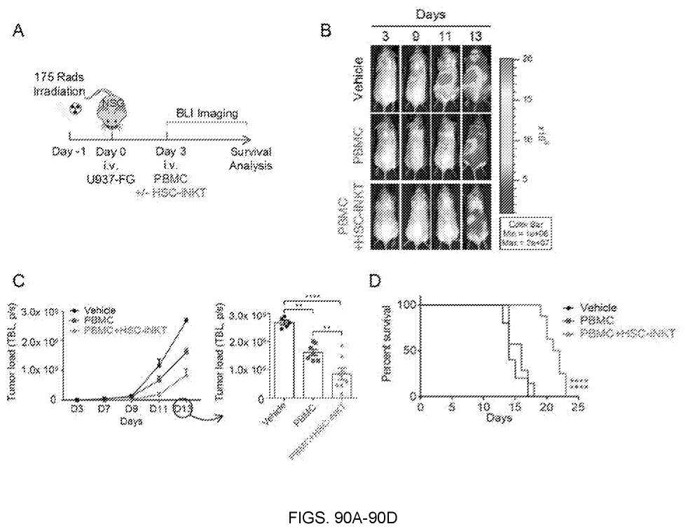

A human mouse xenograft model was used in in vivo studies to demonstrate the efficacy of ^{Allo}HSC-iNKT cells against AML. FIGS. 90A-90D show embodiments and results related to these studies.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A method of preparing a population of T cells comprising:
a) selecting stem or progenitor cells;
b) introducing one or more nucleic acids encoding at least one T-cell receptor (TCR); and
c) culturing the cells to induce the differentiation of the cells into T cells; wherein a), b), and/or c) exclude contacting the cells with a feeder cell or a population of feeder cells.

2-303. (canceled)

304. The method of claim 1, wherein:
c) comprises a culture that is feeder-free;
the stem or progenitor cells comprise CD34+ cells; and/or
cells of a) have been cultured in medium comprising one or more of IL-3, IL-7, IL-6, SCF, MCP-4, EPO, TPO, FLT3L, and/or retronectin.

305. The method of claim 1, wherein the TCR comprises an iNKT TCR.

306. The method of claim 1, wherein the TCR comprises a TCR that specifically recognizes the NY-ESO-1 antigen.

307. The method of claim 1, wherein c) comprises culturing the cells in a differentiation and/or expansion medium.

308. The method of claim 1, wherein c) comprises contacting the cells with one or more of DLL1, DLL4, VCAM1, VCAM5, and/or retronectin.

309. The method of claim 1, wherein the method further comprises stimulation and/or expansion of the cells.

310. The method of claim 1, wherein the method further comprises:
contacting the cells with one or more of human serum antibody, Glutamax, a buffer, an antimicrobial agent, and N-acetyl-L-cysteine; and/or wherein the expansion medium comprises one or more of human serum antibody, Glutamax, a buffer, an antimicrobial agent, and N-acetyl-L-cysteine; and/or
activation of the cells by contacting the cells with anti-CD3 and/or anti-CD28-coated beads.

311. The method of claim 1, wherein the method further comprises transferring a nucleic acid comprising a CAR molecule and/or HLA-E gene into the cells.

312. A cell or population of cells produced by the method of claim 1

313. An engineered invariant natural killer T (iNKT) cell that expresses at least one invariant natural killer (iNKT) T-cell receptor (TCR) and wherein the cell comprises one or more of:
high levels of NKG2D;
low or undetectable expression of KIR; and
high levels of Granzyme B.

314. The engineered cell(s) of claim 313, wherein at least one invariant TCR gene product is expressed from an exogenous nucleic acid.

315. The engineered cell(s) of claim 314, wherein the cells have not undergone cell sorting.

316. The engineered cell(s) of claim 314, wherein (1) the cell(s) comprise an exogenous suicide gene; or (2) the genome of the cell has been altered to eliminate surface expression of at least one HLA-I or HLA-II molecule, wherein the at least one TCR is expressed from an exogenous nucleic acid and/or from an endogenous invariant TCR gene that is under the transcriptional control of a recombinantly modified promoter region.

317. The engineered cell(s) of claim 314, wherein the cell(s) are derived from hematopoietic stem cells from a non-cancerous subject.

318. A method of treating a patient with T cells comprising administering to the patient the cell(s) of claim 314.

319. The method of claim 318, wherein the patient has cancer.

320. The method of claim 318, wherein the cancer comprises multiple myeloma.

321. The method of claim 319, wherein the cancer comprises leukemia.

322. The method of claim 318, wherein the patient has a disease or condition involving inflammation.


SEQUENCE LISTING (94 sequences)

SEQ ID NO: 1 - DNA, 75 bp, Unknown
  Description of Unknown iNKT TCR-alpha chain cloned sequence
gtgggcgatagaggttcagccttagggaggctgcattttggagctgggactcagctgattgtcatacctgacatc

SEQ ID NO: 2 - DNA, 75 bp, Unknown
  Description of Unknown iNKT TCR-beta chain cloned sequence
gccagcggtgatgctcggggggggggaaataccctctattttggaaaaggaagccggctcattgttgtagaggat

SEQ ID NO: 3 - DNA, 75 bp, Unknown
  Description of Unknown iNKT TCR-beta chain cloned sequence
gccagcggggggacagtccattctggaaatacgctctattttggagaaggaagccggctcattgttgtagaggat

SEQ ID NO: 4 - DNA, 75 bp, Unknown
  Description of Unknown iNKT TCR-beta chain cloned sequence
gccagcggtgatacgggacaaacaaacacagaagtcttctttggtaaaggaaccagactcacagttgtagaggat

SEQ ID NO: 5 - DNA, 72 bp, Unknown
  Description of Unknown iNKT TCR-beta chain cloned sequence
gccagcggtgaggggacagcaaacacagaagtcttctttggtaaaggaaccagactcacagttgtagaggat

SEQ ID NO: 6 - DNA, 69 bp, Unknown
  Description of Unknown iNKT TCR-beta chain cloned sequence
gccagcggtgaggcagggaacacagaagtcttctttggtaaaggaaccagactcacagttgtagaggat

SEQ ID NO: 7 - DNA, 78 bp, Unknown
  Description of Unknown iNKT TCR-alpha chain cloned sequence
gtgagcgacagaggctcaaccctggggaggctatactttggaagaggaactcagttgactgtctggcctgatatccag

SEQ ID NO: 8 - DNA, 75 bp, Unknown
  Description of Unknown iNKT TCR-beta chain cloned sequence
agcagtgacctccgaggacagaacacagatacgcagtattttggcccaggcacccggctgacagtgctcgaggac

SEQ ID NO: 9 - DNA, 78 bp, Unknown
  Description of Unknown iNKT TCR-beta chain cloned sequence
agcagtgaattaaaggaaacaggggttcaagagacccagtacttcgggccaggcacgcggctcctggtgctcgaggac

SEQ ID NO: 10 - DNA, 69 bp, Unknown
  Description of Unknown iNKT TCR-beta chain cloned sequence
agcagtgtatctcagggcggcactgaagctttctttggacaaggcaccagactcacagttgtagaggac

SEQ ID NO: 11 - DNA, 69 bp, Unknown; identical to SEQ ID NO: 10
  Description of Unknown iNKT TCR-beta chain cloned sequence

SEQ ID NO: 12 - DNA, 72 bp, Unknown
  Description of Unknown iNKT TCR-beta chain cloned sequence
agcagtgaccggacaggcgtgaacactgaagctttctttggacaaggcaccagactcacagttgtagaggac

SEQ ID NO: 13 - DNA, 72 bp, Unknown
  Description of Unknown iNKT TCR-beta chain cloned sequence
agcagtgaaccggacagggggggggctgaagctttctttggacaaggcaccagactcacagttgtagaggac

SEQ ID NO: 14 - DNA, 831 bp, Unknown
  Description of Unknown Human iNKT TCR-alpha chain cDNA sequence
atgaaaaagcatctgacgaccttcttggtgattttgtggctttatttttatagggggaatggcaaaaaccaagtggagcagagtcctcagtccctgatcatcctggagggaaagaactgcactcttcaatgcaattatacagtgagccccttcagcaacttaaggtggtataagcaagatactgggagaggtcctgtttccctgacaatcatgactttcagtgagaacacaaagtcgaacggaagatatacagcaactctggatgcagacacaaagcaaagctctctgcacatcacagcctcccagctcagcgattcagcctcctacatctgtgtggtgagcgacagaggctcaaccctggggaggctatactttggaagaggaactcagttgactgtctggcctgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 15 - DNA, 831 bp, Unknown
  Description of Unknown Human iNKT TCR-alpha chain cDNA codon-optimized sequence
atgaaaaagcatctgacaacattcctggtcattctgtggctgtacttctaccgaggcaacggcaaaaatcaggtggagcagtccccacagtccctgatcattctggaggggaagaactgcactctgcagtgtaattacaccgtgtctccctttagtaacctgcgctggtataaacaggacaccggacgaggacccgtgagcctgacaatcatgactttctcagagaacacaaagagcaatggacggtacaccgctacactggacgcagataccaaacagagctccctgcacatcacagcatctcagctgtcagatagcgcctcctacatttgcgtggtctctgaccgagggagtaccctgggccgactgtattttggaagggggacccagctgacagtgtggcccgacatccagaacccagatcccgccgtctaccagctgcgcgacagcaagtctagtgataaaagcgtgtgcctgttcacagactttgattctcagactaatgtctctcagagtaaggacagtgacgtgtacattactgacaaaaccgtcctggatatgaggagcatggacttcaagtcaaacagcgccgtggcttggtcaaacaagagcgacttcgcatgcgccaatgcttttaacaattcaatcattccagaggataccttctttcctagcccagaatcaagctgtgacgtgaagctggtcgagaaaagtttcgaaactgataccaacctgaattttcagaacctgtctgtgatcggcttcagaatcctgctgctgaaggtcgccggctttaatctgctgatgacactgagactgtggtcctcttga

SEQ ID NO: 16 - DNA, 336 bp, Unknown
  Description of Unknown Human iNKT TCR-beta chain cDNA (before D/J/N region) sequence
atgactatcaggctcctctgctacatgggcttttattttctgggggcaggcctcatggaagctgacatctaccagaccccaagataccttgttatagggacaggaaagaagatcactctggaatgttctcaaaccatgggccatgacaaaatgtactggtatcaacaagatccaggaatggaactacacctcatccactattcctatggagttaattccacagagaagggagatctttcctctgagtcaacagtctccagaataaggacggagcattttcccctgaccctggagtctgccaggccctcacatacctctcagtacctctgtgccagc

SEQ ID NO: 17 - DNA, 336 bp, Unknown
  Description of Unknown Human iNKT TCR-beta chain cDNA codon-optimized sequence
atgaccatccggctgctgtgctacatgggcttctattttctgggggcaggcctgatggaagccgacatctaccagactcccagatacctggtcatcggaaccgggaagaaaattacactggagtgttcccagacaatgggccacgataagatgtactggtatcagcaggaccctgggatggaactgcacctgatccattactcctatggcgtgaactctaccgagaagggcgacctgagcagcgaatccaccgtctctcgaattaggacagagcactttcctctgactctggaaagcgcccgaccaagtcatacatcacagtacctgtgcgctagc

SEQ ID NO: 18 - DNA, 60 bp, Unknown
  Description of Unknown Human iNKT TCR Beta Chain Diverse Region (D/J/N) sequence
gtagcggttgggccccaagagacccagtacttcgggccaggcacgcggctcctggtgctc

SEQ ID NO: 19 - DNA, 60 bp, Unknown
  Description of Unknown Human iNKT TCR Beta Chain Diverse Region (D/J/N) sequence
gtggcagtcggacctcaggagacccagtacttcggacccggcacccgcctgctggtgctg

SEQ ID NO: 20 - DNA, 54 bp, Unknown
  Description of Unknown Human iNKT TCR Beta Chain Diverse Region (D/J/N) sequence
agtgggccagggtacgagcagtacttcgggccgggcaccaggctcacggtcaca

SEQ ID NO: 21 - DNA, 54 bp, Unknown
  Description of Unknown Human iNKT TCR Beta Chain Diverse Region (D/J/N) sequence
tcaggacccggctacgagcagtatttcggccccggaactcggctgaccgtgacc

SEQ ID NO: 22 - DNA, 57 bp, Unknown
  Description of Unknown Human iNKT TCR Beta Chain Diverse Region (D/J/N) sequence
agtccccaattaaacactgaagctttctttggacaaggcaccagactcacagttgta

SEQ ID NO: 23 - DNA, 57 bp, Unknown
  Description of Unknown Human iNKT TCR Beta Chain Diverse Region (D/J/N) sequence
tctccacagctgaacaccgaggccttcttcgggcagggcacaaggcttaccgtggtg

SEQ ID NO: 24 - DNA, 78 bp, Unknown
  Description of Unknown Human iNKT TCR Beta Chain Diverse Region (D/J/N) sequence
agtgaattgcgggcgctcgggcccagctcctataattcacccctccactttgggaacgggaccaggctcactgtgaca

SEQ ID NO: 25 - DNA, 78 bp, Unknown
  Description of Unknown Human iNKT TCR Beta Chain Diverse Region (D/J/N) sequence
tccgaactccgagccctggggcctagctcctacaatagccccctgcactttggcaacggaaccaggctgacggtcacc

SEQ ID NO: 26 - DNA, 60 bp, Unknown
  Description of Unknown Human iNKT TCR Beta Chain Diverse Region (D/J/N) sequence
agtgaacaggggactactgcgggagctttctttggacaaggcaccagactcacagttgta

SEQ ID NO: 27 - DNA, 60 bp, Unknown
  Description of Unknown Human iNKT TCR Beta Chain Diverse Region (D/J/N) sequence
tccgaacagggaaccacagcaggagccttcttcggtcagggaacaagactgacagtcgtg

SEQ ID NO: 28 - DNA, 66 bp, Unknown
  Description of Unknown Human iNKT TCR Beta Chain Diverse Region (D/J/N) sequence
agtgagtcacgacatgcgacaggaaacaccatatattttggagagggaagttggctcactgttgta

SEQ ID NO: 29 - DNA, 66 bp, Unknown
  Description of Unknown Human iNKT TCR Beta Chain Diverse Region (D/J/N) sequence
agcgagagcaggcacgcaaccgggaacaccatatactttggcgagggctcctggctgactgtggtg

SEQ ID NO: 30 - DNA, 69 bp, Unknown
  Description of Unknown Human iNKT TCR Beta Chain Diverse Region (D/J/N) sequence
agtgtacccgggaacgacaggggcaatgaaaaactgttttttggcagtggaacccagctctctgtcttg

SEQ ID NO: 31 - DNA, 69 bp, Unknown
  Description of Unknown Human iNKT TCR Beta Chain Diverse Region (D/J/N) sequence
tccgtgcctggcaacgatagaggtaacgagaagctgtttttcggatccggcacacagctgtctgtcctg

SEQ ID NO: 32 - DNA, 72 bp, Unknown
  Description of Unknown Human iNKT TCR Beta Chain Diverse Region (D/J/N) sequence
agtgaaggggggggccttaagctagccaaaaacattcagtacttcggcgccgggacccggctctcagtgctg

SEQ ID NO: 33 - DNA, 72 bp, Unknown
  Description of Unknown Human iNKT TCR Beta Chain Diverse Region (D/J/N) sequence
agtgagggagggggactgaagctggctaagaatattcagtacttcggcgccggcactagactgtctgtgctg

SEQ ID NO: 34 - DNA, 69 bp, Unknown
  Description of Unknown Human iNKT TCR Beta Chain Diverse Region (D/J/N) sequence
agtgaattcgcctcttcggtacgtggaaacaccatatattttggagagggaagttggctcactgttgta

SEQ ID NO: 35 - DNA, 69 bp, Unknown
  Description of Unknown Human iNKT TCR Beta Chain Diverse Region (D/J/N) sequence
tctgagttcgcgagcagcgtccggggtaataccatttacttcggggaaggcagctggctgaccgtggtg

SEQ ID NO: 36 - DNA, 60 bp, Unknown
  Description of Unknown Human iNKT TCR Beta Chain Diverse Region (D/J/N) sequence
agtgcggcattaggccgggagacccagtacttcgggccaggcacgcggctcctggtgctc

SEQ ID NO: 37 - DNA, 60 bp, Unknown
  Description of Unknown Human iNKT TCR Beta Chain Diverse Region (D/J/N) sequence
tctgcagcccttggccgagagactcagtacttcggccctggcacaagactgctcgtgctc

SEQ ID NO: 38 - DNA, 63 bp, Unknown
  Description of Unknown Human iNKT TCR Beta Chain Diverse Region (D/J/N) sequence
agtgcctccgggggtgaatcctacgagcagtacttcgggccgggcaccaggctcacggtcaca

SEQ ID NO: 39 - DNA, 63 bp, Unknown
  Description of Unknown Human iNKT TCR Beta Chain Diverse Region (D/J/N) sequence
agcgcctccggaggagagtcatacgaacagtatttcggccctggcacacgcctcactgtgacc

SEQ ID NO: 40 - DNA, 90 bp, Unknown
  Description of Unknown Human iNKT TCR Beta Chain Diverse Region (D/J/N) sequence
agcggtcgggtctcggggggcgattccctcatagcgtttctaggccaagagacccagtacttcgggccaggcacgcggctcctggtgctc

SEQ ID NO: 41 - DNA, 90 bp, Unknown
  Description of Unknown Human iNKT TCR Beta Chain Diverse Region (D/J/N) sequence
tcaggacgagtgtccggaggggatagcctcatcgcatttctggggcaggaaactcagtacttcggacccggaacacgcctcctggtgctg

SEQ ID NO: 42 - DNA, 69 bp, Unknown; identical to SEQ ID NO: 30
  Description of Unknown Human iNKT TCR Beta Chain Diverse Region (D/J/N) sequence

SEQ ID NO: 43 - DNA, 69 bp, Unknown; identical to SEQ ID NO: 31
  Description of Unknown Human iNKT TCR Beta Chain Diverse Region (D/J/N) sequence

SEQ ID NO: 44 - DNA, 534 bp, Unknown
  Description of Unknown Human iNKT TCR-beta chain cDNA (after D/J/N region) sequence
gaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgccaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 45 - DNA, 534 bp, Unknown
  Description of Unknown Human iNKT TCR-beta chain cDNA codon-optimized (after D/J/N region) sequence
gaggacctgaataaggtgttcccccctgaggtggctgtctttgaaccaagtgaggcagaaatttcacatacacagaaagccaccctggtgtgcctggctaccggcttctttcccgatcacgtggagctgagctggtgggtcaacggcaaggaagtgcatagcggagtctccacagacccacagcccctgaaagagcagcctgctctgaatgattccagatactgcctgtctagtagactgcgggtgtctgccaccttctggcagaacccaaggaatcatttcagatgtcaggtgcagttttatggcctgagcgagaacgatgaatggactcaggacagggctaagccagtgacccagatcgtcagcgcagaggcctggggaagagcagactgcgggtttacaagcgtgagctatcagcagggcgtcctgagcgccacaatcctgtacgaaattctgctgggaaaggccactctgtatgctgtgctggtctccgctctggtgctgatggcaatggtcaagcggaaagatttctga

SEQ ID NO: 46 - protein, 276 aa, Unknown
  Description of Unknown Human iNKT TCR-alpha chain sequence
MKKHLTTFLVILWLYFYRGNGKNQVEQSPQSLIILEGKNCTLQCNYTVSPFSNLRWYKQDTGRGPVSLTIMTFSENTKSNGRYTATLDADTKQSSLHITASQLSDSASYICVVSDRGSTLGRLYFGRGTQLTVWPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 47 - protein, 112 aa, Unknown
  Description of Unknown Human iNKT TCR-beta chain sequence
MTIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCAS

SEQ ID NO: 48 - protein, 20 aa, Unknown
  Description of Unknown Human iNKT TCR Beta Chain Diverse Region (D/J/N) sequence
VAVGPQETQYFGPGTRLLVL

SEQ ID NO: 49 - protein, 18 aa, Unknown
  Description of Unknown Human iNKT TCR Beta Chain Diverse Region (D/J/N) sequence
SGPGYEQYFGPGTRLTVT

SEQ ID NO: 50 - protein, 19 aa, Unknown
  Description of Unknown Human iNKT TCR Beta Chain Diverse Region (D/J/N) sequence
SPQLNTEAFFGQGTRLTVV

SEQ ID NO: 51 - protein, 26 aa, Unknown
  Description of Unknown Human iNKT TCR Beta Chain Diverse Region (D/J/N) sequence
SELRALGPSSYNSPLHFGNGTRLTVT

SEQ ID NO: 52 - protein, 20 aa, Unknown
  Description of Unknown Human iNKT TCR Beta Chain Diverse Region (D/J/N) sequence
SEQGTTAGAFFGQGTRLTVV

SEQ ID NO: 53 - protein, 22 aa, Unknown
  Description of Unknown Human iNKT TCR Beta Chain Diverse Region (D/J/N) sequence
SESRHATGNTIYFGEGSWLTVV

SEQ ID NO: 54 - protein, 23 aa, Unknown
  Description of Unknown Human iNKT TCR Beta Chain Diverse Region (D/J/N) sequence
SVPGNDRGNEKLFFGSGTQLSVL

SEQ ID NO: 55 - protein, 24 aa, Unknown
  Description of Unknown Human iNKT TCR Beta Chain Diverse Region (D/J/N) sequence
SEGGGLKLAKNIQYFGAGTRLSVL

SEQ ID NO: 56 - protein, 23 aa, Unknown
  Description of Unknown Human iNKT TCR Beta Chain Diverse Region (D/J/N) sequence
SEFASSVRGNTIYFGEGSWLTVV

SEQ ID NO: 57 - protein, 20 aa, Unknown
  Description of Unknown Human iNKT TCR Beta Chain Diverse Region (D/J/N) sequence
SAALGRETQYFGPGTRLLVL

SEQ ID NO: 58 - protein, 21 aa, Unknown
  Description of Unknown Human iNKT TCR Beta Chain Diverse Region (D/J/N) sequence
SASGGESYEQYFGPGTRLTVT

SEQ ID NO: 59 - protein, 30 aa, Unknown
  Description of Unknown Human iNKT TCR Beta Chain Diverse Region (D/J/N) sequence
SGRVSGGDSLIAFLGQETQYFGPGTRLLVL

SEQ ID NO: 60 - protein, 23 aa, Unknown
  Description of Unknown Human iNKT TCR Beta Chain Diverse Region (D/J/N) sequence
SVPGNDRGNEKLFFGSGTQLSVL

SEQ ID NO: 61 - protein, 177 aa, Unknown
  Description of Unknown Human iNKT TCR-beta chain (after D/J/N region) sequence
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 62 - DNA, 2844 bp, Unknown
  Description of Unknown Beta-2 microglobin (B2M) sequence
agtggaggcgtcgcgctggcgggcattcctgaagctgacagcattcgggccgagatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtggggtaagtcttacattcttttgtaagctgctgaaagttgtgtatgagtagtcatatcataaagctgctttgatataaaaaaggtctatggccatactaccctgaatgagtcccatcccatctgatataaacaatctgcatattgggattgtcagggaatgttcttaaagatcagattagtggcacctgctgagatactgatgcacagcatggtttctgaaccagtagtttccctgcagttgagcagggagcagcagcagcacttgcacaaatacatatacactcttaacacttcttacctactggcttcctctagcttttgtggcagcttcaggtatatttagcactgaacgaacatctcaagaaggtataggcctttgtttgtaagtcctgctgtcctagcatcctataatcctggacttctccagtactttctggctggattggtatctgaggctagtaggaagggcttgttcctgctgggtagctctaaacaatgtattcatgggtaggaacagcagcctattctgccagccttatttctaaccattttagacatttgttagtacatggtattttaaaagtaaaacttaatgtcttccttttttttctccactgtctttttcatagatcgagacatgtaagcagcatcatggaggtaagtttttgaccttgagaaaatgtttttgtttcactgtcctgaggactatttatagacagctctaacatgataaccctcactatgtggagaacattgacagagtaacattttagcagggaaagaagaatcctacagggtcatgttcccttctcctgtggagtggcatgaagaaggtgtatggccccaggtatggccatattactgaccctctacagagagggcaaaggaactgccagtatggtattgcaggataaaggcaggtggttacccacattacctgcaaggctttgatctttcttctgccatttccacattggacatctctgctgaggagagaaaatgaaccactcttttcctttgtataatgttgttttattcttcagacagaagagaggagttatacagctctgcagacatcccattcctgtatggggactgtgtttgcctcttagaggttcccaggccactagaggagataaagggaaacagattgttataacttgatataatgatactataatagatgtaactacaaggagctccagaagcaagagagagggaggaacttggacttctctgcatctttagttggagtccaaaggcttttcaatgaaattctactgcccagggtacattgatgctgaaaccccattcaaatctcctgttatattctagaacagggaattgatttgggagagcatcaggaaggtggatgatctgcccagtcacactgttagtaaattgtagagccaggacctgaactctaatatagtcatgtgttacttaatgacggggacatgttctgagaaatgcttacacaaacctaggtgttgtagcctactacacgcataggctacatggtatagcctattgctcctagactacaaacctgtacagcctgttactgtactgaatactgtgggcagttgtaacacaatggtaagtatttgtgtatctaaacatagaagttgcagtaaaaatatgctattttaatcttatgagaccactgtcatatatacagtccatcattgaccaaaacatcatatcagcattttttcttctaagattttgggagcaccaaagggatacactaacaggatatactctttataatgggtttggagaactgtctgcagctacttcttttaaaaaggtgatctacacagtagaaattagacaagtttggtaatgagatctgcaatccaaataaaataaattcattgctaacctttttcttttcttttcaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttgaactcttcaatctcttgcactcaaagcttgttaagatagttaagcgtgcataagttaacttccaatttacatactctgcttagaatttgggggaaaatttagaaatataattgacaggattattggaaatttgttataatgaatgaaacattttgtcatataagattcatatttacttcttatacatttgataaagtaaggcatggttgtggttaatctggtttatttttgttccacaagttaaataaatcataaaacttga

SEQ ID NO: 63 - DNA, 4654 bp, Unknown
  Description of Unknown Human class II major histocompatibility complex transactivator (CIITA) sequence
ggttagtgatgaggctagtgatgaggctgtgtgcttctgagctgggcatccgaaggcatccttggggaagctgagggcacgaggaggggctgccagactccgggagctgctgcctggctgggattcctacacaatgcgttgcctggctccacgccctgctgggtcctacctgtcagagccccaaggcagctcacagtgtgccaccatggagttggggcccctagaaggtggctacctggagcttcttaacagcgatgctgaccccctgtgcctctaccacttctatgaccagatggacctggctggagaagaagagattgagctctactcagaacccgacacagacaccatcaactgcgaccagttcagcaggctgttgtgtgacatggaaggtgatgaagagaccagggaggcttatgccaatatcgcggaactggaccagtatgtcttccaggactcccagctggagggcctgagcaaggacattttcaagcacataggaccagatgaagtgatcggtgagagtatggagatgccagcagaagttgggcagaaaagtcagaaaagacccttcccagaggagcttccggcagacctgaagcactggaagccagctgagccccccactgtggtgactggcagtctcctagtgggaccagtgagcgactgctccaccctgccctgcctgccactgcctgcgctgttcaaccaggagccagcctccggccagatgcgcctggagaaaaccgaccagattcccatgcctttctccagttcctcgttgagctgcctgaatctccctgagggacccatccagtttgtccccaccatctccactctgccccatgggctctggcaaatctctgaggctggaacaggggtctccagtatattcatctaccatggtgaggtgccccaggccagccaagtaccccctcccagtggattcactgtccacggcctcccaacatctccagaccggccaggctccaccagccccttcgctccatcagccactgacctgcccagcatgcctgaacctgccctgacctcccgagcaaacatgacagagcacaagacgtcccccacccaatgcccggcagctggagaggtctccaacaagcttccaaaatggcctgagccggtggagcagttctaccgctcactgcaggacacgtatggtgccgagcccgcaggcccggatggcatcctagtggaggtggatctggtgcaggccaggctggagaggagcagcagcaagagcctggagcgggaactggccaccccggactgggcagaacggcagctggcccaaggaggcctggctgaggtgctgttggctgccaaggagcaccggcggccgcgtgagacacgagtgattgctgtgctgggcaaagctggtcagggcaagagctattgggctggggcagtgagccgggcctgggcttgtggccggcttccccagtacgactttgtcttctctgtcccctgccattgcttgaaccgtccgggggatgcctatggcctgcaggatctgctcttctccctgggcccacagccactcgtggcggccgatgaggttttcagccacatcttgaagagacctgaccgcgttctgctcatcctagacggcttcgaggagctggaagcgcaagatggcttcctgcacagcacgtgcggaccggcaccggcggagccctgctccctccgggggctgctggccggccttttccagaagaagctgctccgaggttgcaccctcctcctcacagcccggccccggggccgcctggtccagagcctgagcaaggccgacgccctatttgagctgtccggcttctccatggagcaggcccaggcatacgtgatgcgctactttgagagctcagggatgacagagcaccaagacagagccctgacgctcctccgggaccggccacttcttctcagtcacagccacagccctactttgtgccgggcagtgtgccagctctcagaggccctgctggagcttggggaggacgccaagctgccctccacgctcacgggactctatgtcggcctgctgggccgtgcagccctcgacagcccccccggggccctggcagagctggccaagctggcctgggagctgggccgcagacatcaaagtaccctacaggaggaccagttcccatccgcagacgtgaggacctgggcgatggccaaaggcttagtccaacacccaccgcgggccgcagagtccgagctggccttccccagcttcctcctgcaatgcttcctgggggccctgtggctggctctgagtggcgaaatcaaggacaaggagctcccgcagtacctagcattgaccccaaggaagaagaggccctatgacaactggctggagggcgtgccacgctttctggctgggctgatcttccagcctcccgcccgctgcctgggagccctactcgggccatcggcggctgcctcggtggacaggaagcagaaggtgcttgcgaggtacctgaagcggctgcagccggggacactgcgggcgcggcagctgctggagctgctgcactgcgcccacgaggccgaggaggctggaatttggcagcacgtggtacaggagctccccggccgcctctcttttctgggcacccgcctcacgcctcctgatgcacatgtactgggcaaggccttggaggcggcgggccaagacttctccctggacctccgcagcactggcatttgcccctctggattggggagcctcgtgggactcagctgtgtcacccgtttcagggctgccttgagcgacacggtggcgctgtgggagtccctgcagcagcatggggagaccaagctacttcaggcagcagaggagaagttcaccatcgagcctttcaaagccaagtccctgaaggatgtggaagacctgggaaagcttgtgcagactcagaggacgagaagttcctcggaagacacagctggggagctccctgctgttcgggacctaaagaaactggagtttgcgctgggccctgtctcaggcccccaggctttccccaaactggtgcggatcctcacggccttttcctccctgcagcatctggacctggatgcgctgagtgagaacaagatcggggacgagggtgtctcgcagctctcagccaccttcccccagctgaagtccttggaaaccctcaatctgtcccagaacaacatcactgacctgggtgcctacaaactcgccgaggccctgccttcgctcgctgcatccctgctcaggctaagcttgtacaataactgcatctgcgacgtgggagccgagagcttggctcgtgtgcttccggacatggtgtccctccgggtgatggacgtccagtacaacaagttcacggctgccggggcccagcagctcgctgccagccttcggaggtgtcctcatgtggagacgctggcgatgtggacgcccaccatcccattcagtgtccaggaacacctgcaacaacaggattcacggatcagcctgagatgatcccagctgtgctctggacaggcatgttctctgaggacactaaccacgctggaccttgaactgggtacttgtggacacagctcttctccaggctgtatcccatgagcctcagcatcctggcacccggcccctgctggttcagggttggcccctgcccggctgcggaatgaaccacatcttgctctgctgacagacacaggcccggctccaggctcctttagcgcccagttgggtggatgcctggtggcagctgcggtccacccaggagccccgaggccttctctgaaggacattgcggacagccacggccaggccagagggagtgacagaggcagccccattctgcctgcccaggcccctgccaccctggggagaaagtacttctttttttttatttttagacagagtctcactgttgcccaggctggcgtgcagtggtgcgatctgggttcactgcaacctccgcctcttgggttcaagcgattcttctgcttcagcctcccgagtagctgggactacaggcacccaccatcatgtctggctaatttttcatttttagtagagacagggttttgccatgttggccaggctggtctcaaactcttgacctcaggtgatccacccacctcagcctcccaaagtgctgggattacaagcgtgagccactgcaccgggccacagagaaagtacttctccaccctgctctccgaccagacaccttgacagggcacaccgggcactcagaagacactgatgggcaacccccagcctgctaattccccagattgcaacaggctgggcttcagtggcagctgcttttgtctatgggactcaatgcactgacattgttggccaaagccaaagctaggcctggccagatgcaccagcccttagcagggaaacagctaatgggacactaatggggcggtgagaggggaacagactggaagcacagcttcatttcctgtgtcttttttcactacattataaatgtctctttaatgtcacaggcaggtccagggtttgagttcataccctgttaccattttggggtacccactgctctggttatctaatatgtaacaagccaccccaaatcatagtggcttaaaacaacactcacattta

SEQ ID NO: 64 - DNA, 1508 bp, Unknown
  Description of Unknown Human T cell receptor alpha chain (TRAC) sequence
ttttgaaacccttcaaaggcagagacttgtccagcctaacctgcctgctgctcctagctcctgaggctcagggcccttggcttctgtccgctctgctcagggccctccagcgtggccactgctcagccatgctcctgctgctcgtcccagtgctcgaggtgatttttaccctgggaggaaccagagcccagtcggtgacccagcttggcagccacgtctctgtctctgaaggagccctggttctgctgaggtgcaactactcatcgtctgttccaccatatctcttctggtatgtgcaataccccaaccaaggactccagcttctcctgaagtacacatcagcggccaccctggttaaaggcatcaacggttttgaggctgaatttaagaagagtgaaacctccttccacctgacgaaaccctcagcccatatgagcgacgcggctgagtacttctgtgctgtgagtgatctcgaaccgaacagcagtgcttccaagataatctttggatcagggaccagactcagcatccggccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctgagatctgcaagattgtaagacagcctgtgctccctcgctccttcctctgcattgcccctcttctccctctccaaacagagggaactctcctacccccaaggaggtgaaagctgctaccacctctgtgcccccccggtaatgccaccaactggatcctacccgaatttatgattaagattgctgaagagctgccaaacactgctgccaccccctctgttcccttattgctgcttgtcactgcctgacattcacggcagaggcaaggctgctgcagcctcccctggctgtgcacattccctcctgctccccagagactgcctccgccatcccacagatgatggatcttcagtgggttctcttgggctctaggtcctggagaatgttgtgaggggtttatttttttttaatagtgttcataaagaaatacatagtattcttcttctcaagacgtggggggaaattatctcattatcgaggccctgctatgctgtgtgtctgggcgtgttgtatgtcctgctgccgatgccttcattaaaatgatttggaa

SEQ ID NO: 65 - DNA, 2040 bp, Unknown
  Description of Unknown Human T cell receptor beta chain (TRBC1) sequence
tgcatcctagggacagcatagaaaggaggggcaaagtggagagagagcaacagacactgggatggtgaccccaaaacaatgagggcctagaatgacatagttgtgcttcattacggcccattcccagggctctctctcacacacacagagcccctaccagaaccagacagctctcagagcaaccctggctccaacccctcttccctttccagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcaggtgagtggggcctggggagatgcctggaggagattaggtgagaccagctaccagggaaaatggaaagatccaggtagcagacaagactagatccaaaaagaaaggaaccagcgcacaccatgaaggagaattgggcacctgtggttcattcttctcccagattctcagcccaacagagccaagcagctgggtcccctttctatgtggcctgtgtaactctcatctgggtggtgccccccatccccctcagtgctgccacatgccatggattgcaaggacaatgtggctgacatctgcatggcagaagaaaggaggtgctgggctgtcagaggaagctggtctgggcctgggagtctgtgccaactgcaaatctgactttacttttaattgcctatgaaaataaggtctctcatttattttcctctccctgctttctttcagactgtggctttacctcgggtaagtaagcccttccttttcctctccctctctcatggttcttgacctagaaccaaggcatgaagaactcacagacactggagggtggagggtgggagagaccagagctacctgtgcacaggtacccacctgtccttcctccgtgccaacagtgtcctaccagcaaggggtcctgtctgccaccatcctctatgagatcctgctagggaaggccaccctgtatgctgtgctggtcagcgcccttgtgttgatggccatggtaagcaggagggcaggatggggccagcaggctggaggtgacacactgacaccaagcacccagaagtatagagtccctgccaggattggagctgggcagtagggagggaagagatttcattcaggtgcctcagaagataacttgcacctctgtaggatcacagtggaagggtcatgctgggaaggagaagctggagtcaccagaaaacccaatggatgttgtgatgagccttactatttgtgtggtcaatgggccctactactttctctcaatcctcacaactcctggctcttaataacccccaaaactttctcttctgcaggtcaagagaaaggatttctgaaggcagccctggaagtggagttaggagcttctaacccgtcatggtttcaatacacattcttcttttgccagcgcttctgaagagctgctctcacctctctgcatcccaatagatatccccctatgtgcatgcacacctgcacactcacggctgaaatctccctaacccagggggaccttagcatgcctaagtgactaaaccaataaaaatgttctggtctggcctgactctgacttgtgaatgtctggatagctccttggctgtctctgaactccctgtgactctccccattcagtcaggatagaaacaagaggtattcaaggaaaatgcagactcttcacgtaagagggatgaggggcccaccttgagatcaatagcag

SEQ ID NO: 66 - DNA, 2008 bp, Unknown
  Description of Unknown Human TRBC2 T cell receptor beta constant 2 (TCRB2) sequence
atggcgtagtccccaaagaacgaggacctagtaacataattgtgcttcattatggtcctttcccggccttctctctcacacatacacagagcccctaccaggaccagacagctctcagagcaaccctagccccattacctcttccctttccagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcaggtgagtggggcctggggagatgcctggaggagattaggtgagaccagctaccagggaaaatggaaagatccaggtagcggacaagactagatccagaagaaagccagagtggacaaggtgggatgatcaaggttcacagggtcagcaaagcacggtgtgcacttcccccaccaagaagcatagaggctgaatggagcacctcaagctcattcttccttcagatcctgacaccttagagctaagctttcaagtctccctgaggaccagccatacagctcagcatctgagtggtgtgcatcccattctcttctggggtcctggtttcctaagatcatagtgaccacttcgctggcactggagcagcatgagggagacagaaccagggctatcaaaggaggctgactttgtactatctgatatgcatgtgtttgtggcctgtgagtctgtgatgtaaggctcaatgtccttacaaagcagcattctctcatccatttttcttcccctgttttctttcagactgtggcttcacctccggtaagtgagtctctcctttttctctctatctttcgccgtctctgctctcgaaccagggcatggagaatccacggacacaggggcgtgagggaggccagagccacctgtgcacaggtacctacatgctctgttcttgtcaacagagtcttaccagcaaggggtcctgtctgccaccatcctctatgagatcttgctagggaaggccaccttgtatgccgtgctggtcagtgccctcgtgctgatggccatggtaaggaggagggtgggatagggcagatgatgggggcaggggatggaacatcacacatgggcataaaggaatctcagagccagagcacagcctaatatatcctatcacctcaatgaaaccataatgaagccagactggggagaaaatgcagggaatatcacagaatgcatcatgggaggatggagacaaccagcgagccctactcaaattaggcctcagagcccgcctcccctgccctactcctgctgtgccatagcccctgaaaccctgaaaatgttctctcttccacaggtcaagagaaaggattccagaggctagctccaaaaccatcccaggtcattcttcatcctcacccaggattctcctgtacctgctcccaatctgtgttcctaaaagtgattctcactctgcttctcatctcctacttacatgaatacttctctcttttttctgtttccctgaagattgagctcccaacccccaagtacgaaataggctaaaccaataaaaaattgtgtgttgggcctggttgcatttcaggagtgtctgtggagttctgctcatcactgacctatcttctgatttagggaaagcagcattcgcttggacatctgaagtgacagccctctttctctccacccaatgctgctttctcctgttcatcctgatggaagtctcaacaca

SEQ ID NO: 67 - DNA, 996 bp, Unknown
  Description of Unknown sr39TK cDNA sequence
atgcctacactgctgcgggtgtacatcgatggccctcacggcatgggcaagaccacaaccacacagctgctggtggccctgggcagcagggacgatatcgtgtacgtgccagagcccatgacatattggcgcgtgctgggagcatccgagacaatcgccaacatctacaccacacagcacagactggatcagggagagatctccgccggcgacgcagcagtggtcatgaccagcgcccagatcacaatgggcatgccatatgcagtgaccgacgccgtgctggcacctcacatcggaggagaggcaggctctagccacgcaccaccccctgccctgacaatctttctggatcggcaccctatcgccttcatgctgtgctacccagccgccagatatctgatgggcagcatgaccccacaggccgtgctggccttcgtggccctgatcccacccaccctgccaggaacaaatatcgtgctgggcgccctgccagaggacaggcacatcgatagactggccaagaggcagcgccccggagagcggctggacctggcaatgctggcagcaatcaggagagtgtacggcctgctggccaacaccgtgcggtatctgcagtgtggaggctcctggagagaggactggggacagctgtctggaacagcagtgcctccacagggagcagagccacagtccaatgcaggacctaggccacacatcggcgataccctgttcacactgtttcgcgcaccagagctgctggcacctaacggcgatctgtacaacgtgttcgcatgggcactggacgtgctggcaaagcggctgagatctatgcacgtgttcatcctggactacgaccagagcccagccggctgtagagatgccctgctgcagctgacaagcggcatggtgcagacccacgtgaccacacccggctctattccaacaatctgcgacctggctaggacctttgcaagagaaatgggcgaagctaactga

SEQ ID NO: 68 - protein, 331 aa, Unknown
  Description of Unknown sr39TK amino acid sequence
MPTLLRVYIDGPHGMGKTTTTQLLVALGSRDDIVYVPEPMTYWRVLGASETIANIYTTQHRLDQGEISAGDAAVVMTSAQITMGMPYAVTDAVLAPHIGGEAGSSHAPPPALTIFLDRHPIAFMLCYPAARYLMGSMTPQAVLAFVALIPPTLPGTNIVLGALPEDRHIDRLAKRQRPGERLDLAMLAAIRRVYGLLANTVRYLQCGGSWREDWGQLSGTAVPPQGAEPQSNAGPRPHIGDTLFTLFRAPELLAPNGDLYNVFAWALDVLAKRLRSMHVFILDYDQSPAGCRDALLQLTSGMVQTHVTTPGSIPTICDLARTFAREMGEAN

SEQ ID NO: 69 - RNA, 20 nt, Unknown
  Description of Unknown primer
gauauuggcauaagccuccc

SEQ ID NO: 70 - DNA, 1508 bp, Unknown; identical to SEQ ID NO: 64
  Description of Unknown Human T cell receptor alpha chain (TRAC) mRNA sequence

SEQ ID NO: 71 - DNA, 2625 bp, Unknown
  Description of Unknown BCMA CAR with truncated EGFR sequence
atggctctgcctgtgaccgccctgctgctgcctctggctctgctgctgcacgccgctcggcctgacatcgttttgacacaatctcctgcgtcattggccatgagtctcgggaagcgcgcaacaatatcctgtcgcgccagtgaatctgtgtctgtgataggagcgcacttgatccattggtatcagcagaaacctggacaacctcccaagctgctcatctacctcgccagtaaccttgaaacaggagtacctgctcggttttcaggttccgggtcagggacggatttcactttgactatcgacccagttgaggaagacgacgtagccatatatagctgcctgcagtctcggatcttcccgcgcacgttcgggggaggaactaagctggagattaagggcggcgggggttctggtggcggcggcagcggcggtggaggatcacaaatccaactggttcagtccggtccagaactgaaaaagccgggggagacggtgaaaatctcctgtaaggcctcaggttataccttcaccgattacagcatcaattgggtaaagcgggctccagggaaaggtctgaaatggatgggttggatcaacacagaaacccgagaaccagcctatgcttacgactttcgaggtcgattcgctttttccttggaaacttccgcaagcacagcctatctgcaaatcaacaatctcaagtacgaagatacggccacgtatttttgtgccctggattacagctatgcaatggattactggggtcaggggacgtctgttacagtttctagtactacaactccagcacccagaccccctacacctgctccaactatcgcaagtcagcccctgtcactgcgccctgaagcctgtcgccctgctgccgggggagctgtgcatactcggggactggactttgcctgtgatatctacatctgggcgcccttggccgggacttgtggggtccttctcctgtcactggttatcaccctttactgcaggttcagtgtcgtgaagagaggccggaagaagctgctgtacatcttcaagcagcctttcatgaggcccgtgcagactacccaggaggaagatggatgcagctgtagattccctgaagaggaggaaggaggctgtgagctgagagtgaagttctcccgaagcgcagatgccccagcctatcagcagggacagaatcagctgtacaacgagctgaacctgggaagacgggaggaatacgatgtgctggacaaaaggcggggcagagatcctgagatgggcggcaaaccaagacggaagaacccccaggaaggtctgtataatgagctgcagaaagacaagatggctgaggcctactcagaaatcgggatgaagggcgaaagaaggagaggaaaaggccacgacggactgtaccaggggctgagtacagcaacaaaagacacctatgacgctctgcacatgcaggctctgccaccaagacgagctaaacgaggctcaggcgcgacgaactttagtttgctgaagcaagctggggatgtagaggaaaatccgggtcccatgttgctccttgtgacgagcctcctgctctgcgagctgccccatccagccttcctcctcatcccgcggaaggtgtgcaatggcataggcattggcgagtttaaagattctctgagcataaatgctacgaatattaagcatttcaagaattgtacttctattagtggcgacctccatattcttccggttgccttcaggggtgactctttcacccacacacctccattggatccacaagaacttgacatcctgaagacggttaaagagattacaggcttcctccttatccaagcgtggcccgagaacagaacggacttgcacgcctttgagaacctcgaaataatacggggtcggacgaagcaacacggccaatttagccttgcggttgttagtctgaacattacttctctcggccttcgctctttgaaagaaatcagcgacggagatgtcatcattagtggaaacaagaacctgtgctacgcgaacacaatcaactggaagaagctcttcggtacttcaggccaaaagacaaagattattagtaacagaggagagaatagctgtaaggctaccggacaagtttgtcacgccttgtgtagtccagagggttgctggggaccggaaccaagggattgcgtcagttgccggaacgtgagtcgcggacgcgagtgtgtggataagtgcaatcttctggaaggggaaccgcgagagtttgtagaaaattccgaatgtatacagtgtcatcccgagtgtcttccacaagcaatgaatatcacatgtacagggaggggtcctgataactgtatccaatgtgcacactacatagatggtcctcactgtgtaaagacgtgccccgccggagtaatgggtgaaaacaacaccctcgtgtggaagtacgccgatgccgggcatgtctgtcatttgtgtcatcccaactgcacatatggctgtaccggtcctggattggagggctgtccaacaaacgggccgaaaataccgagtatcgcaacaggcatggtgggagcacttttgcttctcctcgttgtcgccctgggcatcggcttgttcatg

SEQ ID NO: 72 - protein, 875 aa, Unknown
  Description of Unknown BCMA CAR with truncated EGFR sequence
MALPVTALLLPLALLLHAARPDIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIKGGGGSGGGGSGGGGSQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPAAGGAVHTRGLDFACDIYIWAPLAGTCGVLLLSLVITLYCRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRGSGATNFSLLKQAGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 73 - DNA, 63 bp, Unknown
  Description of Unknown Leader sequence
atggctctgcctgtgaccgccctgctgctgcctctggctctgctgctgcacgccgctcggcct

SEQ ID NO: 74 - DNA, 729 bp, Unknown
  Description of Unknown BCMA scFv sequence
gacatcgttttgacacaatctcctgcgtcattggccatgagtctcgggaagcgcgcaacaatatcctgtcgcgccagtgaatctgtgtctgtgataggagcgcacttgatccattggtatcagcagaaacctggacaacctcccaagctgctcatctacctcgccagtaaccttgaaacaggagtacctgctcggttttcaggttccgggtcagggacggatttcactttgactatcgacccagttgaggaagacgacgtagccatatatagctgcctgcagtctcggatcttcccgcgcacgttcgggggaggaactaagctggagattaagggcggcgggggttctggtggcggcggcagcggcggtggaggatcacaaatccaactggttcagtccggtccagaactgaaaaagccgggggagacggtgaaaatctcctgtaaggcctcaggttataccttcaccgattacagcatcaattgggtaaagcgggctccagggaaaggtctgaaatggatgggttggatcaacacagaaacccgagaaccagcctatgcttacgactttcgaggtcgattcgctttttccttggaaacttccgcaagcacagcctatctgcaaatcaacaatctcaagtacgaagatacggccacgtatttttgtgccctggattacagctatgcaatggattactggggtcaggggacgtctgttacagtttctagt

SEQ ID NO: 75 - DNA, 141 bp, Unknown
  Description of Unknown CD8 hinge sequence
actacaactccagcacccagaccccctacacctgctccaactatcgcaagtcagcccctgtcactgcgccctgaagcctgtcgccctgctgccgggggagctgtgcatactcggggactggactttgcctgtgatatctac

SEQ ID NO: 76 - DNA, 66 bp, Unknown
  Description of Unknown CD8 transmembrane sequence
atctgggcgcccttggccgggacttgtggggtccttctcctgtcactggttatcaccctttactgc

SEQ ID NO: 77 - DNA, 144 bp, Unknown
  Description of Unknown 4-1BB costimulatory domain sequence
aggttcagtgtcgtgaagagaggccggaagaagctgctgtacatcttcaagcagcctttcatgaggcccgtgcagactacccaggaggaagatggatgcagctgtagattccctgaagaggaggaaggaggctgtgagctgaga

SEQ ID NO: 78 - DNA, 333 bp, Unknown
  Description of Unknown CD3 zeta intracellular signaling domain sequence
gtgaagttctcccgaagcgcagatgccccagcctatcagcagggacagaatcagctgtacaacgagctgaacctgggaagacgggaggaatacgatgtgctggacaaaaggcggggcagagatcctgagatgggcggcaaaccaagacggaagaacccccaggaaggtctgtataatgagctgcagaaagacaagatggctgaggcctactcagaaatcgggatgaagggcgaaagaaggagaggaaaaggccacgacggactgtaccaggggctgagtacagcaacaaaagacacctatgacgctctgcacatgcaggctctgccaccaaga

SEQ ID NO: 79 - DNA, 78 bp, Unknown
  Description of Unknown P2A peptide sequence
cgagctaaacgaggctcaggcgcgacgaactttagtttgctgaagcaagctggggatgtagaggaaaatccgggtccc

SEQ ID NO: 80 - DNA, 1071 bp, Unknown
  Description of Unknown Truncated EGFR sequence
atgttgctccttgtgacgagcctcctgctctgcgagctgccccatccagccttcctcctcatcccgcggaaggtgtgcaatggcataggcattggcgagtttaaagattctctgagcataaatgctacgaatattaagcatttcaagaattgtacttctattagtggcgacctccatattcttccggttgccttcaggggtgactctttcacccacacacctccattggatccacaagaacttgacatcctgaagacggttaaagagattacaggcttcctccttatccaagcgtggcccgagaacagaacggacttgcacgcctttgagaacctcgaaataatacggggtcggacgaagcaacacggccaatttagccttgcggttgttagtctgaacattacttctctcggccttcgctctttgaaagaaatcagcgacggagatgtcatcattagtggaaacaagaacctgtgctacgcgaacacaatcaactggaagaagctcttcggtacttcaggccaaaagacaaagattattagtaacagaggagagaatagctgtaaggctaccggacaagtttgtcacgccttgtgtagtccagagggttgctggggaccggaaccaagggattgcgtcagttgccggaacgtgagtcgcggacgcgagtgtgtggataagtgcaatcttctggaaggggaaccgcgagagtttgtagaaaattccgaatgtatacagtgtcatcccgagtgtcttccacaagcaatgaatatcacatgtacagggaggggtcctgataactgtatccaatgtgcacactacatagatggtcctcactgtgtaaagacgtgccccgccggagtaatgggtgaaaacaacaccctcgtgtggaagtacgccgatgccgggcatgtctgtcatttgtgtcatcccaactgcacatatggctgtaccggtcctggattggagggctgtccaacaaacgggccgaaaataccgagtatcgcaacaggcatggtgggagcacttttgcttctcctcgttgtcgccctgggcatcggcttgttcatg

SEQ ID NO: 81 - protein, 21 aa, Unknown
  Description of Unknown Leader sequence
MALPVTALLLPLALLLHAARP

SEQ ID NO: 82 - protein, 243 aa, Unknown
  Description of Unknown BCMA scFv sequence
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIKGGGGSGGGGSGGGGSQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 83 - protein, 47 aa, Unknown
  Description of Unknown CD8 hinge sequence
TTTPAPRPPTPAPTIASQPLSLRPEACRPAAGGAVHTRGLDFACDIY

SEQ ID NO: 84 - protein, 22 aa, Unknown
  Description of Unknown CD8 transmembrane sequence
IWAPLAGTCGVLLLSLVITLYC

SEQ ID NO: 85 - protein, 48 aa, Unknown
  Description of Unknown 4-1BB costimulatory domain sequence
RFSVVKRGRKKLLYIFKQPFMRPVQTTQEEDGCSCRFPEEEEGGCELR

SEQ ID NO: 86 - protein, 111 aa, Unknown
  Description of Unknown CD3 zeta intracellular signaling domain sequence
VKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 87 - protein, 26 aa, Unknown
  Description of Unknown P2A peptide sequence
RAKRGSGATNFSLLKQAGDVEENPGP

SEQ ID NO: 88 - protein, 357 aa, Unknown
  Description of Unknown Truncated EGFR sequence
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 89 - DNA, 32 bp, Unknown
  Description of Unknown iNKT TCR-apha chain forward primer
gggagatactcagcaactctggataaagatgc

SEQ ID NO: 90 - DNA, 27 bp, Unknown
  Description of Unknown iNKT TCR-apha chain reverse primer
ccagattccatggttttcggcacattg

SEQ ID NO: 91 - DNA, 30 bp, Unknown
  Description of Unknown iNKT TCR-beta chain forward primer
ggagatatccctgatggatacaaggcctcc

SEQ ID NO: 92 - DNA, 30 bp, Unknown
  Description of Unknown iNKT TCR-beta chain reverse primer
gggtagccttttgtttgtttgcaatctctg

SEQ ID NO: 93 - RNA, 20 nt, Unknown
  Description of Unknown primer
cgcgagcacagcuaaggcca

SEQ ID NO: 94 - protein, 9 aa, Unknown
  Description of Unknown NY-ESO-1(157-165) sequence
SLLMWITQC